(19) United States
(12) Patent Application Publication
(10) Pub. No.: US 2025/0368742 A1
(43) Pub. Date: Dec. 4, 2025

(54) ANTI-MICA/B ANTIBODIES AND USES THEREOF

(71) Applicant: D2M Biotherapeutics Limited, Grand Cayman (KY)

(72) Inventors: Dong Zhang, Natick, MA (US); Guangan Hu, Natick, MA (US); Nan Bing, Natick, MA (US); Quanju Zhao, Natick, MA (US); Lai Shi, Natick, MA (US); Xiaodong Jiang, Natick, MA (US)

(21) Appl. No.: 18/853,769

(22) PCT Filed: Apr. 7, 2023
(86) PCT No.: PCT/US2023/017891

§ 371 Date: Oct. 3, 2024

Related U.S. Application Data
(60) Provisional application No. 63/329,221, filed on Apr. 8, 2022.

(51) Int. Cl. C07K 16/28; A61K 39/00; A61P 35/00

(52) U.S. Cl. CPC C07K 16/2833; A61P 35/00; A61K 2039/507; C07K 2317/24; C07K 2317/31; C07K 2317/92; C07K 2317/94

(57) ABSTRACT

This disclosure relates to anti-MICA (major histocompatibility complex class I chain related A) and/or anti-MICB (major histocompatibility complex class I chain related B) antibodies, antigen-binding fragments, and the uses thereof.

### CLAIM OF PRIORITY

This application claims the benefit of U.S. Provisional Application App. No. 63/329,221, filed on Apr. 8, 2022.

### TECHNICAL FIELD

This disclosure relates to antibodies that can bind to major histocompatibility complex class I chain related A (MICA) and antibodies that can bind to major histocompatibility complex class I chain related B (MICA), together referred to as anti-MICA/B antibodies, and the uses thereof.

### BACKGROUND

Major histocompatibility complex class I chain related A and B (MICA/B) are highly polymorphic cell surface proteins related to MHC class I glycoproteins and are ligands to stimulate an activating receptor, NKG2D, expressed on NK cells, CD8+ T cells, and gamma delta (GD) T cells. The engagement of the NKG2D receptor with MICA/B proteins triggers NK cells and co-stimulates T cells, resulting in elimination of cancer cells or damaged cells by effector cells expressing the NKG2D receptor. (Bauer S, et al. Activation of NK cells and T cells by NKG2D, a receptor for stress-inducible MICA. Science. 1999 Jul. 30; 285(5428):727-9; Diefenbach A, et al. Ligands for the murine NKG2D receptor: expression by tumor cells and activation of NK cells and macrophages. Nat Immunol. 2000 August; 1(2):119-26; and Groh V, et al. Costimulation of CD8alphabeta T cells by NKG2D via engagement by MIC induced on virus-infected cells. Nat Immunol. 2001 March; 2(3):255-60.)

MICA/B proteins are constitutively expressed at low levels on myeloid cells, epithelial cells, endothelial cells, and fibroblasts. MICA/B proteins are upregulated or expressed de novo in response to stress, e.g., during carcinogenesis, infections, during the DNA damage response, and in various autoimmune conditions. However, as an escape mechanism in order to prevent the response mediated by NKG2D, tumor cells proteolytically shed MICA/B proteins from the cell surface resulting both in reduction of MICA/B surface density and in generation of soluble MICA/B (sMICA/B). (Groh V, et al. Tumour-derived soluble MIC ligands impair expression of NKG2D and T-cell activation. Nature. 2002 Oct. 17; 419(6908):734-8; and Salih H R, et al. Cutting edge: down-regulation of MICA on human tumors by proteolytic shedding. J Immunol. 2002 Oct. 15; 169(8):4098-102.) Expression of MICA/B has been reported in a wide variety of tumor types, with high expression associated with poor prognosis in patients. (Spear P, et al. NKG2D ligands as therapeutic targets. Cancer Immun. 2013 May 1; 13:8; and Ghadially H, et al. MHC class I chain-related protein A and B (MICA and MICB) are predominantly expressed intracellularly in tumour and normal tissue. Br J Cancer. 2017 Apr. 25; 116(9):1208-1217.) Genome-wide association studies (GWAS) have revealed MICA/B-related signal highly correlated with incidence and poor prognosis of multiple human cancers. It is believed that specifically blocking the shedding of MICA and MICB proteins from cancer cells may restore or enhance NKG2D-dependent activation of NK and T cells in the tumor microenvironment, and therefore, may enhance anti-tumor activity in cancer patients. There is therefore a need to generate anti-MICA/B antibodies that bind and block MICA and MICB shedding and potentiate NK and T cells. The compositions and methods disclosed herein meet that need.

### SUMMARY

This disclosure relates to anti-MICA/B antibodies, antigen-binding fragment thereof, and the uses thereof.

In one aspect, the disclosure is related to an antibody or antigen-binding fragment thereof that binds to MICA (major histocompatibility complex class I chain related A) and/or MICB (major histocompatibility complex class I chain related B), comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are selected from VH CDRS 1, 2, 3 and VL CDRS 1, 2, 3 listed in FIGS. 24A, 24B, 24C, 24D, 24E, 28, 29, 30A, 30B, 30C, 30D, and 30E.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 3, 4, 5, respectively, and the selected VL CDRs 1, 2, 3 amino acid sequences are set forth in SEQ ID NOs: 6, 7, 8, respectively;
(2) the selected VH CDRs 1, 2, 3 amino acid sequences are set forth in SEQ ID NOs: 11, 12, 13, respectively, and the selected VL CDRs 1, 2, 3 amino acid sequences are set forth in SEQ ID NOs: 14, 15, 16, respectively;
(3) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(4) the selected VH CDRs 1, 2, 3 amino acid sequences are set forth in SEQ ID NOs: 27, 28, 29, respectively, and the selected VL CDRs 1, 2, 3 amino acid sequences are set forth in SEQ ID NOs: 30, 31, 32, respectively;
(5) the selected VH CDRs 1, 2, 3 amino acid sequences are set forth in SEQ ID NOs: 35, 36, 37, respectively, and the selected VL CDRs 1, 2, 3 amino acid sequences are set forth in SEQ ID NOs: 38, 39, 40, respectively;
(6) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively;
(7) the selected VH CDRs 1, 2, 3 amino acid sequences are set forth in SEQ ID NOs: 51, 52, 53, respectively, and the selected VL CDRs 1, 2, 3 amino acid sequences are set forth in SEQ ID NOs: 54, 55, 56, respectively;
(8) the selected VH CDRs 1, 2, 3 amino acid sequences are set forth in SEQ ID NOs: 59, 60, 61, respectively, and the selected VL CDRs 1, 2, 3 amino acid sequences are set forth in SEQ ID NOs: 62, 63, 64, respectively;
(9) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(10) the selected VH CDRs 1, 2, 3 amino acid sequences are set forth in SEQ ID NOs: 75, 76, 77, respectively, and the selected VL CDRs 1, 2, 3 amino acid sequences are set forth in SEQ ID NOs: 78, 79, 80, respectively;
(11) the selected VH CDRs 1, 2, 3 amino acid sequences are set forth in SEQ ID NOs: 83, 84, 85, respectively, and the selected VL CDRs 1, 2, 3 amino acid sequences are set forth in SEQ ID NOs: 86, 87, 88, respectively;
(12) the selected VH CDRs 1, 2, 3 amino acid sequences are set forth in SEQ ID NOs: 91, 92, 93, respectively, and the selected VL CDRs 1, 2, 3 amino acid sequences are set forth in SEQ ID NOs: 94, 95, 96, respectively;
(13) the selected VH CDRs 1, 2, 3 amino acid sequences are set forth in SEQ ID NOs: 99, 100, 101, respectively, and the selected VL CDRs 1, 2, 3 amino acid sequences are set forth in SEQ ID NOs: 102, 103, 104, respectively;
(14) the selected VH CDRs 1, 2, 3 amino acid sequences are set forth in SEQ ID NOs: 107, 108, 109, respectively, and the selected VL CDRs 1, 2, 3 amino acid sequences are set forth in SEQ ID NOs: 110, 111, 112, respectively;
(15) the selected VH CDRs 1, 2, 3 amino acid sequences are set forth in SEQ ID NOs: 115, 116, 117, respectively, and the selected VL CDRs 1, 2, 3 amino acid sequences are set forth in SEQ ID NOs: 118, 119, 120, respectively;
(16) the selected VH CDRs 1, 2, 3 amino acid sequences are set forth in SEQ ID NOs: 123, 124, 125, respectively, and the selected VL CDRs 1, 2, 3 amino acid sequences are set forth in SEQ ID NOs: 126, 127, 128, respectively;
(17) the selected VH CDRs 1, 2, 3 amino acid sequences are set forth in SEQ ID NOs: 131, 132, 133, respectively, and the selected VL CDRs 1, 2, 3 amino acid sequences are set forth in SEQ ID NOs: 134, 135, 136, respectively;
(18) the selected VH CDRs 1, 2, 3 amino acid sequences are set forth in SEQ ID NOs: 139, 140, 141, respectively, and the selected VL CDRs 1, 2, 3 amino acid sequences are set forth in SEQ ID NOs: 142, 143, 144, respectively;
(19) the selected VH CDRs 1, 2, 3 amino acid sequences are set forth in SEQ ID NOs: 147, 148, 149, respectively, and the selected VL CDRs 1, 2, 3 amino acid sequences are set forth in SEQ ID NOs: 150, 151, 152, respectively;
(20) the selected VH CDRs 1, 2, 3 amino acid sequences are set forth in SEQ ID NOs: 155, 156, 157, respectively, and the selected VL CDRs 1, 2, 3 amino acid sequences are set forth in SEQ ID NOs: 158, 159, 160, respectively;
(21) the selected VH CDRs 1, 2, 3 amino acid sequences are set forth in SEQ ID NOs: 815, 816, 817, respectively, and the selected VL CDRs 1, 2, 3 amino acid sequences are set forth in SEQ ID NOs: 818, 819, 820, respectively;
(22) the selected VH CDRs 1, 2, 3 amino acid sequences are set forth in SEQ ID NOs: 821, 822, 823, respectively, and the selected VL CDRs 1, 2, 3 amino acid sequences are set forth in SEQ ID NOs: 824, 825, 826, respectively;
(23) the selected VH CDRs 1, 2, 3 amino acid sequences are set forth in SEQ ID NOs: 827, 828, 829, respectively, and the selected VL CDRs 1, 2, 3 amino acid sequences are set forth in SEQ ID NOs: 830, 831, 832, respectively;
(24) the selected VH CDRs 1, 2, 3 amino acid sequences are set forth in SEQ ID NOs: 833, 834, 835, respectively, and the selected VL CDRs 1, 2, 3 amino acid sequences are set forth in SEQ ID NOs: 836, 837, 838, respectively;
(25) the selected VH CDRs 1, 2, 3 amino acid sequences are set forth in SEQ ID NOs: 839, 840, 841, respectively, and the selected VL CDRs 1, 2, 3 amino acid sequences are set forth in SEQ ID NOs: 842, 843, 844, respectively;
(26) the selected VH CDRs 1, 2, 3 amino acid sequences are set forth in SEQ ID NOs: 845, 846, 847, respectively, and the selected VL CDRs 1, 2, 3 amino acid sequences are set forth in SEQ ID NOs: 848, 849, 850, respectively;
(27) the selected VH CDRs 1, 2, 3 amino acid sequences are set forth in SEQ ID NOs: 851, 852, 853, respectively, and the selected VL CDRs 1, 2, 3 amino acid sequences are set forth in SEQ ID NOs: 854, 855, 856, respectively;
(28) the selected VH CDRs 1, 2, 3 amino acid sequences are set forth in SEQ ID NOs: 857, 858, 859, respectively, and the selected VL CDRs 1, 2, 3 amino acid sequences are set forth in SEQ ID NOs: 860, 861, 862, respectively;
(29) the selected VH CDRs 1, 2, 3 amino acid sequences are set forth in SEQ ID NOs: 863, 864, 865, respectively, and the selected VL CDRs 1, 2, 3 amino acid sequences are set forth in SEQ ID NOs: 866, 867, 868, respectively;
(30) the selected VH CDRs 1, 2, 3 amino acid sequences are set forth in SEQ ID NOs: 869, 870, 871, respectively, and the selected VL CDRs 1, 2, 3 amino acid sequences are set forth in SEQ ID NOs: 872, 873, 874, respectively;
(31) the selected VH CDRs 1, 2, 3 amino acid sequences are set forth in SEQ ID NOs: 875, 876, 877, respectively, and the selected VL CDRs 1, 2, 3 amino acid sequences are set forth in SEQ ID NOs: 878, 879, 880, respectively;
(32) the selected VH CDRs 1, 2, 3 amino acid sequences are set forth in SEQ ID NOs: 881, 882, 883, respectively, and the selected VL CDRs 1, 2, 3 amino acid sequences are set forth in SEQ ID NOs: 884, 885, 886, respectively;
(33) the selected VH CDRs 1, 2, 3 amino acid sequences are set forth in SEQ ID NOs: 887, 888, 889, respectively, and the selected VL CDRs 1, 2, 3 amino acid sequences are set forth in SEQ ID NOs: 890, 891, 892, respectively;
(34) the selected VH CDRs 1, 2, 3 amino acid sequences are set forth in SEQ ID NOs: 893, 894, 895, respectively, and the selected VL CDRs 1, 2, 3 amino acid sequences are set forth in SEQ ID NOs: 896, 897, 898, respectively;
(35) the selected VH CDRs 1, 2, 3 amino acid sequences are set forth in SEQ ID NOs: 899, 900, 901, respectively, and the selected VL CDRs 1, 2, 3 amino acid sequences are set forth in SEQ ID NOs: 902, 903, 904, respectively;
(36) the selected VH CDRs 1, 2, 3 amino acid sequences are set forth in SEQ ID NOs: 905, 906, 907, respectively, and the selected VL CDRs 1, 2, 3 amino acid sequences are set forth in SEQ ID NOs: 908, 909, 910, respectively;
(37) the selected VH CDRs 1, 2, 3 amino acid sequences are set forth in SEQ ID NOs: 911, 912, 913, respectively, and the selected VL CDRs 1, 2, 3 amino acid sequences are set forth in SEQ ID NOs: 914, 915, 916, respectively;
(38) the selected VH CDRs 1, 2, 3 amino acid sequences are set forth in SEQ ID NOs: 917, 918, 919, respectively, and the selected VL CDRs 1, 2, 3 amino acid sequences are set forth in SEQ ID NOs: 920, 921, 922, respectively;
(39) the selected VH CDRs 1, 2, 3 amino acid sequences are set forth in SEQ ID NOs: 923, 924 925, respectively, and the selected VL CDRs 1, 2, 3 amino acid sequences are set forth in SEQ ID NOs: 926, 927, 928, respectively; and
(40) the selected VH CDRs 1, 2, 3 amino acid sequences are set forth in SEQ ID NOs: 929, 930 931, respectively, and the selected VL CDRs 1, 2, 3 amino acid sequences are set forth in SEQ ID NOs: 932, 933, 934, respectively.

In some embodiments, CDR is determined by IMGT definition.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 161, 162, 163, respectively, and the selected VL CDRs 1, 2, 3 amino acid sequences are set forth in SEQ ID NOs: 164, 165, 166, respectively;
(2) the selected VH CDRs 1, 2, 3 amino acid sequences are set forth in SEQ ID NOs: 167, 168, 169, respectively, and the selected VL CDRs 1, 2, 3 amino acid sequences are set forth in SEQ ID NOs: 170, 171, 172, respectively;
(3) the selected VH CDRs 1, 2, 3 amino acid sequences are set forth in SEQ ID NOs: 173, 174, 175, respectively, and the selected VL CDRs 1, 2, 3 amino acid sequences are set forth in SEQ ID NOs: 176, 177, 178, respectively;
(4) the selected VH CDRs 1, 2, 3 amino acid sequences are set forth in SEQ ID NOs: 179, 180, 181, respectively, and the selected VL CDRs 1, 2, 3 amino acid sequences are set forth in SEQ ID NOs: 182, 183, 184, respectively;
(5) the selected VH CDRs 1, 2, 3 amino acid sequences are set forth in SEQ ID NOs: 185, 186, 187, respectively, and the selected VL CDRs 1, 2, 3 amino acid sequences are set forth in SEQ ID NOs: 188, 189, 190, respectively;
(6) the selected VH CDRs 1, 2, 3 amino acid sequences are set forth in SEQ ID NOs: 191, 192, 193, respectively, and the selected VL CDRs 1, 2, 3 amino acid sequences are set forth in SEQ ID NOs: 194, 195, 196, respectively;
(7) the selected VH CDRs 1, 2, 3 amino acid sequences are set forth in SEQ ID NOs: 197, 198, 199, respectively, and the selected VL CDRs 1, 2, 3 amino acid sequences are set forth in SEQ ID NOs: 200, 201, 202, respectively;
(8) the selected VH CDRs 1, 2, 3 amino acid sequences are set forth in SEQ ID NOs: 203, 204, 205, respectively, and the selected VL CDRs 1, 2, 3 amino acid sequences are set forth in SEQ ID NOs: 206, 207, 208, respectively;
(9) the selected VH CDRs 1, 2, 3 amino acid sequences are set forth in SEQ ID NOs: 209, 210, 211, respectively, and the selected VL CDRs 1, 2, 3 amino acid sequences are set forth in SEQ ID NOs: 212, 213, 214, respectively;
(10) the selected VH CDRs 1, 2, 3 amino acid sequences are set forth in SEQ ID NOs: 215, 216, 217, respectively, and the selected VL CDRs 1, 2, 3 amino acid sequences are set forth in SEQ ID NOs: 218, 219, 220, respectively;
(11) the selected VH CDRs 1, 2, 3 amino acid sequences are set forth in SEQ ID NOs: 221, 222, 223, respectively, and the selected VL CDRs 1, 2, 3 amino acid sequences are set forth in SEQ ID NOs: 224, 225, 226, respectively;
(12) the selected VH CDRs 1, 2, 3 amino acid sequences are set forth in SEQ ID NOs: 227, 228, 229, respectively, and the selected VL CDRs 1, 2, 3 amino acid sequences are set forth in SEQ ID NOs: 230, 231, 232, respectively;
(13) the selected VH CDRs 1, 2, 3 amino acid sequences are set forth in SEQ ID NOs: 233, 234, 235, respectively, and the selected VL CDRs 1, 2, 3 amino acid sequences are set forth in SEQ ID NOs: 236, 237, 238, respectively;
(14) the selected VH CDRs 1, 2, 3 amino acid sequences are set forth in SEQ ID NOs: 239, 240, 241, respectively, and the selected VL CDRs 1, 2, 3 amino acid sequences are set forth in SEQ ID NOs: 242, 243, 244, respectively;
(15) the selected VH CDRs 1, 2, 3 amino acid sequences are set forth in SEQ ID NOs: 245, 246, 247, respectively, and the selected VL CDRs 1, 2, 3 amino acid sequences are set forth in SEQ ID NOs: 248, 249, 250, respectively;
(16) the selected VH CDRs 1, 2, 3 amino acid sequences are set forth in SEQ ID NOs: 251, 252, 253, respectively, and the selected VL CDRs 1, 2, 3 amino acid sequences are set forth in SEQ ID NOs: 254, 255, 256, respectively;
(17) the selected VH CDRs 1, 2, 3 amino acid sequences are set forth in SEQ ID NOs: 257, 258, 259, respectively, and the selected VL CDRs 1, 2, 3 amino acid sequences are set forth in SEQ ID NOs: 260, 261, 262, respectively;
(18) the selected VH CDRs 1, 2, 3 amino acid sequences are set forth in SEQ ID NOs: 263, 264, 265, respectively, and the selected VL CDRs 1, 2, 3 amino acid sequences are set forth in SEQ ID NOs: 266, 267, 268, respectively;
(19) the selected VH CDRs 1, 2, 3 amino acid sequences are set forth in SEQ ID NOs: 269, 270, 271, respectively, and the selected VL CDRs 1, 2, 3 amino acid sequences are set forth in SEQ ID NOs: 272, 273, 274, respectively;
(20) the selected VH CDRs 1, 2, 3 amino acid sequences are set forth in SEQ ID NOs: 275, 276, 277, respectively, and the selected VL CDRs 1, 2, 3 amino acid sequences are set forth in SEQ ID NOs: 278, 279, 280, respectively;
(21) the selected VH CDRs 1, 2, 3 amino acid sequences are set forth in SEQ ID NOs: 935, 936, 937, respectively, and the selected VL CDRs 1, 2, 3 amino acid sequences are set forth in SEQ ID NOs: 938, 939, 940, respectively;
(22) the selected VH CDRs 1, 2, 3 amino acid sequences are set forth in SEQ ID NOs: 941, 942, 943, respectively, and the selected VL CDRs 1, 2, 3 amino acid sequences are set forth in SEQ ID NOs: 944, 945, 946, respectively;
(23) the selected VH CDRs 1, 2, 3 amino acid sequences are set forth in SEQ ID NOs: 947, 948, 949, respectively, and the selected VL CDRs 1, 2, 3 amino acid sequences are set forth in SEQ ID NOs: 950, 951, 952, respectively;
(24) the selected VH CDRs 1, 2, 3 amino acid sequences are set forth in SEQ ID NOs: 953, 954, 955, respectively, and the selected VL CDRs 1, 2, 3 amino acid sequences are set forth in SEQ ID NOs: 956, 957, 958, respectively;
(25) the selected VH CDRs 1, 2, 3 amino acid sequences are set forth in SEQ ID NOs: 959, 960, 961, respectively, and the selected VL CDRs 1, 2, 3 amino acid sequences are set forth in SEQ ID NOs: 962, 963, 964, respectively;
(26) the selected VH CDRs 1, 2, 3 amino acid sequences are set forth in SEQ ID NOs: 965, 966, 967, respectively, and the selected VL CDRs 1, 2, 3 amino acid sequences are set forth in SEQ ID NOs: 968, 969, 970, respectively;
(27) the selected VH CDRs 1, 2, 3 amino acid sequences are set forth in SEQ ID NOs: 971, 972, 973, respectively, and the selected VL CDRs 1, 2, 3 amino acid sequences are set forth in SEQ ID NOs: 974, 975, 976, respectively;
(28) the selected VH CDRs 1, 2, 3 amino acid sequences are set forth in SEQ ID NOs: 977, 978, 979, respectively, and the selected VL CDRs 1, 2, 3 amino acid sequences are set forth in SEQ ID NOs: 980, 981, 982, respectively;
(29) the selected VH CDRs 1, 2, 3 amino acid sequences are set forth in SEQ ID NOs: 983, 984, 985, respectively, and the selected VL CDRs 1, 2, 3 amino acid sequences are set forth in SEQ ID NOs: 986, 987, 988, respectively;
(30) the selected VH CDRs 1, 2, 3 amino acid sequences are set forth in SEQ ID NOs: 989, 990, 991, respectively, and the selected VL CDRs 1, 2, 3 amino acid sequences are set forth in SEQ ID NOs: 992, 993, 994, respectively;
(31) the selected VH CDRs 1, 2, 3 amino acid sequences are set forth in SEQ ID NOs: 995, 996, 997, respectively, and the selected VL CDRs 1, 2, 3 amino acid sequences are set forth in SEQ ID NOs: 998, 999, 1000, respectively;
(32) the selected VH CDRs 1, 2, 3 amino acid sequences are set forth in SEQ ID NOs: 1001, 1002, 1003, respectively, and the selected VL CDRs 1, 2, 3 amino acid sequences are set forth in SEQ ID NOs: 1004, 1005, 1006, respectively;
(33) the selected VH CDRs 1, 2, 3 amino acid sequences are set forth in SEQ ID NOs: 1007, 1008, 1009, respectively, and the selected VL CDRs 1, 2, 3 amino acid sequences are set forth in SEQ ID NOs: 1010, 1011, 1012, respectively;
(34) the selected VH CDRs 1, 2, 3 amino acid sequences are set forth in SEQ ID NOs: 1013, 1014, 1015, respectively, and the selected VL CDRs 1, 2, 3 amino acid sequences are set forth in SEQ ID NOs: 1016, 1017, 1018, respectively;
(35) the selected VH CDRs 1, 2, 3 amino acid sequences are set forth in SEQ ID NOs: 1019, 1020, 1021, respectively, and the selected VL CDRs 1, 2, 3 amino acid sequences are set forth in SEQ ID NOs: 1022, 1023, 1024, respectively;
(36) the selected VH CDRs 1, 2, 3 amino acid sequences are set forth in SEQ ID NOs: 1025, 1026, 1027, respectively, and the selected VL CDRs 1, 2, 3 amino acid sequences are set forth in SEQ ID NOs: 1028, 1029, 1030, respectively;
(37) the selected VH CDRs 1, 2, 3 amino acid sequences are set forth in SEQ ID NOs: 1031, 1032, 1033, respectively, and the selected VL CDRs 1, 2, 3 amino acid sequences are set forth in SEQ ID NOs: 1034, 1035, 1036, respectively;
(38) the selected VH CDRs 1, 2, 3 amino acid sequences are set forth in SEQ ID NOs: 1037, 1038, 1039, respectively, and the selected VL CDRs 1, 2, 3 amino acid sequences are set forth in SEQ ID NOs: 1040, 1041, 1042, respectively;
(39) the selected VH CDRs 1, 2, 3 amino acid sequences are set forth in SEQ ID NOs: 1043, 1044, 1045, respectively, and the selected VL CDRs 1, 2, 3 amino acid sequences are set forth in SEQ ID NOs: 1046, 1047, 1048, respectively; and
(40) the selected VH CDRs 1, 2, 3 amino acid sequences are set forth in SEQ ID NOs: 1049, 1050, 1051, respectively, and the selected VL CDRs 1, 2, 3 amino acid sequences are set forth in SEQ ID NOs: 1052, 1053, 1054, respectively.

In some embodiments, CDR is determined by Kabat definition.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 281, 282, 283, respectively, and the selected VL CDRs 1, 2, 3 amino acid sequences are set forth in SEQ ID NOs: 284, 285, 286, respectively;
(2) the selected VH CDRs 1, 2, 3 amino acid sequences are set forth in SEQ ID NOs: 287, 288, 289, respectively, and the selected VL CDRs 1, 2, 3 amino acid sequences are set forth in SEQ ID NOs: 290, 291, 292, respectively;
(3) the selected VH CDRs 1, 2, 3 amino acid sequences are set forth in SEQ ID NOs: 293, 294, 295, respectively, and the selected VL CDRs 1, 2, 3 amino acid sequences are set forth in SEQ ID NOs: 296, 297, 298, respectively;
(4) the selected VH CDRs 1, 2, 3 amino acid sequences are set forth in SEQ ID NOs: 299, 300, 301, respectively, and the selected VL CDRs 1, 2, 3 amino acid sequences are set forth in SEQ ID NOs: 302, 303, 304, respectively;
(5) the selected VH CDRs 1, 2, 3 amino acid sequences are set forth in SEQ ID NOs: 305, 306, 307, respectively, and the selected VL CDRs 1, 2, 3 amino acid sequences are set forth in SEQ ID NOs: 308, 309, 310, respectively;
(6) the selected VH CDRs 1, 2, 3 amino acid sequences are set forth in SEQ ID NOs: 311, 312, 313, respectively, and the selected VL CDRs 1, 2, 3 amino acid sequences are set forth in SEQ ID NOs: 314, 315, 316, respectively;
(7) the selected VH CDRs 1, 2, 3 amino acid sequences are set forth in SEQ ID NOs: 317, 318, 319, respectively, and the selected VL CDRs 1, 2, 3 amino acid sequences are set forth in SEQ ID NOs: 320, 321, 322, respectively;
(8) the selected VH CDRs 1, 2, 3 amino acid sequences are set forth in SEQ ID NOs: 323, 324, 325, respectively, and the selected VL CDRs 1, 2, 3 amino acid sequences are set forth in SEQ ID NOs: 326, 327, 328, respectively;
(9) the selected VH CDRs 1, 2, 3 amino acid sequences are set forth in SEQ ID NOs: 329, 330, 331, respectively, and the selected VL CDRs 1, 2, 3 amino acid sequences are set forth in SEQ ID NOs: 332, 333, 334, respectively;
(10) the selected VH CDRs 1, 2, 3 amino acid sequences are set forth in SEQ ID NOs: 335, 336, 337, respectively, and the selected VL CDRs 1, 2, 3 amino acid sequences are set forth in SEQ ID NOs: 338, 339, 340, respectively;
(11) the selected VH CDRs 1, 2, 3 amino acid sequences are set forth in SEQ ID NOs: 341, 342, 343, respectively, and the selected VL CDRs 1, 2, 3 amino acid sequences are set forth in SEQ ID NOs: 344, 345, 346, respectively;
(12) the selected VH CDRs 1, 2, 3 amino acid sequences are set forth in SEQ ID NOs: 347, 348, 349, respectively, and the selected VL CDRs 1, 2, 3 amino acid sequences are set forth in SEQ ID NOs: 350, 351, 352, respectively;
(13) the selected VH CDRs 1, 2, 3 amino acid sequences are set forth in SEQ ID NOs: 353, 354, 355, respectively, and the selected VL CDRs 1, 2, 3 amino acid sequences are set forth in SEQ ID NOs: 356, 357, 358, respectively;
(14) the selected VH CDRs 1, 2, 3 amino acid sequences are set forth in SEQ ID NOs: 359, 360, 361, respectively, and the selected VL CDRs 1, 2, 3 amino acid sequences are set forth in SEQ ID NOs: 362, 363, 364, respectively;
(15) the selected VH CDRs 1, 2, 3 amino acid sequences are set forth in SEQ ID NOs: 365, 366, 367, respectively, and the selected VL CDRs 1, 2, 3 amino acid sequences are set forth in SEQ ID NOs: 368, 369, 370, respectively;
(16) the selected VH CDRs 1, 2, 3 amino acid sequences are set forth in SEQ ID NOs: 371, 372, 373, respectively, and the selected VL CDRs 1, 2, 3 amino acid sequences are set forth in SEQ ID NOs: 374, 375, 376, respectively;
(17) the selected VH CDRs 1, 2, 3 amino acid sequences are set forth in SEQ ID NOs: 377, 378, 379, respectively, and the selected VL CDRs 1, 2, 3 amino acid sequences are set forth in SEQ ID NOs: 380, 381, 382, respectively;
(18) the selected VH CDRs 1, 2, 3 amino acid sequences are set forth in SEQ ID NOs: 383, 384, 385, respectively, and the selected VL CDRs 1, 2, 3 amino acid sequences are set forth in SEQ ID NOs: 386, 387, 388, respectively;
(19) the selected VH CDRs 1, 2, 3 amino acid sequences are set forth in SEQ ID NOs: 389, 390, 391, respectively, and the selected VL CDRs 1, 2, 3 amino acid sequences are set forth in SEQ ID NOs: 392, 393, 394, respectively;
(20) the selected VH CDRs 1, 2, 3 amino acid sequences are set forth in SEQ ID NOs: 395, 396, 397, respectively, and the selected VL CDRs 1, 2, 3 amino acid sequences are set forth in SEQ ID NOs: 398, 399, 400, respectively;
(21) the selected VH CDRs 1, 2, 3 amino acid sequences are set forth in SEQ ID NOs: 1055, 1056, 1057, respectively, and the selected VL CDRs 1, 2, 3 amino acid sequences are set forth in SEQ ID NOs: 1058, 1059, 1060, respectively;
(22) the selected VH CDRs 1, 2, 3 amino acid sequences are set forth in SEQ ID NOs: 1061, 1062, 1063, respectively, and the selected VL CDRs 1, 2, 3 amino acid sequences are set forth in SEQ ID NOs: 1064, 1065, 1066, respectively;
(23) the selected VH CDRs 1, 2, 3 amino acid sequences are set forth in SEQ ID NOs: 1067, 1068, 1069, respectively, and the selected VL CDRs 1, 2, 3 amino acid sequences are set forth in SEQ ID NOs: 1070, 1071, 1072, respectively;
(24) the selected VH CDRs 1, 2, 3 amino acid sequences are set forth in SEQ ID NOs: 1073, 1074, 1075, respectively, and the selected VL CDRs 1, 2, 3 amino acid sequences are set forth in SEQ ID NOs: 1076, 1077, 1078, respectively;
(25) the selected VH CDRs 1, 2, 3 amino acid sequences are set forth in SEQ ID NOs: 1079, 1080, 1081, respectively, and the selected VL CDRs 1, 2, 3 amino acid sequences are set forth in SEQ ID NOs: 1082, 1083, 1084, respectively;
(26) the selected VH CDRs 1, 2, 3 amino acid sequences are set forth in SEQ ID NOs: 1085, 1086, 1087, respectively, and the selected VL CDRs 1, 2, 3 amino acid sequences are set forth in SEQ ID NOs: 1088, 1089, 1090, respectively;
(27) the selected VH CDRs 1, 2, 3 amino acid sequences are set forth in SEQ ID NOs: 1091, 1092, 1093, respectively, and the selected VL CDRs 1, 2, 3 amino acid sequences are set forth in SEQ ID NOs: 1094, 1095, 1096, respectively;
(28) the selected VH CDRs 1, 2, 3 amino acid sequences are set forth in SEQ ID NOs: 1097, 1098, 1099, respectively, and the selected VL CDRs 1, 2, 3 amino acid sequences are set forth in SEQ ID NOs: 1100, 1101, 1102, respectively;
(29) the selected VH CDRs 1, 2, 3 amino acid sequences are set forth in SEQ ID NOs: 1103, 1104, 1105, respectively, and the selected VL CDRs 1, 2, 3 amino acid sequences are set forth in SEQ ID NOs: 1106, 1107, 1108, respectively;
(30) the selected VH CDRs 1, 2, 3 amino acid sequences are set forth in SEQ ID NOs: 1109, 1110, 1111, respectively, and the selected VL CDRs 1, 2, 3 amino acid sequences are set forth in SEQ ID NOs: 1112, 1113, 1114, respectively;
(31) the selected VH CDRs 1, 2, 3 amino acid sequences are set forth in SEQ ID NOs: 1115, 1116, 1117, respectively, and the selected VL CDRs 1, 2, 3 amino acid sequences are set forth in SEQ ID NOs: 1118, 1119, 1120, respectively;
(32) the selected VH CDRs 1, 2, 3 amino acid sequences are set forth in SEQ ID NOs: 1121, 1122, 1123, respectively, and the selected VL CDRs 1, 2, 3 amino acid sequences are set forth in SEQ ID NOs: 1124, 1125, 1126, respectively;
(33) the selected VH CDRs 1, 2, 3 amino acid sequences are set forth in SEQ ID NOs: 1127, 1128, 1129, respectively, and the selected VL CDRs 1, 2, 3 amino acid sequences are set forth in SEQ ID NOs: 1130, 1131, 1132, respectively;
(34) the selected VH CDRs 1, 2, 3 amino acid sequences are set forth in SEQ ID NOs: 1133, 1134, 1135, respectively, and the selected VL CDRs 1, 2, 3 amino acid sequences are set forth in SEQ ID NOs: 1136, 1137, 1138, respectively;
(35) the selected VH CDRs 1, 2, 3 amino acid sequences are set forth in SEQ ID NOs: 1139, 1140, 1141, respectively, and the selected VL CDRs 1, 2, 3 amino acid sequences are set forth in SEQ ID NOs: 1142, 1143, 1144, respectively;
(36) the selected VH CDRs 1, 2, 3 amino acid sequences are set forth in SEQ ID NOs: 1145, 1146, 1147, respectively, and the selected VL CDRs 1, 2, 3 amino acid sequences are set forth in SEQ ID NOs: 1148, 1149, 1150, respectively;
(37) the selected VH CDRs 1, 2, 3 amino acid sequences are set forth in SEQ ID NOs: 1151, 1152, 1153, respectively, and the selected VL CDRs 1, 2, 3 amino acid sequences are set forth in SEQ ID NOs: 1154, 1155, 1156, respectively;
(38) the selected VH CDRs 1, 2, 3 amino acid sequences are set forth in SEQ ID NOs: 1157, 1158, 1159, respectively, and the selected VL CDRs 1, 2, 3 amino acid sequences are set forth in SEQ ID NOs: 1160, 1161, 1162, respectively;
(39) the selected VH CDRs 1, 2, 3 amino acid sequences are set forth in SEQ ID NOs: 1163, 1164, 1165, respectively, and the selected VL CDRs 1, 2, 3 amino acid sequences are set forth in SEQ ID NOs: 1166, 1167, 1168, respectively; and
(40) the selected VH CDRs 1, 2, 3 amino acid sequences are set forth in SEQ ID NOs: 1169, 1170, 1171, respectively, and the selected VL CDRs 1, 2, 3 amino acid sequences are set forth in SEQ ID NOs: 1172, 1173, 1174, respectively.

In some embodiments, CDR is determined by Chothia definition.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 401, 402, 403, respectively, and the selected VL CDRs 1, 2, 3 amino acid sequences are set forth in SEQ ID NOs: 404, 405, 406, respectively;
(2) the selected VH CDRs 1, 2, 3 amino acid sequences are set forth in SEQ ID NOs: 407, 408, 409, respectively, and the selected VL CDRs 1, 2, 3 amino acid sequences are set forth in SEQ ID NOs: 410, 411, 412, respectively;
(3) the selected VH CDRs 1, 2, 3 amino acid sequences are set forth in SEQ ID NOs: 413, 414, 415, respectively, and the selected VL CDRs 1, 2, 3 amino acid sequences are set forth in SEQ ID NOs: 416, 417, 418, respectively;
(4) the selected VH CDRs 1, 2, 3 amino acid sequences are set forth in SEQ ID NOs: 419, 420, 421, respectively, and the selected VL CDRs 1, 2, 3 amino acid sequences are set forth in SEQ ID NOs: 422, 423, 424, respectively;
(5) the selected VH CDRs 1, 2, 3 amino acid sequences are set forth in SEQ ID NOs: 425, 426, 427, respectively, and the selected VL CDRs 1, 2, 3 amino acid sequences are set forth in SEQ ID NOs: 428, 429, 430, respectively;
(6) the selected VH CDRs 1, 2, 3 amino acid sequences are set forth in SEQ ID NOs: 431, 432, 433, respectively, and the selected VL CDRs 1, 2, 3 amino acid sequences are set forth in SEQ ID NOs: 434, 435, 436, respectively;
(7) the selected VH CDRs 1, 2, 3 amino acid sequences are set forth in SEQ ID NOs: 437, 438, 439, respectively, and the selected VL CDRs 1, 2, 3 amino acid sequences are set forth in SEQ ID NOs: 440, 441, 442, respectively;
(8) the selected VH CDRs 1, 2, 3 amino acid sequences are set forth in SEQ ID NOs: 443, 444, 445, respectively, and the selected VL CDRs 1, 2, 3 amino acid sequences are set forth in SEQ ID NOs: 446, 447, 448, respectively;
(9) the selected VH CDRs 1, 2, 3 amino acid sequences are set forth in SEQ ID NOs: 449, 450, 451, respectively, and the selected VL CDRs 1, 2, 3 amino acid sequences are set forth in SEQ ID NOs: 452, 453, 454, respectively;
(10) the selected VH CDRs 1, 2, 3 amino acid sequences are set forth in SEQ ID NOs: 455, 456, 457, respectively, and the selected VL CDRs 1, 2, 3 amino acid sequences are set forth in SEQ ID NOs: 458, 459, 460, respectively;
(11) the selected VH CDRs 1, 2, 3 amino acid sequences are set forth in SEQ ID NOs: 461, 462, 463, respectively, and the selected VL CDRs 1, 2, 3 amino acid sequences are set forth in SEQ ID NOs: 464, 465, 466, respectively;
(12) the selected VH CDRs 1, 2, 3 amino acid sequences are set forth in SEQ ID NOs: 467, 468, 469, respectively, and the selected VL CDRs 1, 2, 3 amino acid sequences are set forth in SEQ ID NOs: 470, 471, 472, respectively;
(13) the selected VH CDRs 1, 2, 3 amino acid sequences are set forth in SEQ ID NOs: 473, 474, 475, respectively, and the selected VL CDRs 1, 2, 3 amino acid sequences are set forth in SEQ ID NOs: 476, 477, 478, respectively;
(14) the selected VH CDRs 1, 2, 3 amino acid sequences are set forth in SEQ ID NOs: 479, 480, 481, respectively, and the selected VL CDRs 1, 2, 3 amino acid sequences are set forth in SEQ ID NOs: 482, 483, 484, respectively;
(15) the selected VH CDRs 1, 2, 3 amino acid sequences are set forth in SEQ ID NOs: 485, 486, 487, respectively, and the selected VL CDRs 1, 2, 3 amino acid sequences are set forth in SEQ ID NOs: 488, 489, 490, respectively;
(16) the selected VH CDRs 1, 2, 3 amino acid sequences are set forth in SEQ ID NOs: 491, 492, 493, respectively, and the selected VL CDRs 1, 2, 3 amino acid sequences are set forth in SEQ ID NOs: 494, 495, 496, respectively;
(17) the selected VH CDRs 1, 2, 3 amino acid sequences are set forth in SEQ ID NOs: 497, 498, 499, respectively, and the selected VL CDRs 1, 2, 3 amino acid sequences are set forth in SEQ ID NOs: 500, 501, 502, respectively;
(18) the selected VH CDRs 1, 2, 3 amino acid sequences are set forth in SEQ ID NOs: 503, 504, 505, respectively, and the selected VL CDRs 1, 2, 3 amino acid sequences are set forth in SEQ ID NOs: 506, 507, 508, respectively;
(19) the selected VH CDRs 1, 2, 3 amino acid sequences are set forth in SEQ ID NOs: 509, 510, 511, respectively, and the selected VL CDRs 1, 2, 3 amino acid sequences are set forth in SEQ ID NOs: 512, 513, 514, respectively;
(20) the selected VH CDRs 1, 2, 3 amino acid sequences are set forth in SEQ ID NOs: 515, 516, 517, respectively, and the selected VL CDRs 1, 2, 3 amino acid sequences are set forth in SEQ ID NOs: 518, 519, 520, respectively;
(21) the selected VH CDRs 1, 2, 3 amino acid sequences are set forth in SEQ ID NOs: 1175, 1176, 1177, respectively, and the selected VL CDRs 1, 2, 3 amino acid sequences are set forth in SEQ ID NOs: 1178, 1179, 1180, respectively;
(22) the selected VH CDRs 1, 2, 3 amino acid sequences are set forth in SEQ ID NOs: 1181, 1182, 1183, respectively, and the selected VL CDRs 1, 2, 3 amino acid sequences are set forth in SEQ ID NOs: 1184, 1185, 1186, respectively;
(23) the selected VH CDRs 1, 2, 3 amino acid sequences are set forth in SEQ ID NOs: 1187, 1188, 1189, respectively, and the selected VL CDRs 1, 2, 3 amino acid sequences are set forth in SEQ ID NOs: 1190, 1191, 1192, respectively;
(24) the selected VH CDRs 1, 2, 3 amino acid sequences are set forth in SEQ ID NOs: 1193, 1194, 1195, respectively, and the selected VL CDRs 1, 2, 3 amino acid sequences are set forth in SEQ ID NOs: 1196, 1197, 1198, respectively;
(25) the selected VH CDRs 1, 2, 3 amino acid sequences are set forth in SEQ ID NOs: 1199, 1200, 1201, respectively, and the selected VL CDRs 1, 2, 3 amino acid sequences are set forth in SEQ ID NOs: 1202, 1203, 1204, respectively;
(26) the selected VH CDRs 1, 2, 3 amino acid sequences are set forth in SEQ ID NOs: 1205, 1206, 1207, respectively, and the selected VL CDRs 1, 2, 3 amino acid sequences are set forth in SEQ ID NOs: 1208, 1209, 1210, respectively;
(27) the selected VH CDRs 1, 2, 3 amino acid sequences are set forth in SEQ ID NOs: 1211, 1212, 1213, respectively, and the selected VL CDRs 1, 2, 3 amino acid sequences are set forth in SEQ ID NOs: 1214, 1215, 1216, respectively;
(28) the selected VH CDRs 1, 2, 3 amino acid sequences are set forth in SEQ ID NOs: 1217, 1218, 1219, respectively, and the selected VL CDRs 1, 2, 3 amino acid sequences are set forth in SEQ ID NOs: 1220, 1221, 1222, respectively;
(29) the selected VH CDRs 1, 2, 3 amino acid sequences are set forth in SEQ ID NOs: 1223, 1224, 1225, respectively, and the selected VL CDRs 1, 2, 3 amino acid sequences are set forth in SEQ ID NOs: 1226, 1227, 1228, respectively;
(30) the selected VH CDRs 1, 2, 3 amino acid sequences are set forth in SEQ ID NOs: 1229, 1230, 1231, respectively, and the selected VL CDRs 1, 2, 3 amino acid sequences are set forth in SEQ ID NOs: 1232, 1233, 1234, respectively;
(31) the selected VH CDRs 1, 2, 3 amino acid sequences are set forth in SEQ ID NOs: 1235, 1236, 1237, respectively, and the selected VL CDRs 1, 2, 3 amino acid sequences are set forth in SEQ ID NOs: 1238, 1239, 1240, respectively;
(32) the selected VH CDRs 1, 2, 3 amino acid sequences are set forth in SEQ ID NOs: 1241, 1242, 1243, respectively, and the selected VL CDRs 1, 2, 3 amino acid sequences are set forth in SEQ ID NOs: 1244, 1245, 1246, respectively;
(33) the selected VH CDRs 1, 2, 3 amino acid sequences are set forth in SEQ ID NOs: 1247, 1248, 1249, respectively, and the selected VL CDRs 1, 2, 3 amino acid sequences are set forth in SEQ ID NOs: 1250, 1251, 1252, respectively;
(34) the selected VH CDRs 1, 2, 3 amino acid sequences are set forth in SEQ ID NOs: 1253, 1254, 1255, respectively, and the selected VL CDRs 1, 2, 3 amino acid sequences are set forth in SEQ ID NOs: 1256, 1257, 1258, respectively;
(35) the selected VH CDRs 1, 2, 3 amino acid sequences are set forth in SEQ ID NOs: 1259, 1260, 1261, respectively, and the selected VL CDRs 1, 2, 3 amino acid sequences are set forth in SEQ ID NOs: 1262, 1263, 1264, respectively;
(36) the selected VH CDRs 1, 2, 3 amino acid sequences are set forth in SEQ ID NOs: 1265, 1266, 1267, respectively, and the selected VL CDRs 1, 2, 3 amino acid sequences are set forth in SEQ ID NOs: 1268, 1269, 1270, respectively;
(37) the selected VH CDRs 1, 2, 3 amino acid sequences are set forth in SEQ ID NOs: 1271, 1272, 1273, respectively, and the selected VL CDRs 1, 2, 3 amino acid sequences are set forth in SEQ ID NOs: 1274, 1275, 1276, respectively;
(38) the selected VH CDRs 1, 2, 3 amino acid sequences are set forth in SEQ ID NOs: 1277, 1278, 1279, respectively, and the selected VL CDRs 1, 2, 3 amino acid sequences are set forth in SEQ ID NOs: 1280, 1281, 1282, respectively;
(39) the selected VH CDRs 1, 2, 3 amino acid sequences are set forth in SEQ ID NOs: 1283, 1284, 1285, respectively, and the selected VL CDRs 1, 2, 3 amino acid sequences are set forth in SEQ ID NOs: 1286, 1287, 1288, respectively; and
(40) the selected VH CDRs 1, 2, 3 amino acid sequences are set forth in SEQ ID NOs: 1289, 1290, 1291, respectively, and the selected VL CDRs 1, 2, 3 amino acid sequences are set forth in SEQ ID NOs: 1292, 1293, 1294, respectively.

In some embodiments, CDR is determined by Aho definition.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 521, 522, 523, respectively, and the selected VL CDRs 1, 2, 3 amino acid sequences are set forth in SEQ ID NOs: 524, 525, 526, respectively;
(2) the selected VH CDRs 1, 2, 3 amino acid sequences are set forth in SEQ ID NOs: 527, 528, 529, respectively, and the selected VL CDRs 1, 2, 3 amino acid sequences are set forth in SEQ ID NOs: 530, 531, 532, respectively;
(3) the selected VH CDRs 1, 2, 3 amino acid sequences are set forth in SEQ ID NOs: 533, 534, 535, respectively, and the selected VL CDRs 1, 2, 3 amino acid sequences are set forth in SEQ ID NOs: 536, 537, 538, respectively;
(4) the selected VH CDRs 1, 2, 3 amino acid sequences are set forth in SEQ ID NOs: 539, 540, 541, respectively, and the selected VL CDRs 1, 2, 3 amino acid sequences are set forth in SEQ ID NOs: 542, 543, 544, respectively;
(5) the selected VH CDRs 1, 2, 3 amino acid sequences are set forth in SEQ ID NOs: 545, 546, 547, respectively, and the selected VL CDRs 1, 2, 3 amino acid sequences are set forth in SEQ ID NOs: 548, 549, 550, respectively;
(6) the selected VH CDRs 1, 2, 3 amino acid sequences are set forth in SEQ ID NOs: 551, 552, 553, respectively, and the selected VL CDRs 1, 2, 3 amino acid sequences are set forth in SEQ ID NOs: 554, 555, 556, respectively;
(7) the selected VH CDRs 1, 2, 3 amino acid sequences are set forth in SEQ ID NOs: 557, 558, 559, respectively, and the selected VL CDRs 1, 2, 3 amino acid sequences are set forth in SEQ ID NOs: 560, 561, 562, respectively;
(8) the selected VH CDRs 1, 2, 3 amino acid sequences are set forth in SEQ ID NOs: 563, 564, 565, respectively, and the selected VL CDRs 1, 2, 3 amino acid sequences are set forth in SEQ ID NOs: 566, 567, 568, respectively;
(9) the selected VH CDRs 1, 2, 3 amino acid sequences are set forth in SEQ ID NOs: 569, 570, 571, respectively, and the selected VL CDRs 1, 2, 3 amino acid sequences are set forth in SEQ ID NOs: 572, 573, 574, respectively;
(10) the selected VH CDRs 1, 2, 3 amino acid sequences are set forth in SEQ ID NOs: 575, 576, 577, respectively, and the selected VL CDRs 1, 2, 3 amino acid sequences are set forth in SEQ ID NOs: 578, 579, 580, respectively;
(11) the selected VH CDRs 1, 2, 3 amino acid sequences are set forth in SEQ ID NOs: 581, 582, 583, respectively, and the selected VL CDRs 1, 2, 3 amino acid sequences are set forth in SEQ ID NOs: 584, 585, 586, respectively;
(12) the selected VH CDRs 1, 2, 3 amino acid sequences are set forth in SEQ ID NOs: 587, 588, 589, respectively, and the selected VL CDRs 1, 2, 3 amino acid sequences are set forth in SEQ ID NOs: 590, 591, 592, respectively;
(13) the selected VH CDRs 1, 2, 3 amino acid sequences are set forth in SEQ ID NOs: 593, 594, 595, respectively, and the selected VL CDRs 1, 2, 3 amino acid sequences are set forth in SEQ ID NOs: 596, 597, 598, respectively;
(14) the selected VH CDRs 1, 2, 3 amino acid sequences are set forth in SEQ ID NOs: 599, 600, 601, respectively, and the selected VL CDRs 1, 2, 3 amino acid sequences are set forth in SEQ ID NOs: 602, 603, 604, respectively;
(15) the selected VH CDRs 1, 2, 3 amino acid sequences are set forth in SEQ ID NOs: 605, 606, 607, respectively, and the selected VL CDRs 1, 2, 3 amino acid sequences are set forth in SEQ ID NOs: 608, 609, 610, respectively;
(16) the selected VH CDRs 1, 2, 3 amino acid sequences are set forth in SEQ ID NOs: 611, 612, 613, respectively, and the selected VL CDRs 1, 2, 3 amino acid sequences are set forth in SEQ ID NOs: 614, 615, 616, respectively;
(17) the selected VH CDRs 1, 2, 3 amino acid sequences are set forth in SEQ ID NOs: 617, 618, 619, respectively, and the selected VL CDRs 1, 2, 3 amino acid sequences are set forth in SEQ ID NOs: 620, 621, 622, respectively;
(18) the selected VH CDRs 1, 2, 3 amino acid sequences are set forth in SEQ ID NOs: 623, 624, 625, respectively, and the selected VL CDRs 1, 2, 3 amino acid sequences are set forth in SEQ ID NOs: 626, 627, 628, respectively;
(19) the selected VH CDRs 1, 2, 3 amino acid sequences are set forth in SEQ ID NOs: 629, 630, 631, respectively, and the selected VL CDRs 1, 2, 3 amino acid sequences are set forth in SEQ ID NOs: 632, 633, 634, respectively;
(20) the selected VH CDRs 1, 2, 3 amino acid sequences are set forth in SEQ ID NOs: 635, 636, 637, respectively, and the selected VL CDRs 1, 2, 3 amino acid sequences are set forth in SEQ ID NOs: 638, 639, 640, respectively;
(21) the selected VH CDRs 1, 2, 3 amino acid sequences are set forth in SEQ ID NOs: 1295, 1296, 1297, respectively, and the selected VL CDRs 1, 2, 3 amino acid sequences are set forth in SEQ ID NOs: 1298, 1299, 1300, respectively;
(22) the selected VH CDRs 1, 2, 3 amino acid sequences are set forth in SEQ ID NOs: 1301, 1302, 1303, respectively, and the selected VL CDRs 1, 2, 3 amino acid sequences are set forth in SEQ ID NOs: 1304, 1305, 1306, respectively;
(23) the selected VH CDRs 1, 2, 3 amino acid sequences are set forth in SEQ ID NOs: 1307, 1308, 1309, respectively, and the selected VL CDRs 1, 2, 3 amino acid sequences are set forth in SEQ ID NOs: 1310, 1311, 1312, respectively;
(24) the selected VH CDRs 1, 2, 3 amino acid sequences are set forth in SEQ ID NOs: 1313, 1314, 1315, respectively, and the selected VL CDRs 1, 2, 3 amino acid sequences are set forth in SEQ ID NOs: 1316, 1317, 1318, respectively;
(25) the selected VH CDRs 1, 2, 3 amino acid sequences are set forth in SEQ ID NOs: 1319, 1320, 1321, respectively, and the selected VL CDRs 1, 2, 3 amino acid sequences are set forth in SEQ ID NOs: 1322, 1323, 1324, respectively;
(26) the selected VH CDRs 1, 2, 3 amino acid sequences are set forth in SEQ ID NOs: 1325, 1326, 1327, respectively, and the selected VL CDRs 1, 2, 3 amino acid sequences are set forth in SEQ ID NOs: 1328, 1329, 1330, respectively;
(27) the selected VH CDRs 1, 2, 3 amino acid sequences are set forth in SEQ ID NOs: 1331, 1332, 1333, respectively, and the selected VL CDRs 1, 2, 3 amino acid sequences are set forth in SEQ ID NOs: 1334, 1335, 1336, respectively;
(28) the selected VH CDRs 1, 2, 3 amino acid sequences are set forth in SEQ ID NOs: 1337, 1338, 1339, respectively, and the selected VL CDRs 1, 2, 3 amino acid sequences are set forth in SEQ ID NOs: 1340, 1341, 1342, respectively;
(29) the selected VH CDRs 1, 2, 3 amino acid sequences are set forth in SEQ ID NOs: 1343, 1344, 1345, respectively, and the selected VL CDRs 1, 2, 3 amino acid sequences are set forth in SEQ ID NOs: 1346, 1347, 1348, respectively;
(30) the selected VH CDRs 1, 2, 3 amino acid sequences are set forth in SEQ ID NOs: 1349, 1350, 1351, respectively, and the selected VL CDRs 1, 2, 3 amino acid sequences are set forth in SEQ ID NOs: 1352, 1353, 1354, respectively;
(31) the selected VH CDRs 1, 2, 3 amino acid sequences are set forth in SEQ ID NOs: 1355, 1356, 1357, respectively, and the selected VL CDRs 1, 2, 3 amino acid sequences are set forth in SEQ ID NOs: 1358, 1359, 1360, respectively;
(32) the selected VH CDRs 1, 2, 3 amino acid sequences are set forth in SEQ ID NOs: 1361, 1362, 1363, respectively, and the selected VL CDRs 1, 2, 3 amino acid sequences are set forth in SEQ ID NOs: 1364, 1365, 1366, respectively;
(33) the selected VH CDRs 1, 2, 3 amino acid sequences are set forth in SEQ ID NOs: 1367, 1368, 1369, respectively, and the selected VL CDRs 1, 2, 3 amino acid sequences are set forth in SEQ ID NOs: 1370, 1371, 1372, respectively;
(34) the selected VH CDRs 1, 2, 3 amino acid sequences are set forth in SEQ ID NOs: 1373, 1374, 1375, respectively, and the selected VL CDRs 1, 2, 3 amino acid sequences are set forth in SEQ ID NOs: 1376, 1377, 1378, respectively;
(35) the selected VH CDRs 1, 2, 3 amino acid sequences are set forth in SEQ ID NOs: 1379, 1380, 1381, respectively, and the selected VL CDRs 1, 2, 3 amino acid sequences are set forth in SEQ ID NOs: 1382, 1383, 1384, respectively;
(36) the selected VH CDRs 1, 2, 3 amino acid sequences are set forth in SEQ ID NOs: 1385, 1386, 1387, respectively, and the selected VL CDRs 1, 2, 3 amino acid sequences are set forth in SEQ ID NOs: 1388, 1389, 1390, respectively;
(37) the selected VH CDRs 1, 2, 3 amino acid sequences are set forth in SEQ ID NOs: 1391, 1392, 1393, respectively, and the selected VL CDRs 1, 2, 3 amino acid sequences are set forth in SEQ ID NOs: 1394, 1395, 1396, respectively;
(38) the selected VH CDRs 1, 2, 3 amino acid sequences are set forth in SEQ ID NOs: 1397, 1398, 1399, respectively, and the selected VL CDRs 1, 2, 3 amino acid sequences are set forth in SEQ ID NOs: 1400, 1401, 1402, respectively;
(39) the selected VH CDRs 1, 2, 3 amino acid sequences are set forth in SEQ ID NOs: 1403, 1404, 1405, respectively, and the selected VL CDRs 1, 2, 3 amino acid sequences are set forth in SEQ ID NOs: 1406, 1407, 1408, respectively; and
(40) the selected VH CDRs 1, 2, 3 amino acid sequences are set forth in SEQ ID NOs: 1409, 1410, 1411, respectively, and the selected VL CDRs 1, 2, 3 amino acid sequences are set forth in SEQ ID NOs: 1412, 1413, 1414, respectively.

In some embodiments, CDR is determined by North definition.

In some embodiments, the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 708, 709, 710, respectively, and the selected VL CDRs 1, 2, 3 amino acid sequences are set forth in SEQ ID NOs: 711, 712, 713, respectively;
(2) the selected VH CDRs 1, 2, 3 amino acid sequences are set forth in SEQ ID NOs: 714, 715, 716, respectively, and the selected VL CDRs 1, 2, 3 amino acid sequences are set forth in SEQ ID NOs: 717, 718, 719, respectively;
(3) the selected VH CDRs 1, 2, 3 amino acid sequences are set forth in SEQ ID NOs: 720, 721, 722, respectively, and the selected VL CDRs 1, 2, 3 amino acid sequences are set forth in SEQ ID NOs: 723, 724, 725, respectively;
(4) the selected VH CDRs 1, 2, 3 amino acid sequences are set forth in SEQ ID NOs: 726, 727, 728, respectively, and the selected VL CDRs 1, 2, 3 amino acid sequences are set forth in SEQ ID NOs: 729, 730, 731, respectively;
(5) the selected VH CDRs 1, 2, 3 amino acid sequences are set forth in SEQ ID NOs: 732, 733, 734, respectively, and the selected VL CDRs 1, 2, 3 amino acid sequences are set forth in SEQ ID NOs: 735, 736, 737, respectively;
(6) the selected VH CDRs 1, 2, 3 amino acid sequences are set forth in SEQ ID NOs: 738, 739, 740, respectively, and the selected VL CDRs 1, 2, 3 amino acid sequences are set forth in SEQ ID NOs: 741, 742, 743, respectively;
(7) the selected VH CDRs 1, 2, 3 amino acid sequences are set forth in SEQ ID NOs: 744, 745, 746, respectively, and the selected VL CDRs 1, 2, 3 amino acid sequences are set forth in SEQ ID NOs: 747, 748, 749, respectively;
(8) the selected VH CDRs 1, 2, 3 amino acid sequences are set forth in SEQ ID NOs: 750, 751, 752, respectively, and the selected VL CDRs 1, 2, 3 amino acid sequences are set forth in SEQ ID NOs: 753, 754, 755, respectively;
(9) the selected VH CDRs 1, 2, 3 amino acid sequences are set forth in SEQ ID NOs: 756, 757, 758, respectively, and the selected VL CDRs 1, 2, 3 amino acid sequences are set forth in SEQ ID NOs: 759, 760, 761, respectively;
(10) the selected VH CDRs 1, 2, 3 amino acid sequences are set forth in SEQ ID NOs: 762, 763, 764, respectively, and the selected VL CDRs 1, 2, 3 amino acid sequences are set forth in SEQ ID NOs: 765, 766, 767, respectively;
(11) the selected VH CDRs 1, 2, 3 amino acid sequences are set forth in SEQ ID NOs: 768, 769, 770, respectively, and the selected VL CDRs 1, 2, 3 amino acid sequences are set forth in SEQ ID NOs: 771, 772, 773, respectively;
(12) the selected VH CDRs 1, 2, 3 amino acid sequences are set forth in SEQ ID NOs: 774, 775, 776, respectively, and the selected VL CDRs 1, 2, 3 amino acid sequences are set forth in SEQ ID NOs: 777, 778, 779, respectively;
(13) the selected VH CDRs 1, 2, 3 amino acid sequences are set forth in SEQ ID NOs: 780, 781, 782, respectively, and the selected VL CDRs 1, 2, 3 amino acid sequences are set forth in SEQ ID NOs: 783, 784, 785, respectively;
(14) the selected VH CDRs 1, 2, 3 amino acid sequences are set forth in SEQ ID NOs: 786, 787, 788, respectively, and the selected VL CDRs 1, 2, 3 amino acid sequences are set forth in SEQ ID NOs: 789, 790, 791, respectively;
(15) the selected VH CDRs 1, 2, 3 amino acid sequences are set forth in SEQ ID NOs: 792, 793, 794, respectively, and the selected VL CDRs 1, 2, 3 amino acid sequences are set forth in SEQ ID NOs: 795, 796, 797, respectively;
(16) the selected VH CDRs 1, 2, 3 amino acid sequences are set forth in SEQ ID NOs: 798, 799, 800, respectively, and the selected VL CDRs 1, 2, 3 amino acid sequences are set forth in SEQ ID NOs: 801, 802, 803, respectively;
(17) the selected VH CDRs 1, 2, 3 amino acid sequences are set forth in SEQ ID NOs: 804, 805, 806, respectively, and the selected VL CDRs 1, 2, 3 amino acid sequences are set forth in SEQ ID NOs: 807, 808, 809, respectively; and
(18) the selected VH CDRs 1, 2, 3 amino acid sequences are set forth in SEQ ID NOs: 810, 811, 812, respectively, and the selected VL CDRs 1, 2, 3 amino acid sequences are set forth in SEQ ID NOs: 813, 814, 815, respectively.

In some embodiments, the antibody or antigen-binding fragment thereof specifically binds to human MICA and/or human MICB. In some embodiments, the antibody or antigen-binding fragment thereof specifically binds to monkey MICA and/or monkey MICB. In some embodiments, the antibody or antigen-binding fragment thereof can block the shedding of MICA and/or MICB polypeptide from the surfaces of cancer cells. In some embodiments, the antibody or antigen-binding fragment thereof is a humanized antibody or antigen-binding fragment thereof. In some embodiments, the antibody or antigen-binding fragment thereof is a single-chain variable fragment (scFV) or a multi-specific antibody (e.g., a bispecific antibody).

In one aspect, the disclosure is related to an antibody or antigen-binding fragment thereof that binds to MICA and/or MICB comprising a heavy chain variable region (VH) comprising an amino acid sequence that is at least 90% identical to a selected VH sequence, and a light chain variable region (VL) comprising an amino acid sequence that is at least 90% identical to a selected VL sequence, in some embodiments, the selected VH sequence and the selected VL sequence are selected from FIG. 23, 25, 26, 27, 28, or 29.

In some embodiments, the selected VH sequence and the selected VL sequence are one of the following:
(1) the selected VH sequence is SEQ ID NO: 1, and the selected VL sequence is SEQ ID NO: 2;
(2) the selected VH sequence is SEQ ID NO: 9, and the selected VL sequence is SEQ ID NO: 10;
(3) the selected VH sequence is SEQ ID NO: 17, and the selected VL sequence is SEQ ID NO: 18;
(4) the selected VH sequence is SEQ ID NO: 25, and the selected VL sequence is SEQ ID NO: 26;
(5) the selected VH sequence is SEQ ID NO: 33, and the selected VL sequence is SEQ ID NO: 34;
(6) the selected VH sequence is SEQ ID NO: 41, and the selected VL sequence is SEQ ID NO: 42;
(7) the selected VH sequence is SEQ ID NO: 49, and the selected VL sequence is SEQ ID NO: 50;
(8) the selected VH sequence is SEQ ID NO: 57, and the selected VL sequence is SEQ ID NO: 58;
(9) the selected VH sequence is SEQ ID NO: 65, and the selected VL sequence is SEQ ID NO: 66;
(10) the selected VH sequence is SEQ ID NO: 73, and the selected VL sequence is SEQ ID NO: 74;
(11) the selected VH sequence is SEQ ID NO: 81, and the selected VL sequence is SEQ ID NO: 82;
(12) the selected VH sequence is SEQ ID NO: 89, and the selected VL sequence is SEQ ID NO: 90;
(13) the selected VH sequence is SEQ ID NO: 97, and the selected VL sequence is SEQ ID NO: 98;
(14) the selected VH sequence is SEQ ID NO: 105, and the selected VL sequence is SEQ ID NO: 106;
(15) the selected VH sequence is SEQ ID NO: 113, and the selected VL sequence is SEQ ID NO: 114;
(16) the selected VH sequence is SEQ ID NO: 121, and the selected VL sequence is SEQ ID NO: 122;
(17) the selected VH sequence is SEQ ID NO: 129, and the selected VL sequence is SEQ ID NO: 130;
(18) the selected VH sequence is SEQ ID NO: 137, and the selected VL sequence is SEQ ID NO: 138;
(19) the selected VH sequence is SEQ ID NO: 145, and the selected VL sequence is SEQ ID NO: 146; and
(20) the selected VH sequence is SEQ ID NO: 153, and the selected VL sequence is SEQ ID NO: 154.

In some embodiments, the selected VH sequence is SEQ ID NO: 641, 642, 643, 644, 645, 646, 647, 648, 649, 650, 651, 652, 690, 692, 694, 696, 698, 700, 702, 704, or 706, and the selected VL sequence is SEQ ID NO: 653, 654, 655, 656, 657, 658, 691, 693, 695, 697, 699, 701, 703, 705, or 707. In some embodiments, the selected VH sequence is SEQ ID NO: 641, 642, 643, 644, 645, 646, 647, 648, 649, 650, 651, or 652, and the selected VL sequence is SEQ ID NO: 653, 654, 655, 656, 657, or 658. In some embodiments, the selected VH sequence is SEQ ID NO: 659, and the selected VL sequence is SEQ ID NO: 660. In some embodiments, the selected VH sequence is SEQ ID NO: 661, 662, 663, 664, 665, 666, 667, 668, 669, 670, 671, 672, 673, 674, 675, 676, 677, 678, 679, or 680, and the selected VL sequence is SEQ ID NO: 681, 682, 683, 684, 685, 686, 687, 688, or 689. In some embodiments, the VH comprises the sequence of SEQ ID NO: 668, and the VL comprises the sequence of SEQ ID NO: 681. In some embodiments, the VH comprises the sequence of SEQ ID NO: 669, and the VL comprises the sequence of SEQ ID NO: 681. In some embodiments, the VH comprises the sequence of SEQ ID NO: 671, and the VL comprises the sequence of SEQ ID NO: 681. In some embodiments, the VH comprises the sequence of SEQ ID NO: 665, and the VL comprises the sequence of SEQ ID NO: 681. In some embodiments, the VH comprises the sequence of SEQ ID NO: 666, and the VL comprises the sequence of SEQ ID NO: 681. In some embodiments, the VH comprises the sequence of SEQ ID NO: 670, and the VL comprises the sequence of SEQ ID NO: 681. In some embodiments, the VH comprises the sequence of SEQ ID NO: 674, and the VL comprises the sequence of SEQ ID NO: 682. In some embodiments, the VH comprises the sequence of SEQ ID NO: 675, and the VL comprises the sequence of SEQ ID NO: 683. In some embodiments, the VH comprises the sequence of SEQ ID NO: 676, and the VL comprises the sequence of SEQ ID NO: 682. In some embodiments, the VH comprises the sequence of SEQ ID NO: 678, and the VL comprises the sequence of SEQ ID NO: 682. In some embodiments, the VH comprises the sequence of SEQ ID NO: 677, and the VL comprises the sequence of SEQ ID NO: 684. In some embodiments, the VH comprises the sequence of SEQ ID NO: 672, and the VL comprises the sequence of SEQ ID NO: 681. In some embodiments, the VH comprises the sequence of SEQ ID NO: 673, and the VL comprises the sequence of SEQ ID NO: 681. In some embodiments, the VH comprises the sequence of SEQ ID NO: 667, and the VL comprises the sequence of SEQ ID NO: 681. In some embodiments, the VH comprises the sequence of SEQ ID NO: 659, and the VL comprises the sequence of SEQ ID NO: 660. In some embodiments, the VH comprises the sequence of SEQ ID NO: 680, and the VL comprises the sequence of SEQ ID NO: 687. In some embodiments, the VH comprises the sequence of SEQ ID NO: 661, and the VL comprises the sequence of SEQ ID NO: 685. In some embodiments, the VH comprises the sequence of SEQ ID NO: 662, and the VL comprises the sequence of SEQ ID NO: 685. In some embodiments, the VH comprises the sequence of SEQ ID NO: 663, and the VL comprises the sequence of SEQ ID NO: 685. In some embodiments, the VH comprises the sequence of SEQ ID NO: 664, and the VL comprises the sequence of SEQ ID NO: 685. In some embodiments, the VH comprises the sequence of SEQ ID NO: 665, and the VL comprises the sequence of SEQ ID NO: 685. In some embodiments, the VH comprises the sequence of SEQ ID NO: 665, and the VL comprises the sequence of SEQ ID NO: 688. In some embodiments, the VH comprises the sequence of SEQ ID NO: 665, and the VL comprises the sequence of SEQ ID NO: 689. In some embodiments, the VH comprises the sequence of SEQ ID NO: 665, and the VL comprises the sequence of SEQ ID NO: 686. In some embodiments, the VH comprises the sequence of SEQ ID NO: 679, and the VL comprises the sequence of SEQ ID NO: 686. In some embodiments, the VH comprises the sequence of SEQ ID NO: 641, and the VL comprises the sequence of SEQ ID NO: 653. In some embodiments, the VH comprises the sequence of SEQ ID NO: 642, and the VL comprises the sequence of SEQ ID NO: 653. In some embodiments, the VH comprises the sequence of SEQ ID NO: 643, and the VL comprises the sequence of SEQ ID NO: 653. In some embodiments, the VH comprises the sequence of SEQ ID NO: 644, and the VL comprises the sequence of SEQ ID NO: 653. In some embodiments, the VH comprises the sequence of SEQ ID NO: 647, and the VL comprises the sequence of SEQ ID NO: 654. In some embodiments, the VH comprises the sequence of SEQ ID NO: 648, and the VL comprises the sequence of SEQ ID NO: 654. In some embodiments, the VH comprises the sequence of SEQ ID NO: 649, and the VL comprises the sequence of SEQ ID NO: 654. In some embodiments, the VH comprises the sequence of SEQ ID NO: 650, and the VL comprises the sequence of SEQ ID NO: 654. In some embodiments, the VH comprises the sequence of SEQ ID NO: 645, and the VL comprises the sequence of SEQ ID NO: 653. In some embodiments, the VH comprises the sequence of SEQ ID NO: 646, and the VL comprises the sequence of SEQ ID NO: 653. In some embodiments, the VH comprises the sequence of SEQ ID NO: 651, and the VL comprises the sequence of SEQ ID NO: 654. In some embodiments, the VH comprises the sequence of SEQ ID NO: 650, and the VL comprises the sequence of SEQ ID NO: 655. In some embodiments, the VH comprises the sequence of SEQ ID NO: 650, and the VL comprises the sequence of SEQ ID NO: 656. In some embodiments, the VH comprises the sequence of SEQ ID NO: 650, and the VL comprises the sequence of SEQ ID NO: 657. In some embodiments, the VH comprises the sequence of SEQ ID NO: 647, and the VL comprises the sequence of SEQ ID NO: 655. In some embodiments, the VH comprises the sequence of SEQ ID NO: 647, and the VL comprises the sequence of SEQ ID NO: 657. In some embodiments, the VH comprises the sequence of SEQ ID NO: 651, and the VL comprises the sequence of SEQ ID NO: 656. In some embodiments, the VH comprises the sequence of SEQ ID NO: 651, and the VL comprises the sequence of SEQ ID NO: 657. In some embodiments, the VH comprises the sequence of SEQ ID NO: 652, and the VL comprises the sequence of SEQ ID NO: 656. In some embodiments, the VH comprises the sequence of SEQ ID NO: 652, and the VL comprises the sequence of SEQ ID NO: 657. In some embodiments, the VH comprises the sequence of SEQ ID NO: 651, and the VL comprises the sequence of SEQ ID NO: 658. In some embodiments, the VH comprises the sequence of SEQ ID NO: 652, and the VL comprises the sequence of SEQ ID NO: 658. In some embodiments, the VH comprises the sequence of SEQ ID NO: 647, and the VL comprises the sequence of SEQ ID NO: 658. In some embodiments, the VH comprises the sequence of SEQ ID NO: 690, and the VL comprises the sequence of SEQ ID NO: 691. In some embodiments, the VH comprises the sequence of SEQ ID NO: 692, and the VL comprises the sequence of SEQ ID NO: 693. In some embodiments, the VH comprises the sequence of SEQ ID NO: 694, and the VL comprises the sequence of SEQ ID NO: 695. In some embodiments, the VH comprises the sequence of SEQ ID NO: 696, and the VL comprises the sequence of SEQ ID NO: 697. In some embodiments, the VH comprises the sequence of SEQ ID NO: 698, and the VL comprises the sequence of SEQ ID NO: 699. In some embodiments, the VH comprises the sequence of SEQ ID NO: 700, and the VL comprises the sequence of SEQ ID NO: 701. In some embodiments, the VH comprises the sequence of SEQ ID NO: 702, and the VL comprises the sequence of SEQ ID NO: 703. In some embodiments, the VH comprises the sequence of SEQ ID NO: 704, and the VL comprises the sequence of SEQ ID NO: 705. In some embodiments, the VH comprises the sequence of SEQ ID NO: 706, and the VL comprises the sequence of SEQ ID NO: 707.

In some embodiments, the antibody or antigen-binding fragment specifically binds to human MICA and/or MICB. In some embodiments, the antibody or antigen-binding fragment is a humanized antibody or antigen-binding fragment thereof. In some embodiments, the antibody or antigen-binding fragment is a single-chain variable fragment (scFV) or a multi-specific antibody (e.g., a bispecific antibody).

In one aspect, the disclosure is related to an antibody or antigen-binding fragment thereof that cross-competes with the antibody or antigen-binding fragment thereof as described herein.

In one aspect, the disclosure is related to an antibody or antigen-binding fragment thereof comprising a heavy chain variable region (VH) comprising VH CDR1, VH CDR2, and VH CDR3, and a light chain variable region (VL) comprising VL CDR1, VL CDR2, and VL CDR3, in some embodiments, the VH CDR1, VH CDR2, VH CDR3, VL CDR1, VL CDR2, and VL CDR3 are identical to VH CDR1, VH CDR2, VH CDR3, VL CDR1, VL CDR2, and VL CDR3 of the antibody or antigen-binding fragment thereof as described herein.

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof, or the antibody-drug conjugate as described herein, to the subject. In some embodiments, the subject has a solid tumor or hematological cancer. In some embodiments, the cancer is melanoma, neuroblastoma, prostate cancer, kidney cancer, multiple myeloma, or chronic lymphocytic leukemia.

In one aspect, the disclosure is related to a method of decreasing the rate of tumor growth, the method comprising contacting a tumor cell with an effective amount of a composition comprising the antibody or antigen-binding fragment thereof, or the antibody-drug conjugate as described herein.

In one aspect, the disclosure is related to a method of killing a tumor cell, the method comprising contacting a tumor cell with an effective amount of a composition comprising the antibody or antigen-binding fragment thereof, or the antibody-drug conjugate as described herein.

In one aspect, the disclosure is related to a pharmaceutical composition comprising the antibody or antigen-binding fragment thereof, or the antibody-drug conjugate as described herein, and a pharmaceutically acceptable carrier.

In one aspect, the disclosure is related to a nucleic acid comprising a polynucleotide encoding a polypeptide comprising: (1) an immunoglobulin heavy chain or a fragment thereof comprising a heavy chain variable region (VH) comprising complementarity determining regions (CDRs) 1, 2, and 3 comprising VH CDR 1, 2, 3 set forth in FIG. 24A, 24B, 24C, 24D, 24E, 28, 29, 30A, 30B, 30C, 30D, or 30E, and in some embodiments, the VH, when paired with a corresponding light chain variable region (VL) binds to MICA and/or MICB; or (2) an immunoglobulin light chain or a fragment thereof comprising a VL comprising CDRs 1, 2, and 3 comprising VL CDR 1, 2, 3 set forth in FIG. 24A, 24B, 24C, 24D, 24E, 28, 29, 30A, 30B, 30C, 30D, or 30E, when paired with a corresponding VH binds to MICA and/or MICB.

In some embodiments, one of the following is true:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 3, 4, and 5, respectively, and in some embodiments, the VH, when paired with a light chain variable region (VL) comprising the amino acid sequence set forth in SEQ ID NO: 2 binds to MICA and/or MICB;
(2) an immunoglobulin heavy chain or a fragment thereof comprising a VH comprising CDRs 1, 2, and 3 comprising the amino acid sequences set forth in SEQ ID NOs: 11, 12, and 13, respectively, and in some embodiments, the VH, when paired with a VL comprising the amino acid sequence set forth in SEQ ID NO: 10 binds to MICA and/or MICB;
(3) an immunoglobulin heavy chain or a fragment thereof comprising a VH comprising CDRs 1, 2, and 3 comprising the amino acid sequences set forth in SEQ ID NOs: 19, 20, and 21, respectively, and in some embodiments, the VH, when paired with a VL comprising the amino acid sequence set forth in SEQ ID NO: 18 binds to MICA and/or MICB;
(4) an immunoglobulin heavy chain or a fragment thereof comprising a VH comprising CDRs 1, 2, and 3 comprising the amino acid sequences set forth in SEQ ID NOs: 27, 28, and 29, respectively, and in some embodiments, the VH, when paired with a VL comprising the amino acid sequence set forth in SEQ ID NO: 26 binds to MICA and/or MICB;
(5) an immunoglobulin heavy chain or a fragment thereof comprising a VH comprising CDRs 1, 2, and 3 comprising the amino acid sequences set forth in SEQ ID NOs: 35, 36, and 37, respectively, and in some embodiments, the VH, when paired with a VL comprising the amino acid sequence set forth in SEQ ID NO: 34 binds to MICA and/or MICB;
(6) an immunoglobulin heavy chain or a fragment thereof comprising a VH comprising CDRs 1, 2, and 3 comprising the amino acid sequences set forth in SEQ ID NOs: 43, 44, and 45, respectively, and in some embodiments, the VH, when paired with a VL comprising the amino acid sequence set forth in SEQ ID NO: 42 binds to MICA and/or MICB;
(7) an immunoglobulin heavy chain or a fragment thereof comprising a VH comprising CDRs 1, 2, and 3 comprising the amino acid sequences set forth in SEQ ID NOs: 51, 52, and 53, respectively, and in some embodiments, the VH, when paired with a VL comprising the amino acid sequence set forth in SEQ ID NO: 50 binds to MICA and/or MICB;
(8) an immunoglobulin heavy chain or a fragment thereof comprising a VH comprising CDRs 1, 2, and 3 comprising the amino acid sequences set forth in SEQ ID NOs: 59, 60, and 61, respectively, and in some embodiments, the VH, when paired with a VL comprising the amino acid sequence set forth in SEQ ID NO: 58 binds to MICA and/or MICB;
(9) an immunoglobulin heavy chain or a fragment thereof comprising a VH comprising CDRs 1, 2, and 3 comprising the amino acid sequences set forth in SEQ ID NOs: 67, 68, and 69, respectively, and in some embodiments, the VH, when paired with a VL comprising the amino acid sequence set forth in SEQ ID NO: 66 binds to MICA and/or MICB;
(10) an immunoglobulin heavy chain or a fragment thereof comprising a VH comprising CDRs 1, 2, and 3 comprising the amino acid sequences set forth in SEQ ID NOs: 75, 76, and 77, respectively, and in some embodiments, the VH, when paired with a VL comprising the amino acid sequence set forth in SEQ ID NO: 74 binds to MICA and/or MICB;
(11) an immunoglobulin heavy chain or a fragment thereof comprising a VH comprising CDRs 1, 2, and 3 comprising the amino acid sequences set forth in SEQ ID NOs: 83, 84, and 85, respectively, and in some embodiments, the VH, when paired with a VL comprising the amino acid sequence set forth in SEQ ID NO: 82 binds to MICA and/or MICB;
(12) an immunoglobulin heavy chain or a fragment thereof comprising a VH comprising CDRs 1, 2, and 3 comprising the amino acid sequences set forth in SEQ ID NOs: 91, 92, and 93, respectively, and in some embodiments, the VH, when paired with a VL comprising the amino acid sequence set forth in SEQ ID NO: 90 binds to MICA and/or MICB;
(13) an immunoglobulin heavy chain or a fragment thereof comprising a VH comprising CDRs 1, 2, and 3 comprising the amino acid sequences set forth in SEQ ID NOs: 99, 100, and 101, respectively, and in some embodiments, the VH, when paired with a VL comprising the amino acid sequence set forth in SEQ ID NO: 98 binds to MICA and/or MICB;
(14) an immunoglobulin heavy chain or a fragment thereof comprising a VH comprising CDRs 1, 2, and 3 comprising the amino acid sequences set forth in SEQ ID NOs: 107, 108, and 109, respectively, and in some embodiments, the VH, when paired with a VL comprising the amino acid sequence set forth in SEQ ID NO: 106 binds to MICA and/or MICB;
(15) an immunoglobulin heavy chain or a fragment thereof comprising a VH comprising CDRs 1, 2, and 3 comprising the amino acid sequences set forth in SEQ ID NOs: 115, 116, and 117, respectively, and in some embodiments, the VH, when paired with a VL comprising the amino acid sequence set forth in SEQ ID NO: 114 binds to MICA and/or MICB;
(16) an immunoglobulin heavy chain or a fragment thereof comprising a VH comprising CDRs 1, 2, and 3 comprising the amino acid sequences set forth in SEQ ID NOs: 123, 124, and 125, respectively, and in some embodiments, the VH, when paired with a VL comprising the amino acid sequence set forth in SEQ ID NO: 122 binds to MICA and/or MICB;
(17) an immunoglobulin heavy chain or a fragment thereof comprising a VH comprising CDRs 1, 2, and 3 comprising the amino acid sequences set forth in SEQ ID NOs: 131, 132, and 133, respectively, and in some embodiments, the VH, when paired with a VL comprising the amino acid sequence set forth in SEQ ID NO: 130 binds to MICA and/or MICB;
(18) an immunoglobulin heavy chain or a fragment thereof comprising a VH comprising CDRs 1, 2, and 3 comprising the amino acid sequences set forth in SEQ ID NOs: 139, 140, and 141, respectively, and in some embodiments, the VH, when paired with a VL comprising the amino acid sequence set forth in SEQ ID NO: 138 binds to MICA and/or MICB;
(19) an immunoglobulin heavy chain or a fragment thereof comprising a VH comprising CDRs 1, 2, and 3 comprising the amino acid sequences set forth in SEQ ID NOs: 147, 148, and 149, respectively, and in some embodiments, the VH, when paired with a VL comprising the amino acid sequence set forth in SEQ ID NO: 146 binds to MICA and/or MICB;
(20) an immunoglobulin heavy chain or a fragment thereof comprising a VH comprising CDRs 1, 2, and 3 comprising the amino acid sequences set forth in SEQ ID NOs: 155, 156, and 157, respectively, and in some embodiments, the VH, when paired with a VL comprising the amino acid sequence set forth in SEQ ID NO: 154 binds to MICA and/or MICB;
(21) an immunoglobulin light chain or a fragment thereof comprising a VL comprising CDRs 1, 2, and 3 comprising the amino acid sequences set forth in SEQ ID NOs: 6, 7, and 8, respectively, and in some embodiments, the VL, when paired with a VH comprising the amino acid sequence set forth in SEQ ID NO: 1 binds to MICA and/or MICB;
(22) an immunoglobulin light chain or a fragment thereof comprising a VL comprising CDRs 1, 2, and 3 comprising the amino acid sequences set forth in SEQ ID NOs: 14, 15, and 16, respectively, and in some embodiments, the VL, when paired with a VH comprising the amino acid sequence set forth in SEQ ID NO: 9 binds to MICA and/or MICB;
(23) an immunoglobulin light chain or a fragment thereof comprising a VL comprising CDRs 1, 2, and 3 comprising the amino acid sequences set forth in SEQ ID NOs: 22, 23, and 24, respectively, and in some embodiments, the VL, when paired with a VH comprising the amino acid sequence set forth in SEQ ID NO: 17 binds to MICA and/or MICB;
(24) an immunoglobulin light chain or a fragment thereof comprising a VL comprising CDRs 1, 2, and 3 comprising the amino acid sequences set forth in SEQ ID NOs: 30, 31, and 32, respectively, and in some embodiments, the VL, when paired with a VH comprising the amino acid sequence set forth in SEQ ID NO: 25 binds to MICA and/or MICB;
(25) an immunoglobulin light chain or a fragment thereof comprising a VL comprising CDRs 1, 2, and 3 comprising the amino acid sequences set forth in SEQ ID NOs: 38, 39, and 40, respectively, and in some embodiments, the VL, when paired with a VH comprising the amino acid sequence set forth in SEQ ID NO: 33 binds to MICA and/or MICB;
(26) an immunoglobulin light chain or a fragment thereof comprising a VL comprising CDRs 1, 2, and 3 comprising the amino acid sequences set forth in SEQ ID NOs: 46, 47, and 48, respectively, and in some embodiments, the VL, when paired with a VH comprising the amino acid sequence set forth in SEQ ID NO: 41 binds to MICA and/or MICB;
(27) an immunoglobulin light chain or a fragment thereof comprising a VL comprising CDRs 1, 2, and 3 comprising the amino acid sequences set forth in SEQ ID NOs: 54, 55, and 56, respectively, and in some embodiments, the VL, when paired with a VH comprising the amino acid sequence set forth in SEQ ID NO: 49 binds to MICA and/or MICB;
(28) an immunoglobulin light chain or a fragment thereof comprising a VL comprising CDRs 1, 2, and 3 comprising the amino acid sequences set forth in SEQ ID NOs: 62, 63, and 64, respectively, and in some embodiments, the VL, when paired with a VH comprising the amino acid sequence set forth in SEQ ID NO: 57 binds to MICA and/or MICB;
(29) an immunoglobulin light chain or a fragment thereof comprising a VL comprising CDRs 1, 2, and 3 comprising the amino acid sequences set forth in SEQ ID NOs: 70, 71, and 72, respectively, and in some embodiments, the VL, when paired with a VH comprising the amino acid sequence set forth in SEQ ID NO: 65 binds to MICA and/or MICB;
(30) an immunoglobulin light chain or a fragment thereof comprising a VL comprising CDRs 1, 2, and 3 comprising the amino acid sequences set forth in SEQ ID NOs: 78, 79, and 80, respectively, and in some embodiments, the VL, when paired with a VH comprising the amino acid sequence set forth in SEQ ID NO: 73 binds to MICA and/or MICB;
(31) an immunoglobulin light chain or a fragment thereof comprising a VL comprising CDRs 1, 2, and 3 comprising the amino acid sequences set forth in SEQ ID NOs: 86, 87, and 88, respectively, and in some embodiments, the VL, when paired with a VH comprising the amino acid sequence set forth in SEQ ID NO: 81 binds to MICA and/or MICB;
(32) an immunoglobulin light chain or a fragment thereof comprising a VL comprising CDRs 1, 2, and 3 comprising the amino acid sequences set forth in SEQ ID NOs: 94, 95, and 96, respectively, and in some embodiments, the VL, when paired with a VH comprising the amino acid sequence set forth in SEQ ID NO: 89 binds to MICA and/or MICB;
(33) an immunoglobulin light chain or a fragment thereof comprising a VL comprising CDRs 1, 2, and 3 comprising the amino acid sequences set forth in SEQ ID NOs: 102, 103, and 104, respectively, and in some embodiments, the VL, when paired with a VH comprising the amino acid sequence set forth in SEQ ID NO: 97 binds to MICA and/or MICB;
(34) an immunoglobulin light chain or a fragment thereof comprising a VL comprising CDRs 1, 2, and 3 comprising the amino acid sequences set forth in SEQ ID NOs: 110, 111, and 112, respectively, and in some embodiments, the VL, when paired with a VH comprising the amino acid sequence set forth in SEQ ID NO: 105 binds to MICA and/or MICB;
(35) an immunoglobulin light chain or a fragment thereof comprising a VL comprising CDRs 1, 2, and 3 comprising the amino acid sequences set forth in SEQ ID NOs: 118, 119, and 120, respectively, and in some embodiments, the VL, when paired with a VH comprising the amino acid sequence set forth in SEQ ID NO: 113 binds to MICA and/or MICB;
(36) an immunoglobulin light chain or a fragment thereof comprising a VL comprising CDRs 1, 2, and 3 comprising the amino acid sequences set forth in SEQ ID NOs: 126, 127, and 128, respectively, and in some embodiments, the VL, when paired with a VH comprising the amino acid sequence set forth in SEQ ID NO: 121 binds to MICA and/or MICB;
(37) an immunoglobulin light chain or a fragment thereof comprising a VL comprising CDRs 1, 2, and 3 comprising the amino acid sequences set forth in SEQ ID NOs: 134, 135, and 136, respectively, and in some embodiments, the VL, when paired with a VH comprising the amino acid sequence set forth in SEQ ID NO: 129 binds to MICA and/or MICB;
(38) an immunoglobulin light chain or a fragment thereof comprising a VL comprising CDRs 1, 2, and 3 comprising the amino acid sequences set forth in SEQ ID NOs: 142, 143, and 144, respectively, and in some embodiments, the VL, when paired with a VH comprising the amino acid sequence set forth in SEQ ID NO: 137 binds to MICA and/or MICB;
(39) an immunoglobulin light chain or a fragment thereof comprising a VL comprising CDRs 1, 2, and 3 comprising the amino acid sequences set forth in SEQ ID NOs: 150, 151, and 152, respectively, and in some embodiments, the VL, when paired with a VH comprising the amino acid sequence set forth in SEQ ID NO: 145 binds to MICA and/or MICB;
(40) an immunoglobulin light chain or a fragment thereof comprising a VL comprising CDRs 1, 2, and 3 comprising the amino acid sequences set forth in SEQ ID NOs: 158, 159, and 160, respectively, and in some embodiments, the VL, when paired with a VH comprising the amino acid sequence set forth in SEQ ID NO: 153 binds to MICA and/or MICB;
(41) an immunoglobulin heavy chain or a fragment thereof comprising a VH comprising CDRs 1, 2, and 3 comprising the amino acid sequences set forth in SEQ ID NOs: 815, 816, and 817, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(42) an immunoglobulin heavy chain or a fragment thereof comprising a VH comprising CDRs 1, 2, and 3 comprising the amino acid sequences set forth in SEQ ID NOs: 821, 822, and 823, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(43) an immunoglobulin heavy chain or a fragment thereof comprising a VH comprising CDRs 1, 2, and 3 comprising the amino acid sequences set forth in SEQ ID NOs: 827, 828, and 829, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(44) an immunoglobulin heavy chain or a fragment thereof comprising a VH comprising CDRs 1, 2, and 3 comprising the amino acid sequences set forth in SEQ ID NOs: 833, 834, and 835, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(45) an immunoglobulin heavy chain or a fragment thereof comprising a VH comprising CDRs 1, 2, and 3 comprising the amino acid sequences set forth in SEQ ID NOs: 839, 840, and 841, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(46) an immunoglobulin heavy chain or a fragment thereof comprising a VH comprising CDRs 1, 2, and 3 comprising the amino acid sequences set forth in SEQ ID NOs: 845, 846, and 847, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(47) an immunoglobulin heavy chain or a fragment thereof comprising a VH comprising CDRs 1, 2, and 3 comprising the amino acid sequences set forth in SEQ ID NOs: 851, 852, and 853, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(48) an immunoglobulin heavy chain or a fragment thereof comprising a VH comprising CDRs 1, 2, and 3 comprising the amino acid sequences set forth in SEQ ID NOs: 857, 858, and 859, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(49) an immunoglobulin heavy chain or a fragment thereof comprising a VH comprising CDRs 1, 2, and 3 comprising the amino acid sequences set forth in SEQ ID NOs: 863, 864, and 865, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(50) an immunoglobulin heavy chain or a fragment thereof comprising a VH comprising CDRs 1, 2, and 3 comprising the amino acid sequences set forth in SEQ ID NOs: 869, 870, and 871, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(51) an immunoglobulin heavy chain or a fragment thereof comprising a VH comprising CDRs 1, 2, and 3 comprising the amino acid sequences set forth in SEQ ID NOs: 875, 876, and 877, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(52) an immunoglobulin heavy chain or a fragment thereof comprising a VH comprising CDRs 1, 2, and 3 comprising the amino acid sequences set forth in SEQ ID NOs: 881, 882, and 883, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(53) an immunoglobulin heavy chain or a fragment thereof comprising a VH comprising CDRs 1, 2, and 3 comprising the amino acid sequences set forth in SEQ ID NOs: 887, 888, and 889, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(54) an immunoglobulin heavy chain or a fragment thereof comprising a VH comprising CDRs 1, 2, and 3 comprising the amino acid sequences set forth in SEQ ID NOs: 893, 894, and 895, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(55) an immunoglobulin heavy chain or a fragment thereof comprising a VH comprising CDRs 1, 2, and 3 comprising the amino acid sequences set forth in SEQ ID NOs: 899, 900, and 901, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(56) an immunoglobulin heavy chain or a fragment thereof comprising a VH comprising CDRs 1, 2, and 3 comprising the amino acid sequences set forth in SEQ ID NOs: 905, 906, and 907, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(57) an immunoglobulin heavy chain or a fragment thereof comprising a VH comprising CDRs 1, 2, and 3 comprising the amino acid sequences set forth in SEQ ID NOs: 911, 912, and 913, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(58) an immunoglobulin heavy chain or a fragment thereof comprising a VH comprising CDRs 1, 2, and 3 comprising the amino acid sequences set forth in SEQ ID NOs: 917, 918, and 919, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(59) an immunoglobulin heavy chain or a fragment thereof comprising a VH comprising CDRs 1, 2, and 3 comprising the amino acid sequences set forth in SEQ ID NOs: 923, 924, and 925, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(60) an immunoglobulin heavy chain or a fragment thereof comprising a VH comprising CDRs 1, 2, and 3 comprising the amino acid sequences set forth in SEQ ID NOs: 929, 930, and 931, respectively, and in some embodiments, the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(61) an immunoglobulin light chain or a fragment thereof comprising a VL comprising CDRs 1, 2, and 3 comprising the amino acid sequences set forth in SEQ ID NOs: 818, 819, and 820, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(62) an immunoglobulin light chain or a fragment thereof comprising a VL comprising CDRs 1, 2, and 3 comprising the amino acid sequences set forth in SEQ ID NOs: 824, 825, and 826, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(63) an immunoglobulin light chain or a fragment thereof comprising a VL comprising CDRs 1, 2, and 3 comprising the amino acid sequences set forth in SEQ ID NOs: 830, 831, and 832, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(64) an immunoglobulin light chain or a fragment thereof comprising a VL comprising CDRs 1, 2, and 3 comprising the amino acid sequences set forth in SEQ ID NOs: 836, 837, and 838, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(65) an immunoglobulin light chain or a fragment thereof comprising a VL comprising CDRs 1, 2, and 3 comprising the amino acid sequences set forth in SEQ ID NOs: 842, 843, and 844, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(66) an immunoglobulin light chain or a fragment thereof comprising a VL comprising CDRs 1, 2, and 3 comprising the amino acid sequences set forth in SEQ ID NOs: 848, 849, and 850, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(67) an immunoglobulin light chain or a fragment thereof comprising a VL comprising CDRs 1, 2, and 3 comprising the amino acid sequences set forth in SEQ ID NOs: 854, 855, and 856, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(68) an immunoglobulin light chain or a fragment thereof comprising a VL comprising CDRs 1, 2, and 3 comprising the amino acid sequences set forth in SEQ ID NOs: 860, 861, and 862, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(69) an immunoglobulin light chain or a fragment thereof comprising a VL comprising CDRs 1, 2, and 3 comprising the amino acid sequences set forth in SEQ ID NOs: 866, 867, and 868, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(70) an immunoglobulin light chain or a fragment thereof comprising a VL comprising CDRs 1, 2, and 3 comprising the amino acid sequences set forth in SEQ ID NOs: 872, 873, and 874, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(71) an immunoglobulin light chain or a fragment thereof comprising a VL comprising CDRs 1, 2, and 3 comprising the amino acid sequences set forth in SEQ ID NOs: 878, 879, and 880, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(72) an immunoglobulin light chain or a fragment thereof comprising a VL comprising CDRs 1, 2, and 3 comprising the amino acid sequences set forth in SEQ ID NOs: 884, 885, and 886, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(73) an immunoglobulin light chain or a fragment thereof comprising a VL comprising CDRs 1, 2, and 3 comprising the amino acid sequences set forth in SEQ ID NOs: 890, 891, and 892, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(74) an immunoglobulin light chain or a fragment thereof comprising a VL comprising CDRs 1, 2, and 3 comprising the amino acid sequences set forth in SEQ ID NOs: 896, 897, and 898, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(75) an immunoglobulin light chain or a fragment thereof comprising a VL comprising CDRs 1, 2, and 3 comprising the amino acid sequences set forth in SEQ ID NOs: 902, 903, and 904, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(76) an immunoglobulin light chain or a fragment thereof comprising a VL comprising CDRs 1, 2, and 3 comprising the amino acid sequences set forth in SEQ ID NOs: 908, 909, and 910, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(77) an immunoglobulin light chain or a fragment thereof comprising a VL comprising CDRs 1, 2, and 3 comprising the amino acid sequences set forth in SEQ ID NOs: 914, 915, and 916, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(78) an immunoglobulin light chain or a fragment thereof comprising a VL comprising CDRs 1, 2, and 3 comprising the amino acid sequences set forth in SEQ ID NOs: 920, 921, and 922, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(79) an immunoglobulin light chain or a fragment thereof comprising a VL comprising CDRs 1, 2, and 3 comprising the amino acid sequences set forth in SEQ ID NOs: 926, 927, and 928, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB; or
(80) an immunoglobulin light chain or a fragment thereof comprising a VL comprising CDRs 1, 2, and 3 comprising the amino acid sequences set forth in SEQ ID NOs: 932, 933, and 934, respectively, and in some embodiments, the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB.

In some embodiments, the VH when paired with a VL specifically binds to human MICA and/or MICB; or the VL when paired with a VH specifically binds to human MICA and/or MICB. In some embodiments, the immunoglobulin heavy chain or the fragment thereof is a humanized immunoglobulin heavy chain or a fragment thereof, and the immunoglobulin light chain or the fragment thereof is a humanized immunoglobulin light chain or a fragment thereof.

In some embodiments, the nucleic acid encodes a single-chain variable fragment (scFv) or a multi-specific antibody (e.g., a bispecific antibody). In some embodiments, the nucleic acid is cDNA.

In one aspect, the disclosure is related to a vector comprising one or more of the nucleic acids as described herein. In one aspect, the disclosure is related to a vector comprising two of the nucleic acids as described herein, in some embodiments, the vector encodes the VL region and the VH region that together bind to MICA and/or MICB. In one aspect, the disclosure is related to a pair of vectors, in some embodiments, each vector comprises one of the nucleic acids as described herein, in some embodiments, together the pair of vectors encodes the VL region and the VH region that together bind to MICA and/or MICB.

In one aspect, the disclosure is related to a cell comprising the vector or the pair of vectors as described herein. In some embodiments, the cell is a CHO cell. In one aspect, the disclosure is related to a cell comprising one or more of the nucleic acids as described herein. In one aspect, the disclosure is related to a cell comprising two of the nucleic acids as described herein. In some embodiments, the two nucleic acids together encode the VL region and the VH region that together bind to MICA and/or MICB.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., MICA or MICB) to at least one second target molecule (e.g., MICA or MICB) on the surface of a mammalian cell (e.g., a human T-cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., MICA and/or MICB) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a MICA and/or MICB molecule may be referred to as a MICA and/or MICB-specific antibody or an anti-MICA/B antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A shows the binding affinities of chimeric anti-MICA/B antibodies against MICB005, MICA002, MICA004 and MICA008 polypeptides measured by the Carterra® high-throughput antibody characterization platform. Anti-MICA/B antibody 1D5, 3F9, 13A9, 6E1.1.12, B10G5 are reference antibodies and used for benchmarking purposes.

FIG. 1B shows the binding affinities of humanized anti-MICA/B antibodies against MICB005, MICA002, MICA004 and MICA008 polypeptides measured by the Carterra® high-throughput antibody characterization platform. Anti-MICA/B antibody 1D5, 3F9 and 13A9 are reference antibodies.

FIG. 1C summarizes the binding affinities of humanized and optimized anti-MICA/B antibodies against MICB005, MICA002, MICA004, MICA008, MICA009 and Cynomolgus monkey MICA (cynoMICA) polypeptides measured by the GatorPrime™ biolayer interferometry platform. Anti-MICA/B antibody 1D5, 3F9, 13A9, 6E1.1.12, and B10G5-2 are reference antibodies and used for benchmarking purposes.

FIG. 2A shows chimeric anti-MICA/B antibodies binding to MICA/B polypeptides in CHO-MICA002 cells. CHO-MICA002 cells were incubated with titrated anti-MICA/B antibodies and then APC-labeled anti-human Fc antibody. Anti-MICA/B antibody 1D5, 3F9, 13A9 and 6E1.1.12 are reference antibodies. IgG1 is an isotype control (IgG). The antibody binding signals of APC (gMFI) to MICA/B were measured by flow cytometry.

FIG. 2B shows chimeric anti-MICA/B antibodies binding to MICA/B polypeptides in CHO-MICA004 cells. CHO-MICA004 cells were incubated with titrated anti-MICA/B antibodies and then APC-labeled anti-human Fc antibody. Anti-MICA/B antibody 1D5, 3F9, 13A9 and 6E1.1.12 are reference antibodies. IgG1 is an isotype control (IgG). The antibody binding signals of APC (gMFI) to MICA/B were measured by flow cytometry.

FIG. 2C shows chimeric anti-MICA/B antibodies binding to MICA/B polypeptides in CHO-MICA008 cells. CHO-MICA008 cells were incubated with titrated anti-MICA/B antibodies and then APC-labeled anti-human Fc antibody. Anti-MICA/B antibody 1D5, 3F9, 13A9 and 6E1.1.12 are reference antibodies. IgG1 is an isotype control (IgG). The antibody binding signals of APC (gMFI) to MICA/B were measured by flow cytometry.

FIG. 2D shows chimeric anti-MICA/B antibodies binding to MICA/B polypeptides in CHO-MICB005 cells. CHO-MICB005 cells were incubated with titrated anti-MICA/B antibodies and then APC-labeled anti-human Fc antibody. Anti-MICA/B antibody 1D5, 3F9, 13A9 and 6E1.1.12 are reference antibodies. IgG1 is an isotype control (IgG). The antibody binding signals of APC (gMFI) to MICA/B were measured by flow cytometry.

FIG. 2E shows chimeric anti-MICA/B antibodies binding to MICA/B polypeptides in HeLa cells expressing MICA008. HeLa cells were incubated with titrated anti-MICA/B antibodies and then APC-labeled anti-human Fc antibody. Anti-MICA/B antibody 1D5, 3F9, 13A9 and 6E1.1.12 are reference antibodies. IgG1 is an isotype control (IgG). The antibody binding signals of APC (gMFI) to MICA/B were measured by flow cytometry.

FIG. 2F is a table showing the maximum MFI and calculated EC50 of chimeric anti-MICA/B antibodies against MICA/B polypeptides.

FIG. 3A shows humanized anti-MICA/B antibodies binding to MICA/B polypeptides in CHO-MICA002 cells, which were incubated with titrated anti-MICA/B antibodies and then APC-labeled anti-human Fc antibody. Anti-MICA/B antibody 1D5 is the reference antibody. IgG1 is an isotype control (IgG). The antibody binding signals of APC (gMFI) to MICA/B were measured by flow cytometry.

FIG. 3B shows humanized anti-MICA/B antibodies binding to MICA/B polypeptides in CHO-MICA004 cells, which were incubated with titrated anti-MICA/B antibodies and then APC-labeled anti-human Fc antibody. Anti-MICA/B antibody 1D5 is the reference antibody. IgG1 is an isotype control (IgG). The antibody binding signals of APC (gMFI) to MICA/B were measured by flow cytometry.

FIG. 3C shows humanized anti-MICA/B antibodies binding to MICA/B polypeptides in CHO-MICA008 cells, which were incubated with titrated anti-MICA/B antibodies and then APC-labeled anti-human Fc antibody. Anti-MICA/B antibody 1D5 is the reference antibody. IgG1 is an isotype control (IgG). The antibody binding signals of APC (gMFI) to MICA/B were measured by flow cytometry.

FIG. 3D shows humanized anti-MICA/B antibodies binding to MICA/B polypeptides in CHO-MICA005 cells, which were incubated with titrated anti-MICA/B antibodies and then APC-labeled anti-human Fc antibody. Anti-MICA/B antibody 1D5 is the reference antibody. IgG1 is an isotype control (IgG). The antibody binding signals of APC (gMFI) to MICA/B were measured by flow cytometry.

FIG. 3E shows humanized anti-MICA/B antibodies binding to MICA/B polypeptides in CHO-MICA002 cells, which were incubated with titrated anti-MICA/B antibodies and then APC-labeled anti-human Fc antibody. Anti-MICA/B antibody 1D5 and 3F9 are reference antibodies. IgG1 is an isotype control (IgG). The antibody binding signals of APC (gMFI) to MICA/B were measured by flow cytometry.

FIG. 3F shows humanized anti-MICA/B antibodies binding to MICA/B polypeptides in CHO-MICB004 cells, which were incubated with titrated anti-MICA/B antibodies and then APC-labeled anti-human Fc antibody. Anti-MICA/B antibody 1D5 and 3F9 are reference antibodies. IgG1 is an isotype control (IgG). The antibody binding signals of APC (gMFI) to MICA/B were measured by flow cytometry.

FIG. 3G shows humanized anti-MICA/B antibodies binding to MICA/B polypeptides in CHO-MICA008 cells, which were incubated with titrated anti-MICA/B antibodies and then APC-labeled anti-human Fc antibody. Anti-MICA/B antibody 1D5 and 3F9 are reference antibodies. IgG1 is an isotype control (IgG). The antibody binding signals of APC (gMFI) to MICA/B were measured by flow cytometry.

FIG. 3H shows humanized anti-MICA/B antibodies binding to MICA/B polypeptides in CHO-MICB005 cells, which were incubated with titrated anti-MICA/B antibodies and then APC-labeled anti-human Fc antibody. Anti-MICA/B antibody 1D5 and 3F9 are reference antibodies. IgG1 is an isotype control (IgG). The antibody binding signals of APC (gMFI) to MICA/B were measured by flow cytometry.

FIG. 3I shows optimized h36B3 variants binding to MICA/B polypeptides in CHO-ICA002 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3J shows optimized h36B3 variants binding to MICA/B polypeptides in CHO-ICA004 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3K shows optimized h36B3 variants binding to MICA/B polypeptides in CHO-ICA008 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3L shows optimized h36B3 variants binding to MICA/B polypeptides in CHO-MICB005 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3M shows humanized anti-MICA/B antibodies binding to MICA/B polypeptides in HeLa cells, which were incubated with titrated anti-MICA/B antibodies and then APC-labeled anti-human Fc antibody. Anti-MICA/B antibody 1D5 and 3F9 are reference antibodies. IgG1 is an isotype control (IgG). The antibody binding signals of APC (gMFI) to MICA/B were measured by flow cytometry.

FIG. 3N shows optimized h36B3 variants binding to MICA/B polypeptides in HeLa cells, which were incubated with titrated anti-MICA/B antibodies and then APC-labeled anti-human Fc antibody. The antibody binding signals of APC (gMFI) to MICA/B were measured by flow cytometry.

FIG. 3O shows optimized h36B3 variants binding to MICA/B polypeptides in CHO-ICA002 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3P shows optimized h36B3 variants binding to MICA/B polypeptides in CHO-ICA004 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3Q shows optimized h36B3 variants binding to MICA/B polypeptides in CHO-ICA008 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3R shows optimized h36B3 variants binding to MICA/B polypeptides in CHO-MICB005 cells, which were incubated with titrated anti-MICA/B antibodies and then APC-labeled anti-human Fc antibody. IgG1 is an isotype control (IgG). The antibody binding signals of APC (gMFI) to MICA/B were measured by flow cytometry.

FIG. 3S is a table showing the maximum MFI and calculated EC50 of humanized anti-MICA/B antibodies against MICA/B polypeptides expressed by CHO cells.

FIG. 3T is a table showing the maximum MFI and calculated EC50 of humanized anti-MICA/B antibodies against MICA/B polypeptides expressed by CHO cells or HeLa cells.

FIG. 3U is a table showing the maximum MFI and calculated EC50 of optimized h36B3 variants against MICA/B polypeptides expressed by CHO cells.

FIG. 4A is a plot showing that anti-MICA/B antibodies can inhibit the shedding of MICA/B polypeptides from CHO-MICA002 cells. The shed MICA/B polypeptides in the cell culture medium were quantified by sandwich ELISA.

FIG. 4B is a plot showing that anti-MICA/B antibodies can inhibit the shedding of MICA/B polypeptides from C1R-MICA004 cells. The shed MICA/B polypeptides in the cell culture medium were quantified by sandwich ELISA.

FIG. 4C is a plot showing that anti-MICA/B antibodies can inhibit the shedding of MICA/B polypeptides from C1R-MICA008 cells. The shed MICA/B polypeptides in the cell culture medium were quantified by sandwich ELISA.

FIG. 4D is a plot showing that anti-MICA/B antibodies can inhibit the shedding of MICA/B polypeptides from C1R-MICB005 cells. The shed MICA/B polypeptides in the cell culture medium were quantified by sandwich ELISA.

FIG. 4E is a plot showing that anti-MICA/B antibodies can inhibit the shedding of MICA/B polypeptides from HeLa cells expressing MICA008 polypeptides. The shed MICA/B polypeptides in the cell culture medium were quantified by sandwich ELISA.

FIG. 4F is a table showing the maximum shedding inhibition and calculated IC50 of chimeric anti-MICA/B antibodies against MICA/B polypeptides.

FIG. 5A is a plot showing the expression of surface MICA/B in C1R-MICA002 cells treated with chimeric anti-MICA/B antibodies. The MICA/B signals were quantified by flow cytometry with PE-anti-MICA/B 6D4 which does not compete with our anti-MICA/B.

FIG. 5B is a plot showing the expression of surface MICA/B in C1R-MICA004 cells treated with chimeric anti-MICA/B antibodies. The MICA/B signals were quantified by flow cytometry with PE-anti-MICA/B 6D4 which does not compete with our anti-MICA/B.

FIG. 5C is a plot showing the expression of surface MICA/B in C1R-MICA008 cells treated with chimeric anti-MICA/B antibodies. The MICA/B signals were quantified by flow cytometry with PE-anti-MICA/B 6D4 which does not compete with our anti-MICA/B.

FIG. 5D is a plot showing the expression of surface MICA/B in C1R-MICA005 cells treated with chimeric anti-MICA/B antibodies. The MICA/B signals were quantified by flow cytometry with PE-anti-MICA/B 6D4 which does not compete with our anti-MICA/B.

FIG. 5E is a plot showing the expression of surface MICA/B in HeLa cells treated with chimeric anti-MICA/B antibodies. The MICA/B signals were quantified by flow cytometry with PE-anti-MICA/B 6D4 which does not compete with our anti-MICA/B.

FIG. 5F is a table showing increased expression of MICA/B and calculated EC50 of chimeric anti-MICA/B antibodies against MICA/B polypeptides.

FIG. 6A is a plot showing that anti-MICA/B antibodies can inhibit the shedding of MICA/B polypeptides from C1R-MICA002 cells. The shed MICA/B polypeptides in the cell culture medium were quantified by sandwich ELISA.

FIG. 6B is a plot showing that anti-MICA/B antibodies can inhibit the shedding of MICA/B polypeptides from C1R-MICA004 cells. The shed MICA/B polypeptides in the cell culture medium were quantified by sandwich ELISA.

FIG. 6C is a plot showing that anti-MICA/B antibodies can inhibit the shedding of MICA/B polypeptides from C1R-MICA008 cells. The shed MICA/B polypeptides in the cell culture medium were quantified by sandwich ELISA.

FIG. 6D is a plot showing that anti-MICA/B antibodies can inhibit the shedding of MICA/B polypeptides from C1R-MICB005 cells. The shed MICA/B polypeptides in the cell culture medium were quantified by sandwich ELISA.

FIG. 6E is a plot showing that anti-MICA/B antibodies can inhibit the shedding of MICA/B polypeptides from C1R-MICA002 cells. The shed MICA/B polypeptides in the cell culture medium were quantified by sandwich ELISA.

FIG. 6F is a plot showing that anti-MICA/B antibodies can inhibit the shedding of MICA/B polypeptides from C1R-MICA004 cells. The shed MICA/B polypeptides in the cell culture medium were quantified by sandwich ELISA.

FIG. 6G is a plot showing that anti-MICA/B antibodies can inhibit the shedding of MICA/B polypeptides from C1R-MICA008 cells. The shed MICA/B polypeptides in the cell culture medium were quantified by sandwich ELISA.

FIG. 6H is a plot showing that anti-MICA/B antibodies can inhibit the shedding of MICA/B polypeptides from C1R-MICB005 cells. The shed MICA/B polypeptides in the cell culture medium were quantified by sandwich ELISA.

FIG. 6I is a plot showing that optimized h36B3 variants can inhibit the shedding of MICA/B polypeptides from C1R-MICA002 cells. The shed MICA/B polypeptides in the cell culture medium were quantified by sandwich ELISA.

FIG. 6J is a plot showing that optimized h36B3 variants can inhibit the shedding of MICA/B polypeptides from C1R-MICA004 cells. The shed MICA/B polypeptides in the cell culture medium were quantified by sandwich ELISA.

FIG. 6K is a plot showing that optimized h36B3 variants can inhibit the shedding of MICA/B polypeptides from C1R-MICA008 cells. The shed MICA/B polypeptides in the cell culture medium were quantified by sandwich ELISA.

FIG. 6L is a plot showing that optimized h36B3 variants can inhibit the shedding of MICA/B polypeptides from C1R-MICB005 cells. The shed MICA/B polypeptides in the cell culture medium were quantified by sandwich ELISA.

FIG. 6M is a plot showing that optimized h36B3 variants can inhibit the shedding of MICA/B polypeptides from C1R-MICA002 cells. The shed MICA/B polypeptides in the cell culture medium were quantified by sandwich ELISA.

FIG. 6N is a plot showing that optimized h36B3 variants can inhibit the shedding of MICA/B polypeptides from C1R-MICA004 cells. The shed MICA/B polypeptides in the cell culture medium were quantified by sandwich ELISA.

FIG. 6O is a plot showing that optimized h36B3 variants can inhibit the shedding of MICA/B polypeptides from C1R-MICA008 cells. The shed MICA/B polypeptides in the cell culture medium were quantified by sandwich ELISA.

FIG. 6P is a plot showing that optimized h36B3 variants can inhibit the shedding of MICA/B polypeptides from C1R-MICB005 cells. The shed MICA/B polypeptides in the cell culture medium were quantified by sandwich ELISA.

FIG. 6Q is a table showing the maximum shedding inhibition and calculated IC50 of humanized anti-MICA/B antibodies against several MICA/B polypeptides.

FIG. 6R is a table showing the maximum shedding inhibition and calculated IC50 of humanized anti-MICA/B antibodies against several MICA/B polypeptides.

FIG. 6S is a table showing the maximum shedding inhibition and calculated IC50 of optimized h36B3 variants against several MICA/B polypeptides.

FIG. 6T is a table showing the maximum shedding inhibition and calculated IC50 of optimized h36B3 variants against several MICA/B polypeptides.

FIG. 6U is a plot comparing the abilities of D2M001-209 and benchmark anti-MICA/B to inhibit the shedding of MICA/B polypeptides from C1R-MICB005 cells. The shed MICA/B polypeptides in the cell culture medium were quantified by sandwich ELISA. The maximum shedding inhibition and calculated IC50 are shown below the plot.

FIG. 6V is a plot comparing the abilities of D2M001-209 and benchmark anti-MICA/B to inhibit the shedding of MICA/B polypeptides from C1R-MICA002 cells. The shed MICA/B polypeptides in the cell culture medium were quantified by sandwich ELISA. The maximum shedding inhibition and calculated IC50 are shown below the plot.

FIG. 6W is a plot comparing the abilities of D2M001-209 and benchmark anti-MICA/B to inhibit the shedding of MICA/B polypeptides from C1R-MICA004 cells. The shed MICA/B polypeptides in the cell culture medium were quantified by sandwich ELISA. The maximum shedding inhibition and calculated IC50 are shown below the plot.

FIG. 6X is a plot comparing the abilities of D2M001-209 and benchmark anti-MICA/B to inhibit the shedding of MICA/B polypeptides from C1R-MICA008 cells. The shed MICA/B polypeptides in the cell culture medium were quantified by sandwich ELISA. The maximum shedding inhibition and calculated IC50 are shown below the plot.

FIG. 7A is a plot showing the expression of surface MICA/B in C1R-MICA002 cells treated with humanized anti-MICA/B antibodies. The MICA/B signals were quantified by flow cytometry with PE-anti-MICA/B 6D4.

FIG. 7B is a plot showing the expression of surface MICA/B in C1R-MICA004 cells treated with humanized anti-MICA/B antibodies. The MICA/B signals were quantified by flow cytometry with PE-anti-MICA/B 6D4.

FIG. 7C is a plot showing the expression of surface MICA/B in C1R-MICA008 cells treated with humanized anti-MICA/B antibodies. The MICA/B signals were quantified by flow cytometry with PE-anti-MICA/B 6D4.

FIG. 7D is a plot showing the expression of surface MICA/B in C1R-MICB005 cells treated with humanized anti-MICA/B antibodies. The MICA/B signals were quantified by flow cytometry with PE-anti-MICA/B 6D4.

FIG. 7E is a plot showing the expression of surface MICA/B in C1R-MICA002 cells treated with humanized anti-MICA/B antibodies. The MICA/B signals were quantified by flow cytometry with PE-anti-MICA/B 6D4.

FIG. 7F is a plot showing the expression of surface MICA/B in C1R-MICA004 cells treated with humanized anti-MICA/B antibodies. The MICA/B signals were quantified by flow cytometry with PE-anti-MICA/B 6D4.

FIG. 7G is a plot showing the expression of surface MICA/B in C1R-MICA008 cells treated with humanized anti-MICA/B antibodies. The MICA/B signals were quantified by flow cytometry with PE-anti-MICA/B 6D4.

FIG. 7H is a plot showing the expression of surface MICA/B in C1R-MICB005 cells treated with humanized anti-MICA/B antibodies. The MICA/B signals were quantified by flow cytometry with PE-anti-MICA/B 6D4.

FIG. 7I is a plot showing the expression of surface MICA/B in C1R-MICA002 cells treated with optimized h36B3 variants. The MICA/B signals were quantified by flow cytometry with PE-anti-MICA/B 6D4.

FIG. 7J is a plot showing the expression of surface MICA/B in C1R-MICA004 cells treated with optimized h36B3 variants. The MICA/B signals were quantified by flow cytometry with PE-anti-MICA/B 6D4.

FIG. 7K is a plot showing the expression of surface MICA/B in C1R-MICA008 cells treated with optimized h36B3 variants. The MICA/B signals were quantified by flow cytometry with PE-anti-MICA/B 6D4.

FIG. 7L is a plot showing the expression of surface MICA/B in C1R-MICB005 cells treated with optimized h36B3 variants. The MICA/B signals were quantified by flow cytometry with PE-anti-MICA/B 6D4.

FIG. 7M is a plot showing the expression of surface MICA/B in C1R-MICA002 cells treated with optimized h36B3 variants. The MICA/B signals were quantified by flow cytometry with PE-anti-MICA/B 6D4.

FIG. 7N is a plot showing the expression of surface MICA/B in C1R-MICA004 cells treated with optimized h36B3 variants. The MICA/B signals were quantified by flow cytometry with PE-anti-MICA/B 6D4.

FIG. 7O is a plot showing the expression of surface MICA/B in C1R-MICA008 cells treated with optimized h36B3 variants. The MICA/B signals were quantified by flow cytometry with PE-anti-MICA/B 6D4.

FIG. 7P is a plot showing the expression of surface MICA/B in C1R-MICB005 cells treated with optimized h36B3 variants. The MICA/B signals were quantified by flow cytometry with PE-anti-MICA/B 6D4.

FIG. 7Q is a table showing the maximum increased expression of MICA/B and calculated EC50 of humanized anti-MICA/B antibodies against several MICA/B polypeptides.

FIG. 7R is a table showing the maximum increased expression of MICA/B and calculated EC50 of humanized anti-MICA/B antibodies against several MICA/B polypeptides.

FIG. 7S is a table showing the maximum increased expression of MICA/B and calculated EC50 of optimized h36B3 variants against several MICA/B polypeptides.

FIG. 7T is a table showing the maximum increased expression of MICA/B and calculated EC50 of optimized h36B3 variants against several MICA/B polypeptides.

FIG. 7U is a plot comparing the abilities of D2M001-209 and benchmark anti-MICA/B to stabilize surface MICA/B polypeptides on C1R-MICB005 cells. The MICA/B signals were quantified by flow cytometry with PE-anti-MICA/B 6D4. The calculated EC50, TopMFI and Increases (%) of surface MICB005 are shown below the plot.

FIG. 7V is a plot comparing the abilities of D2M001-209 and benchmark anti-MICA/B to stabilize surface MICA/B polypeptides on C1R-MICA002 cells. The MICA/B signals were quantified by flow cytometry with PE-anti-MICA/B 6D4. The calculated EC50, TopMFI and Increases (%) of surface MICA002 are shown below the plot.

FIG. 7W is a plot comparing the abilities of D2M001-209 and benchmark anti-MICA/B to stabilize surface MICA/B polypeptides on C1R-MICA004 cells. The MICA/B signals were quantified by flow cytometry with PE-anti-MICA/B 6D4. The calculated EC50, TopMFI and Increases (%) of surface MICA004 are shown below the plot.

FIG. 7X is a plot comparing the abilities of D2M001-209 and benchmark anti-MICA/B to stabilize surface MICA/B polypeptides on C1R-MICA008 cells. The MICA/B signals were quantified by flow cytometry with PE-anti-MICA/B 6D4. The calculated EC50, TopMFI and Increases (%) of surface MICA008 are shown below the plot.

FIG. 8A is a plot showing that chimeric anti-MICA/B antibodies can capture MICA002 polypeptides shed from cells. The shed/bound MICA/B polypeptides were from the conditional growth medium of the CHO-MICA002 cells.

FIG. 8B is a plot showing that chimeric anti-MICA/B antibodies can capture MICA004 polypeptides shed from cells. The shed/bound MICA/B polypeptides were from the conditional growth medium of the CHO-MICA004 cells.

FIG. 8C is a plot showing that chimeric anti-MICA/B antibodies can capture MICA008 polypeptides shed from cells. The shed/bound MICA/B polypeptides were from the conditional growth medium of the CHO-MICA008 cells.

FIG. 8D is a plot showing that chimeric anti-MICA/B antibodies can capture ICB005 polypeptides shed from cells. The shed/bound MICA/B polypeptides were from the conditional growth medium of the CHO-MICB005 cells.

FIG. 8E is a table showing the maximum OD450 and calculated EC50 of chimeric anti-MICA/B antibodies against various shed MICA/B polypeptides.

FIG. 9A is a plot showing that humanized anti-MICA/B antibodies (#39 variants) can capture ICA002 polypeptides shed from cells. The shed/bound MICA/B polypeptides were from the conditional growth medium of the CHO-ICA002 cells.

FIG. 9B is a plot showing that humanized anti-MICA/B antibodies (#39 variants) can capture ICA004 polypeptides shed from cells. The shed/bound MICA/B polypeptides were from the conditional growth medium of the CHO-MICA004 cells.

FIG. 9C is a plot showing that humanized anti-MICA/B antibodies (#39 variants) can capture ICA008 polypeptides shed from cells. The shed/bound MICA/B polypeptides were from the conditional growth medium of the CHO-MICA008 cells.

FIG. 9D is a plot showing that humanized anti-MICA/B antibodies (#39 variants) can capture MICB005 polypeptides shed from cells. The shed/bound MICA/B polypeptides were from the conditional growth medium of the CHO-MICB005 cells.

FIG. 9E is a plot showing that humanized anti-MICA/B antibodies can capture soluble MICA002 polypeptides shed from cells. The shed/bound MICA/B polypeptides were from the conditional growth medium of the CHO-ICA002 cells.

FIG. 9F is a plot showing that humanized anti-MICA/B antibodies can capture soluble MICA004 polypeptides shed from cells. The shed/bound MICA/B polypeptides were from the conditional growth medium of the CHO-ICA004 cells.

FIG. 9G is a plot showing that humanized anti-MICA/B antibodies can capture soluble MICA008 polypeptides shed from cells. The shed/bound MICA/B polypeptides were from the conditional growth medium of the CHO-ICA008 cells.

FIG. 9H is a plot showing that humanized anti-MICA/B antibodies can capture soluble MICB005 polypeptides shed from cells. The shed/bound MICA/B polypeptides were from the conditional growth medium of the CHO-MICB005 cells.

FIG. 9I is a table showing the maximum OD450 and calculated EC50 of humanized anti-MICA/B antibodies (#39 variants) against several shed MICA/B polypeptides.

FIG. 9J is a table showing the maximum OD450 and calculated EC50 of humanized anti-MICA/B antibodies against several shed MICA/B polypeptides.

FIG. 9K is a plot showing that humanized anti-MICA/B antibodies (optimized h36B3 variants) can capture soluble MICA002 polypeptides shed from cells. The shed/bound MICA/B polypeptides were from the conditional growth medium of the CHO-MICA008 cells.

FIG. 9L is a plot showing that humanized anti-MICA/B antibodies (optimized h36B3 variants) can capture soluble MICA004 polypeptides shed from cells. The shed/bound MICA/B polypeptides were from the conditional growth medium of the CHO-MICB005 cells.

FIG. 9M is a plot showing that humanized anti-MICA/B antibodies (optimized h36B3 variants) can capture soluble MICA008 polypeptides shed from cells. The shed/bound MICA/B polypeptides were from the conditional growth medium of the CHO-MICA008 cells.

FIG. 9N is a plot showing that humanized anti-MICA/B antibodies (optimized h36B3 variants) can capture soluble MICB005 polypeptides shed from cells. The shed/bound MICA/B polypeptides were from the conditional growth medium of the CHO-MICB005 cells.

FIG. 9O is a table showing the maximum OD450 and calculated EC50 of humanized anti-MICA/B antibodies (optimized h36B3 variants) against several shed MICA/B polypeptides.

FIG. 10A is a plot showing that the interaction of NKG2D with synthetic MICA/B polypeptides measured by ELISA.

FIG. 10B is a plot showing the interaction of NKG2D with the immunocomplex of humanized anti-MICA/B antibody (#39 variants) and shed MICA002 polypeptide measured by ELISA.

FIG. 10C is a plot showing the interaction of NKG2D with the immunocomplex of humanized anti-MICA/B antibody (#39 variants) and shed MICA004 polypeptide measured by ELISA.

FIG. 10D is a table showing the maximum OD450 and calculated EC50 of humanized anti-MICA/B antibodies (#39 variants) against shed MICA002 and MICA004 polypeptides.

FIG. 10E is a plot showing the IL2 concentration in Hut-78 T cells treated with the immunocomplex of chimeric anti-MICA/B antibody and shed MICA002 polypeptide in the presence of anti-CD3 stimulation.

FIG. 10F is a plot showing the IL2 concentration in Hut-78 T cells treated with the immunocomplex of chimeric anti-MICA/B antibody and shed MICB005 polypeptide in the presence of anti-CD3 stimulation.

FIG. 10G is a plot showing the IL2 concentration in Hut-78 T cells treated with the immunocomplex of humanized anti-MICA/B antibody and shed soluble MICA002 polypeptide in the presence of anti-CD3 stimulation.

FIG. 10H is a plot showing the IL2 concentration in Hut-78 T cells treated with the immunocomplex of humanized anti-MICA/B antibody and shed soluble MICA004 polypeptide in the presence of anti-CD3 stimulation.

FIG. 10I is a plot showing the IL2 concentration in Hut-78 T cells treated with the immunocomplex of humanized anti-MICA/B antibody and shed soluble MICA008 polypeptide in the presence of anti-CD3 stimulation.

FIG. 10J is a plot showing the IL2 concentration in Hut-78 T cells treated with the immunocomplex of humanized anti-MICA/B antibody and shed soluble MICB005 polypeptide in the presence of anti-CD3 stimulation.

FIG. 10K is a plot showing the IL2 concentration in Hut-78 T cells treated with the immunocomplex of humanized anti-MICA/B antibody (h36B3 variants) and shed soluble MICA002 polypeptide in the presence of anti-CD3 stimulation.

FIG. 10L is a plot showing the IL2 concentration in Hut-78 T cells treated with the immunocomplex of humanized anti-MICA/B antibody (h36B3 variants) and shed soluble MICA004 polypeptide in the presence of anti-CD3 stimulation.

FIG. 10M is a plot showing the IL2 concentration in Hut-78 T cells treated with the immunocomplex of humanized anti-MICA/B antibody (h36B3 variants) and shed soluble MICA008 polypeptide in the presence of anti-CD3 stimulation.

FIG. 10N is a plot showing the IL2 concentration in Hut-78 T cells treated with the immunocomplex of humanized anti-MICA/B antibody and shed soluble MICB005 polypeptide in the presence of anti-CD3 stimulation.

FIG. 11A is a plot showing IFN-γ production in human primary NK cells from multiple donors treated with the immunocomplex of humanized anti-MICA/B antibody and shed soluble MICA002 polypeptides. Each datapoint represents NK cells from one donor.

FIG. 11B is a plot showing IFN-γ production in human primary NK cells from multiple donors treated with the immunocomplex of humanized anti-MICA/B antibody and shed soluble MICA004 polypeptides. Each datapoint represents NK cells from one donor.

FIG. 11C is a plot showing IFN-γ production in human primary NK cells from multiple donors treated with the immunocomplex of humanized anti-MICA/B antibody and shed soluble MICA008 polypeptides. Each datapoint represents NK cells from one donor.

FIG. 11D is a plot showing IFN-γ production in human primary NK cells from multiple donors treated with the immunocomplex of humanized anti-MICA/B antibody and shed soluble MICB005 polypeptides. Each datapoint represents NK cells from one donor.

FIG. 11E is a plot showing IFN-γ production in human primary NK cells from multiple donors treated with the immunocomplex of humanized anti-MICA/B antibody (h36B3 variants) and shed soluble MICA002 polypeptides. Each datapoint represents NK cells from one donor.

FIG. 11F is a plot showing IFN-γ production in human primary NK cells from multiple donors treated with the immunocomplex of humanized anti-MICA/B antibody (h36B3 variants) and shed soluble MICA004 polypeptides. Each datapoint represents NK cells from one donor.

FIG. 11G is a plot showing IFN-γ production in human primary NK cells from multiple donors treated with the immunocomplex of humanized anti-MICA/B antibody (h36B3 variants) and shed soluble MICA008 polypeptides. Each datapoint represents NK cells from one donor.

FIG. 11H is a plot showing IFN-γ production in human primary NK cells from multiple donors treated with the immunocomplex of humanized anti-MICA/B antibody (h36B3 variants) and shed soluble MICB005 polypeptides. Each datapoint represents NK cells from one donor.

FIG. 12A is a plot showing the expression levels of IFN-γ in NK cells co-cultured with CIR tumor cells expressing MICA004 polypeptides in the presence of chimeric anti-MICA/B antibodies.

FIG. 12B is a plot showing the expression levels of IFN-γ in NK cells co-cultured with CIR tumor cells expressing MICA008 polypeptides in the presence of chimeric anti-MICA/B antibodies.

FIG. 12C is a plot showing the expression levels of IFN-γ in NK cells co-cultured with CIR tumor cells expressing MICB005 polypeptides in the presence of chimeric anti-MICA/B antibodies.

FIG. 12D is a plot showing the expression levels of CD107a in NK cells co-cultured with C1R tumor cells expressing MICA004 polypeptides in the presence of chimeric anti-MICA/B antibodies.

FIG. 12E is a plot showing the expression levels of CD107a in NK cells co-cultured with C1R tumor cells expressing MICA008 polypeptides in the presence of chimeric anti-MICA/B antibodies.

FIG. 12F is a plot showing the expression levels of CD107a in NK cells co-cultured with C1R tumor cells expressing MICB005 polypeptides in the presence of chimeric anti-MICA/B antibodies.

FIG. 12G is a plot showing the expression levels of IFN-γ in NK cells co-cultured with C1R tumor cells expressing MICA002 polypeptides in the presence of humanized anti-MICA/B antibodies.

FIG. 12H is a plot showing the expression levels of IFN-γ in NK cells co-cultured with C1R tumor cells expressing MICA004 polypeptides in the presence of humanized anti-MICA/B antibodies.

FIG. 12I is a plot showing the expression levels of IFN-γ in NK cells co-cultured with C1R tumor cells expressing MICA008 polypeptides in the presence of humanized anti-MICA/B antibodies.

FIG. 12J is a plot showing the expression levels of IFN-γ in NK cells co-cultured with C1R tumor cells expressing MICB005 polypeptides in the presence of humanized anti-MICA/B antibodies.

FIG. 12K is a plot showing the expression levels of CD107a in NK cells co-cultured with C1R tumor cells expressing MICA002 polypeptides in the presence of humanized anti-MICA/B antibodies.

FIG. 12L is a plot showing the expression levels of CD107a in NK cells co-cultured with C1R tumor cells expressing MICA004 polypeptides in the presence of humanized anti-MICA/B antibodies.

FIG. 12M is a plot showing the expression levels of CD107a in NK cells co-cultured with CIR tumor cells expressing MICA008 polypeptides in the presence of humanized anti-MICA/B antibodies.

FIG. 12N is a plot showing the expression levels of CD107a in NK cells co-cultured with C1R tumor cells expressing MICB005 polypeptides in the presence of humanized anti-MICA/B antibodies.

FIG. 12O is a plot showing the expression levels of IFN-γ in NK cells co-cultured with C1R tumor cells expressing MICA002 polypeptides in the presence of humanized anti-MICA/B antibodies (h36B3 variants) from two independent donors.

FIG. 12P is a plot showing the expression levels of IFN-γ in NK cells co-cultured with C1R tumor cells expressing MICA004 polypeptides in the presence of humanized anti-MICA/B antibodies (h36B3 variants) from two independent donors.

FIG. 12Q is a plot showing the expression levels of IFN-γ in NK cells co-cultured with C1R tumor cells expressing MICA008 polypeptides in the presence of humanized anti-MICA/B antibodies (h36B3 variants) from two independent donors.

FIG. 12R is a plot showing the expression levels of IFN-γ in NK cells co-cultured with C1R tumor cells expressing MICB005 polypeptides in the presence of humanized anti-MICA/B antibodies (h36B3 variants) from two independent donors.

FIG. 12S is a plot showing the expression levels of CD107a in NK cells co-cultured with C1R tumor cells expressing MICA002 polypeptides in the presence of humanized anti-MICA/B antibodies (h36B3 variants) from two independent donors.

FIG. 12T is a plot showing the expression levels of CD107a in NK cells co-cultured with C1R tumor cells expressing MICA004 polypeptides in the presence of humanized anti-MICA/B antibodies (h36B3 variants) from two independent donors.

FIG. 12U is a plot showing the expression levels of CD107a in NK cells co-cultured with C1R tumor cells expressing MICA008 polypeptides in the presence of humanized anti-MICA/B antibodies (h36B3 variants) from two independent donors.

FIG. 12V is a plot showing the expression levels of CD107a in NK cells co-cultured with C1R tumor cells expressing MICB005 polypeptides in the presence of humanized anti-MICA/B antibodies (h36B3 variants) from two independent donors.

FIG. 13A is a plot showing the percentage of CIR tumor cells expressing MICA004 polypeptides killed by human NK cells in the presence of chimeric anti-MICA/B antibodies.

FIG. 13B is a plot showing the percentage of CIR tumor cells expressing MICA008 polypeptides killed by human NK cells in the presence of chimeric anti-MICA/B antibodies.

FIG. 13C is a plot showing the percentage of CIR tumor cells expressing MICB005 polypeptides killed by human NK cells in the presence of chimeric anti-MICA/B antibodies.

FIG. 13D is a plot showing the percentage of CIR tumor cells expressing MICA002 polypeptides killed by human NK cells in the presence of humanized anti-MICA/B antibodies.

FIG. 13E is a plot showing the percentage of CIR tumor cells expressing MICA004 polypeptides killed by human NK cells in the presence of humanized anti-MICA/B antibodies.

FIG. 13F is a plot showing the percentage of CIR tumor cells expressing MICA008 polypeptides killed by human NK cells in the presence of humanized anti-MICA/B antibodies.

FIG. 13G is a plot showing the percentage of C1R tumor cells expressing MICB005 polypeptides killed by human NK cells in the presence of humanized anti-MICA/B antibodies.

FIG. 13H is a plot showing the percentage of CIR tumor cells expressing MICA002 polypeptides killed by human NK cells in the presence of humanized anti-MICA/B antibodies (h36B3 variants).

FIG. 13I is a plot showing the percentage of CIR tumor cells expressing ICA004 polypeptides killed by human NK cells in the presence of humanized anti-MICA/B antibodies (h36B3 variants).

FIG. 13J is a plot showing the percentage of C1R tumor cells expressing MICA008 polypeptides killed by human NK cells in the presence of humanized anti-MICA/B antibodies (h36B3 variants).

FIG. 13K is a plot showing the percentage of C1R tumor cells expressing MICB005 polypeptides killed by human NK cells in the presence of humanized anti-MICA/B antibodies (h36B3 variants).

FIG. 14A is a plot showing the thermostability of full IgG of humanized anti-MICA/B antibodies. The melting curves were measured by a protein thermal shift dye kit using a qPCR thermocycler. Fluorescence intensity was subtracted by the lowest intensity (bottom) before the peak and normalized to the amplitude of peak to bottom.

FIG. 14B is a plot showing the thermostability of the F(ab′)₂ fragment of humanized anti-MICA/B antibodies. The melting curves were measured by a protein thermal shift dye kit using a qPCR thermocycler. Fluorescence intensity was subtracted by the lowest intensity (bottom) before the peak and normalized to the amplitude of peak to bottom.

FIG. 14C is a plot showing the thermostability of full IgG of humanized anti-MICA/B antibodies. The melting curves were measured by a protein thermal shift dye kit using a qPCR thermocycler. Fluorescence intensity was subtracted by the lowest intensity (bottom) before the peak and normalized to the amplitude of peak to bottom.

FIG. 14D is a plot showing the thermostability of the F(ab′)₂ fragment of humanized anti-MICA/B antibodies. The melting curves were measured by a protein thermal shift dye kit using a qPCR thermocycler. Fluorescence intensity was subtracted by the lowest intensity (bottom) before the peak and normalized to the amplitude of peak to bottom.

FIG. 14E summarizes the Tm of full IgG and F(ab)₂ fragment of humanized anti-MICA/B antibodies, reference antibodies, and isotype control IgG1 based on the temperature of 50% of amplitude from bottom to peak of the plots shown in FIGS. 14A-14D. Mean and +/−standard deviation were calculated from two experiments with two replicates each.

FIG. 15 is a table showing the binding affinities of humanized anti-MICA/B antibodies stressed in low and high pH buffers, respectively, against various MICA/B polypeptides. The antibodies were stressed in pH 5.5 or pH 8.5 buffer, respectively, at 40° C. for two weeks. Binding kinetics were measured by the GatorPrime™ biolayer interferometry instrument.

FIG. 16 is a table showing clone self-interaction (CSI) of humanized and optimized anti-MICA/B antibodies measured by the GatorPrime™ biolayer interferometry instrument.

FIG. 17A is a plot showing non-specific binding, over background, of humanized and optimized anti-MICA/B antibodies to whole cell lysates produced from CHO cells, with antibodies tested at a concentration of 15 μg/mL by sandwich ELISA.

FIG. 17B is a plot showing non-specific binding, over background, of humanized and optimized anti-MICA/B antibodies to whole cell lysates produced from CHO cells, with antibodies tested at a concentration of 1.5 μg/mL by sandwich ELISA.

FIG. 17C is a plot showing non-specific binding, over background, of humanized and optimized anti-MICA/B antibodies to whole cell lysates produced from C1R cancer cells, with antibodies tested at a concentration of 15 μg/mL by sandwich ELISA.

FIG. 17D is a plot showing non-specific binding, over background, of humanized and optimized anti-MICA/B antibodies to whole cell lysates produced from C1R cancer cells, with antibodies tested at a concentration of 1.5 μg/mL by sandwich ELISA.

FIG. 18A is a plot showing the binding affinity of humanized and optimized anti-MICA/B antibodies in CHO-MICB005 that had been stressed in human serum at 37° C. for 1 week.

FIG. 18B is a plot showing the binding affinity of humanized and optimized anti-MICA/B antibodies in CHO-MICB005 that had been stressed in human serum at 37° C. for 2 weeks.

FIG. 18C is a table showing calculated EC50 for the humanized and optimized anti-MICA/B antibodies plotted in FIGS. 18A-18B.

FIG. 18D is a plot showing the binding affinity of D2M001-209 in CHO-MICA002 that had been stressed in human plasma or PBS at 37° C. for 2 weeks.

FIG. 18E is a plot showing the binding affinity of D2M001-209 in CHO-MICA004 that had been stressed in human plasma or PBS at 37° C. for 2 weeks.

FIG. 18F is a plot showing the binding affinity of D2M001-209 in CHO-MICA008 that had been stressed in human plasma or PBS at 37° C. for 2 weeks.

FIG. 18G is a plot showing the binding affinity of D2M001-209 in CHO-MICB005 that had been stressed in human plasma or PBS at 37° C. for 2 weeks.

FIG. 19A is a plot of the abundance of humanized anti-MICB antibody (D2M001-010) in vivo in mice over time for a ten-day experiment. Antibodies were quantified by a sandwich ELISA using MICB recombinant protein or anti-human Fab as capture reagent and anti-human FC as detection reagent.

FIG. 19B is a plot of the abundance of humanized anti-MICB antibody (D2M001-h39F53GS) in vivo in mice over time for a ten-day experiment. Antibodies were quantified by a sandwich ELISA using MICB recombinant protein or anti-human Fab as capture reagent and anti-human FC as detection reagent.

FIG. 19C is a plot of the abundance of humanized anti-MICB antibody (D2M001-h36B3) in vivo in mice over time for a ten-day experiment. Antibodies were quantified by a sandwich ELISA using MICB recombinant protein or anti-human Fab as capture reagent and anti-human FC as detection reagent.

FIG. 19D is a plot of the abundance of humanized anti-MICB antibody (D2M001-h36B3GS) in vivo in mice over time for a ten-day experiment. Antibodies were quantified by a sandwich ELISA using MICB recombinant protein or anti-human Fab as capture reagent and anti-human FC as detection reagent.

FIG. 19E is a plot of the kinetic abundance of D2M001-209 in vivo in B16F110-MICAB tumor-bearing mice. Blood samples were collected over time for a nine-day experiment with one-dose of 10 mg/kg or 30 mg/kg. Antibodies were quantified by a sandwich ELISA using anti-human F(ab)₂ as capture reagent and anti-human FC as detection reagent. The samples were used for MICB quantification in GIG. 19F.

FIG. 19F is a plot of the kinetic abundance of plasma soluble MICB of tumor-bearing mice treated with D2M001-209 normalized to the mice treated with PBS (untreated). B16F10-MICAB tumor-bearing mice were treated with PBS or one-dose of 10 mg/kg or 30 mg/kg. Blood samples were collected over time for a nine-day experiment at the same time points as described in FIG. 19E. MICB in plasma were quantified by a sandwich ELISA.

FIG. 20A is a plot showing that transgenic mouse tumor cells are capable of shedding MICA in vitro and the inhibition of shedding of MICA polypeptides from transgenic mouse tumor cells by humanized and optimized anti-MICA/B antibodies in vitro.

FIG. 20B is a plot showing that transgenic mouse tumor cells are capable of shedding MICB in vitro and the inhibition of shedding of MICB polypeptides from transgenic mouse tumor cells by humanized and optimized anti-MICA/B antibodies in vitro.

FIG. 20C is a plot showing the reduction of shedding of soluble MICA polypeptides, measured by soluble MICA polypeptides, from transgenic mouse tumor cells by humanized and optimized anti-MICA/B antibody h36B3 in vivo.

FIG. 20D is a plot showing the reduction of shedding of soluble MICA polypeptides, measured by soluble MICB polypeptides, from transgenic mouse tumor cells by humanized and optimized anti-MICA/B antibody h36B3 in vivo.

FIG. 21A is a plot of tumor area (mm²) in response to treatment with humanized and optimized anti-MICA/B antibody D2M001-010, control IgG alone, anti-PD1 antibody alone, or combination of anti-MICA/B antibody D2M001-010 and anti-PD1 antibody. Statistical analysis was performed using t-test. P-values were calculated by t-test. * P<0.05, ** P<0.01, *** P<0.001, **** P<0.0001, n.s. not significant.

FIG. 21B is a plot showing the inhibition of shedding of soluble MICB polypeptides from transgenic mouse tumor cells by humanized and optimized anti-MICA/B antibodies in vivo. Statistical analysis was performed using t-test. P-values were calculated by t-test. * P<0.05, ** P<0.01, *** P<0.001, **** P<0.0001, n.s. not significant.

FIG. 21C is a plot of tumor area (mm²) in response to treatment with humanized and optimized anti-MICA/B antibody h36B3, control IgG alone, anti-PD1 antibody alone, or combination of anti-MICA/B antibody h36B3 and anti-PD1 antibody. Statistical analysis was performed using t-test. P-values were calculated by t-test. * P<0.05, ** P<0.01, n.s. not significant.

FIG. 21D is a plot showing the inhibition of shedding of soluble MICB polypeptides from transgenic mouse tumor cells by humanized and optimized anti-MICA/B antibodies in vivo.

FIG. 21E is a plot of tumor area (mm²) in response to treatment with Fc function null anti-MICA/B antibody h36B3 (h36B3-LALAPA), control IgG alone, anti-PD1 antibody alone, or combination of h36B3-LALAPA and anti-PD1 antibody. Statistical analysis was performed using t-test. P-values were calculated by t-test. * P<0.05, ** P<0.01, n.s. not significant.

FIG. 21F is a plot showing the inhibition of shedding of soluble MICB polypeptides from transgenic mouse tumor cells by humanized and optimized anti-MICA/B antibodies in vivo. Statistical analysis was performed using t-test. P-values were calculated by t-test. * P<0.05, ** P<0.01, n.s. not significant.

FIG. 21G is a plot of tumor volume (mm³) in response to treatment with a single dose of 30 mg/kg D2M001-209, reference antibody 3F9 or control PBS. Statistical analysis was performed using Two-way ANOVA, Tukey's comparison test.

FIG. 21H is a plot of tumor volume (mm³) in response to treatment with humanized and optimized anti-MICA/B antibody D2M001-209, PBS alone, anti-PD1 antibody alone, or combination of D2M001-209 and anti-PD1 antibody. P-values were calculated by two-way ANOVA. * P<0.05, **** P<0.0001, n.s. not significant.

FIG. 21I shows that D2M001-209 increased the surface expression of MICA on the tumor cells in vivo. The residual tumor cells were harvested from tumor-bearing animals treated with anti-MICA/B D2M001-209 antibody and PBS controls in FIG. 21H. P-values were calculated by t-test. * P<0.05, *** P<0.001.

FIG. 21J shows that D2M001-209 increased the surface expression of MICB on tumor cells in vivo. The residual cells were harvested from tumor-bearing animals treated with anti-MICA/B D2M001-209 antibody and PBS controls in FIG. 21H. P-values were calculated by t-test. * P<0.05, *** P<0.001.

FIG. 21K shows that D2M001-209 reduced the concentration of soluble MICA peptides in plasma from animals treated with anti-MICA/B D2M001-209 antibody, PBS alone, anti-PD1 antibody alone, or combination of anti-MICA/B antibody D2M001-209 and anti-PD1 antibody in FIG. 21H. P-values were calculated by t-test. * P<0.05, ** P<0.01, *** P<0.001.

FIG. 21L shows that D2M001-209 reduced the concentration of soluble MICB peptides in plasma from animals treated with anti-MICA/B D2M001-209 antibody, PBS alone, anti-PD1 antibody alone, or combination of anti-MICA/B antibody D2M001-209 and anti-PD1 antibody in FIG. 21H. P-values were calculated by t-test. * P<0.05, ** P<0.01, *** P<0.001.

FIG. 22A is a series of photographs of lungs collected from B6 mice bearing metastatic B16F10-MICA/B tumor cells. Treatment group animals were treated with the anti-MICA/B D2M001-010 antibody or h36B3 and control group animals were treated with IgG isotype controls. Animals were sacrificed and photos were taken 14 days after treatment.

FIG. 22B shows lung tumor nodule counts for animals treated with the anti-MICA/B D2M001-010 antibody, animals treated with the anti-MICA/B h36B3 antibody, and control group animals treated with IgG isotype controls. P-values were calculated by t-test. **** P<0.0001.

FIG. 22C shows the plasma concentration of soluble MICA peptides for animals treated with the anti-MICA/B D2M001-010 antibody and control group animals treated with IgG isotype controls. P-values were calculated by t-test. * P<0.05, ** P<0.01, **** P<0.0001.

FIG. 22D shows the plasma concentration of soluble MICB peptides for animals treated with the anti-MICA/B D2M001-010 antibody and control group animals treated with IgG isotype controls. P-values were calculated by t-test. * P<0.05, ** P<0.01, **** P<0.0001.

FIG. 22E shows lung tumor nodule counts for B6 mice bearing metastatic B16F10-MICA/B tumor cells. Treatment group animals were treated with the anti-MICA/B h36B3 antibody, Fc function null h36B3 antibody (h36B3-LALAPA), and IgG1 isotype controls. P-values were calculated by t-test. * P<0.05, **** P<0.0001.

FIG. 22F shows the plasma concentration of soluble MICA peptide for animals treated with the anti-MICA/B h36B3 antibody, Fc function null h36B3 antibody (h36B3-LALAPA), and IgG1 isotype controls. P-values were calculated by t-test. ** P<0.01, *** P<0.001, **** P<0.0001.

FIG. 22G shows the plasma concentration of soluble MICB peptide for animals treated with the anti-MICA/B h36B3 antibody, Fc function null h36B3 antibody (h36B3-LALAPA), and IgG1 isotype controls. P-values were calculated by t-test. * P<0.05, **** P<0.0001.

FIG. 22H is a plot of tumor volume (mm³) in A375 tumor-bearing SCID mice in response to treatment with humanized and optimized anti-MICA/B antibody D2M001-209 and IgG1 isotype controls. Statistical analysis was performed using t-test. P-values were calculated by t-test. ** P<0.01, *** P<0.001, **** P<0.0001, n.s. not significant.

FIG. 22I shows lung tumor nodule counts for SCID mice bearing metastatic A375 tumor cells. Treatment group animals were treated with the anti-MICA/B D2M001-209 antibody and IgG1 isotype controls. P-values were calculated by t-test. ** P<0.01.

FIG. 23 shows VH and VL sequences of several antibodies.

FIGS. 24A-24E provide the CDR sequences of several antibodies.

FIG. 25 shows the humanized VH and VL sequences.

FIG. 26 shows the humanized VH and VL sequences for hit ID #38 and #39.

FIG. 27 shows the humanized VH and VL sequences for hit ID #36.

FIG. 28 shows the CDR sequences for the humanization of hit ID #36.

FIG. 29 shows the CDR sequences for the humanization of hit ID #38 and #39.

FIGS. 30A-30E provide the CDR sequences of several humanized antibodies.

FIG. 31 provides the protein sequences of reference antibodies.

FIG. 32 provides the protein sequences of full-length MICA/B stably expressed by transgenic cell lines FIG. 33 provides the protein sequences expressed by DNA immunogens used in immunization.

FIG. 34 provides the sequences of protein immunogens used in immunization.

FIG. 35 provides the sequences of recombinant ECD proteins used in affinity measurement.

### DETAILED DESCRIPTION

The present disclosure provides examples of antibodies, antigen-binding fragment thereof, that bind to MICA (major histocompatibility complex class I chain related A) and/or MICB (major histocompatibility complex class I chain related B). In some embodiments, the antibodies or antigen-binding fragments thereof bind to MICA. In some embodiments, the antibodies or antigen-binding fragments thereof bind to MICB. In some embodiments, the antibodies or antigen-binding fragments thereof bind to both MICA and MICB.

### MICA and MICB

Major histocompatibility complex (MHC) class I polypeptide-related A and B (MICA/B) are highly polymorphic cell surface proteins related to MHC class I glycoproteins and are ligands to stimulate an activating receptor, NKG2D, expressed on natural killer (NK) cells, CD8+ T cells, and γδ T cells. The engagement of NKG2D with MICA/B triggers NK cells and co-stimulates T cells, resulting in elimination of cancer cells or damaged cells by effector cells expressing NKG2D receptor.

MICA/B proteins are constitutively expressed at low levels on myeloid cells, epithelial cells, endothelial cells, and fibroblasts. MICA/B proteins are upregulated or expressed de novo in response to stress, e.g., during carcinogenesis, infections, during the DNA damage response, and in various autoimmune conditions. Expression of MICA/B can tag cells for elimination by cytotoxic lymphocytes through NKG2D receptor activation. However, as an escape mechanism in order to prevent the response mediated by NKG2D, tumor cells can proteolytically shed MICA/B proteins from the cell surface resulting both in reduction of MICA/B surface density and in generation of soluble MICA/B (sMICA/B). High serum concentrations of shed MICA polypeptides are associated with disease progression in many human cancers, including melanoma, neuroblastoma, prostate cancer, kidney cancer, multiple myeloma, and chronic lymphocytic leukemia. Expression of MICA/B has also been reported in a wide variety of tumor types, with high expression associated with poor prognosis in patients.

It is believed that specifically blocking the shedding of MICA and MICB proteins from cancer cells may restore or enhance NKG2D-dependent activation of NK and T cells in the tumor microenvironment, and therefore, may enhance anti-tumor activity in cancer patients. Within the MICA/B polypeptide, the ectodomains of MICA/B consist of three C-type Ig-like domains termed alpha-1, alpha-2, and alpha-3 domains. The alpha-1 and alpha-2 domains are distant from the cell membrane, while the alpha-3 domain is proximal to the cell membrane. A six-amino acid motif within the alpha-3 domain was reported as the proteolytic cleavage site, from where MICA/B polypeptides can be shed from the surfaces of cancer cells. A detailed description can be found, e.g., in Wang, X., et al. “An six-amino acid motif in the α3 domain of MICA is the cancer therapeutic target to inhibit shedding.” Biochemical and Biophysical Research Communications 387.3 (2009): 476-481, which is incorporated herein by reference in its entirety.

The function of MICA/B and its relationship with various diseases are described e.g., in Groh V, et al. Tumour-derived soluble MIC ligands impair expression of NKG2D and T-cell activation. Nature. 2002 Oct. 17; 419(6908):734-8.; Salih H R, et al. Cutting edge: down-regulation of MICA on human tumors by proteolytic shedding. J Immunol. 2002 Oct. 15; 169(8):4098-102; Spear P, et al. NKG2D ligands as therapeutic targets. Cancer Immun. 2013 May 1; 13:8.; Ghadially H, et al. MHC class I chain-related protein A and B (MICA and MICB) are predominantly expressed intracellularly in tumour and normal tissue. Br J Cancer. 2017 Apr. 25; 116(9):1208-1217; Xing S, Ferrari de Andrade L. NKG2D and MICA/B shedding: a ‘tag game’ between NK cells and malignant cells. Clin Transl Immunology. 2020 Dec. 22; 9(12):e1230; each of which is incorporated herein by reference in its entirety.

Antibodies that recognize MICA/B polypeptides on the surfaces of cancer cells, in particular specific epitopes required for the initiation of proteolytic shedding, i.e., the alpha-3 domain, may block the shedding of MICA/B polypeptides from the surfaces of cancer cells and reduce the abundance of soluble MICA/B polypeptides released from cancer cells. The present disclosure provides several anti-MICA/B antibodies, antigen-binding fragments thereof, and methods of using these antibodies and antigen-binding fragments to inhibit the shedding of MICA/B polypeptides from cancer cells, inhibit tumor growth, and treat cancers.

### Anti-MICA/B Antibodies and Antigen-Binding Fragments

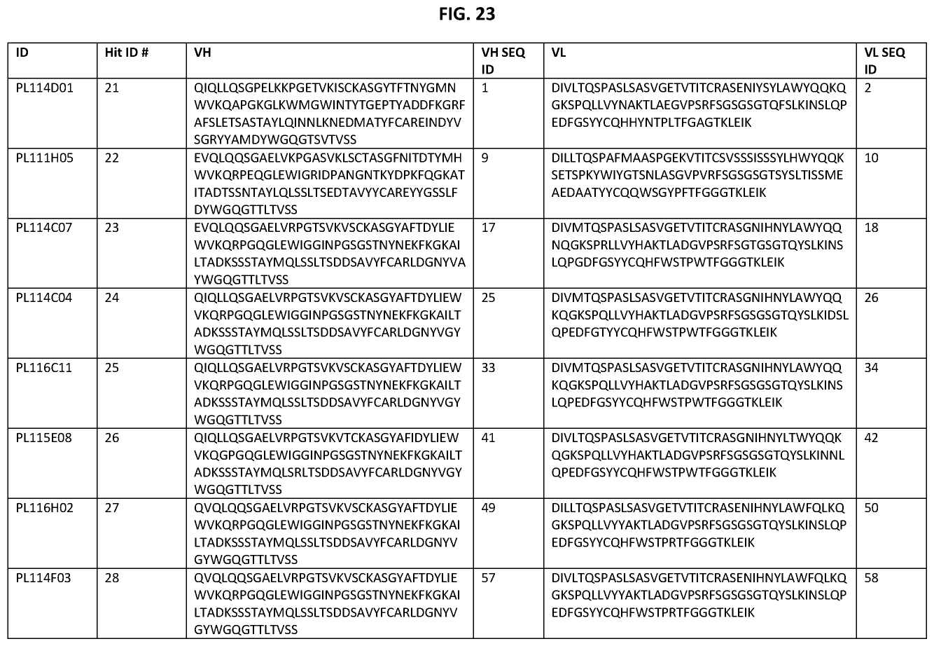

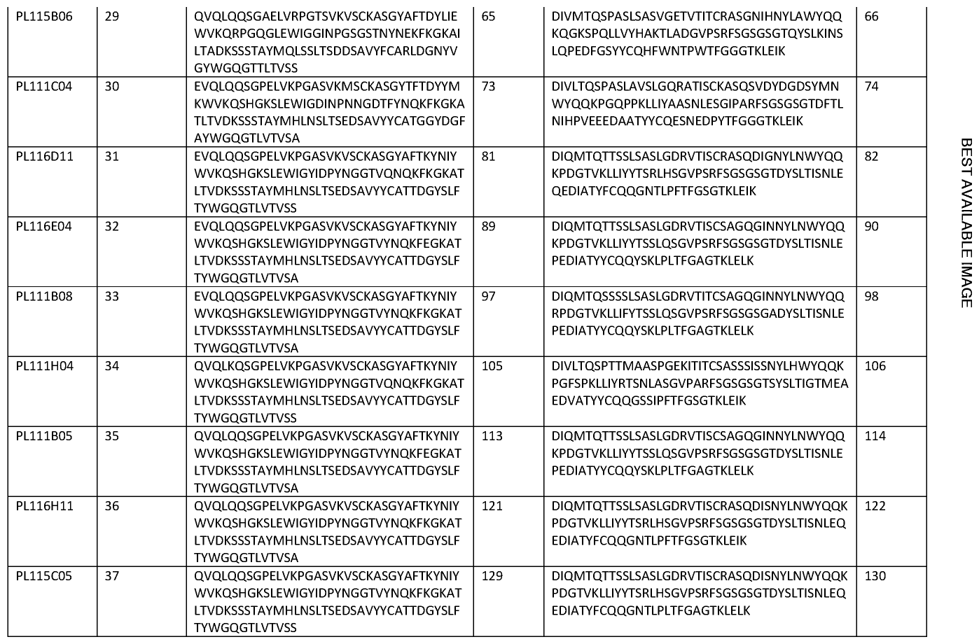

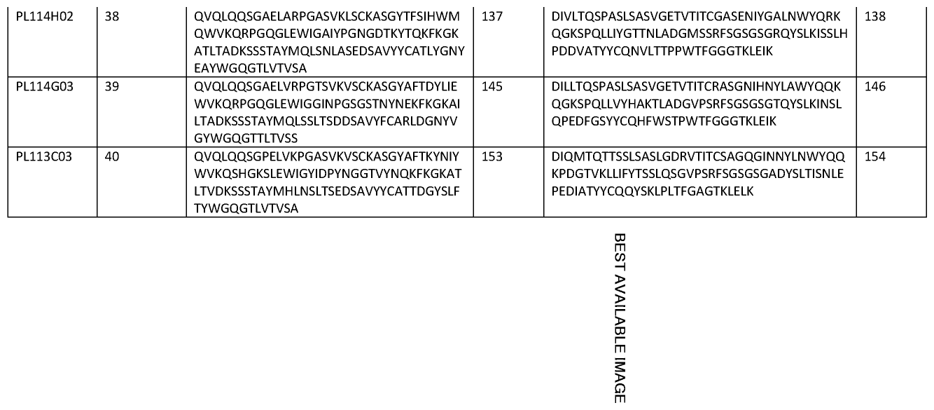

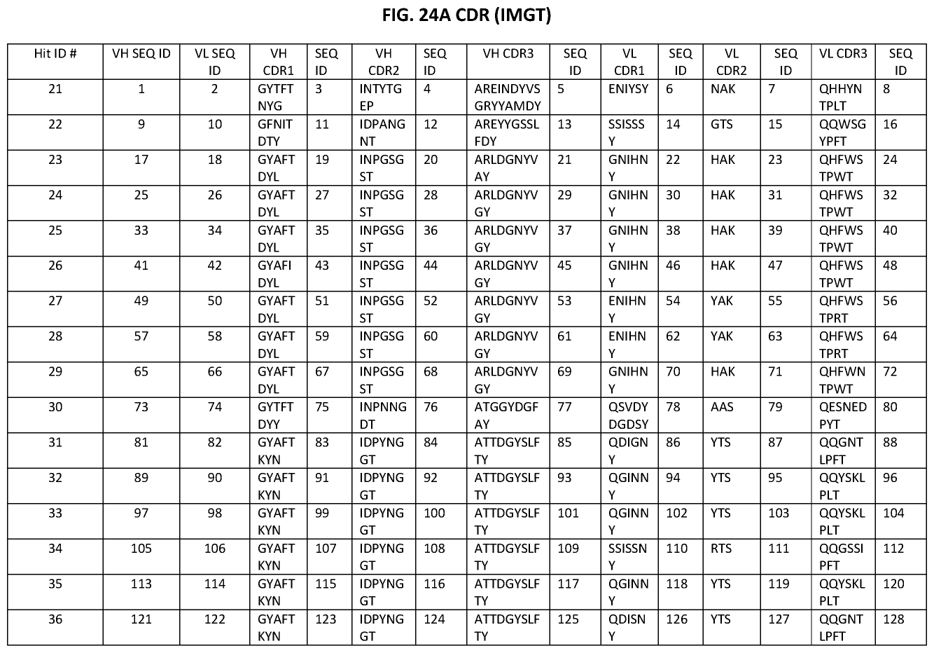

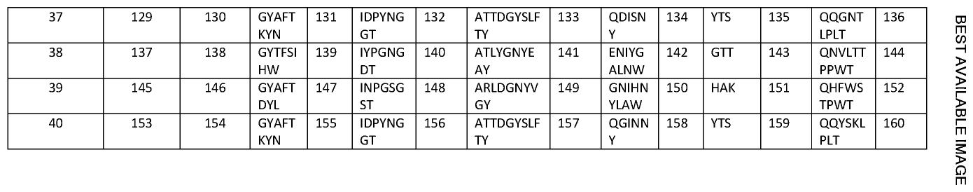

The disclosure provides antibodies and antigen-binding fragments thereof that specifically bind to MICA and/or MICB. The antibodies and antigen-binding fragments described herein are capable of binding to MICA and/or MICB polypeptides, and can bind and block the shedding of MICA/B polypeptides from the surface of cancer cells and potentiate NK and T cells. The disclosure provides e.g., anti-MICA/B antibodies PL114D01 (“#21”), PL111H05 (“#22”), PL114C07 (“#23”), PL114C04 (“#24”), PL116C11 (“#25”), PL115E08 (“#26”), PL116H02 (“#27”), PL114F03 (“#28”), PL115B06 (“#29”), PL111C04 (“#30”), PL116D11 (“#31”), PL116E04 (“32”), PL111B08 (“#33”), PL111H04 (“#34”), PL111B05 (“#35”), PL116H11 (“#36”), PL115C05 (“#37”), PL114H02 (“#38”), PL114G03 (“#39”), PL113C03 (“#40”), D2M001-001, D2M001-002, D2M001-003, D2M001-004, D2M001-005, D2M001-006, D2M001-007, D2M001-008, D2M001-009, D2M001-010, D2M001-011, D2M001-201, D2M001-202, D2M001-203, D2M001-204, D2M001-205, D2M001-206, D2M001-207, D2M001-208, D2M001-209, D2M001-210, D2M001-211, D2M001-212, D2M001-213, h36, h36B3, h36B3GS, h36B3(DE), h36B3(GA), h36D26, h36D28, h39F53, h39F53GS, h39-max-E3S-P3-P2 #1, h39-max-E3S-P3-P2 #4, h39-010F3S-P2-P2 #7, h39-010F3F53E1, h39-010F3F53E2(GS), h39-010F3S-P2-P2 #4, h39-010F3F53E2, D2M001-101, D2M001-102, D2M001-103, D2M001-104, D2M001-105, D2M001-106, D2M001-107, chimeric antibodies thereof, and humanized antibodies thereof. The CDR sequences, VH, and VL of these antibodies or antibodies derived therefrom are shown in FIGS. 23-30.

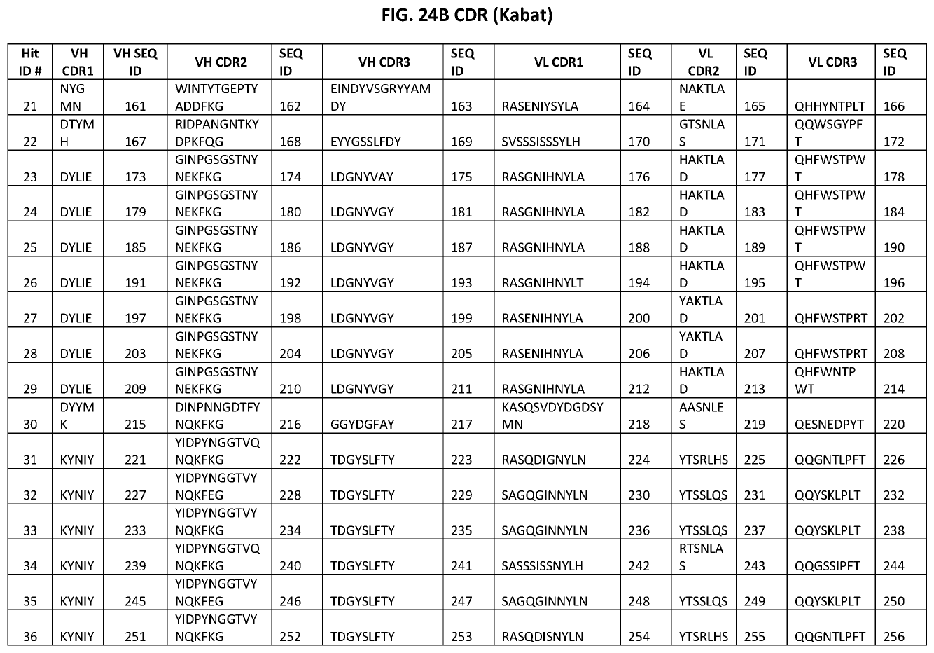

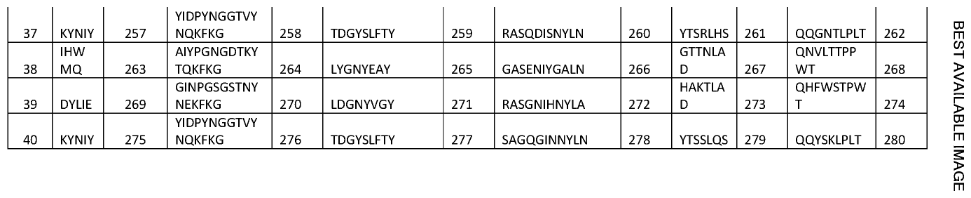

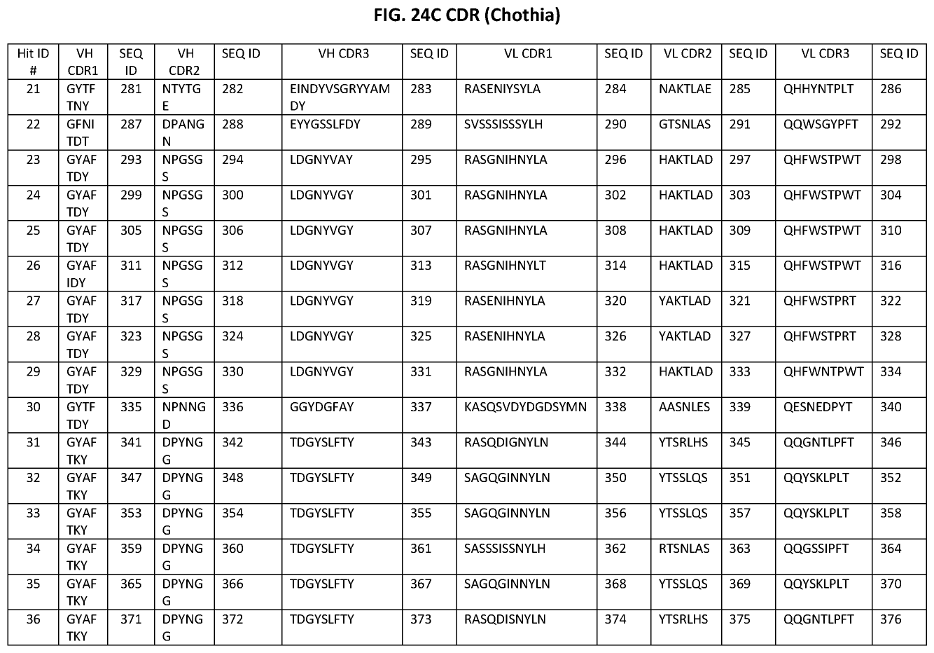

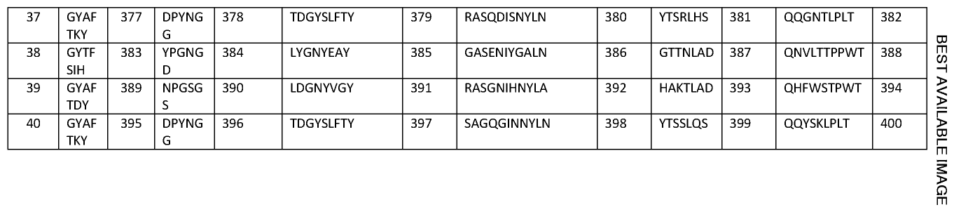

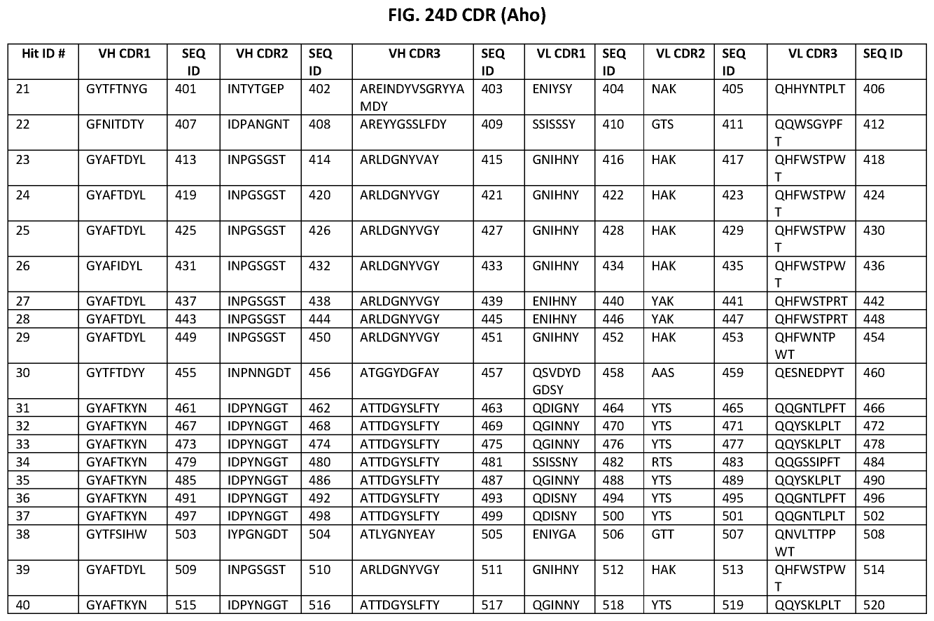

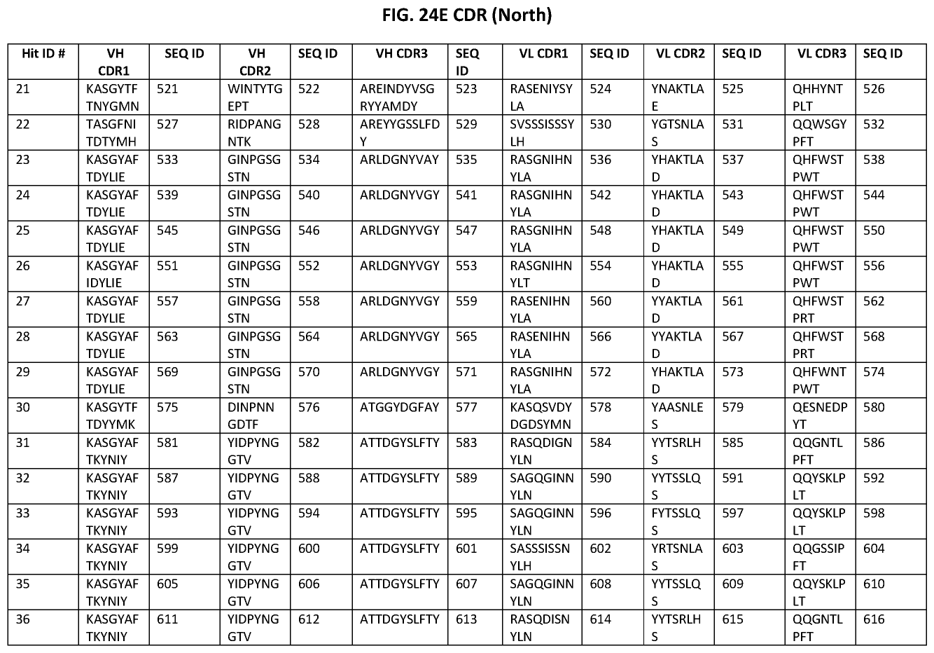

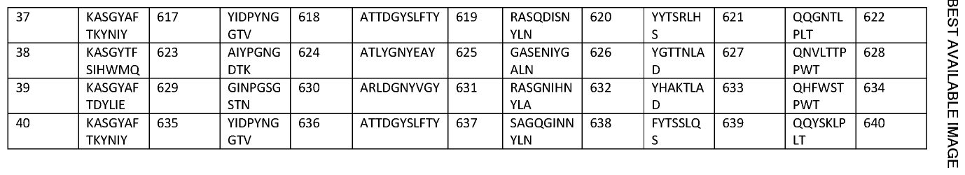

For example, the CDR sequences for “#21”, and “#21” derived antibodies (e.g., humanized antibodies) include CDRs of the heavy chain variable domain, SEQ ID NOs: 3, 4, 5, and CDRs of the light chain variable domain, SEQ ID NOs: 6, 7, 8, e.g., as defined by IMGT numbering. The CDR based on other definitions for these antibodies are shown in FIG. 24B-24E.

For example, the CDR sequences for “#22”, and “#22” derived antibodies (e.g., humanized antibodies) include CDRs of the heavy chain variable domain, SEQ ID NOs: 11, 12, 13, and CDRs of the light chain variable domain, SEQ ID NOs: 14, 15, 16, e.g., as defined by IMGT numbering. The CDR based on other definitions for these antibodies are shown in FIG. 24B-24E.

For example, the CDR sequences for “#23”, and “#23” derived antibodies (e.g., humanized antibodies) include CDRs of the heavy chain variable domain, SEQ ID NOs: 19, 20, 21, and CDRs of the light chain variable domain, SEQ ID NOs: 22, 23, 24, e.g., as defined by IMGT numbering. The CDR based on other definitions for these antibodies are shown in FIG. 24B-24E.

For example, the CDR sequences for “#24”, and “#24” derived antibodies (e.g., humanized antibodies) include CDRs of the heavy chain variable domain, SEQ ID NOs: 27, 28, 29, and CDRs of the light chain variable domain, SEQ ID NOs: 30, 31, 32, e.g., as defined by IMGT numbering. The CDR based on other definitions for these antibodies are shown in FIG. 24B-24E.

For example, the CDR sequences for “#25”, and “#25” derived antibodies (e.g., humanized antibodies) include CDRs of the heavy chain variable domain, SEQ ID NOs: 35, 36, 37, and CDRs of the light chain variable domain, SEQ ID NOs: 38, 39, 40, e.g., as defined by IMGT numbering. The CDR based on other definitions for these antibodies are shown in FIG. 24B-24E.

For example, the CDR sequences for “#26”, and “#26” derived antibodies (e.g., humanized antibodies) include CDRs of the heavy chain variable domain, SEQ ID NOs: 43, 44, 45, and CDRs of the light chain variable domain, SEQ ID NOs: 46, 47, 48, e.g., as defined by IMGT numbering. The CDR based on other definitions for these antibodies are shown in FIG. 24B-24E.

For example, the CDR sequences for “#27”, and “#27” derived antibodies (e.g., humanized antibodies) include CDRs of the heavy chain variable domain, SEQ ID NOs: 51, 52, 53, and CDRs of the light chain variable domain, SEQ ID NOs: 54, 55, 56, e.g., as defined by IMGT numbering. The CDR based on other definitions for these antibodies are shown in FIG. 24B-24E.

For example, the CDR sequences for “#28”, and “#28” derived antibodies (e.g., humanized antibodies) include CDRs of the heavy chain variable domain, SEQ ID NOs: 59, 60, 61, and CDRs of the light chain variable domain, SEQ ID NOs: 62, 63, 64, e.g., as defined by IMGT numbering. The CDR based on other definitions for these antibodies are shown in FIG. 24B-24E.

For example, the CDR sequences for “#29”, and “#29” derived antibodies (e.g., humanized antibodies) include CDRs of the heavy chain variable domain, SEQ ID NOs: 67, 68, 69, and CDRs of the light chain variable domain, SEQ ID NOs: 70, 71, 72, e.g., as defined by IMGT numbering. The CDR based on other definitions for these antibodies are shown in FIG. 24B-24E.

For example, the CDR sequences for “#30”, and “#30” derived antibodies (e.g., humanized antibodies) include CDRs of the heavy chain variable domain, SEQ ID NOs: 75, 76, 77, and CDRs of the light chain variable domain, SEQ ID NOs: 78, 79, 80, e.g., as defined by IMGT numbering. The CDR based on other definitions for these antibodies are shown in FIG. 24B-24E.

For example, the CDR sequences for “#31”, and “#31” derived antibodies (e.g., humanized antibodies) include CDRs of the heavy chain variable domain, SEQ ID NOs: 83, 84, 85, and CDRs of the light chain variable domain, SEQ ID NOs: 86, 87, 88, e.g., as defined by IMGT numbering. The CDR based on other definitions for these antibodies are shown in FIG. 24B-24E.

For example, the CDR sequences for “#32”, and “#32” derived antibodies (e.g., humanized antibodies) include CDRs of the heavy chain variable domain, SEQ ID NOs: 91, 92, 93, and CDRs of the light chain variable domain, SEQ ID NOs: 94, 95, 96, e.g., as defined by IMGT numbering. The CDR based on other definitions for these antibodies are shown in FIG. 24B-24E.

For example, the CDR sequences for “#33”, and “#33” derived antibodies (e.g., humanized antibodies) include CDRs of the heavy chain variable domain, SEQ ID NOs: 99, 100, 101, and CDRs of the light chain variable domain, SEQ ID NOs: 102, 103, 104, e.g., as defined by IMGT numbering. The CDR based on other definitions for these antibodies are shown in FIG. 24B-24E.

For example, the CDR sequences for “#34”, and “#34” derived antibodies (e.g., humanized antibodies) include CDRs of the heavy chain variable domain, SEQ ID NOs: 107, 108, 109, and CDRs of the light chain variable domain, SEQ ID NOs: 110, 111, 112, e.g., as defined by IMGT numbering. The CDR based on other definitions for these antibodies are shown in FIG. 24B-24E.

For example, the CDR sequences for “#35”, and “#35” derived antibodies (e.g., humanized antibodies) include CDRs of the heavy chain variable domain, SEQ ID NOs: 115, 116, 117, and CDRs of the light chain variable domain, SEQ ID NOs: 118, 119, 120, e.g., as defined by IMGT numbering. The CDR based on other definitions for these antibodies are shown in FIG. 24B-24E.

For example, the CDR sequences for “#36”, and “#36” derived antibodies (e.g., humanized antibodies) include CDRs of the heavy chain variable domain, SEQ ID NOs: 123, 124, 125, and CDRs of the light chain variable domain, SEQ ID NOs: 126, 127, 128, e.g., as defined by IMGT numbering. The CDR based on other definitions for these antibodies are shown in FIG. 24B-24E.

For example, the CDR sequences for “#37”, and “#37” derived antibodies (e.g., humanized antibodies) include CDRs of the heavy chain variable domain, SEQ ID NOs: 131, 132, 133, and CDRs of the light chain variable domain, SEQ ID NOs: 134, 135, 136, e.g., as defined by IMGT numbering. The CDR based on other definitions for these antibodies are shown in FIG. 24B-24E.

For example, the CDR sequences for “#38”, and “#38” derived antibodies (e.g., humanized antibodies) include CDRs of the heavy chain variable domain, SEQ ID NOs: 139, 140, 141, and CDRs of the light chain variable domain, SEQ ID NOs: 142, 143, 144, e.g., as defined by IMGT numbering. The CDR based on other definitions for these antibodies are shown in FIG. 24B-24E.

For example, the CDR sequences for “#39”, and “#39” derived antibodies (e.g., humanized antibodies) include CDRs of the heavy chain variable domain, SEQ ID NOs: 147, 148, 149, and CDRs of the light chain variable domain, SEQ ID NOs: 150, 151, 152, e.g., as defined by IMGT numbering. The CDR based on other definitions for these antibodies are shown in FIG. 24B-24E.

For example, the CDR sequences for “#40”, and “#40” derived antibodies (e.g., humanized antibodies) include CDRs of the heavy chain variable domain, SEQ ID NOs: 155, 156, 157, and CDRs of the light chain variable domain, SEQ ID NOs: 158, 159, 160, e.g., as defined by IMGT numbering. The CDR based on other definitions for these antibodies are shown in FIG. 24B-24E.

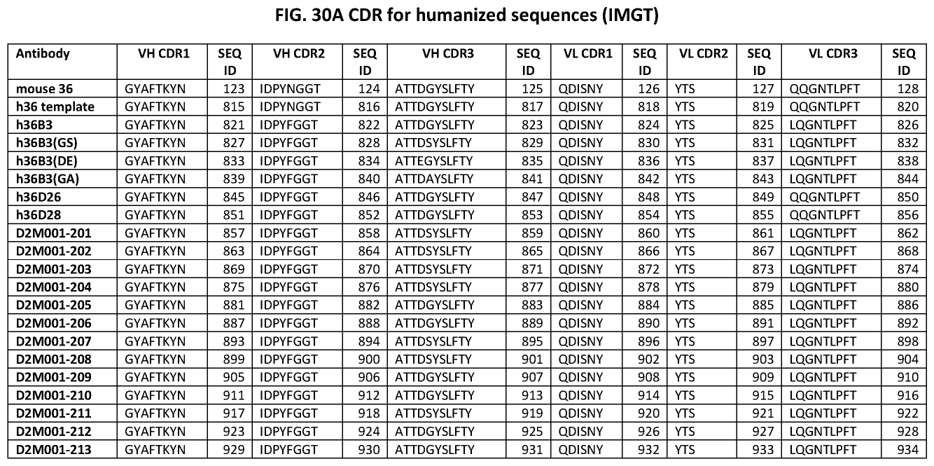

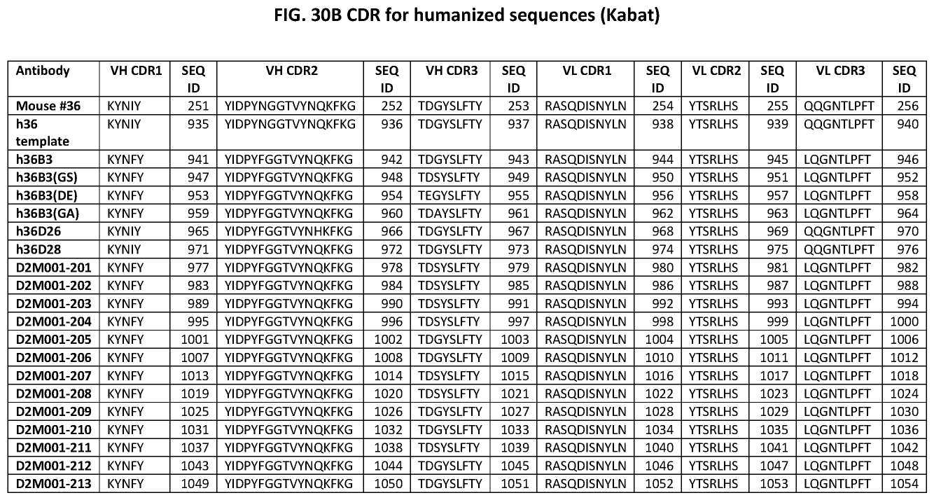

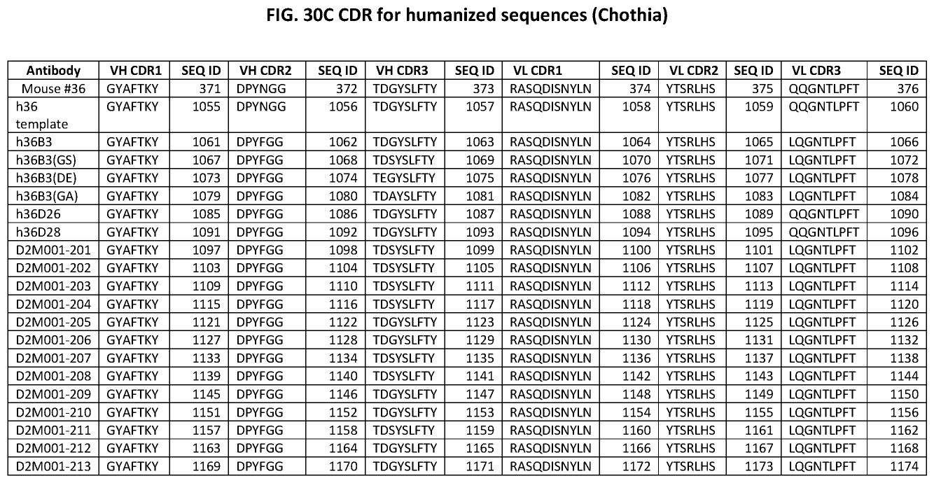

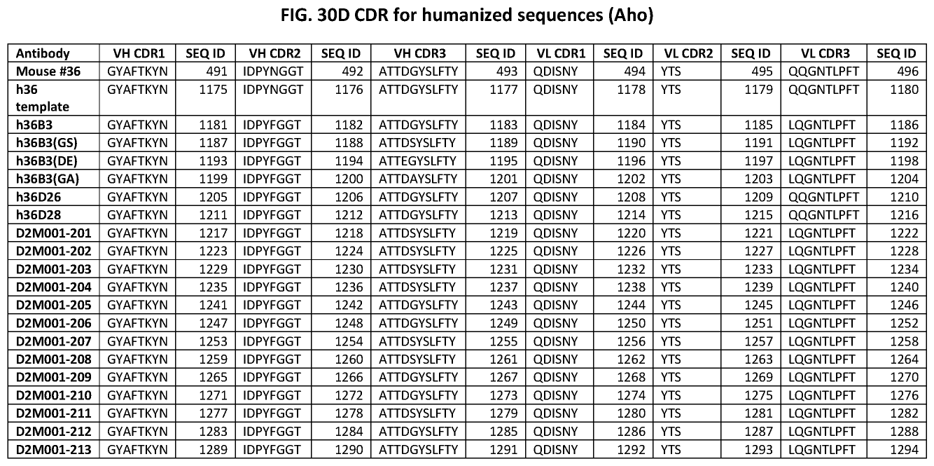

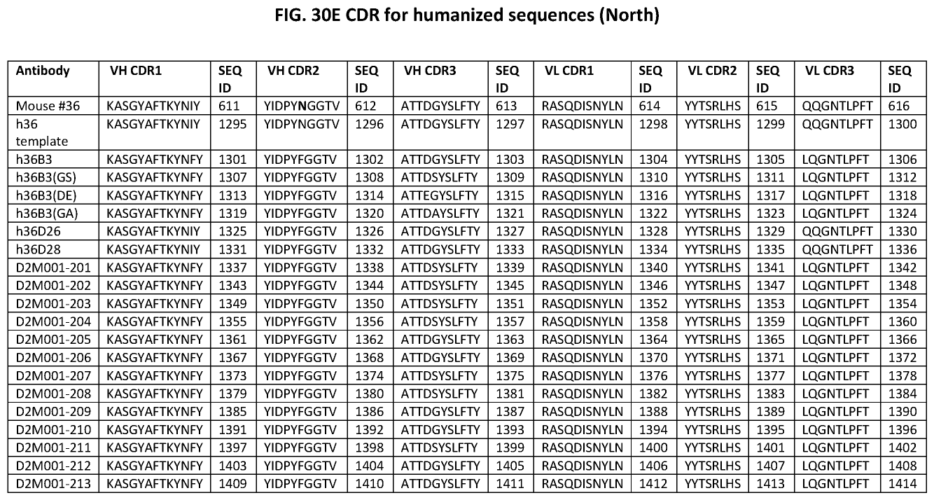

For example, the CDR sequences for “h36 template”, and “h36 template” derived antibodies (e.g., humanized antibodies) include CDRs of the heavy chain variable domain, SEQ ID NOs: 815, 816, 817, and CDRs of the light chain variable domain, SEQ ID NOs: 818, 819, 820, e.g., as defined by IMGT numbering. The CDR based on other definitions for these antibodies are shown in FIG. 30B-30E.

For example, the CDR sequences for “h36B3”, and “h36B3” derived antibodies (e.g., humanized antibodies) include CDRs of the heavy chain variable domain, SEQ ID NOs: 821, 822, 823, and CDRs of the light chain variable domain, SEQ ID NOs: 824, 825, 826, e.g., as defined by IMGT numbering. The CDR based on other definitions for these antibodies are shown in FIG. 30B-30E.

For example, the CDR sequences for “h36B3(GS)”, and “h36B3(GS)” derived antibodies (e.g., humanized antibodies) include CDRs of the heavy chain variable domain, SEQ ID NOs: 827, 828, 829, and CDRs of the light chain variable domain, SEQ ID NOs: 830, 831, 832, e.g., as defined by IMGT numbering. The CDR based on other definitions for these antibodies are shown in FIG. 30B-30E.

For example, the CDR sequences for “h36B3(DE)”, and “h36B3(DE)” derived antibodies (e.g., humanized antibodies) include CDRs of the heavy chain variable domain, SEQ ID NOs: 833, 834, 835, and CDRs of the light chain variable domain, SEQ ID NOs: 836, 837, 838, e.g., as defined by IMGT numbering. The CDR based on other definitions for these antibodies are shown in FIG. 30B-30E.

For example, the CDR sequences for “h36B3(GA)”, and “h36B3(GA)” derived antibodies (e.g., humanized antibodies) include CDRs of the heavy chain variable domain, SEQ ID NOs: 839, 840, 841, and CDRs of the light chain variable domain, SEQ ID NOs: 842, 843, 844, e.g., as defined by IMGT numbering. The CDR based on other definitions for these antibodies are shown in FIG. 30B-30E.

For example, the CDR sequences for “h36D26”, and “h36D26” derived antibodies (e.g., humanized antibodies) include CDRs of the heavy chain variable domain, SEQ ID NOs: 845, 846, 847, and CDRs of the light chain variable domain, SEQ ID NOs: 848, 849, 850, e.g., as defined by IMGT numbering. The CDR based on other definitions for these antibodies are shown in FIG. 30B-30E.

For example, the CDR sequences for “h36D28”, and “h36D28” derived antibodies (e.g., humanized antibodies) include CDRs of the heavy chain variable domain, SEQ ID NOs: 851, 852, 853, and CDRs of the light chain variable domain, SEQ ID NOs: 854, 855, 856, e.g., as defined by IMGT numbering. The CDR based on other definitions for these antibodies are shown in FIG. 30B-30E.

For example, the CDR sequences for “D2M001-201”, and “D2M001-201” derived antibodies (e.g., humanized antibodies) include CDRs of the heavy chain variable domain, SEQ ID NOs: 857, 858, 859, and CDRs of the light chain variable domain, SEQ ID NOs: 860, 861, 862, e.g., as defined by IMGT numbering. The CDR based on other definitions for these antibodies are shown in FIG. 30B-30E.

For example, the CDR sequences for “D2M001-202”, and “D2M001-202” derived antibodies (e.g., humanized antibodies) include CDRs of the heavy chain variable domain, SEQ ID NOs: 863, 864, 865, and CDRs of the light chain variable domain, SEQ ID NOs: 866, 867, 868, e.g., as defined by IMGT numbering. The CDR based on other definitions for these antibodies are shown in FIG. 30B-30E.

For example, the CDR sequences for “D2M001-203”, and “D2M001-203” derived antibodies (e.g., humanized antibodies) include CDRs of the heavy chain variable domain, SEQ ID NOs: 869, 870, 871, and CDRs of the light chain variable domain, SEQ ID NOs: 872, 873, 874, e.g., as defined by IMGT numbering. The CDR based on other definitions for these antibodies are shown in FIG. 30B-30E.

For example, the CDR sequences for “D2M001-204”, and “D2M001-204” derived antibodies (e.g., humanized antibodies) include CDRs of the heavy chain variable domain, SEQ ID NOs: 875, 876, 877, and CDRs of the light chain variable domain, SEQ ID NOs: 878, 879, 880, e.g., as defined by IMGT numbering. The CDR based on other definitions for these antibodies are shown in FIG. 30B-30E.

For example, the CDR sequences for “D2M001-205”, and “D2M001-205” derived antibodies (e.g., humanized antibodies) include CDRs of the heavy chain variable domain, SEQ ID NOs: 881, 882, 883, and CDRs of the light chain variable domain, SEQ ID NOs: 884, 885, 886, e.g., as defined by IMGT numbering. The CDR based on other definitions for these antibodies are shown in FIG. 30B-30E.

For example, the CDR sequences for “D2M001-206”, and “D2M001-206” derived antibodies (e.g., humanized antibodies) include CDRs of the heavy chain variable domain, SEQ ID NOs: 887, 888, 889, and CDRs of the light chain variable domain, SEQ ID NOs: 890, 891, 892, e.g., as defined by IMGT numbering. The CDR based on other definitions for these antibodies are shown in FIG. 30B-30E.

For example, the CDR sequences for “D2M001-207”, and “D2M001-207” derived antibodies (e.g., humanized antibodies) include CDRs of the heavy chain variable domain, SEQ ID NOs: 893, 894, 895, and CDRs of the light chain variable domain, SEQ ID NOs: 896, 897, 898, e.g., as defined by IMGT numbering. The CDR based on other definitions for these antibodies are shown in FIG. 30B-30E.

For example, the CDR sequences for “D2M001-208”, and “D2M001-208” derived antibodies (e.g., humanized antibodies) include CDRs of the heavy chain variable domain, SEQ ID NOs: 899, 900, 901, and CDRs of the light chain variable domain, SEQ ID NOs: 902, 903, 904, e.g., as defined by IMGT numbering. The CDR based on other definitions for these antibodies are shown in FIG. 30B-30E.

For example, the CDR sequences for “D2M001-209”, and “D2M001-209” derived antibodies (e.g., humanized antibodies) include CDRs of the heavy chain variable domain, SEQ ID NOs: 905, 906, 907, and CDRs of the light chain variable domain, SEQ ID NOs: 908, 909, 910, e.g., as defined by IMGT numbering. The CDR based on other definitions for these antibodies are shown in FIG. 30B-30E.

For example, the CDR sequences for “D2M001-210”, and “D2M001-210” derived antibodies (e.g., humanized antibodies) include CDRs of the heavy chain variable domain, SEQ ID NOs: 911, 912, 913, and CDRs of the light chain variable domain, SEQ ID NOs: 914, 915, 916, e.g., as defined by IMGT numbering. The CDR based on other definitions for these antibodies are shown in FIG. 30B-30E.

For example, the CDR sequences for “D2M001-211”, and “D2M001-211” derived antibodies (e.g., humanized antibodies) include CDRs of the heavy chain variable domain, SEQ ID NOs: 917, 918, 919, and CDRs of the light chain variable domain, SEQ ID NOs: 920, 921, 922, e.g., as defined by IMGT numbering. The CDR based on other definitions for these antibodies are shown in FIG. 30B-30E.

For example, the CDR sequences for “D2M001-212”, and “D2M001-212” derived antibodies (e.g., humanized antibodies) include CDRs of the heavy chain variable domain, SEQ ID NOs: 923, 924, 925, and CDRs of the light chain variable domain, SEQ ID NOs: 926, 927, 928, e.g., as defined by IMGT numbering. The CDR based on other definitions for these antibodies are shown in FIG. 30B-30E.

For example, the CDR sequences for “D2M001-213”, and “D2M001-213” derived antibodies (e.g., humanized antibodies) include CDRs of the heavy chain variable domain, SEQ ID NOs: 929, 930, 931, and CDRs of the light chain variable domain, SEQ ID NOs: 932, 933, 934, e.g., as defined by IMGT numbering. The CDR based on other definitions for these antibodies are shown in FIG. 30B-30E.

For example, the CDR sequences for “h36 template”, and “h36 template” derived antibodies (e.g., humanized antibodies) include CDRs of the heavy chain variable domain, SEQ ID NOs: 708, 709, 710, and CDRs of the light chain variable domain, SEQ ID NOs: 711, 712, 713.

For example, the CDR sequences for “h36B3”, and “h36B3” derived antibodies (e.g., humanized antibodies) include CDRs of the heavy chain variable domain, SEQ ID NOs: 714, 715, 716, and CDRs of the light chain variable domain, SEQ ID NOs: 717, 718, 719.

For example, the CDR sequences for “h36B3(GS)”, and “h36B3(GS)” derived antibodies (e.g., humanized antibodies) include CDRs of the heavy chain variable domain, SEQ ID NOs: 720, 721, 722, and CDRs of the light chain variable domain, SEQ ID NOs: 723, 724, 725.

For example, the CDR sequences for “h36B3(DE)”, and “h36B3(DE)” derived antibodies (e.g., humanized antibodies) include CDRs of the heavy chain variable domain, SEQ ID NOs: 726, 727, 728, and CDRs of the light chain variable domain, SEQ ID NOs: 729, 730, 731.

For example, the CDR sequences for “h36B3(GA)”, and “h36B3(GA)” derived antibodies (e.g., humanized antibodies) include CDRs of the heavy chain variable domain, SEQ ID NOs: 732, 733, 734, and CDRs of the light chain variable domain, SEQ ID NOs: 735, 736, 737.

For example, the CDR sequences for “h36D26”, and “h36D26” derived antibodies (e.g., humanized antibodies) include CDRs of the heavy chain variable domain, SEQ ID NOs: 738, 739, 740, and CDRs of the light chain variable domain, SEQ ID NOs: 741, 742, 743.

For example, the CDR sequences for “h36D28”, and “h36D28” derived antibodies (e.g., humanized antibodies) include CDRs of the heavy chain variable domain, SEQ ID NOs: 744, 745, 746, and CDRs of the light chain variable domain, SEQ ID NOs: 747, 748, 749.

For example, the CDR sequences for “D2M001-001”, and “D2M001-001” derived antibodies (e.g., humanized antibodies) include CDRs of the heavy chain variable domain, SEQ ID NOs: 750, 751, 752, and CDRs of the light chain variable domain, SEQ ID NOs: 753, 754, 755.

For example, the CDR sequences for “D2M001-003”, and “D2M001-003” derived antibodies (e.g., humanized antibodies) include CDRs of the heavy chain variable domain, SEQ ID NOs: 756, 757, 758, and CDRs of the light chain variable domain, SEQ ID NOs: 759, 760, 761.

For example, the CDR sequences for “D2M001-010”, and “D2M001-010” derived antibodies (e.g., humanized antibodies) include CDRs of the heavy chain variable domain, SEQ ID NOs: 762, 763, 764, and CDRs of the light chain variable domain, SEQ ID NOs: 765, 766, 767.

For example, the CDR sequences for “D2M001-004”, and “D2M001-004” derived antibodies (e.g., humanized antibodies) include CDRs of the heavy chain variable domain, SEQ ID NOs: 768, 769, 770, and CDRs of the light chain variable domain, SEQ ID NOs: 771, 772, 773.

For example, the CDR sequences for “hu39-max-E3S-P3-P2 #1”, and “hu39-max-E3S-P3-P2 #1” derived antibodies (e.g., humanized antibodies) include CDRs of the heavy chain variable domain, SEQ ID NOs: 774, 775, 776, and CDRs of the light chain variable domain, SEQ ID NOs: 777, 778, 779.

For example, the CDR sequences for “hu39-max-E3S-P3-P2 #4”, and “hu39-max-E3S-P3-P2 #4” derived antibodies (e.g., humanized antibodies) include CDRs of the heavy chain variable domain, SEQ ID NOs: 780, 781, 782, and CDRs of the light chain variable domain, SEQ ID NOs: 783, 784, 785.

For example, the CDR sequences for “h39-010F3S-P2-P2 #7”, and “h39-010F3S-P2-P2 #7” derived antibodies (e.g., humanized antibodies) include CDRs of the heavy chain variable domain, SEQ ID NOs: 786, 787, 788, and CDRs of the light chain variable domain, SEQ ID NOs: 789, 790, 791.

For example, the CDR sequences for “h39-010F3F53E1”, and “h39-010F3F53E1” derived antibodies (e.g., humanized antibodies) include CDRs of the heavy chain variable domain, SEQ ID NOs: 792, 793, 794, and CDRs of the light chain variable domain, SEQ ID NOs: 795, 796, 797.

For example, the CDR sequences for “h39-010F3F53E2(GS)”, and “h39-010F3F53E2(GS)” derived antibodies (e.g., humanized antibodies) include CDRs of the heavy chain variable domain, SEQ ID NOs: 798, 799, 800, and CDRs of the light chain variable domain, SEQ ID NOs: 801, 802, 803.

For example, the CDR sequences for “h39-010F3S-P2-P2 #4”, and “h39-010F3S-P2-P2 #4” derived antibodies (e.g., humanized antibodies) include CDRs of the heavy chain variable domain, SEQ ID NOs: 804, 805, 806, and CDRs of the light chain variable domain, SEQ ID NOs: 807, 808, 809.

For example, the CDR sequences for “h39-010F3F53E2”, and “h39-010F3F53E2” derived antibodies (e.g., humanized antibodies) include CDRs of the heavy chain variable domain, SEQ ID NOs: 810, 811, 812, and CDRS of the light chain variable domain, SEQ ID NOs: 813, 814, 815.

Thus, in one aspect, the disclosure provides an antibody or antigen-binding fragment thereof that binds to ICA and/or MICB comprising: a heavy chain variable region (VH) comprising complementarity determining regions (CDRs) 1, 2, and 3, wherein the VH CDR1 region comprises an amino acid sequence that is at least 70%, 80%, 90%, or 100% identical to a selected VH CDR1 amino acid sequence, the VH CDR2 region comprises an amino acid sequence that is at least 70%, 80%, 90%, or 100% identical to a selected VH CDR2 amino acid sequence, and the VH CDR3 region comprises an amino acid sequence that is at least 70%, 80%, 90%, or 100% identical to a selected VH CDR3 amino acid sequence; and a light chain variable region (VL) comprising CDRs 1, 2, and 3, wherein the VL CDR1 region comprises an amino acid sequence that is at least 70%, 80%, 90%, or 100% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 70%, 80%, 90%, or 100% identical to a selected VL CDR3 amino acid sequence, wherein the selected VH CDRs 1, 2, and 3 amino acid sequences and the selected VL CDRs, 1, 2, and 3 amino acid sequences are selected from VH CDRS 1, 2, 3 and VL CDRS 1, 2, 3 listed in FIGS. 24A, 24B, 24C, 24D, 24E, 28, 29, 30A, 30B, 30C, 30D, and 30E.

The amino acid sequence for heavy chain variable region and light variable region of anti-ICA/B antibodies are also provided. These VH and VL sequences are shown in FIG. 23, 25, 26, 27, 28, or 29.

For example, the VH and VL of Hit ID 21 are set forth in SEQ ID NO: 1 and 2. For example, the VH and VL of Hit ID 22 are set forth in SEQ ID NO: 9 and 10. For example, the VH and VL of Hit ID 23 are set forth in SEQ ID NO: 17 and 18. For example, the VH and VL of Hit ID 24 are set forth in SEQ ID NO: 25 and 26. For example, the VH and VL of Hit ID 25 are set forth in SEQ ID NO: 33 and 34. For example, the VH and VL of Hit ID 26 are set forth in SEQ ID NO: 41 and 42. For example, the VH and VL of Hit ID 27 are set forth in SEQ ID NO: 49 and 50. For example, the VH and VL of Hit ID 28 are set forth in SEQ ID NO: 57 and 58. For example, the VH and VL of Hit ID 29 are set forth in SEQ ID NO: 65 and 66. For example, the VH and VL of Hit ID 30 are set forth in SEQ ID NO: 73 and 74. For example, the VH and VL of Hit ID 31 are set forth in SEQ ID NO: 81 and 82. For example, the VH and VL of Hit ID 32 are set forth in SEQ ID NO: 89 and 90. For example, the VH and VL of Hit ID 33 are set forth in SEQ ID NO: 97 and 98. For example, the VH and VL of Hit ID 34 are set forth in SEQ ID NO: 105 and 106. For example, the VH and VL of Hit ID 35 are set forth in SEQ ID NO: 113 and 114. For example, the VH and VL of Hit ID 36 are set forth in SEQ ID NO: 121 and 122. For example, the VH and VL of Hit ID 37 are set forth in SEQ ID NO: 129 and 130. For example, the VH and VL of Hit ID 38 are set forth in SEQ ID NO: 137 and 138. For example, the VH and VL of Hit ID 39 are set forth in SEQ ID NO: 145 and 146. For example, the VH and VL of Hit ID 40 are set forth in SEQ ID NO: 153 and 154.

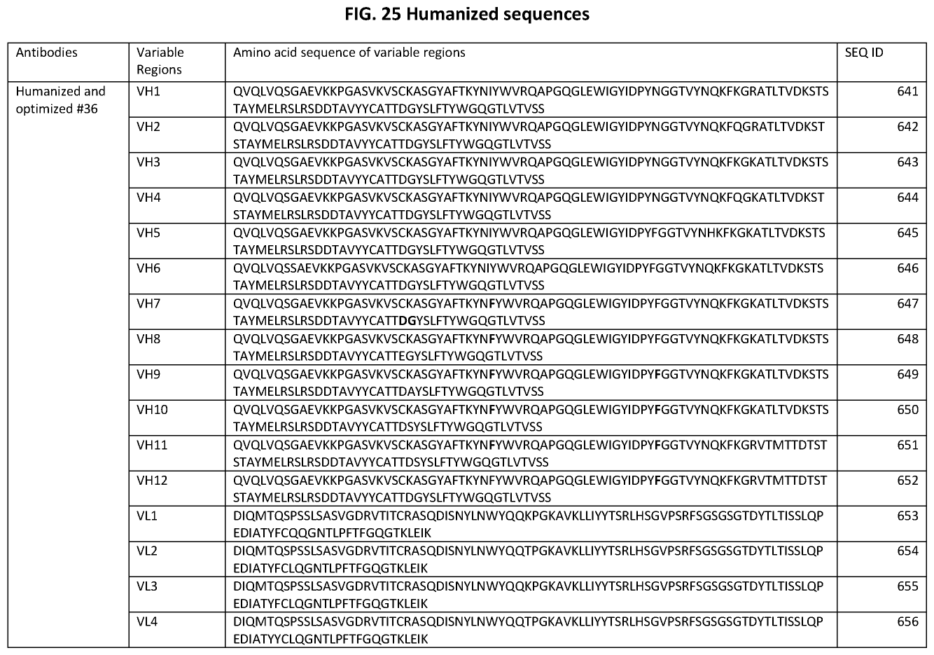

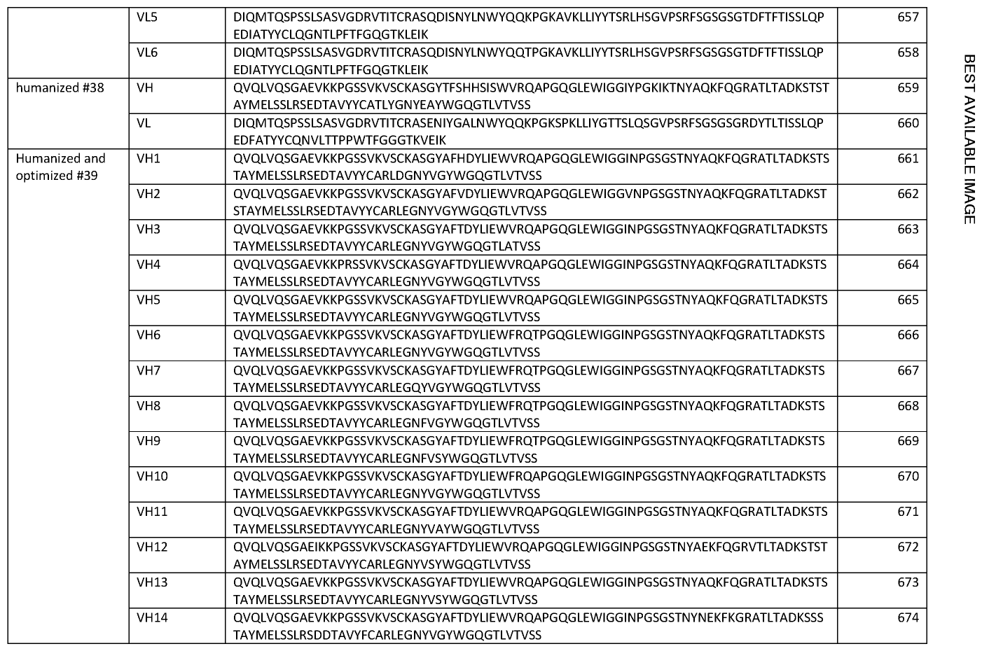

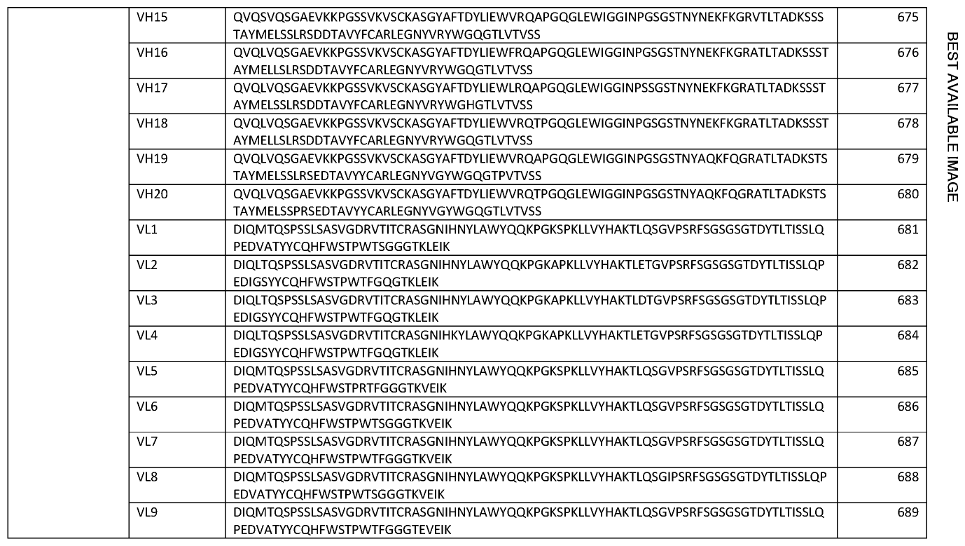

These antibodies can be humanized. The humanized VH and VL sequences are shown in in FIG. 25, 26, 27, 28, or 29.

With respect to humanized antibodies derived from #36, any of these heavy chain variable region sequences (e.g., SEQ ID NOs: 641, 642, 643, 644, 645, 646, 647, 648, 649, 650, 651, 652, 690, 692, 694, 696, 698, 700, 702, 704, or 706) can be paired with any of these light chain variable region sequences (e.g., SEQ ID NOs: 653, 654, 655, 656, 657, 658, 691, 693, 695, 697, 699, 701, 703, 705, or 707).

With respect to humanized antibodies derived from #39, any of these heavy chain variable region sequences (e.g., SEQ ID NOs: 661, 662, 663, 664, 665, 666, 667, 668, 669, 670, 671, 672, 673, 674, 675, 676, 677, 678, 679, or 680) can be paired with any of these light chain variable region sequences (e.g., SEQ ID NOs 681, 682, 683, 684, 685, 686, 687, 688, or 689).

In some embodiments, humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, Tim D., et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects.

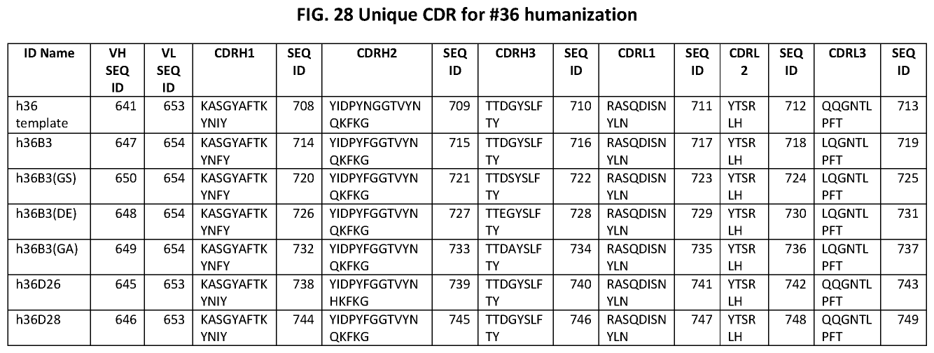

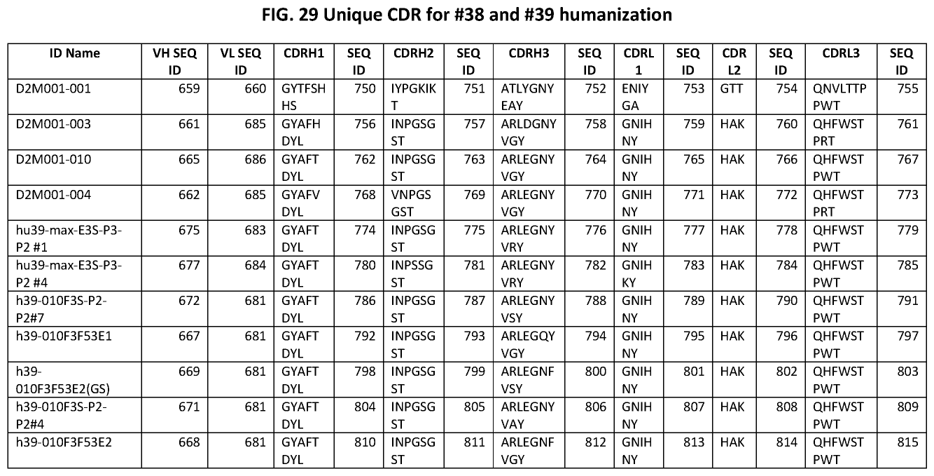

Furthermore, in some embodiments, the antibodies or antigen-binding fragments thereof described herein can also contain one, two, or three heavy chain variable region CDRs selected from FIGS. 24A-24E, 28, 29, FIGS. 30A-30E; and/or one, two, or three light chain variable region CDRs selected from FIGS. 24A-24E, 28, 29, FIGS. 30A-30E. In some embodiments, the antibodies or antigen-binding fragments thereof described herein can also contain one, two, or three heavy chain variable region CDRs and/or one, two, or three light chain variable region CDRs as shown in FIGS. 24A and 30A, under IMGT numbering scheme. In some embodiments, the antibodies or antigen-binding fragments thereof described herein can also contain one, two, or three heavy chain variable region CDRs and/or one, two, or three light chain variable region CDRs as shown in FIGS. 24B and 30B, under Kabat numbering scheme. In some embodiments, the antibodies or antigen-binding fragments thereof described herein can also contain one, two, or three heavy chain variable region CDRs and/or one, two, or three light chain variable region CDRs as shown in FIGS. 24C and 30C, under Chothia numbering scheme. In some embodiments, the antibodies or antigen-binding fragments thereof described herein can also contain one, two, or three heavy chain variable region CDRs and/or one, two, or three light chain variable region CDRs as shown in FIGS. 24D and 30D, under Aho numbering scheme. In some embodiments, the antibodies or antigen-binding fragments thereof described herein can also contain one, two, or three heavy chain variable region CDRs and/or one, two, or three light chain variable region CDRs as shown in FIGS. 24E and 30E, under North numbering scheme. In some embodiments, the antibodies or antigen-binding fragments thereof described herein can also contain one, two, or three heavy chain variable region CDRs and/or one, two, or three light chain variable region CDRs as shown in FIGS. 28 and 29, under IMGT or a combination numbering scheme, e.g., based on the predicted function of the sequences.

In some embodiments, the antibody or an antigen-binding fragment described herein can contain a heavy chain variable domain containing one, two, or three of any one of the VH CDR1 shown in FIGS. 24A-24E, 28, 29, FIGS. 30A-30E with zero, one or two amino acid insertions, deletions, or substitutions; any one of the VH CDR2 shown in FIGS. 24A-24E, 28, 29, FIGS. 30A-30E with zero, one or two amino acid insertions, deletions, or substitutions; any one of the VH CDR3 shown in FIGS. 24A-24E, 28, 29, FIGS. 30A-30E with zero, one or two amino acid insertions, deletions, or substitutions.

In some embodiments, the antibody or an antigen-binding fragment described herein can contain a light chain variable domain containing one, two, or three of any one of the VL CDR1 shown in FIGS. 24A-24E, 28, 29, FIGS. 30A-30E with zero, one or two amino acid insertions, deletions, or substitutions; any one of the VL CDR2 shown in FIGS. 24A-24E, 28, 29, FIGS. 30A-30E with zero, one or two amino acid insertions, deletions, or substitutions; any one of the VL CDR3 shown in FIGS. 24A-24E, 28, 29, FIGS. 30A-30E with zero, one or two amino acid insertions, deletions, or substitutions.

The insertions, deletions, and substitutions can be within the CDR sequence, or at one or both terminal ends of the CDR sequence.

The disclosure also provides antibodies or antigen-binding fragments thereof that bind to MICA and/or MICB. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 1, 9, 17, 25, 33, 41, 49, 57, 65, 73, 81, 89, 97, 105, 113, 121, 129, 137, 145, 153, 641, 642, 643, 644, 645, 646, 647, 648, 649, 650, 651, 652, 690, 692, 694, 696, 698, 700, 702, 704, 706, 659, 661, 662, 663, 664, 665, 666, 667, 668, 669, 670, 671, 672, 673, 674, 675, 676, 677, 678, 679, or 680. In some embodiments, the selected VL sequence is SEQ ID NO: 2, 10, 18, 26, 34, 42, 50, 58, 66, 74, 82, 90, 98, 106, 114, 122, 130, 138, 146, 154, 653, 654, 655, 656, 657, 658, 691, 693, 695, 697, 699, 701, 703, 705, 707, 660, 681, 682, 683, 684, 685, 686, 687, 688, or 689.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin heavy chain. The immunoglobulin heavy chain or immunoglobulin light chain comprises CDRs (under Kabat, Chothia, IMGT, North, or Aho numbering) as shown in FIGS. 24A-24E, 28, 29, FIGS. 30A-30E. When the polypeptides are paired with corresponding polypeptide (e.g., a corresponding heavy chain variable region or a corresponding light chain variable region), the paired polypeptides bind to MICA and/or MICB.

The anti-MICA/B antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to MICA and/or MICB will retain an ability to bind to MICA and/or MICB. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG₁ molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94: 7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

### Antibodies and Antigen Binding Fragments

The present disclosure provides anti-MICA/B antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132: 211-250; Martin et al., Methods Enzymol. 203:121-53 (1991); Morea et al., Biophys Chem. 68(1-3):9-16 (October 1997); Morea et al., J Mol Biol. 275(2):269-94 (January 1998); Chothia et al., Nature 342(6252):877-83 (December 1989); Ponomarenko and Bourne, BMC Structural Biology 7:64 (2007); each of which is incorporated herein by reference in its entirety. Other definitions are also known in the art, including e.g., IMGT, Aho (Honneger's Numbering Scheme) and North. In some embodiments, a combination of CDR definitions is used.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)₂, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain.

### Antibody Characteristics

The antibodies or antigen-binding fragments thereof described herein can block the release of MICA and/or MICB polypeptides from the surface of cancer cells and promote the potentiation of NK and T cells.

MICA and MICB are ligands of NKG2D and can be expressed in response to cellular stress including DNA damage, unfolded protein response, hypoxia, and carcinogenesis. Expression of MICA/B on the surfaces of cancer cells can tag them for NK-cell-mediated destruction. However, MICA/B proteins can be downregulated by tumor cells or proteolytically shed from the surface of tumor cells, causing effective escape from NKG2D recognition and subsequent progression of cancer proliferation. In some embodiments, by binding to MICA and/or MICB, the antibodies described herein can block the proteolytic shedding of MICA/MICB polypeptides from cancer cells, restoring the potentiation of the NKG2D recognition and NK-cell response.

In some embodiments, the antibodies or antigen binding fragments thereof can bind to alpha-1, alpha-2, and/or alpha-3 domains. In some embodiments, the antibodies or antigen binding fragments thereof can bind to the alpha-3 domain.

In some embodiments, the antibodies or antigen-binding fragments thereof as described herein can reduce the shedding of MICA/B polypeptides from the surfaces of cancer cells to less than 90%, 80%, 70%, 60%, 50%, 40%, 30%, 20%, 10%, 9%, 8%, 7%, 6%, 5%, 4%, 3%, 2%, or 1%. In some embodiments, the antibodies or antigen-binding fragments thereof as described herein can stabilized MICA/B on cell surface by preventing shedding. In some embodiments, the antibodies or antigen-binding fragments thereof as described herein can reduce the abundance of soluble MICA/B polypeptides in serum to less than 90%, 80%, 70%, 60%, 50%, 40%, 30%, 20%, 10%, 9%, 8%, 7%, 6%, 5%, 4%, 3%, 2%, or 1%. In some embodiments, the antibodies or antigen-binding fragments thereof as described herein can reduce the abundance of soluble MICA/B polypeptides in the cell culture medium of cancer cells grown in culture to less than 90%, 80%, 70%, 60%, 50%, 40%, 30%, 20%, 10%, 9%, 8%, 7%, 6%, %, 4%, 3%, 2%, or 1%. In some embodiments, the antibodies or antigen-binding fragments thereof as described herein can efficiently capture shed MICA/B to form immunocomplex, thereby stimulating NKG2D and co-activating NK cells and/or T cells. In some embodiments, the antibodies or antigen-binding fragments thereof as described herein can potentiate NK cells to kill tumor cells. In some embodiments, the antibodies or antigen-binding fragments thereof as described herein can mediate effector functions (e.g., ADCC) to kill MICA/B-expressing tumor cells. In some embodiments, the antibodies or antigen-binding fragments thereof as described herein can reduce the growth of tumors or tumor metastasis to less than 90%, 80%, 70%, 60%, 50%, 40%, 30%, 20%, 10%, 9%, 8%, 7%, 6%, 5%, 4%, 3%, 2%, or 1%.

In some implementations, the antibody (or antigen-binding fragments thereof) specifically binds to MICA (e.g., human MICA, monkey MICA, and/or chimeric MICA) or MICB (e.g., human MICB, monkey MICB, and/or chimeric MICB) with a dissociation rate (koff) of less than 0.1 s⁻¹, less than 0.01 s⁻¹, less than 0.001 s⁻¹, less than 0.0001 s⁻¹, or less than 0.0001 s⁻¹. In some embodiments, the dissociation rate (koff) is greater than 0.01 s⁻¹, greater than 0.001 s⁻¹, greater than 0.0001 s⁻¹, greater than 0.0001 s⁻¹, or greater than 0.00001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, or less than 1×10⁻¹⁰ M. In some embodiments, the KD is less than 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻⁴ M, or greater than 1×10⁻¹² M.

General techniques for measuring the affinity of an antibody for an antigen include, e.g., ELISA, RIA, and surface plasmon resonance (SPR). In some embodiments, the measurement is conducted using Carterra® SPR imaging system or Gator Prime BLI system. In some embodiments, the antibody binds to human MICA, monkey MICA (e.g., cynomolgus MICA), and/or chimeric MICA. In some embodiments, the antibody binds to human MICB, monkey MICB (e.g., cynomolgus MICB), and/or chimeric MICB.

In some embodiments, the antibodies described herein can bind to human or monkey MICA or MICB with an EC50 value of less than 10 μg/mL, less than 9 μg/mL, less than 8 μg/mL, less than 7 μg/mL, less than 6 μg/mL, less than 5 μg/mL, less than 4 μg/mL, less than 3 μg/mL, less than 2 μg/mL, less than 1 μg/mL, less than 0.9 μg/mL, less than 0.8 μg/mL, less than 0.7 μg/mL, less than 0.6 μg/mL, less than 0.5 μg/mL, less than 0.4 μg/mL, less than 0.3 μg/mL, less than 0.2 μg/mL, less than 0.1 μg/mL, less than 0.09 μg/mL, less than 0.08 μg/mL, less than 0.07 μg/mL, less than 0.06 μg/mL, less than 0.05 μg/mL, less than 0.04 μg/mL, less than 0.03 μg/mL, less than 0.02 μg/mL, or less than 0.01 μg/mL. In some embodiments, EC50 is less than 0.01 μg/mL. In some embodiments, EC50 is less than 0.02 μg/mL. In some embodiments, EC50 is less than 0.03 μg/mL. In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. As IgG can be described as a multi-domain protein, the melting curve sometimes shows two transitions, with a first denaturation temperature, Tm D1, and a second denaturation temperature Tm D2. The presence of these two peaks often indicates the denaturation of the Fc domains (Tm D1) and Fab domains (Tm D2), respectively. When there are two peaks, Tm usually refers to Tm D2.

Thus, in some embodiments, the antibodies or antigen binding fragments as described herein has a Tm D1 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the antibodies or antigen binding fragments as described herein has a Tm D2 greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, Tm, Tm D1, Tm D2 are less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( T i - T 0 ) / ( Vi - V 0 ) ] \times 100$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the antibody or antigen-binding fragment thereof described herein has a functional Fc. In some embodiments, the Fc is from human IgG1, human IgG2, human IgG3, or human IgG4. In some embodiments, effector function of a functional Fc is antibody-dependent cell-mediated cytotoxicity (ADCC). In some embodiments, effector function of a functional Fc is phagocytosis (e.g., antibody-dependent cellular phagocytosis, or ADCP). In some embodiments, effector function of a functional Fc is ADCC and phagocytosis. In some embodiments, the antibody or antigen-binding fragment thereof as described herein have an Fc region without effector function. In some embodiments, the Fc is a human IgG4 Fc. In some embodiments, the Fc is a human IgG1 Fc. In some embodiments, the Fc does not have a functional Fc region. For example, the Fc region has LALA mutations (L234A and L235A mutations in EU numbering), LALAPA mutations (L234A, L235A, P329A mutations in EU numbering), or LALAPG mutations (L234A, L235A, P329G mutations in EU numbering). In some embodiments, the Fc region includes an amino acid sequence that is at least 80%, at least 85%, at least 90%, at least 95%, or 100% identical to SEQ ID NO: 801, 802, 803, 804, or 805. For example, the antibodies or antigen binding fragments are Fab, Fab′, F(ab′)₂, and Fv fragments.

In some embodiments, the anti-MICA/B antibodies described herein can only bind to MICA, therefore can also be named as an anti-MICA antibody. In some embodiments, the anti-MICA/B antibodies described herein can only bind to MICB, therefore can also be named as an anti-MICB antibody.

In some embodiments, the anti-MICA/B antibodies described herein can inhibit the shedding of MICA/B polypeptides from the surfaces of tumor cells. As a result, the NKG2D-dependent activation of NK cells can be increased by at least 10%, at least 20%, at least 30%, at least 40%, at least 50%, at least 60%, at least 70%, at least 80%, at least 90%, or at least 100% as compared to that of negative control cells.

In some embodiments, the anti-MICA/B antibodies described herein can increase anti-tumor activity (e.g., destruction of tumor cells by NK cells or T cells) in cancer patients after treatment with a therapeutically effective amount of the anti-MICA/B antibodies by at least 10%, at least 20%, at least 30%, at least 40%, at least 50%, at least 60%, at least 70%, at least 80%, at least 90%, or at least 100% as compared to no treatment with the anti-MICA/B antibodies.

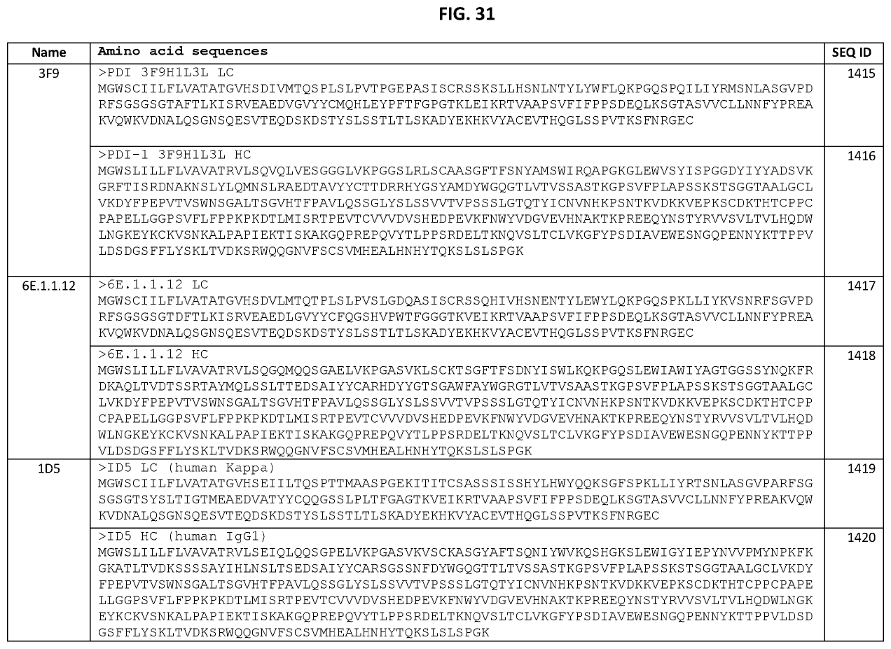

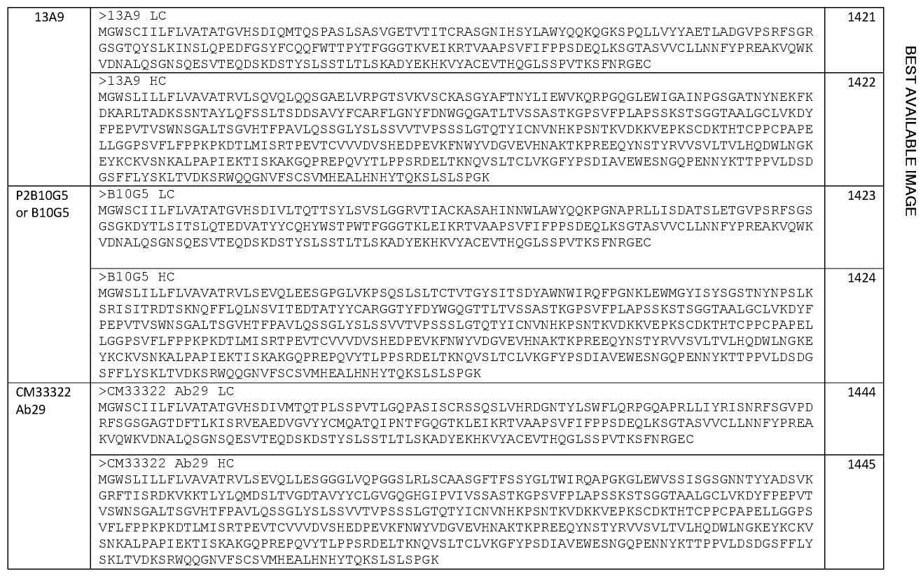

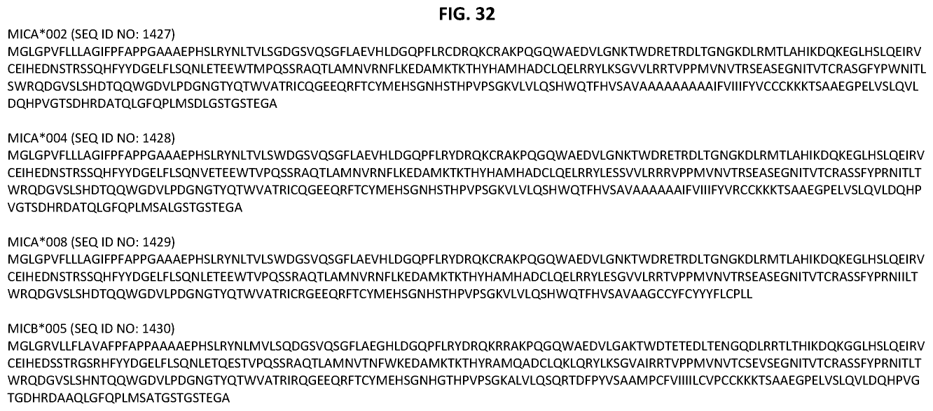

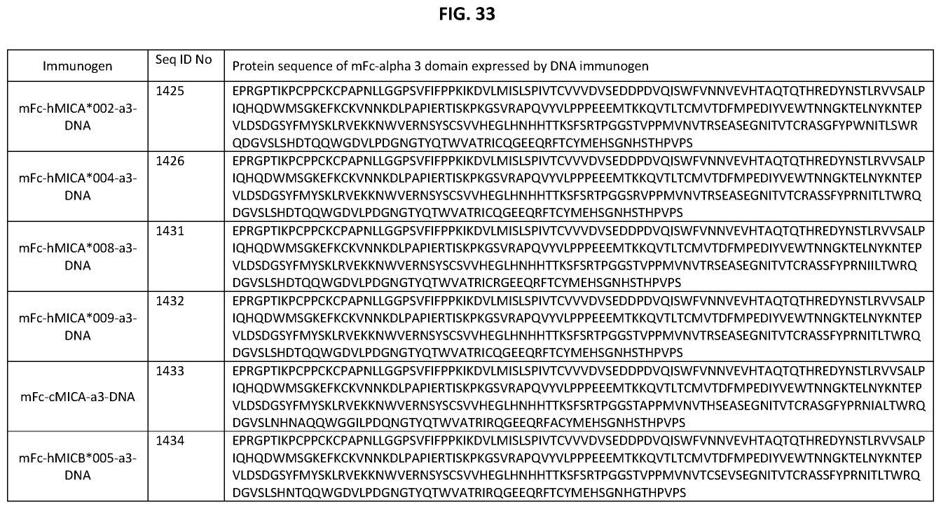

In some embodiments, the anti-MICA/B antibodies described herein are more effective (e.g., at least 10%, 20%, 30%, 40%, 50%, 60%, 70%, 80%, 90%, 1 fold, 2 folds, 5 folds, or 10 folds more effective) than a reference antibodies. Some of the reference antibodies (e.g., 3F9, 6E.1.1.12, ID5, 13A9, P2B10G5, or CM33322) are shown in FIG. 31.

### Methods of Making Anti-MICA/B Antibodies

An isolated fragment of human MICA and/or MICB can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of MICA and/or MICB, and encompasses an epitope of the protein such that an antibody raised against the peptide forms a specific immune complex with the protein. As described above, the full length sequence of human MICA is known in the art (e.g., SEQ ID NO: 1427, 1428, or 1429), and the full length sequence of human MICB is known in the art (e.g., SEQ ID NO: 11430).

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human MICA and/or MICB). The preparation can further include an adjuvant, such as Freund's complete or incomplete adjuvant, or a similar immunostimulatory agent.

Polyclonal antibodies can be prepared as described above by immunizing a suitable subject with MICA and/or MICB polypeptides, or antigenic peptides thereof (e.g., part of MICA and/or MICB) as immunogens. The antibody titer in the immunized subject can be monitored over time by standard techniques, such as with an enzyme-linked immunosorbent assay (ELISA) using the immobilized MICA and/or MICB polypeptides or peptides. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target proteins, e.g., MICA and/or MICB. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

In some embodiments, yeast display is performed to achieve affinity maturation. Details can be found, e.g., in Boder, E. T., et al. “Yeast surface display for screening combinatorial polypeptide libraries.” Nature Biotechnology 15.6 (1997): 553-557; Feldhaus, M. J., et al. “Flow-cytometric isolation of human antibodies from a nonimmune Saccharomyces cerevisiae surface display library.” Nature Biotechnology 21.2 (2003): 163-170; and Chao, G., et al. “Isolating and engineering human antibodies using yeast surface display.” Nature Protocols 1.2 (2006): 755-768; each of which is incorporated herein by reference in its entirety.

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

In some embodiments, humanization of the anti-MICA/B antibodies or antigen-binding fragments thereof described herein is achieved in silicon, e.g., using MOE computer software.

Ordinarily, amino acid sequence variants of the human, humanized, or chimeric anti-MICA/B antibody will contain an amino acid sequence having at least 75%, 80%, 85%, 90%, 95%, 96%, 97%, 98%, or 99% percent identity with a sequence present in the light or heavy chain of the original antibody.

Identity or homology with respect to an original sequence is usually the percentage of amino acid residues present within the candidate sequence that are identical with a sequence present within the human, humanized, or chimeric anti-MICA/B antibody or fragment, after aligning the sequences and introducing gaps, if necessary, to achieve the maximum percent sequence identity, and not considering any conservative substitutions as part of the sequence identity.

Additional modifications to the anti-MICA/B antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-MICA/B antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153: 516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antibody or antigen-binding fragments thereof of the present disclosure can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor or hematological cancer. In some embodiments, the cancer is squamous cell carcinoma of the head and neck (SCCHN), renal cell carcinoma (RCC), triple-negative breast cancer (TNBC), or colorectal carcinoma. In some embodiments, the subject has melanoma, neuroblastoma, prostate cancer, kidney cancer, multiple myeloma, or chronic lymphocytic leukemia.

In some embodiments, the cancer is glioma, thyroid cancer, lung cancer, colorectal cancer, head and neck cancer, stomach cancer, liver cancer, pancreatic cancer, renal cancer, urothelial cancer, prostate cancer, testis cancer, breast cancer, cervical cancer, endometrial cancer, ovarian cancer, or melanoma.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-PD1 antibody, an anti-PD-L1 antibody, an anti-LAG-3 antibody, an anti-TIGIT antibody, an anti-BTLA antibody, an anti-CTLA-4 antibody, or an anti-GITR antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells ) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1: Generation of Anti-MICA/B Antibodies

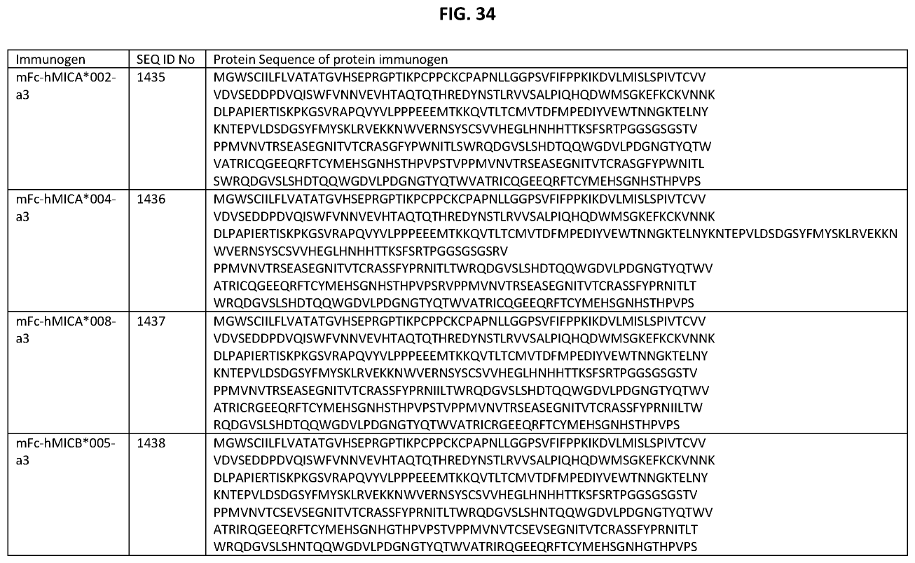

This example describes the strategies that were used to generate anti-MICA/B antibodies. A panel of antibodies that selectively bind human and cynomolgus (cyno) monkey MICA/B antigens were generated in BALB/c mice using two immunization strategies. The selection of DNA or protein antigens maximized the coverage in human population due to genetic polymorphisms in the human population (Tables 1 and 2). In these experiments, DNA immunization was followed by protein immunization. For DNA immunization, pools of DNA encoding MICA/B alpha 3 domains of human MICA*002, MICA*004, MICA*008, MICA*009 and MICB*05 as well as cMICA (cynoMICA, Cynomolgus monkeys (Macaca fascicularis) MICA fused to an Fc region of mouse IgG2a (exact sequences disclosed in FIG. 33) as immunogen were injected into mice using the Helios gene gun system (Bio-rad). This step was followed by injection of a pool of human MICA-ECD fused to human Fc (Sino Biologicals, Cat #: 12302-H02H), human MICA-ECD-HIS (Sino Biologicals, Cat #: HPLC-12302-H08H), human MICB-ECD-HIS (Sino Biologicals, Cat #: 10759-H08H), and recombinant human MICA/B-alpha3 (human MICA*002, 004, 008 and MICB*05) fused to mouse Fc protein (sequences disclosed in FIG. 34). Protein immunogens were resuspended with RIBI adjuvant in PBS and injected intraperitoneally.

TABLE 1

| | | Identical in | | | |
|---|---|---|---|---|---|
| | Allele | a3 domain of | | | Adjusted |
| | Frequency in | antigen used | Population | | Population |
| MICA | population | (yes = 1; | Coverage | | Coverage |
| Alleles | (%) | No = 0) | (%) | Adjust | (%) |
| *008 | 42.3 | 1 | 42.3 | 1 | 42.3 |
| *002 | 11.7 | 1 | 11.7 | 1 | 11.7 |
| *009 | 8.8 | 1 | 8.8 | 1 | 8.8 |
| *010 | 7.7 | 0 | 0 | 1^{a} | 7.7 |
| *004 | 6.5 | 1 | 6.5 | 1 | 6.5 |
| *007 | 4.7 | 1 | 4.7 | 1 | 4.7 |
| *018 | 3.7 | 1 | 3.7 | 1 | 3.7 |
| *017 | 3.4 | 1 | 3.4 | 1 | 3.4 |
| *027 | 2.6 | 1 | 2.6 | 1 | 2.6 |
| *012 | 2.3 | 1 | 2.3 | 1 | 2.3 |
| *016 | 1.9 | 0 | 0 | 0 | 0 |
| *011 | 1.7 | 1 | 1.7 | 1 | 1.7 |
| *019 | 0.8 | 0 | 0 | 1 | 0.8 |
| *001 | 0.7 | 1 | 0.7 | 1 | 0.7 |
| *006 | 0.5 | 0 | 0 | 0 | 0 |
| Total | 99.3 | | 88.4 | | 96.9 |

^{a}the polymorphic residuals present in MICA*002, MICA*004, MICA*008 or MICA*009, so the antibody is likely to recognize MICA*010.

TABLE 2

| | | Identical in | | | |
|---|---|---|---|---|---|
| | Allele | a3 domain of | | | Adjusted |
| | Frequency in | antigen used | Population | | Population |
| MICB | population | (yes = 1; | Coverage | | Coverage |
| Alleles | (%) | No = 0) | (%) | Adjust | (%) |
| *005 | 43.9 | 1 | 43.9 | 1 | 43.9 |
| *004 | 21.7 | 1 | 21.7 | 1 | 21.7 |
| *002 | 18.9 | 1 | 18.9 | 1 | 18.9 |
| *008 | 11 | 1 | 11 | 1 | 11 |
| *014 | 2.2 | 0 | 0 | 1 ^{a} | 2.2 |
| *013 | 1.4 | 0 | 0 | 1 ^{a} | 1.4 |
| Total | 99.1 | | 95.5 | | 99.1 |

^{a} the unique residual locates at the end of alpha 3 domains, would unlikely influence recognition by the antibody.

Mice that showed sufficient serum titers were sacrificed. Spleens and bone marrow were collected. Splenocytes or plasma B cells were isolated using mouse CD138+ Plasma Cell Isolation Kit (Miltenyi Biotec Inc., Cat #: 130-092-530) and were separately processed to isolate single cells. mRNA was isolated from single cells. Nucleic acid sequences encoding the heavy chain variable region (VH) and light chain variable region (VL) were amplified by RT-PCR. The amplified PCR products encoding MICA/B-specific single-chain variable fragment (scFV) were engineered into yeast libraries.

The two yeast libraries described above went through four rounds of selection using Miltenyi MidiMACS™ system with biotinylated immunogens and fluorescence-activated cell sorting (FACS) with either biotinylated Ag or native protein or whole cell selection. The methods are described, e.g., in Chao et al. Isolating and engineering human antibodies using yeast surface display. Nat Protoc. 2006; 1(2):755-68, which is incorporated herein by reference in its entirety. Briefly, the libraries were first isolated using Miltenyi MidiMACS™ system. Isolated yeast went through two rounds of sorting using a FACS ARIAII sorter (BD Biosciences) with biotinylated MICA and MICB-alpha3-mFc fusion antigens. Sorted MICA/MICB binding yeast cells were incubated with CHO cells expressing MICA/B. After washing off the unbound cells, CHO cells with bound yeast were detached, resuspended in yeast growth medium and plated to form single yeast colonies on standard yeast growth media.

Single yeast colonies were picked and inoculated into a 96-well plate with yeast growth medium for scFv production. Supernatants of the yeast cultures were harvested and screened for protein binding using ELISA and Carterra®. Nucleic acid sequences encoding the heavy chain variable region (VH) and light chain variable region (VL) of MICA/B-specific antibodies were amplified by RT-PCR. Nucleic acid sequences encoding the VH and VL of the hits were obtained by sequencing. Afterwards, the sequences encoding VH and VL were cloned into a vector expressing human IgG1 (with mouse variable regions and human constant regions) and expressed in HEK or CHO cells. The expressed antibodies were subjected to further screening and characterization. Some of these antibodies are shown in FIG. 23. Their CDR sequences are shown in FIGS. 24A-24E.

To humanize the antibodies identified above, two approaches were used. First, humanization templates were designed in silico using Molecular Operating Environment (MOE); Second, humanization templates were designed by grafting the 6 CDRs to the human germline VH and VL frameworks (FR) and keeping a limited number of murine FR residues adjacent to CDRs. Human germlines were selected based on homology to mouse. CDRs were defined based on one or the combination of numbering schemes known in the art. The humanization templates were synthesized. DNA fragments of the humanization templates were amplified and introduced into yeast for display.

To improve affinity and control for manufacturing challenges, mutation libraries were constructed by mutagenesis in CDRs either by design using MOE or random mutagenesis by error-prone PCR. The designed libraries were synthesized. DNA fragments of the library sequences were amplified and introduced into yeast for display. Yeast screenings were performed similarly to the selection process as described above, except that a more stringent threshold was used to select for higher binding affinities.

A selected number of humanized and optimized hits with desirable characteristics were further engineered to reduce manufacturing challenges by user-defined point mutations and/or to de-immunize by back-germline mutations on frameworks, and maximally maintain desirable characteristics. The sequences of the humanized and/or optimized antibodies are shown in FIGS. 25, 26, and 27. Their CDR sequences are listed in FIGS. 28, 29, and 30A-30E.

### Example 2. Biochemical Characterization of Anti-MICA/B Antibodies

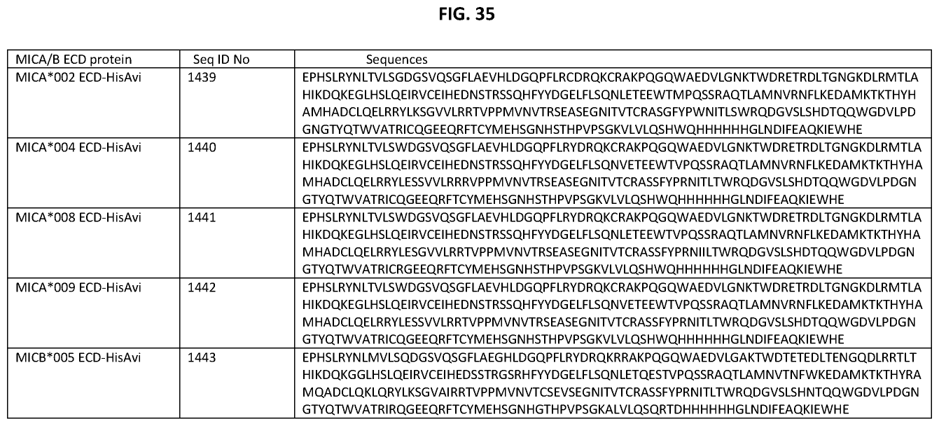

Binding kinetics of the antibodies identified in Example 1 were measured using Carterra® SPR imaging system (Carterra USA). Specifically, the anti-MICA/B antibodies were captured by anti-human Fc (SouthernBiotech, Cat #: 2047-01) immobilized on a HC200M-Polycarboxylate chip (Carterra Bio, Cat #: HC200M) or a HC30M-Polycarboxylate chip (Carterra Bio, Cat #: HC30M) via amine-based coupling. Next, as disclosed in FIG. 35, the corresponding monomeric antigen MICA002-ECD-HISAvi (BioIntron, Project code #: B662201), MICA004-ECD-HISAvi (BioIntron, Project code #: B662202), MICA008-ECD-HISAvi (Sino Bio, Cat #: 12302-H08H1), or MICB005-ECD-HISAvi (Sino Bio Cat #: 10759-H08H) were injected for 2-3 minutes at 25° C. in running buffer. The dissociation kinetics were monitored for approximately 10 minutes. The chip surface was regenerated between binding cycles with 10 mM glycine HCl, pH 1.7. Binding kinetics were analyzed using software supplied by the manufacturer.

The binding kinetics of the antibodies were also measured using the GatorPrime BLI (Biolayer Interferometry) system (Gator Bio, USA). Specifically, the anti-MICA/B antibodies were loaded with HFC (Anti-hIgG Fc, Gator Bio, Cat #: 160003). Next, probes were incubated with corresponding monomeric antigens MICA002-ECD-HISAvi (BioIntron, Project code #: B662201), MICA004-ECD-HISAvi (BioIntron Project code #: B662202), MICA008-ECD-HISAvi (BioIntron Project code #: B21016107), MICB005-ECD-HISAvi (BioIntron Project code #: B21016109), MICA009Avi (BioIntron Project code #: B21016108), as disclosed in FIG. 35, or Cynomolgus monkey MICA (cynoMICA) (Sino Bio, Cat #: 90924-C08H) for 3-4 minutes at 25° C. in K buffer (Gator Bio, Cat #: 120011). The dissociation kinetics were monitored for approximately 10 minutes. The probes were regenerated between binding cycles with Regeneration Buffer (Gator Bio, Cat #: 120012). Binding kinetics were analyzed using software supplied by the manufacturer.

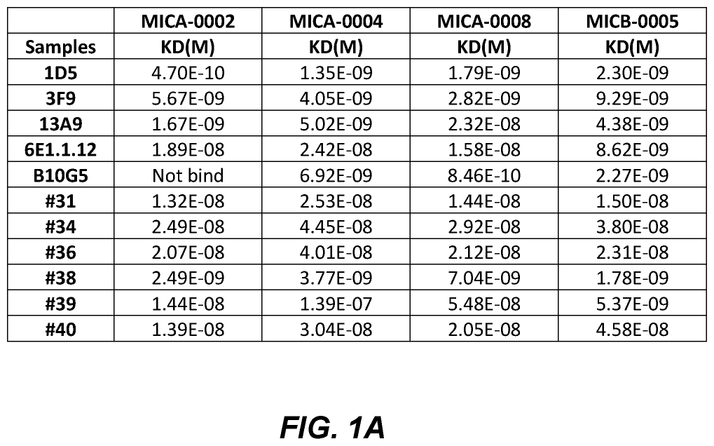

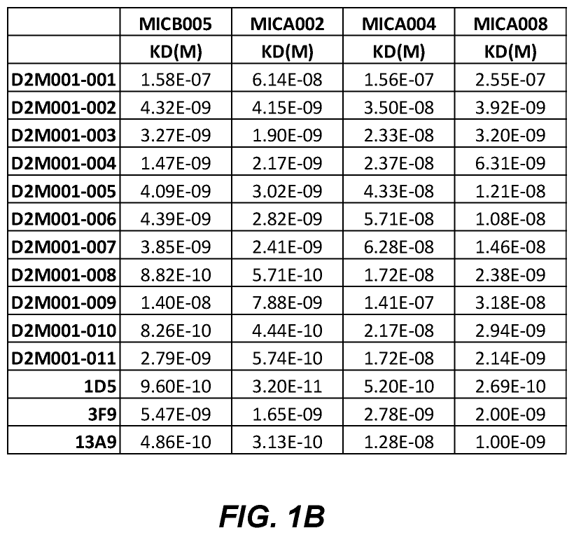

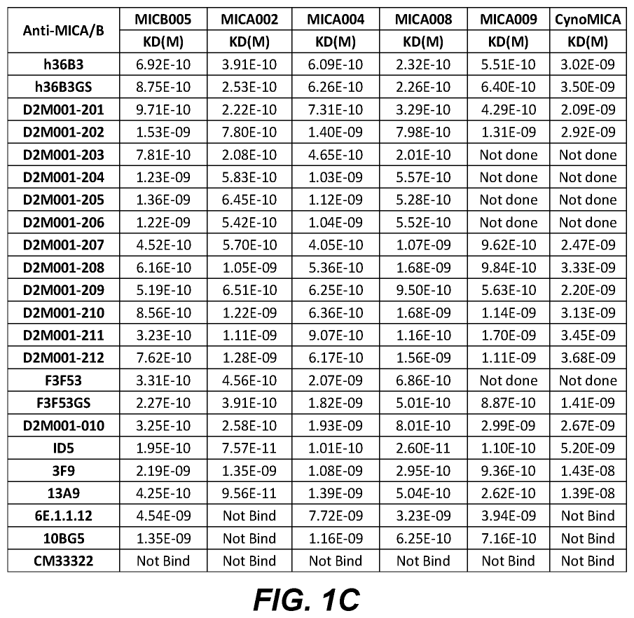

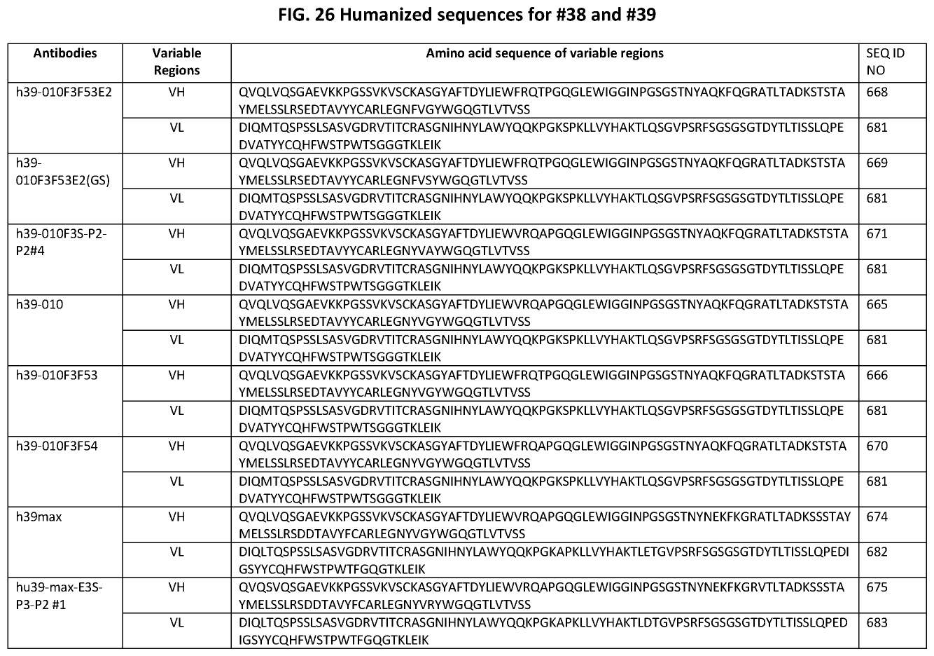

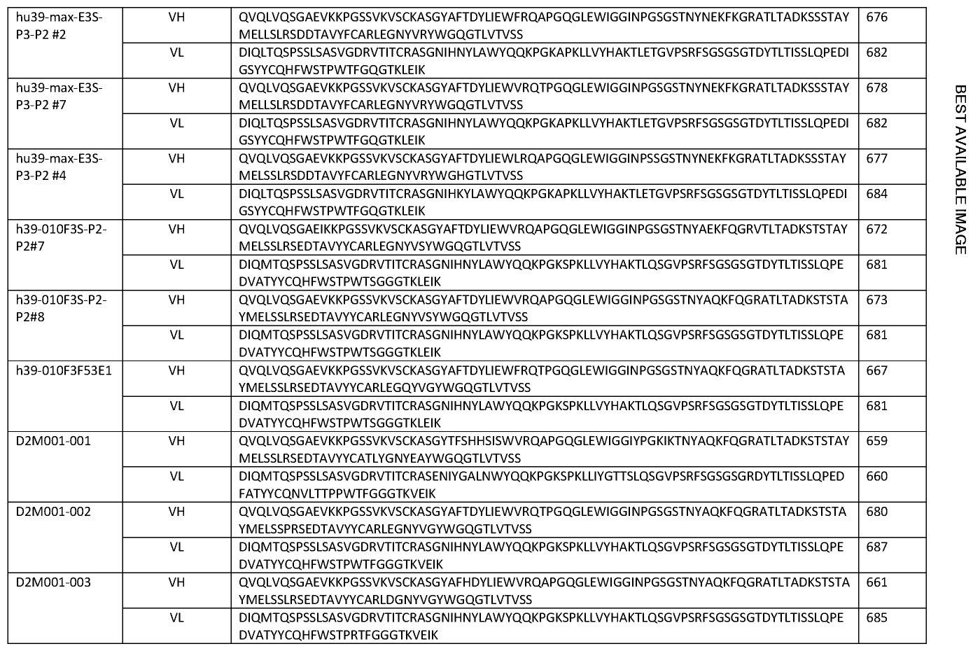

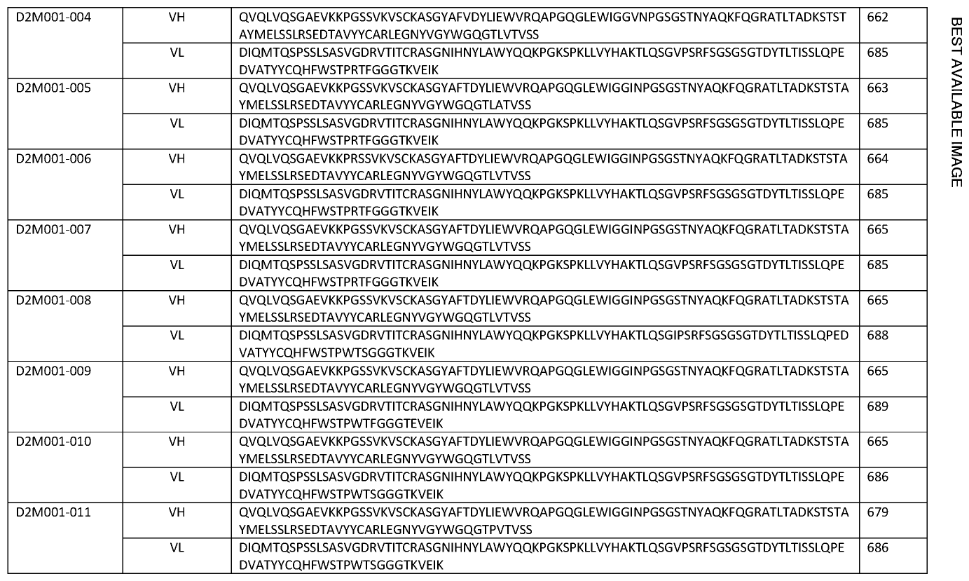

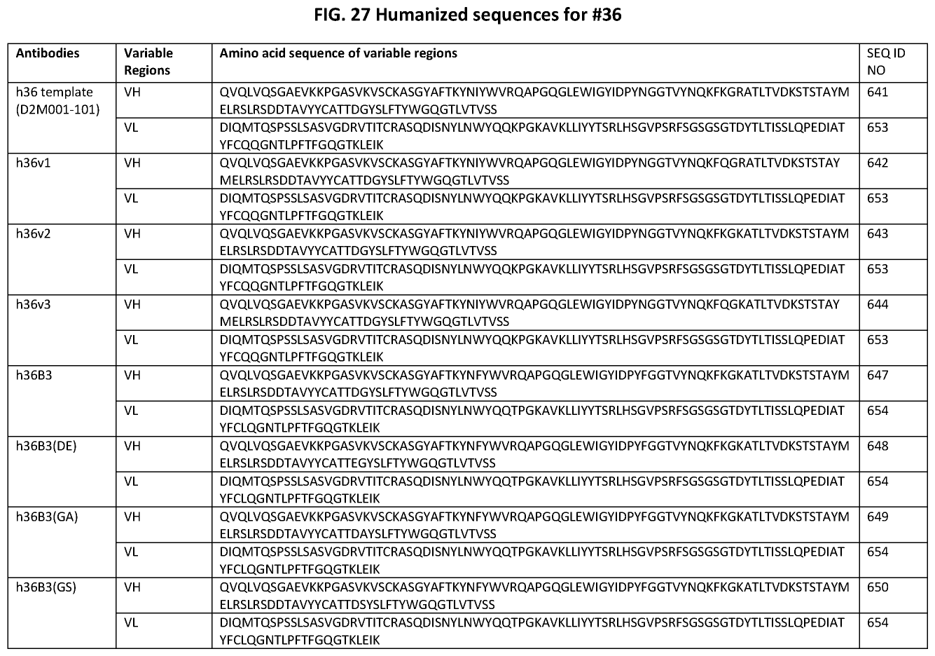

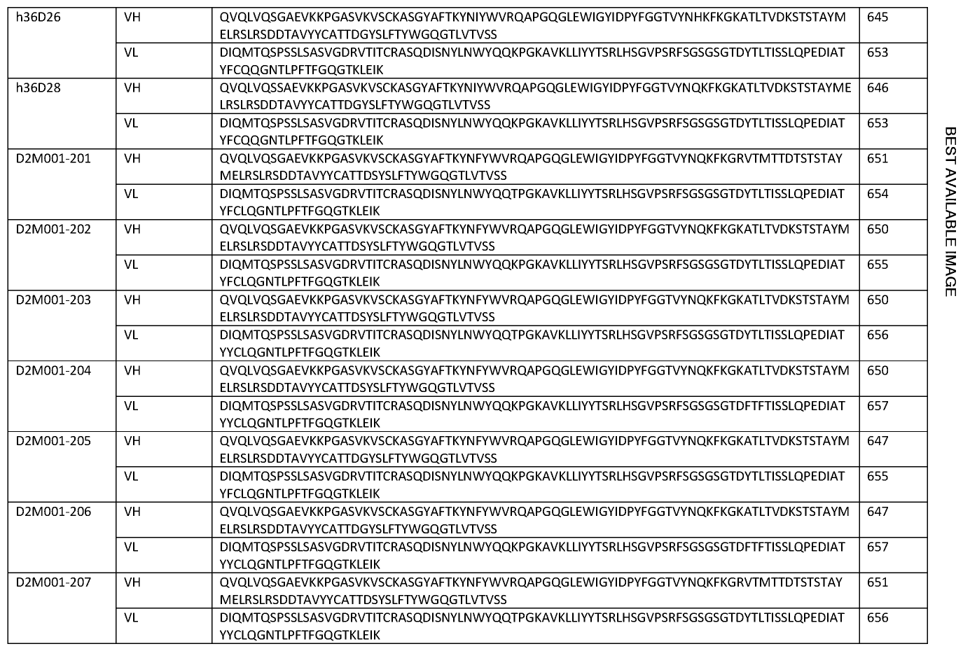

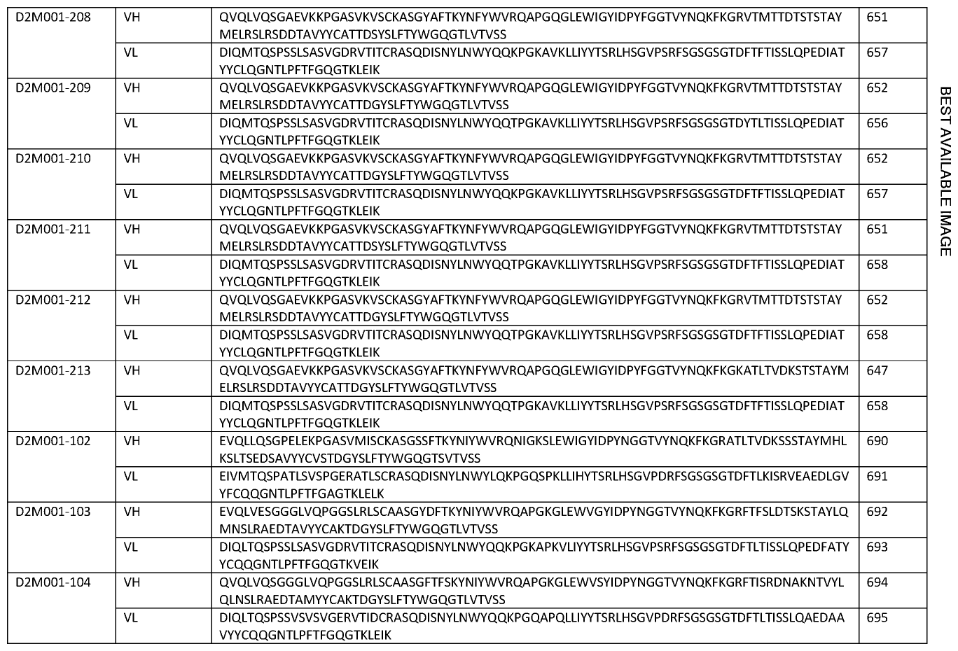

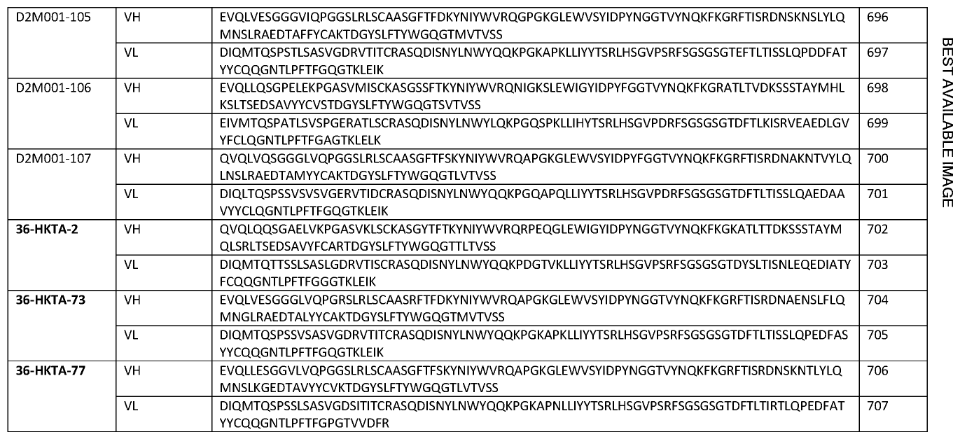

The binding affinities of chimeric antibodies measured by the Carterra® system are shown in FIG. 1A and the binding affinities of humanized and optimized antibodies from #38 and #39 lineages are shown in FIG. 1B. The binding affinities of humanized antibodies #36 variants and D2M001-010 variants measured by the GatorPrime® system and are shown in FIG. 1C. Their sequences are listed in FIGS. 26-27.

As indicated by these data, the optimized and humanized #36 lineage antibodies and the D2M001-010 variants demonstrated at least low single digit nM affinities across all major human MICA/B alleles as well as cyno monkey MICA. Back-to-germline mutations in framework regions did not significantly change the binding affinity.

FIG. 1C summarizes the binding profiles of in-house optimized and humanized anti-MICA/B antibodies and reference anti-MICAB antibodies. As shown in FIG. 1C, the lead candidate D2M001-209 exhibited broad binding profiles as compared to CM33322 (LC: SEQ ID NO: 1444, HC: SEQ ID NO: 1445), 6E1.1.12 (LC: SEQ ID NO: 1417, HC: SEQ ID NO: 1418), and B10G5 (LC: SEQ ID NO: 1423, HC: SEQ ID NO: 1424). The binding of CM33322 be to all tested alleles of MICA and MICB cannot be detected in our experiments. 6E1.1.12 and B10G5 appeared to bind to MICB005, MICA004, and MICA008, but not to MICA002 and cynoMICA.

Among all tested reference antibodies, 1D5 (LC: SEQ ID NO: 1419, HC: SEQ ID NO: 1420) showed good binding affinities to all tested allotypes. However, 1D5 is still a mouse anti-human MICA/B antibody, and no information indicated that it has been successfully humanized. Considering its performance among reference antibodies, it was selected as a benchmark. As shown in FIG. 1C, D2M001-209 showed a comparable binding affinity profile relative to 1D5.

3F9 (LC: SEQ ID NO: 1415, HC: SEQ ID NO: 1416) is speculated to likely represent the only anti-MICA/B (CLN-619) which is currently evaluated in clinical trials. Therefore, 3F9 was selected as a benchmark. As shown in FIG. 1C, D2M001-209 showed a better binding affinity profile than the benchmark 3F9 for all tested allotypes except MICA008.

D2M001-209 has shown the best binding affinity to cyno MIC protein compared to all reference anti-MICA/B antibodies. For example, 3F9 and 13A9 (LC: SEQ ID NO: 1421, HC: SEQ ID NO: 1422) showed double digit nM affinities to cyno MIC, which was one magnitude less than the affinity of D2M001-209. More importantly, the difference of binding affinities between human and monkey orthologues of D2M001-209 are the least as compared to the difference of all reference antibodies. For example, the difference is within 5-fold. Comparable binding affinities between human and monkey orthologues is important for an immunotherapeutic antibody drug in drug discovery and development, as it may determine whether nonclinical studies in nonhuman primate are relevant or not.

Overall, D2M001-209 exhibited a superior binding profile to reference anti-MICA/B antibodies. In particular, the binding kinetic data have well differentiated D2M001-209 from all the reference anti-MICAB antibodies.

### Example 3: Cell-Based Characterization of Anti-MICA/B Antibodies Binding to MICA/B Variants

Cell-based antibody binding affinities were measured by incubating titrated anti-MICA/B antibodies, reference antibodies, or an isotype control antibody (IgG1) with engineered CHO cells overexpressing human MICA002, MICA004, MICA008, or MICB005 (i.e., CHO-MICA002, CHO-MICA004, CHO-MICA008, and CHO-MICB005, respectively), or Hela cells which are known to be homozygous for the MICA008 allele. 50 μL CHO-MICA, CHO-MICB, or Hela cells (4.0×10⁴ cells) were added to each well of a 96-well U-bottom plate. Then, 50 μL titrated anti-MICA/B antibodies were added to wells to a final concentration of 30 μg/mL, 10 μg/mL, 3.33 μg/mL, 1.11 μg/mL, 0.370 μg/mL, 0.123 μg/mL, 0.041 μg/mL, 0.0137 μg/mL, 0.0046 μg/mL, 0.0015 μg/mL, or 0 μg/mL at room temperature. After an incubation time of 30 minutes, the plate was centrifuged and cells were washed with PBS three times. 50 μL Alexa Fluor® 647 AffiniPure F(ab′)₂ Fragment Goat Anti-Human IgG, Fcγ fragment specific (Jackson ImmunoResearch Laboratories Inc., Cat #: 109-606-098) was added to each well. After mixing for 30 minutes at room temperature, cells were washed with PBS twice. Fluorescent signal was detected and quantified by flow cytometry (Attune™ CytPix™, ThermoFisher) within the population of live cells (viability dye PI negative).

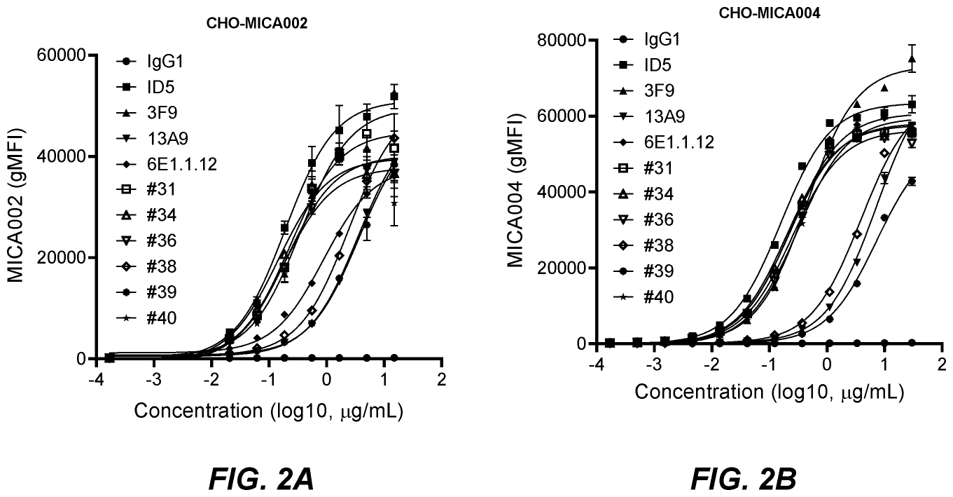

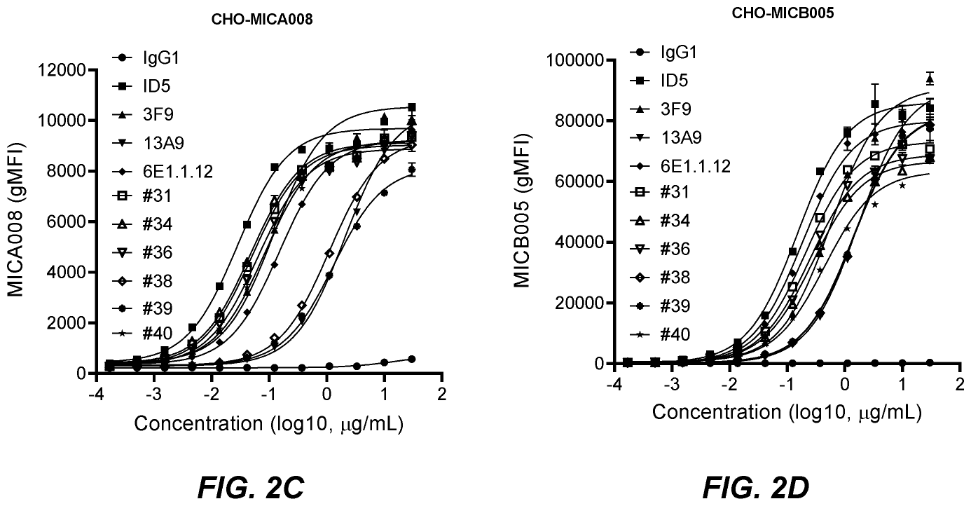

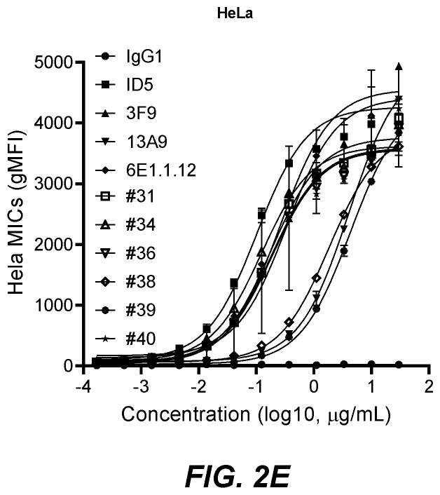

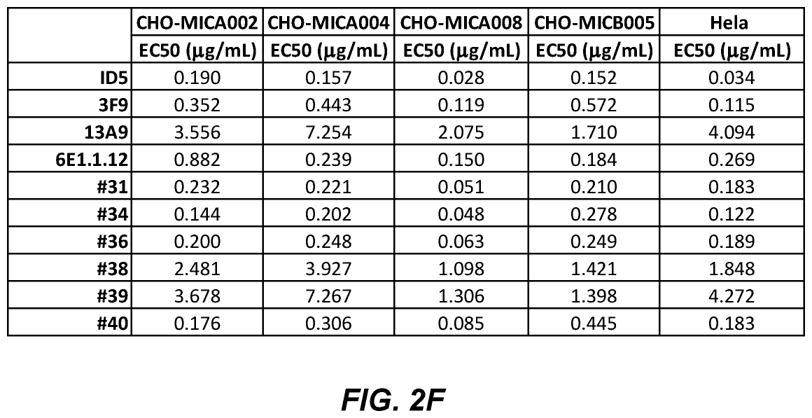

As shown in FIGS. 2A-2E, the chimeric anti-MICA/B antibodies and the reference antibodies (1D5, 3F9. 13A9, and 6E1.1.12) exhibited strong binding abilities to all four MICA/B alleles expressed on both CHO cells (FIGS. 2A-2D) and Hela cells (FIG. 2E) while the isotype control antibody IgG1 did not. EC50 values are shown in FIG. 2F and were calculated based on non-linear fitting curves by GraphPad Prism software.

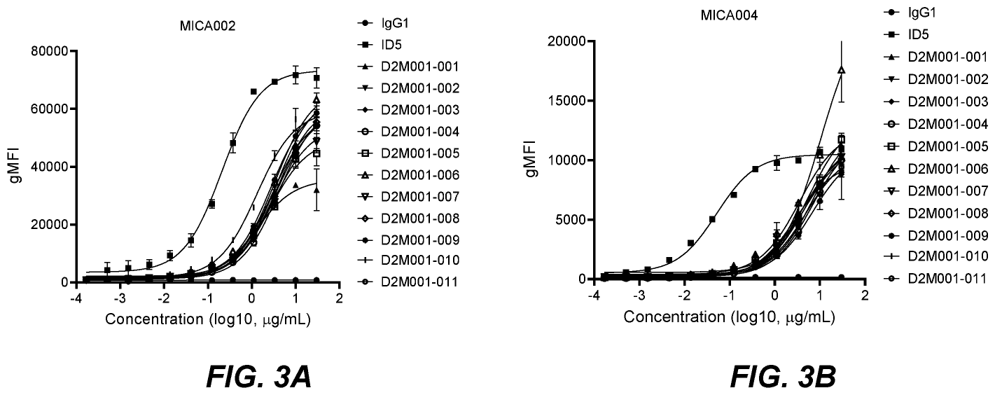

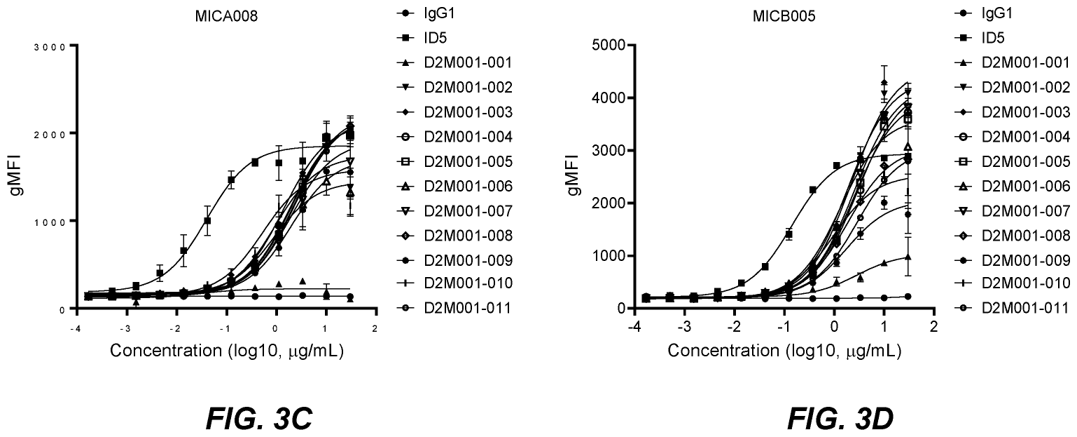

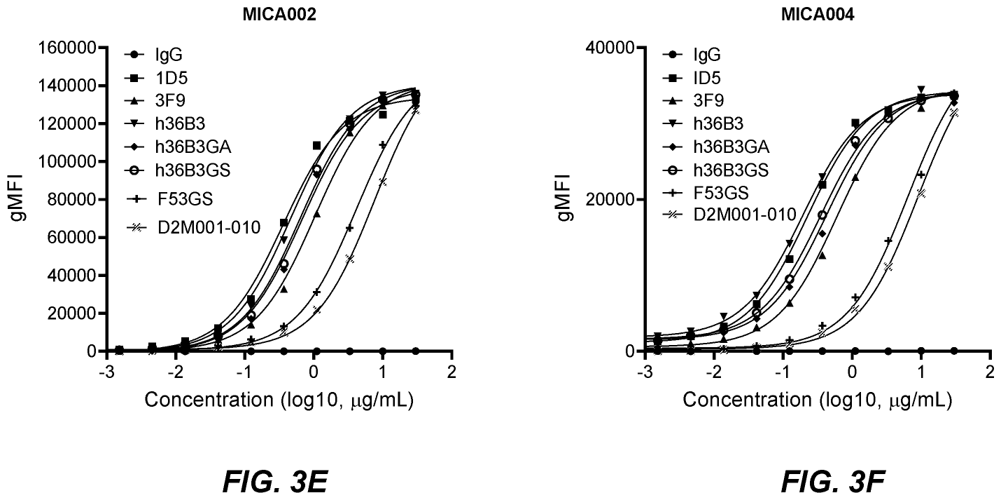

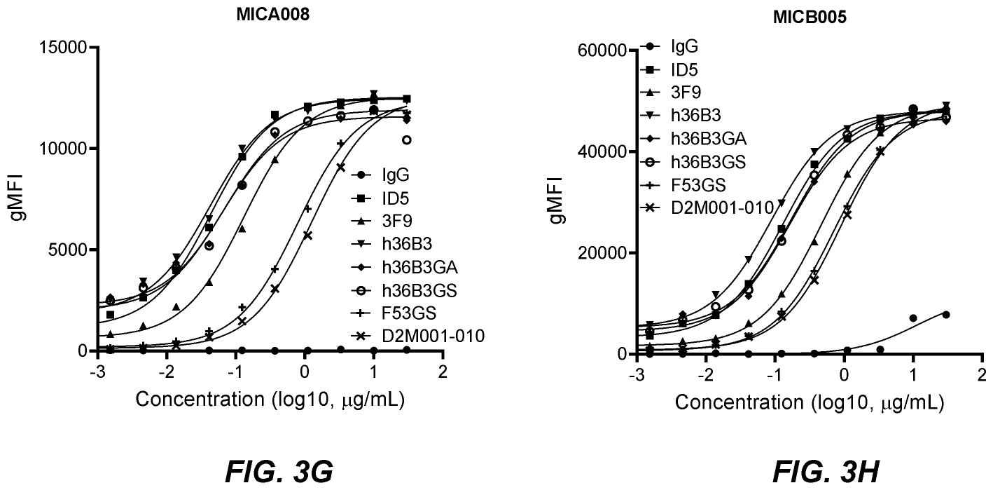

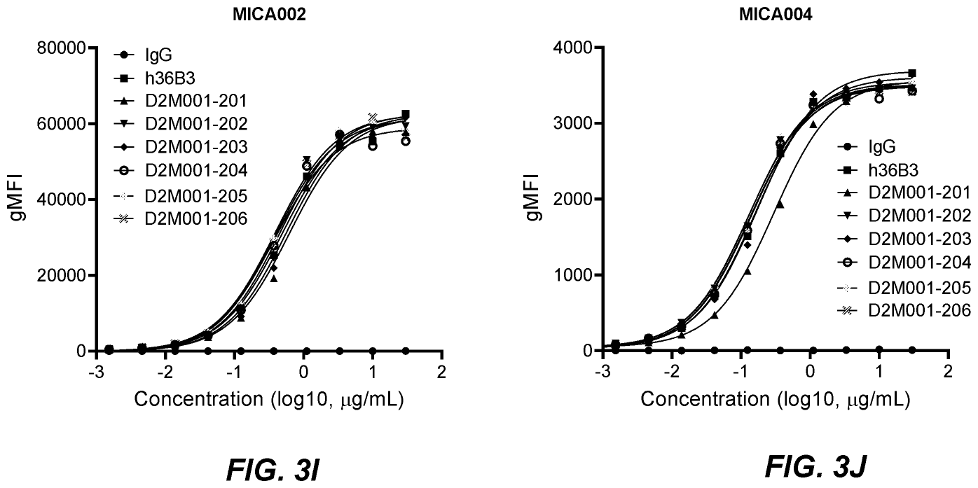

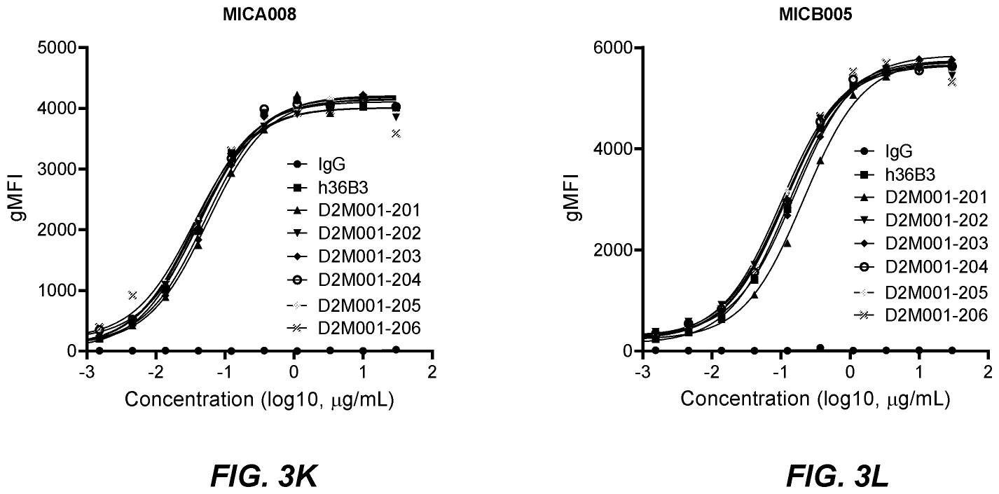

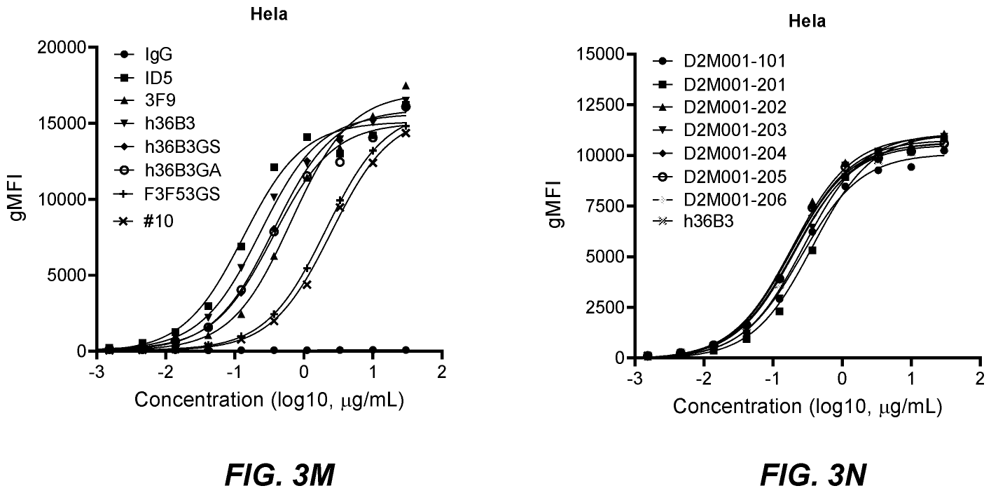

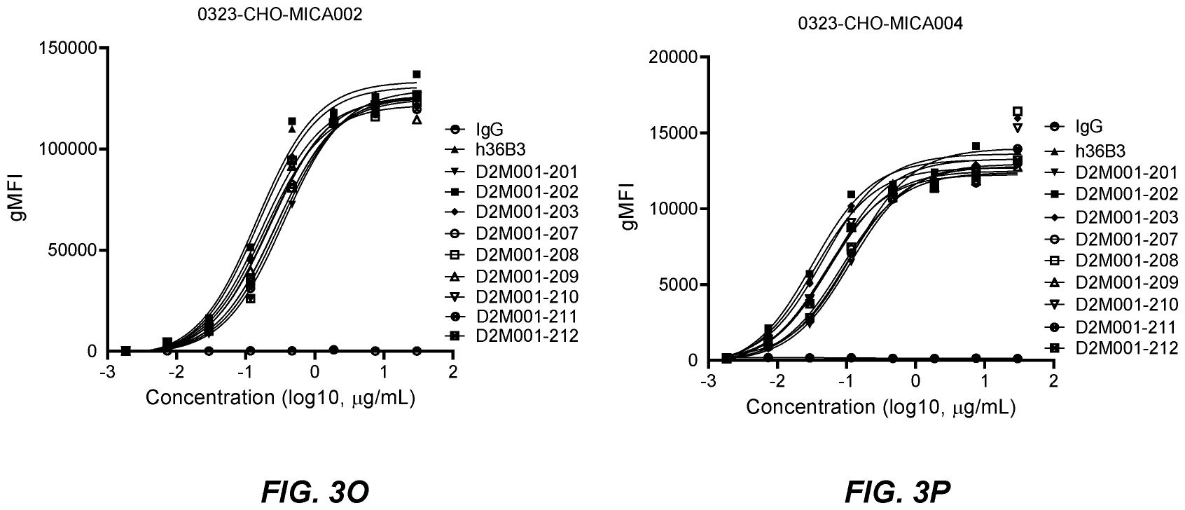

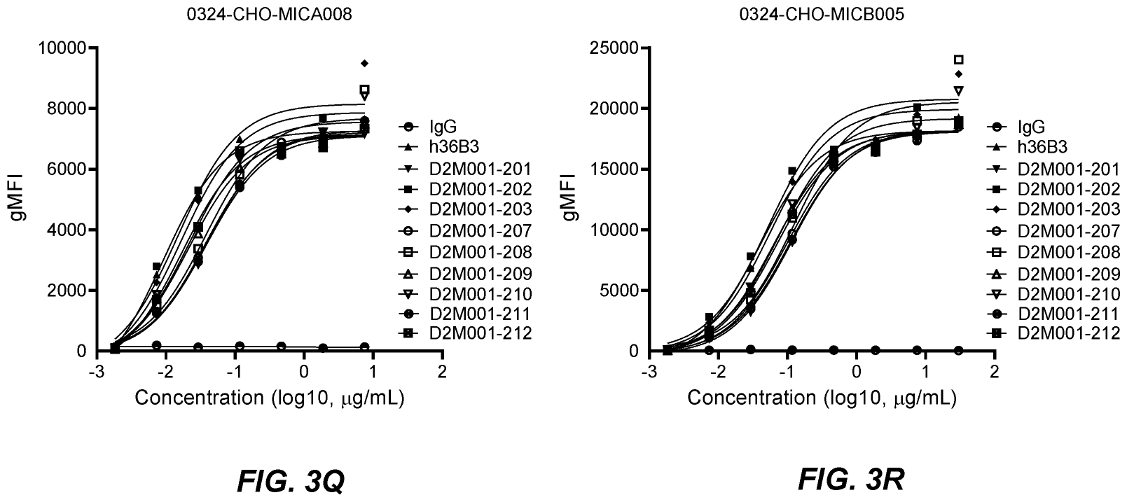

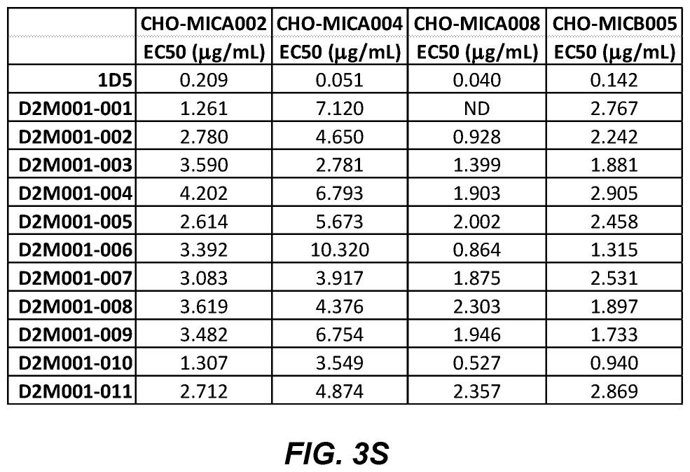

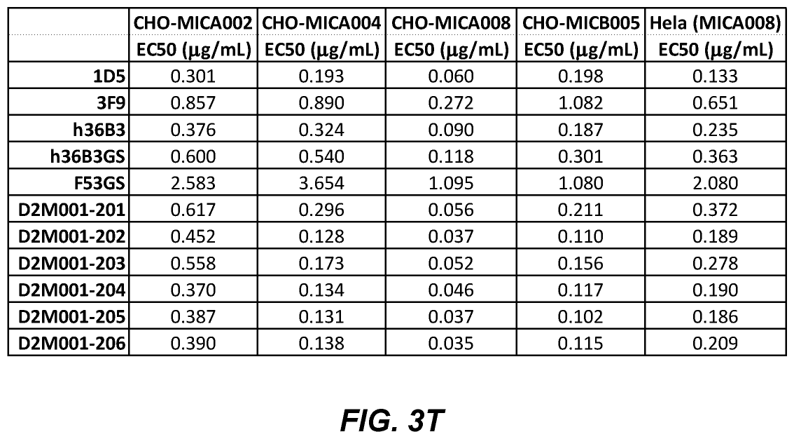

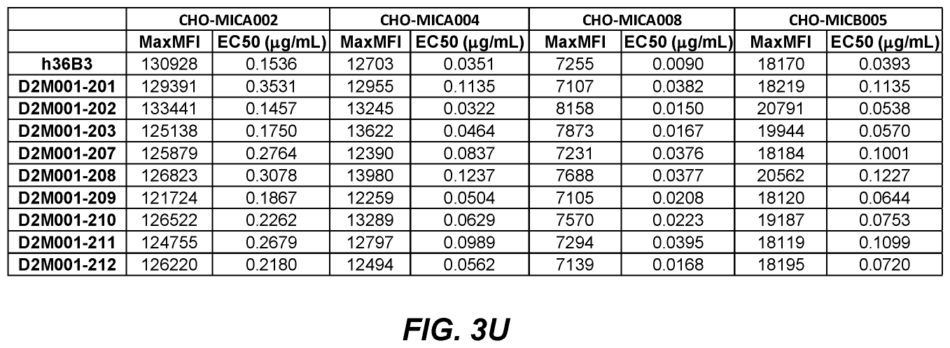

As shown in FIGS. 3A-3R, the humanized anti-MICA/B antibodies and the reference antibodies (1D5 and 3F9) exhibited strong binding affinities to all four MICA/B alleles expressed on both CHO cells and Hela cells, while the isotype control antibody IgG1 did not. EC50 values shown in FIGS. 3S-3U were calculated based on non-linear fitting curves by GraphPad Prism software. Humanized and optimized #36 variant “h36B3” showed a high binding affinity to MICA/B expressing cells, and was comparable to reference mouse antibody 1D5. In addition, H36B3 and D2M001-209 bound to MICA/B expressing cells significantly better than reference antibody 3F9, particularly to MICA008 or MICB005 expressing cells. The superior binding to MICB is important and well differentiates D2M001-209 and 3F9, as indicated in Example 6 that MICB may play more important role in NKG2D biology.

### Example 4: Anti-MICA/B Antibodies Inhibit Shedding of MICA/B Polypeptides in Tumor Cell Lines

It has been hypothesized that anti-MICA/B antibodies may inhibit the shedding of MICA/B polypeptides by binding to the MICA and MICB alpha 3 domain that promotes the initiation of shedding from the cell surface. To investigate the capability of anti-MICA/B antibodies to inhibit the shedding of MICA/B polypeptides from cells, shedding assays were performed with titrated anti-MICA/B antibodies, reference antibodies (1D5 and 3F9), or an isotype control antibody (IgG1) and engineered C1R cells overexpressing four human MICA/B variants (C1R-MICA002; C1R-MICA004; C1R-MICA008; or C1R-MICB005), or Hela cells expressing the MICA008 variant. The concentrations of shed MICA/B polypeptides were measured in the growth media by proprietary assays and commercial sandwich ELISA kits. Surface expression of MICA/B on cells was detected by flow cytometry with PE anti-MICA/B mAb 6D4 (BioLegend, Cat #: 320906). The anti-MICA/B 6D4 was used to detect the total MICA/B and shed MICA/B as it binds outside the MICA/B alpha 3 domain and does not inhibit MICA/B shedding.

50 μL C1R-MICA/B or Hela cells (approximately 1.0×10⁵ cells), or CHO-MICA002 cells (approximately 5×10⁴ cells) were added to each well of a 96-well U-bottom plate. 50 μL titrated antibodies were added to final concentrations of 30 μg/mL, 10 μg/mL, 3.33 μg/mL, 1.11 μg/mL, 0.370 μg/mL, 0.123 μg/mL, 0.041 μg/mL, 0.0137 μg/mL, 0.0046 μg/mL, 0.0015 μg/mL and 0 μg/mL. After an incubation period at room temperature for 24 hours, the plate was centrifuged and 60 μL culture medium was sampled to measure the concentrations of the soluble MICA or/and MICB separately by ELISA. The shed MICA/B concentrations were measured by sandwich ELISA assays: a 96-well assay plate was coated with 100 μL of 2 μg/mL of AMO1 anti-human MICA antibody (BAMOMAB, Cat #: BOB-AMO1-500) overnight. The plate was then washed with PBS twice and blocked with 300 μL 1% BSA for one hour at room temperature. After two additional PBS washes, 100 μL diluted culture medium was added and the plates was incubated for two hours at room temperature. Next, the plate was washed with PBS twice and 100 μL of 1 μg/mL biotinylated anti-MICA/B 6D4 (BioLegend, Cat #: 320904) was added and incubated for 90 minutes at room temperature. 100 μL 1:100 diluted Streptavidin-HRP solution (R&D Systems, Cat #: DY998) was added and incubated for 1 hour at room temperature. After washes, the plate was developed with TMB (Surmodics, Cat #: TMBS-1000-01) and stopped with BioFX 450 nM Liquid stop solution (Surmodics, Cat #: LSTP-1000-01). OD450 was measured using in the Varioskan™ Lux plate reader (ThermoFisher Scientific, Cat #: VLBL00D1, SN #: 3020-80467) to detect the concentrations of shed MICA polypeptides based on the standard of synthetic protein of MICA018-ECD-His (Sino Biological, Cat #: HPLC-12302-H08H).

The shed MICB concentrations were measured by the human MICB sandwich ELISA kit (R&D Systems, Cat #: DY1599). The remaining cells in the plate were then washed with PBS once and 100 μL diluted phycoerythrin (PE) anti-MICA/B 6D4 (BioLegend, Cat #: 320906) was added. After mixing and incubation at room temperature for 30 minutes, the plate was washed with PBS twice. Signals of PE were determined by flow cytometry (Attune™ CytPix™ ThermoFisher) within the population of live cells (viability dye DAPI negative).

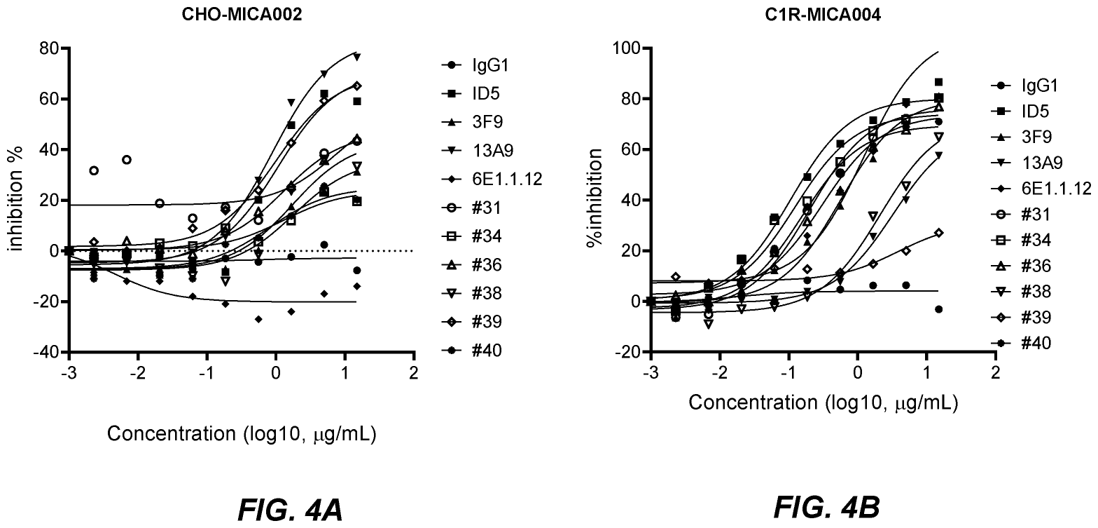

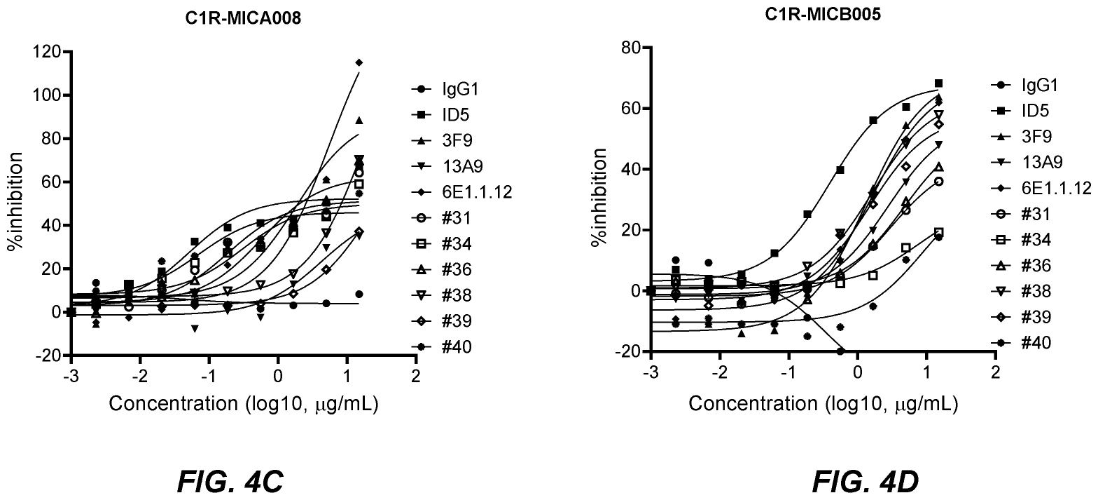

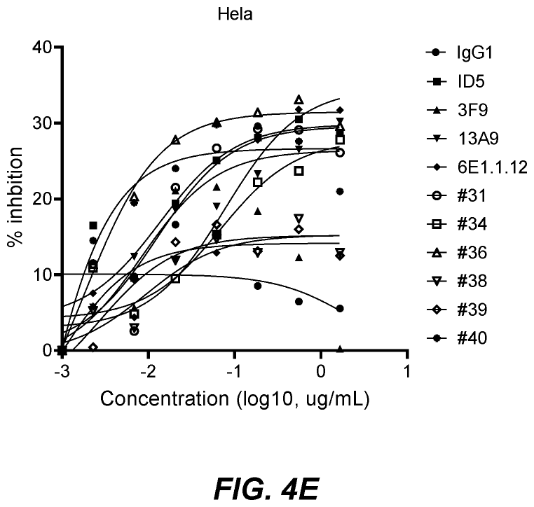

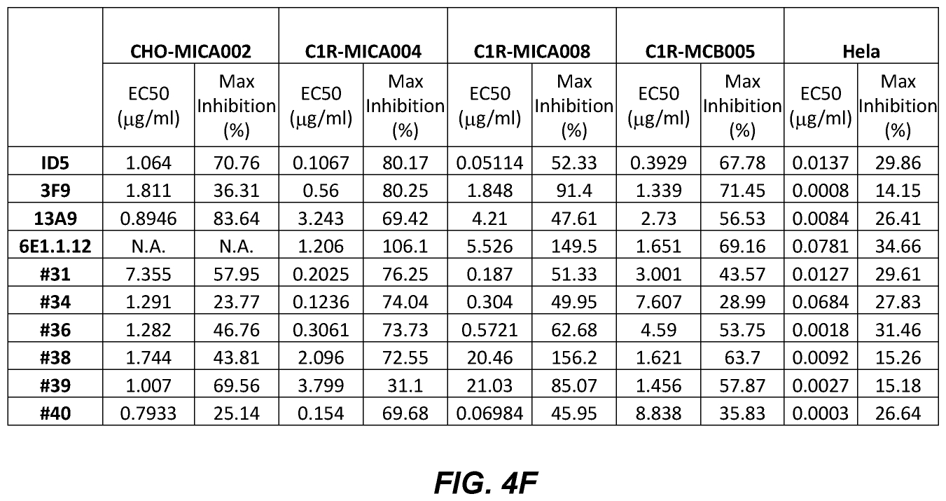

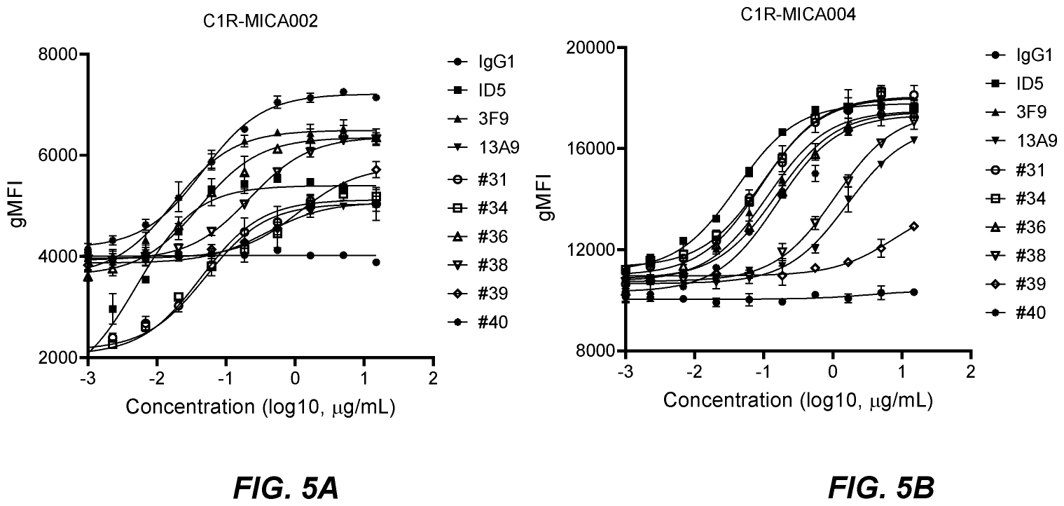

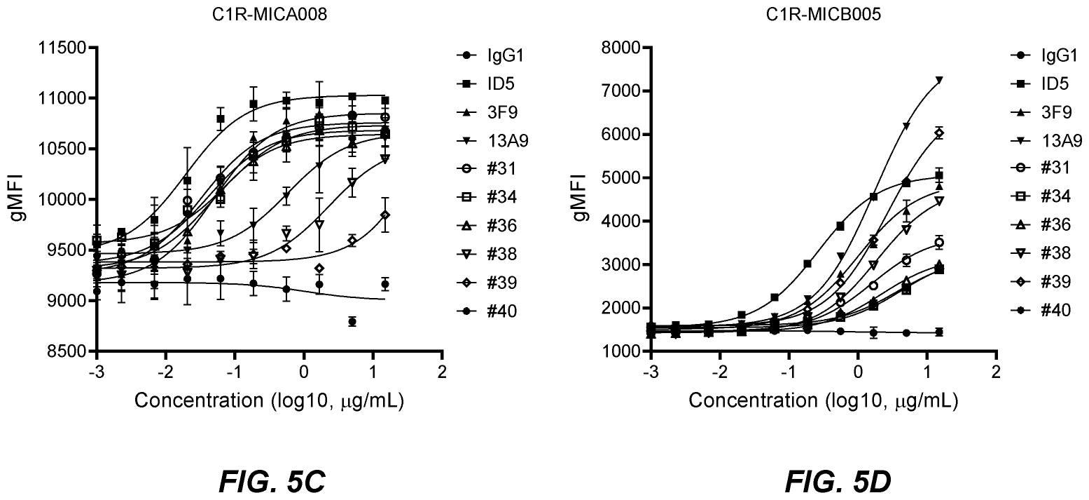

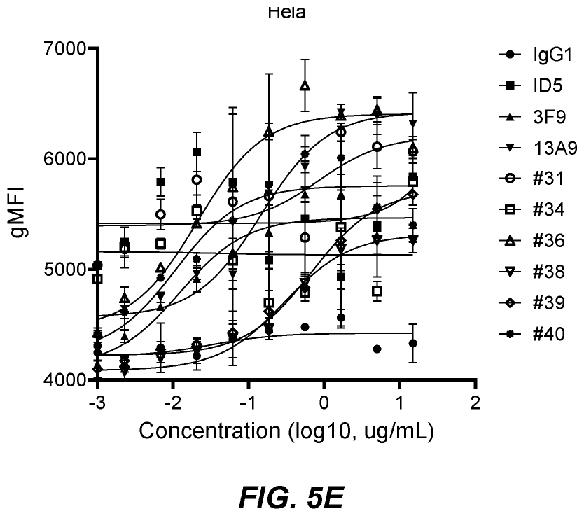

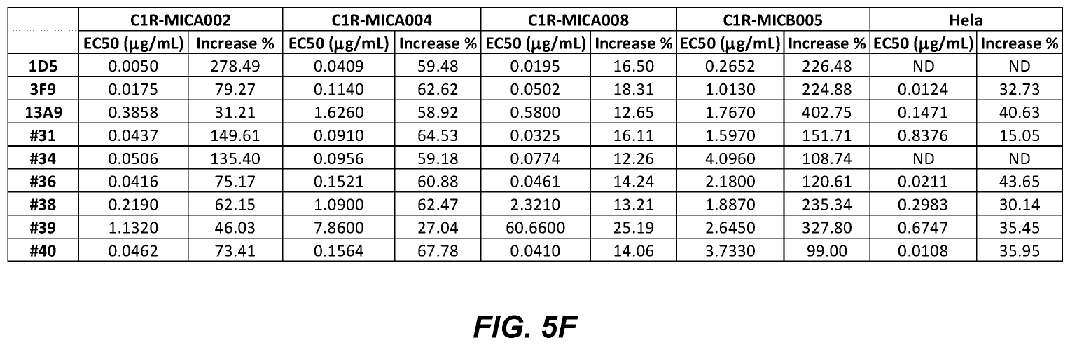

As shown in FIGS. 4A-4E, depicting results of chimeric anti-MICA/B treatment of CHO-MICA002, C1R-MICA/B, or Hela cells, the shed MICA/B polypeptides decreased significantly in the cell culture medium as a result of treatment with anti-MICA/B antibodies. Further, as shown in FIGS. 5A-5E, the surface expression of MICA/B polypeptides increased significantly in the C1R-MICA/B cells or Hela cells, indicating the chimeric anti-MICA/B antibodies and the reference anti-MICA/B antibodies exhibited strong inhibition of the shedding of all four MICA/B variants expressed on both C1R cells and Hela cells, while the isotype control antibody IgG1 did not. The maximum shedding inhibition and IC50 values of shed MICA/B (shown in FIG. 4F) were calculated based on non-linear fitting curves by GraphPad Prism software. The maximum changes of surface MICA/B expression and EC50 values (shown in FIG. 5F) were calculated based on non-linear fitting curves by GraphPad Prism software.

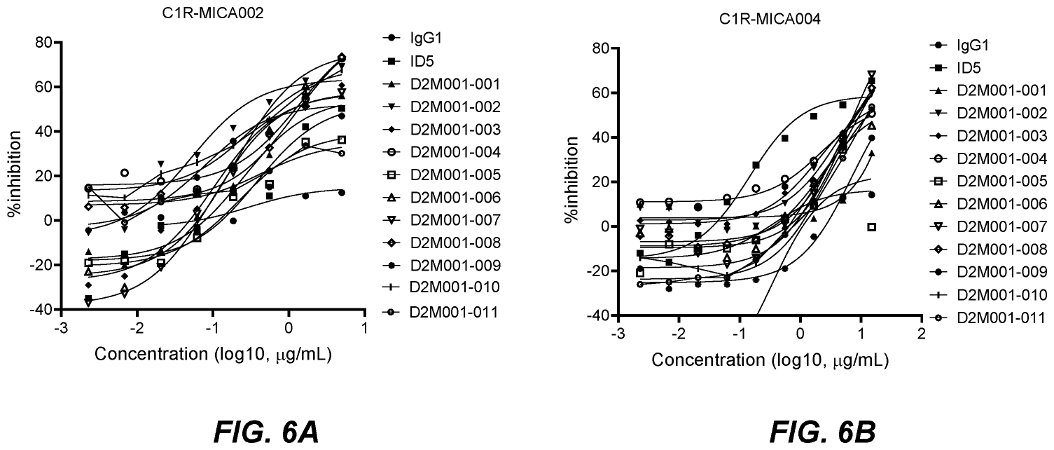

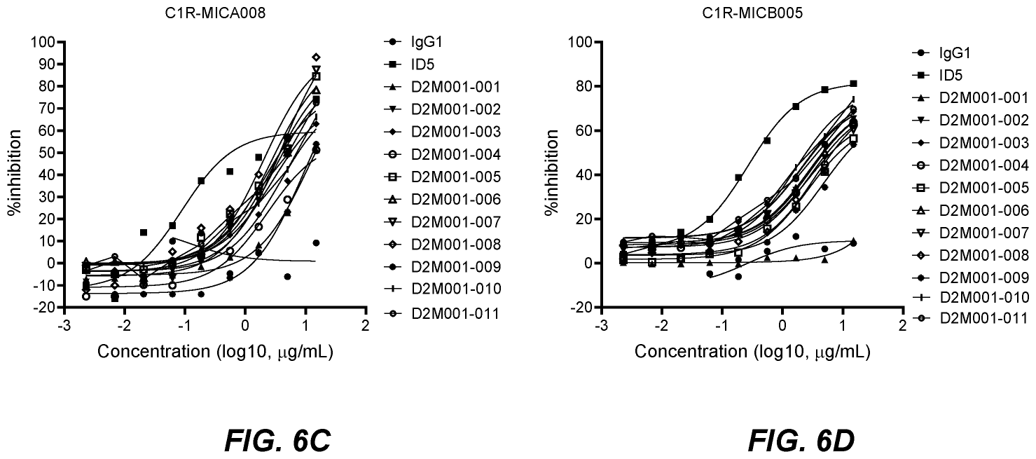

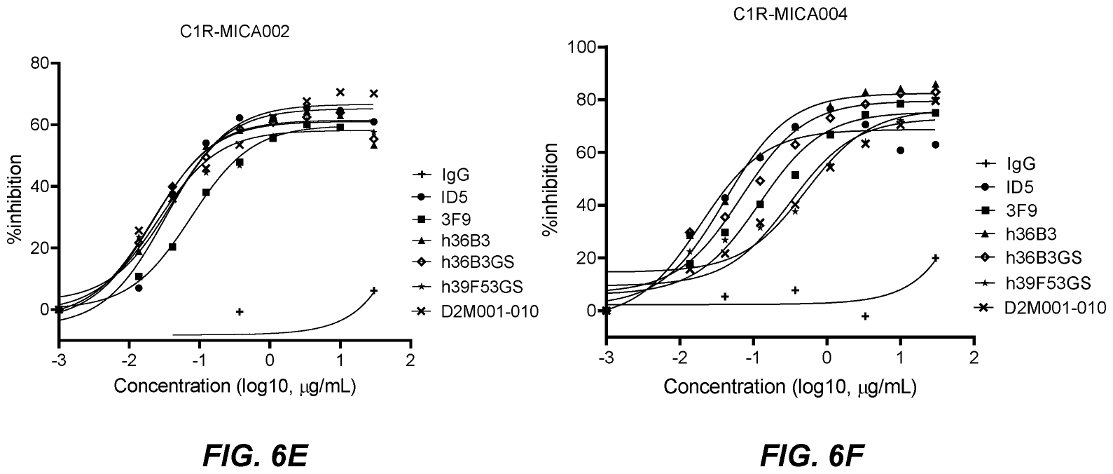

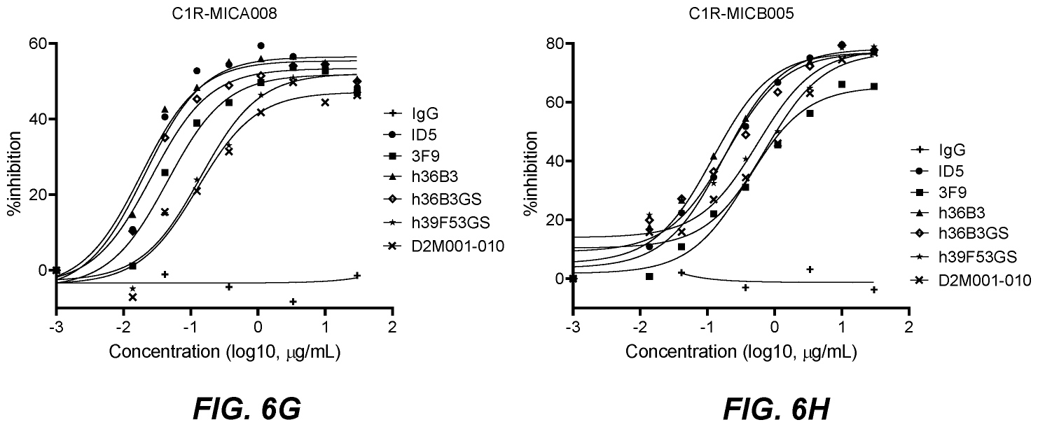

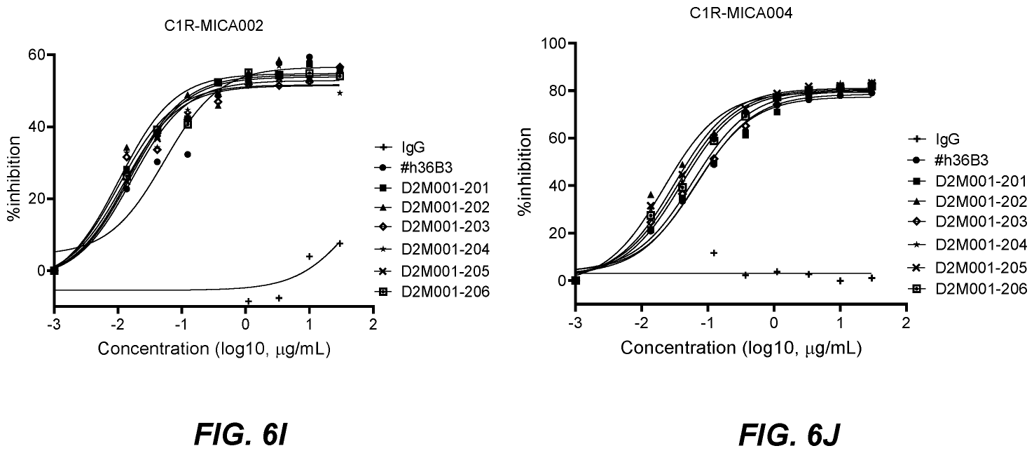

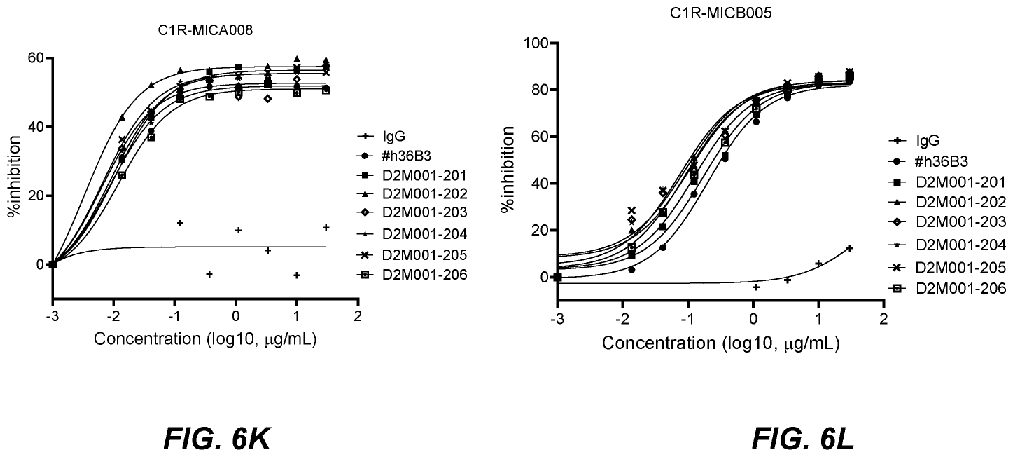

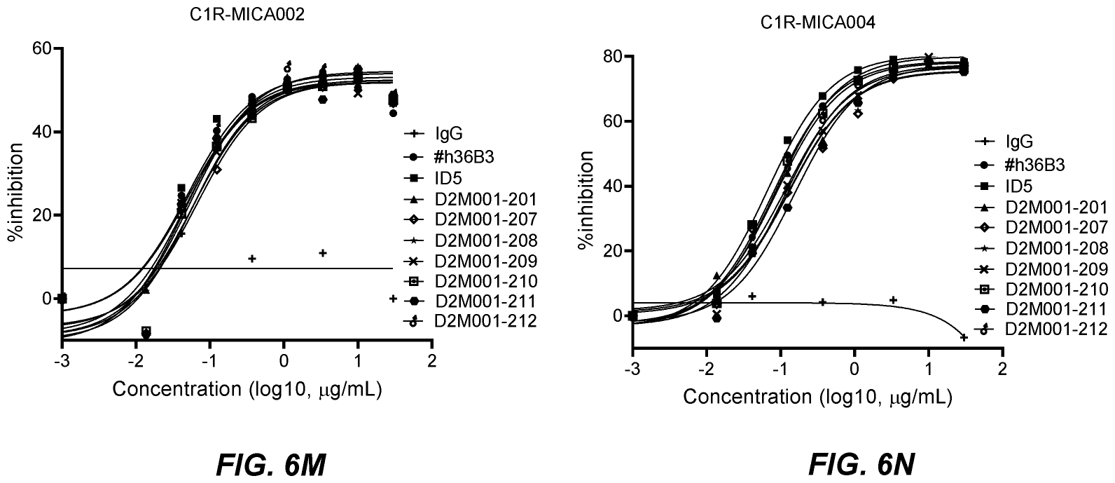

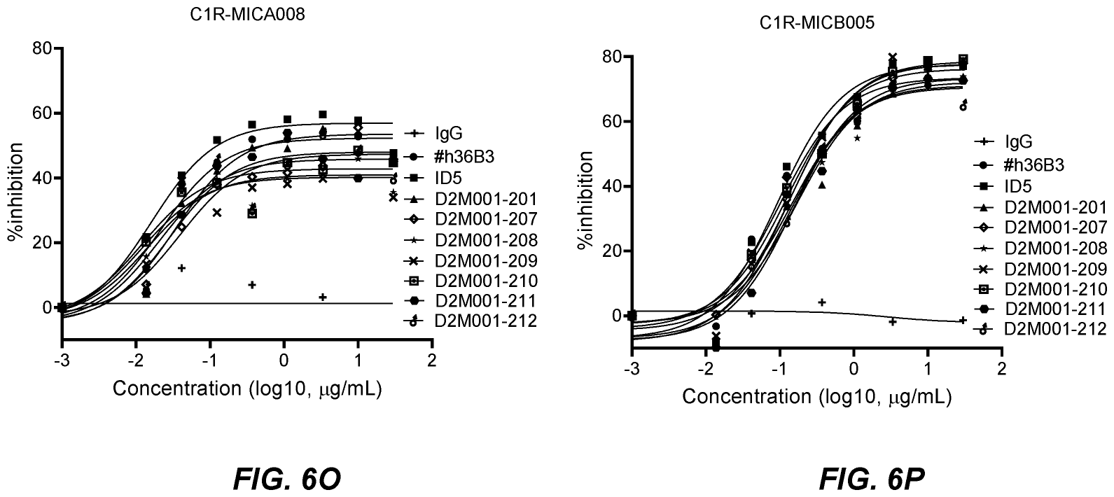

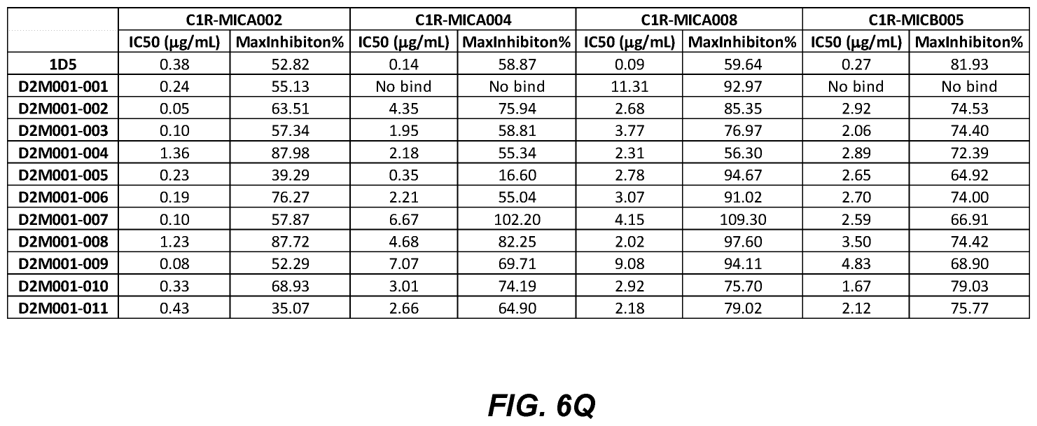

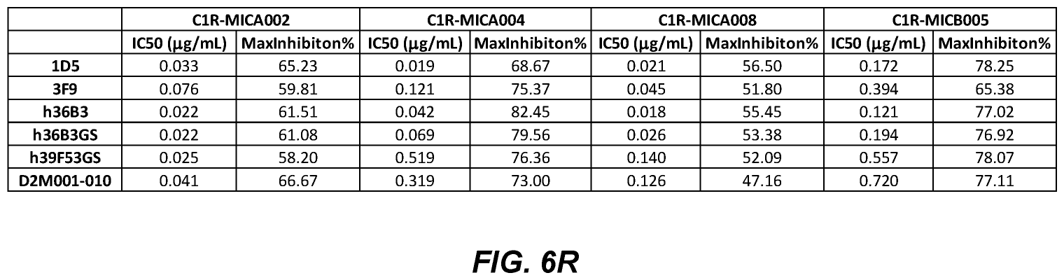

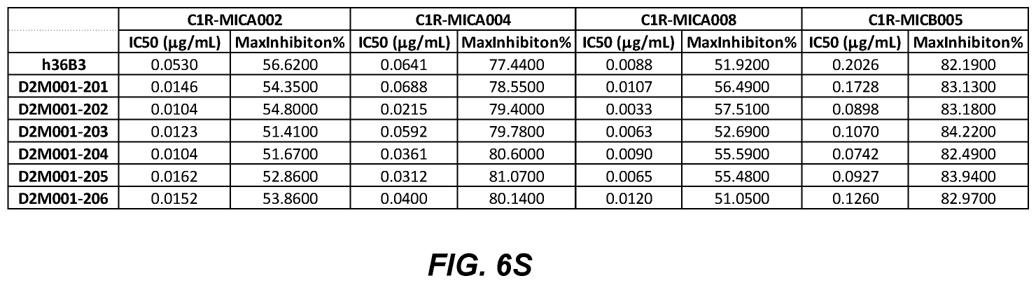

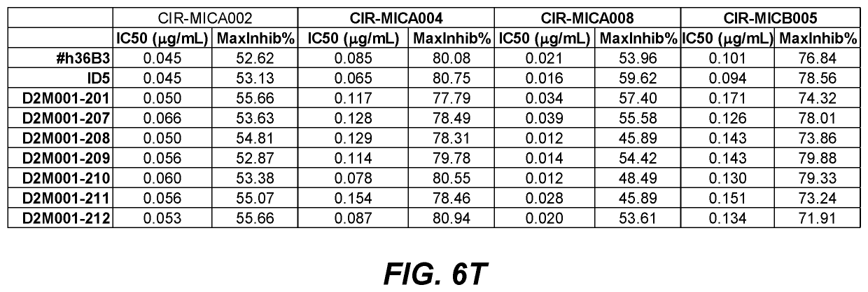

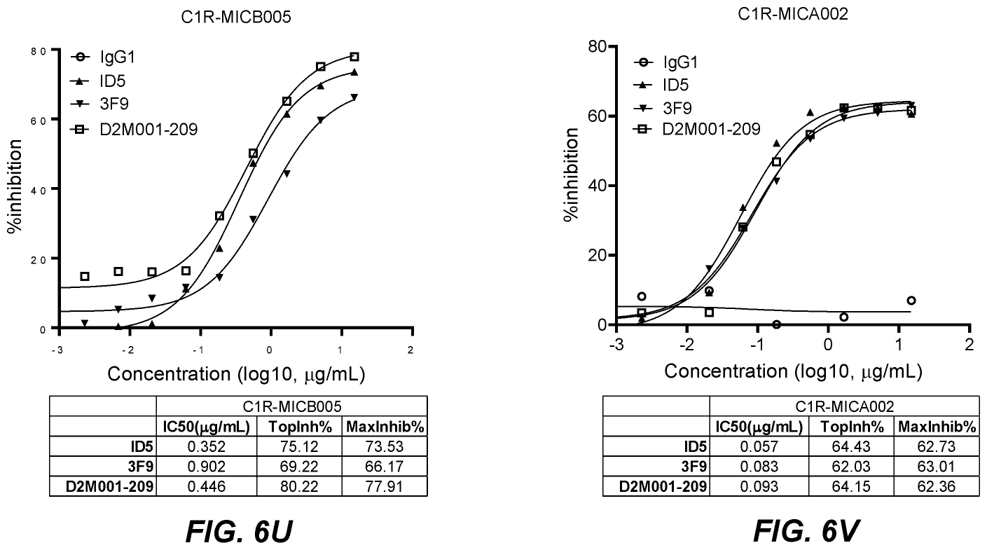

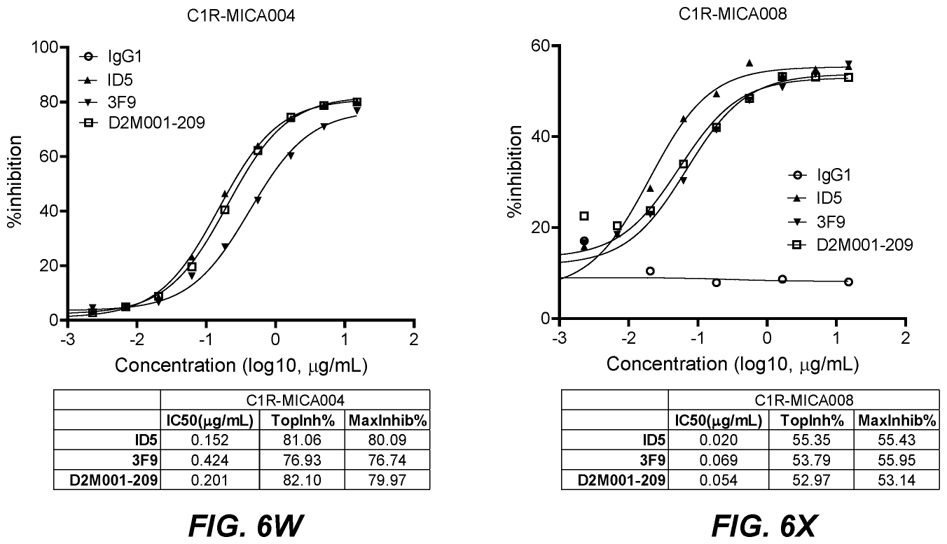

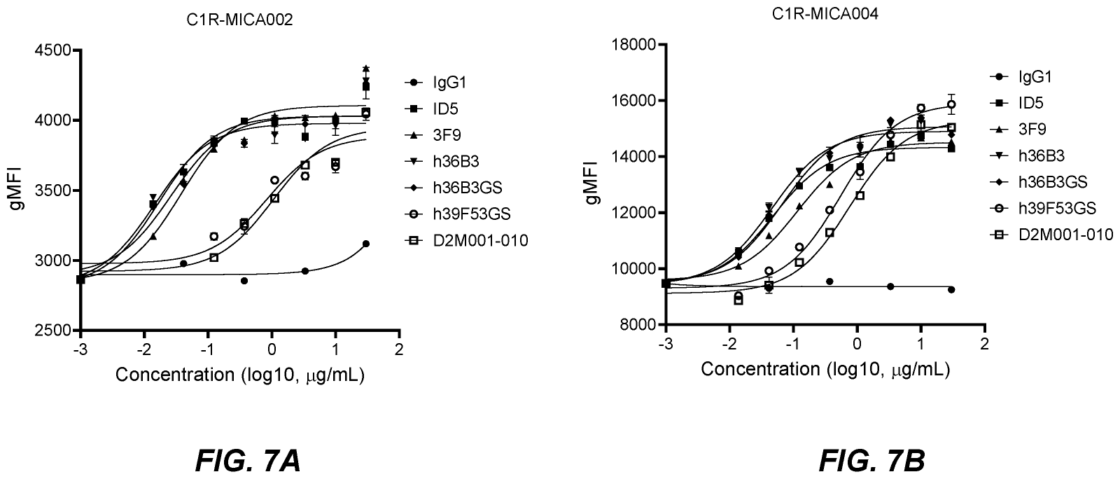

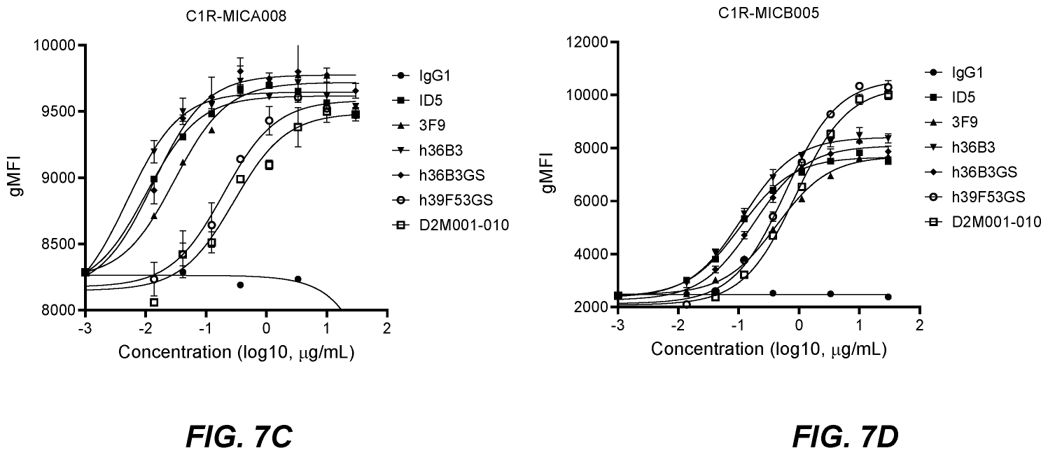

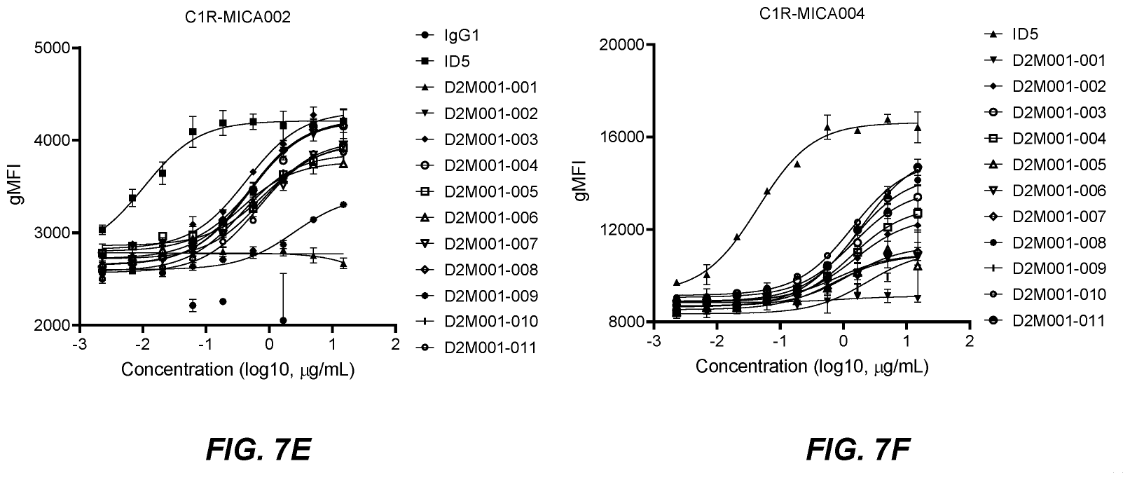

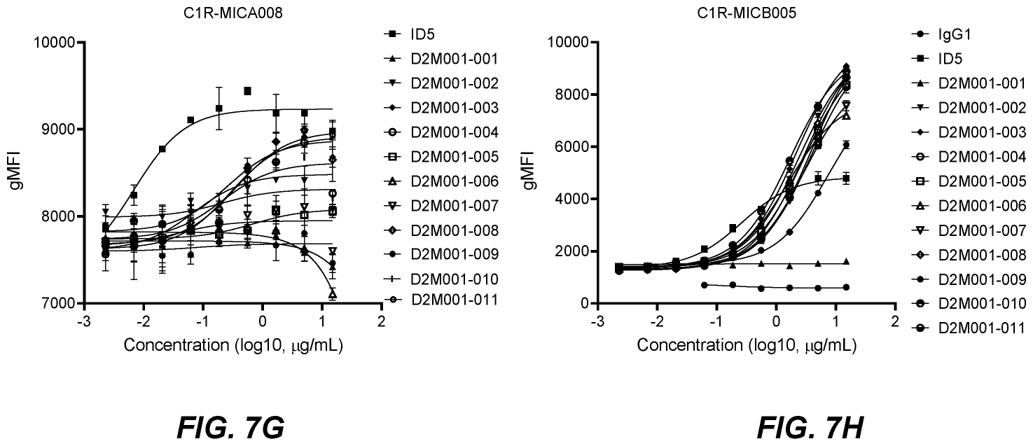

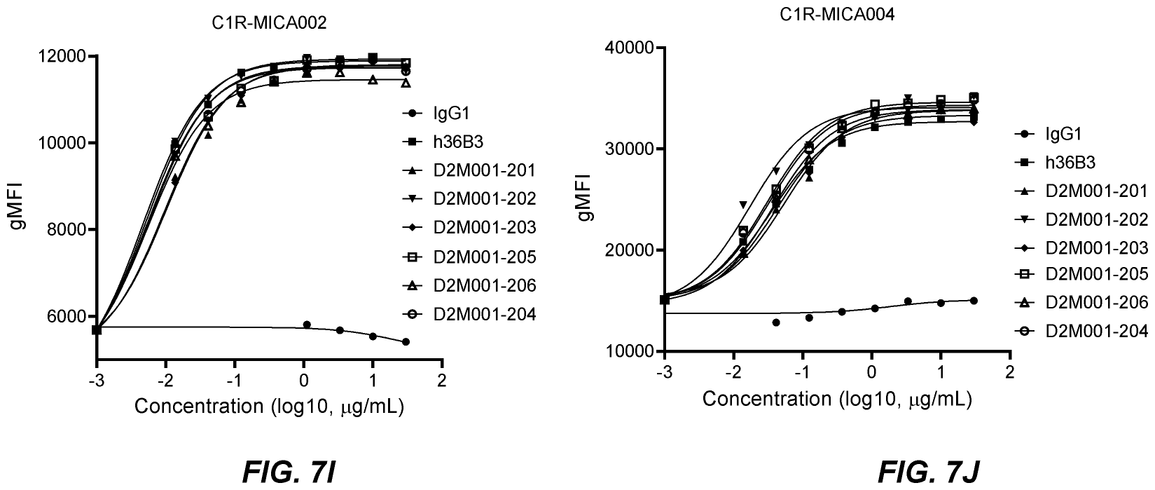

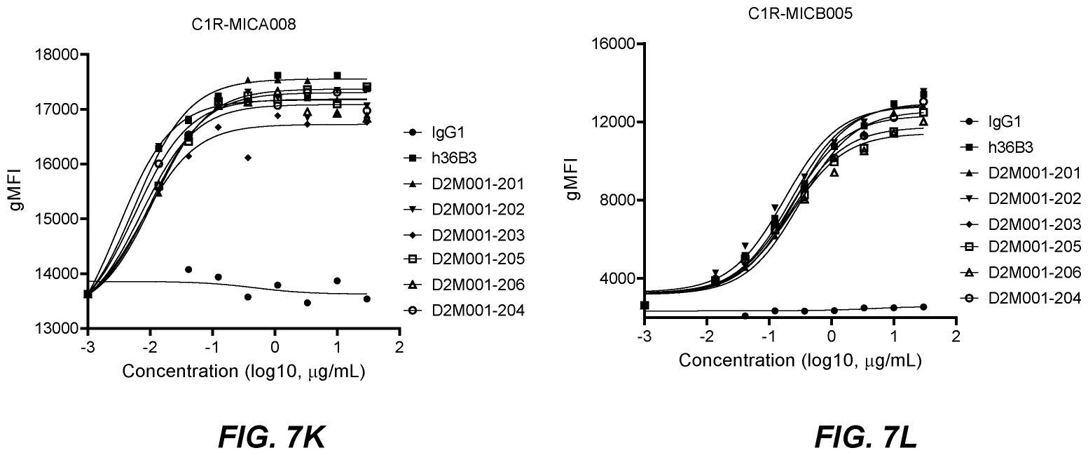

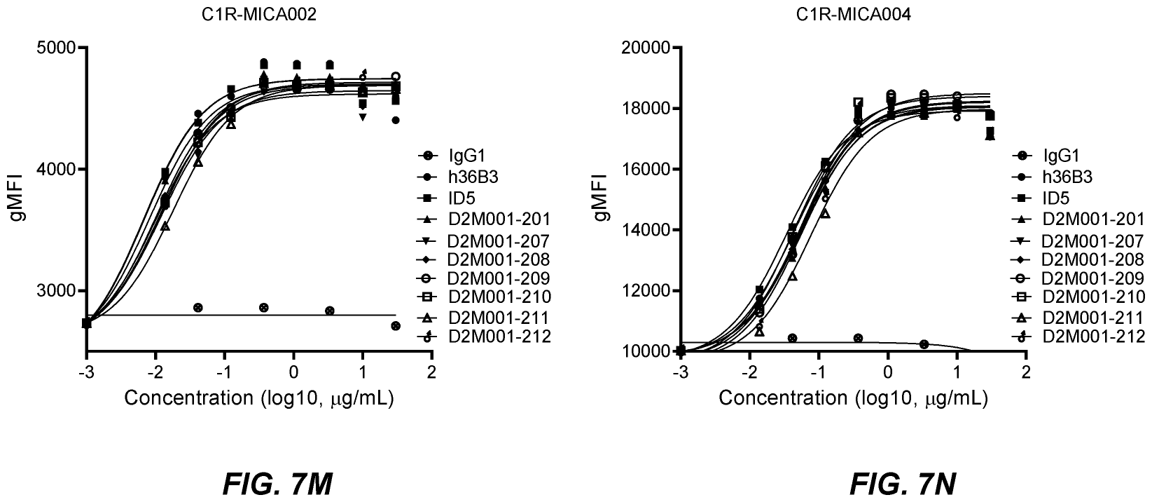

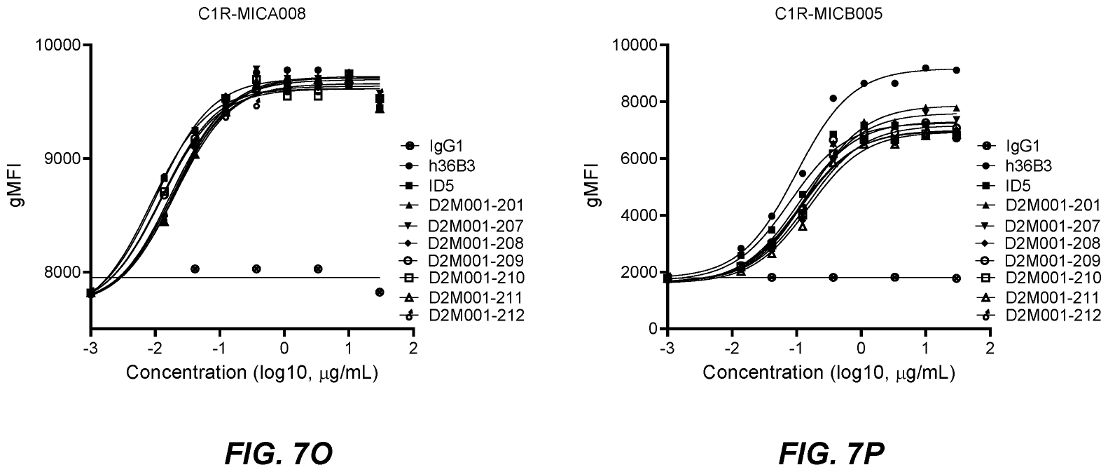

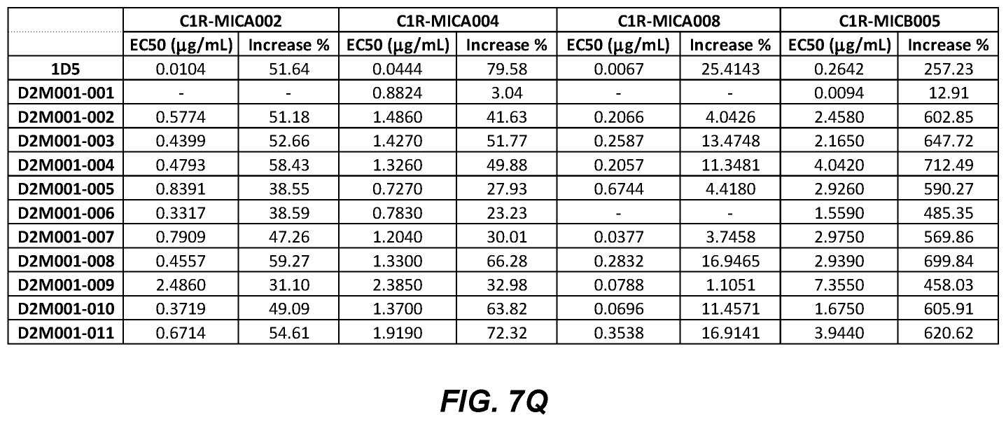

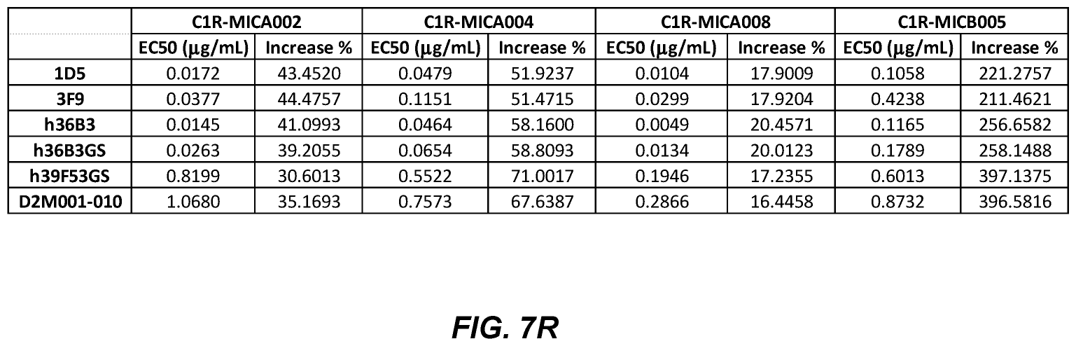

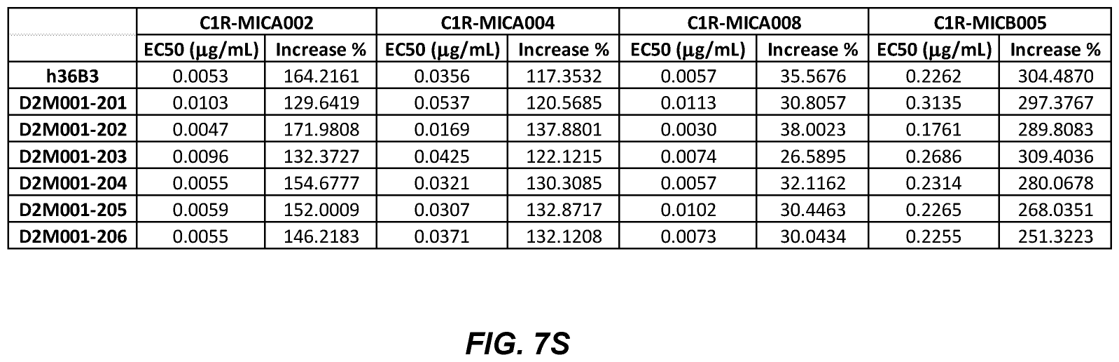

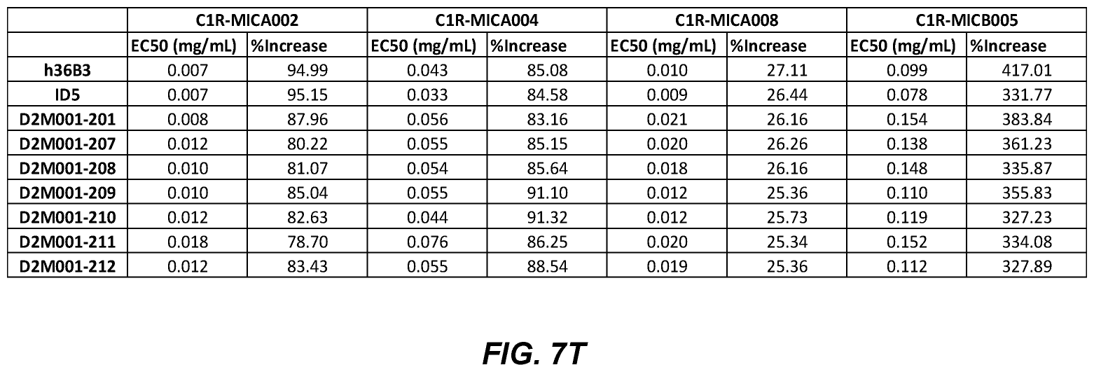

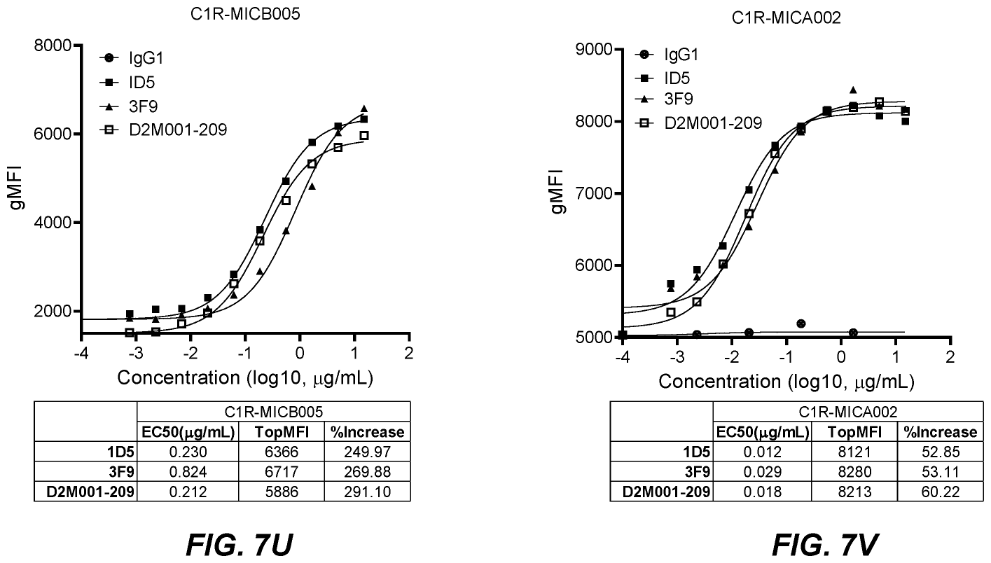

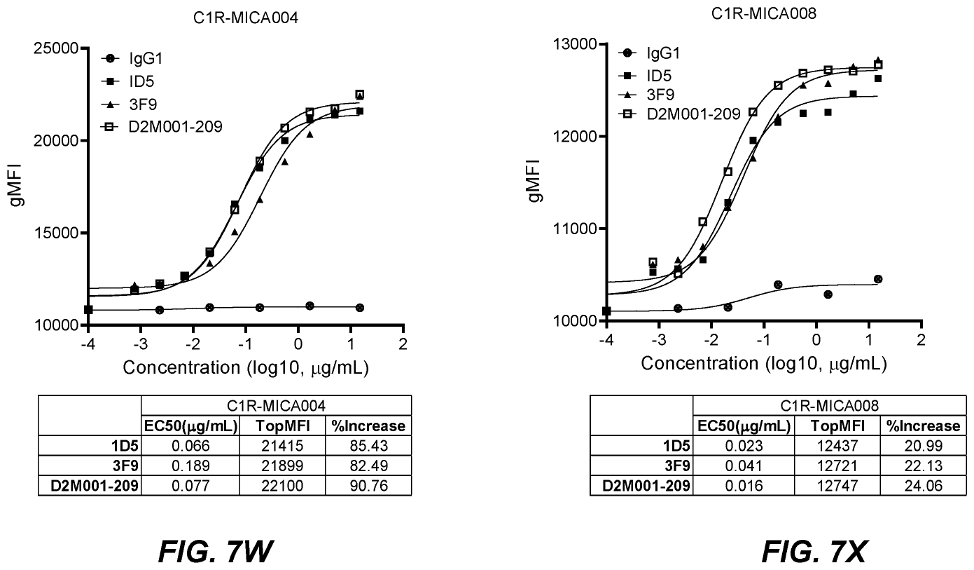

As shown in FIGS. 6A-6X, after treatment of C1R-MICA/B with humanized anti-MICA/B antibodies, the shed MICA/B polypeptides decreased significantly in the cell culture medium. Further, as shown in FIGS. 7A-7P, the surface expression of MICA/B alleles increased significantly in the C1R-MICA/B cells, indicating the humanized anti-MICA/B antibodies and the reference antibodies (1D5 and 3F9) exhibited strong inhibition of the shedding of all four MICA/B alleles expressed on C1R cells, while the isotype control antibody IgG1 did not. The maximum shedding inhibition and IC50 values of shed MICA/B (shown in FIGS. 6Q-6T) were calculated based on non-linear fitting curves by GraphPad Prism software. The maximum changes of surface MICA/B expression and EC50 values (shown in FIGS. 7Q-7T) were calculated based on the non-linear fitting curves by GraphPad Prism software.

As shown in FIGS. 6U-6X, D2M001-209 was further compared with benchmark anti-MICA/B in their abilities to inhibit the shedding of MICA/B polypeptides and stabilize the surface expression of MICA/B. D2M001-209 demonstrated the ability to inhibit MICA/B shedding comparable to 1D5 and more potent than 3F9 in term of IC50 and maximum effects.

As shown in FIGS. 7U-7X, D2M001-209 was further compared with benchmark anti-MICA/B in their abilities to inhibit the shedding of MICA/B polypeptides and stabilize the surface expression of MICA/B. D2M001-209 demonstrated the ability to stabilize surface MICA/B better than 1D5 and 3F9 in term of EC50 and/or percentage of increase.

### Example 5: Anti-MICA/B Antibodies Capture MICA/B Polypeptides Shed from the Surface of Cells

As discussed in further detail below, the anti-MICA/B antibodies identified in Example 1 not only inhibit the shedding of MICA/B polypeptides from cells, but also bind to shed MICA/B polypeptides that are released from tumor cells. To investigate the binding affinities of anti-MICA/B antibodies against shed MICA/B polypeptides, shed MICA/B polypeptides were concentrated from conditional medium of CHO-MICA002, CHO-MICA004, CHO-MICA008, and CHO-MICB005, using the Spin-X concentrator (Corning, Cat #: 431489). Each well of a 96-well plate was coated with 100 μL 5 μg/mL anti-hFec (Jackson ImmunoResearch, Cat #: 109-005-098) overnight, and the plate was washed with PBS twice and blocked with 1% BSA for 1 hour at room temperature. After two additional PBS washes, 100 μL each of titrated anti-MICA/B antibodies, reference antibodies (1D5, 13A9, 6E1.1.12, and 3F9), or an isotype control antibody (IgG1) were added to the wells of the plate to final concentrations of 15 μg/mL, 5 μg/mL, 1.667 μg/mL, 0.556 μg/mL, 0.185 μg/mL, 0.062 μg/mL, 0.021 μg/mL, 0.007 μg/mL and 0 μg/mL, and incubated for 30 minutes at room temperature. The plate was washed with PBS twice and 100 μL of shed MICA/B polypeptides at a concentration of 80 ng/mL were added to each well of the plate and incubated for 2 hours at room temperature. After two additional PBS washes, 100 μL of 1 μg/mL biotinylated anti-MICA/B 6D4 (BioLegend, Cat #: 320904) was added to each well of the plates and incubated for 1.5 hours at room temperature. 100 μL Streptavidin-HRP solution (R&D Systems, Cat #: DY998, 200×) was added and incubated at room temperature for 1 hour. After washes, the plate was developed with TMB and stopped with BioFX 450 nM Liquid stop solution. OD450 was measured using the Varioskan™ Lux plate reader.

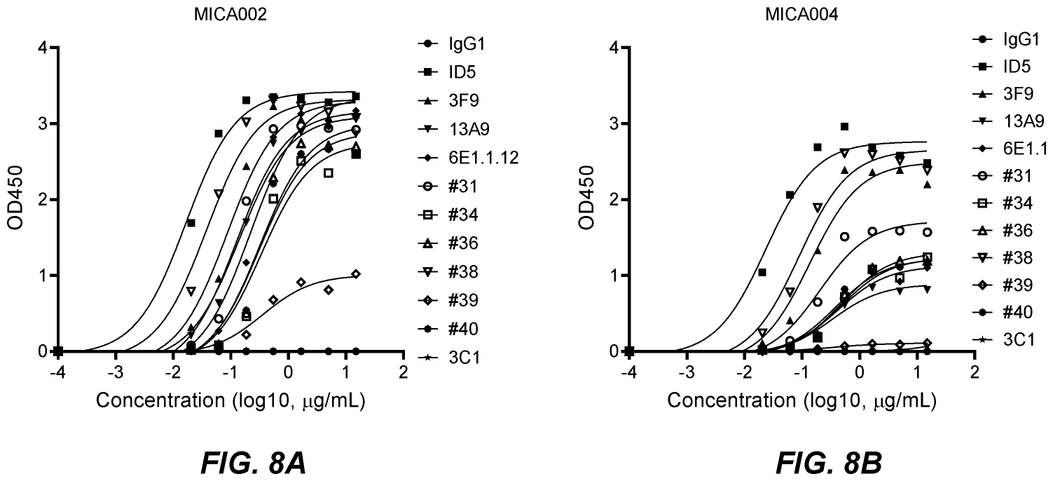

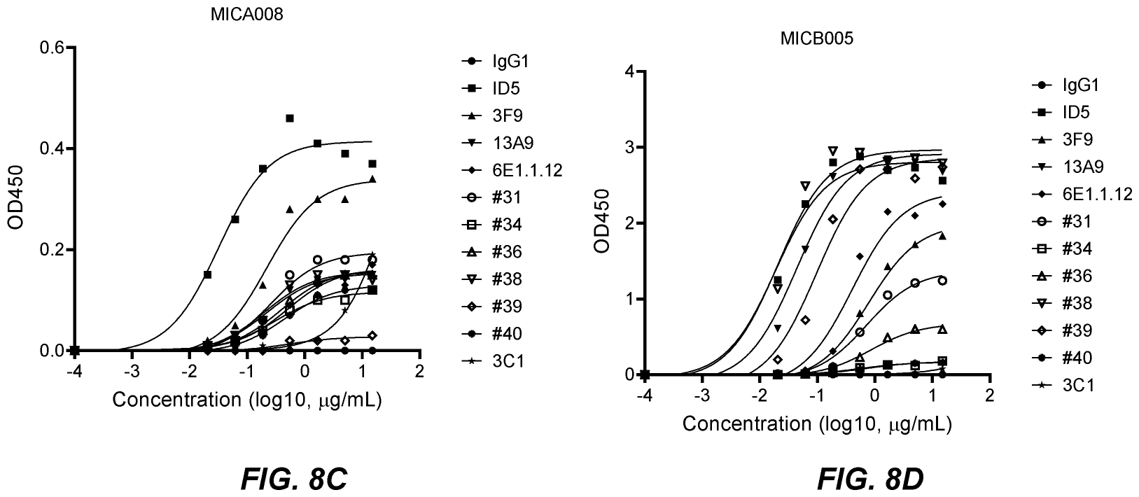

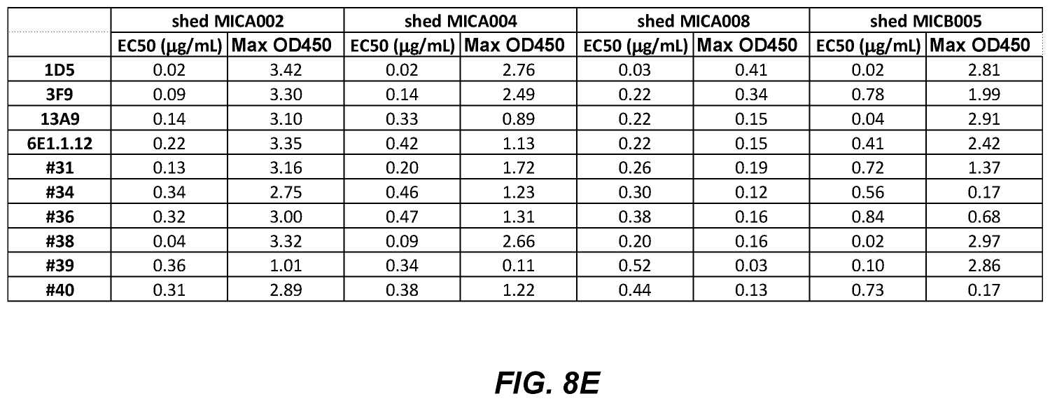

As shown in FIGS. 8A-8E, the chimeric anti-MICA/B antibodies and the reference antibodies exhibited strong binding affinities to all four shed MICA/B polypeptide variants released from CHO cells overexpressing MICA/B alleles while the isotype control antibody IgG1 did not. The maximum binding signals (OD450) and EC50 values of shed MICA/B binding (shown in the left panel of FIG. 8E) were calculated based on non-linear fitting curves by GraphPad Prism software.

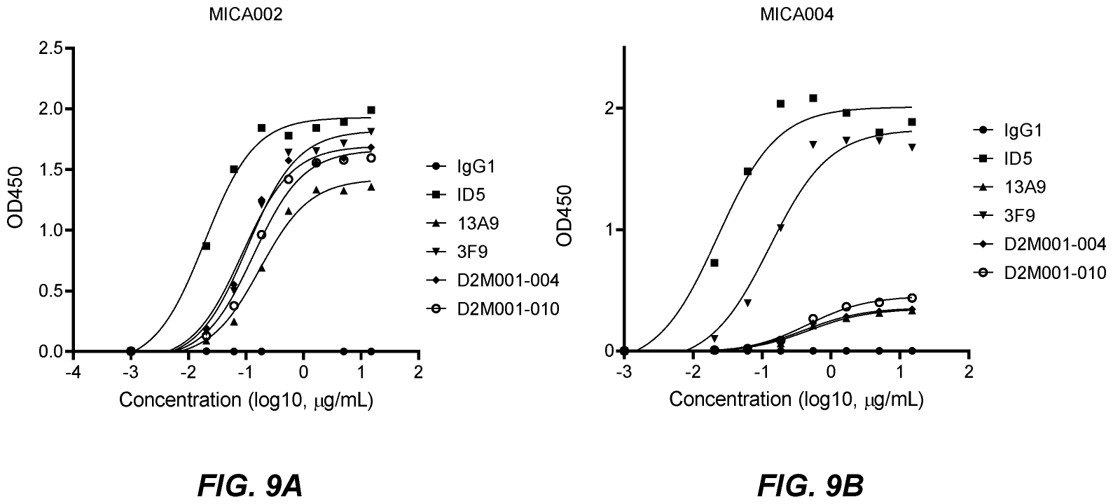

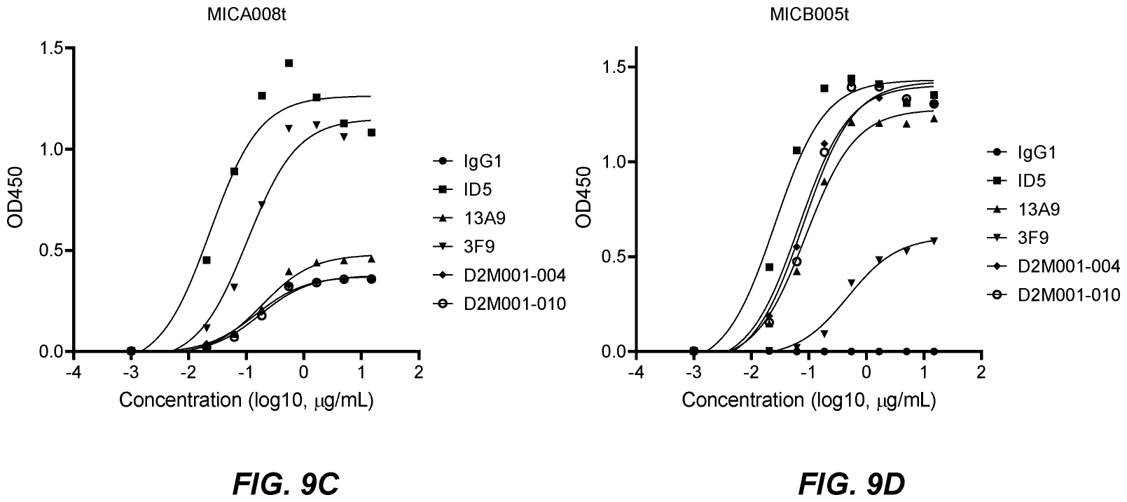

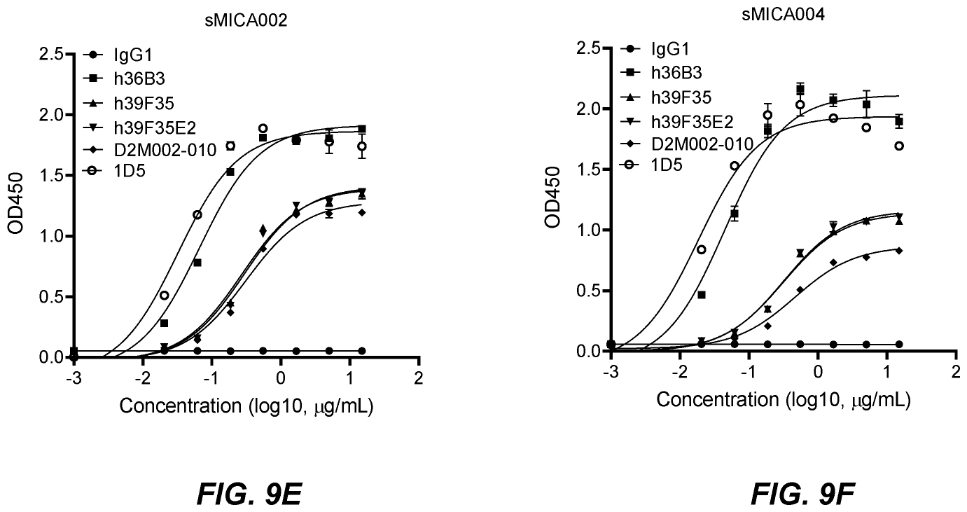

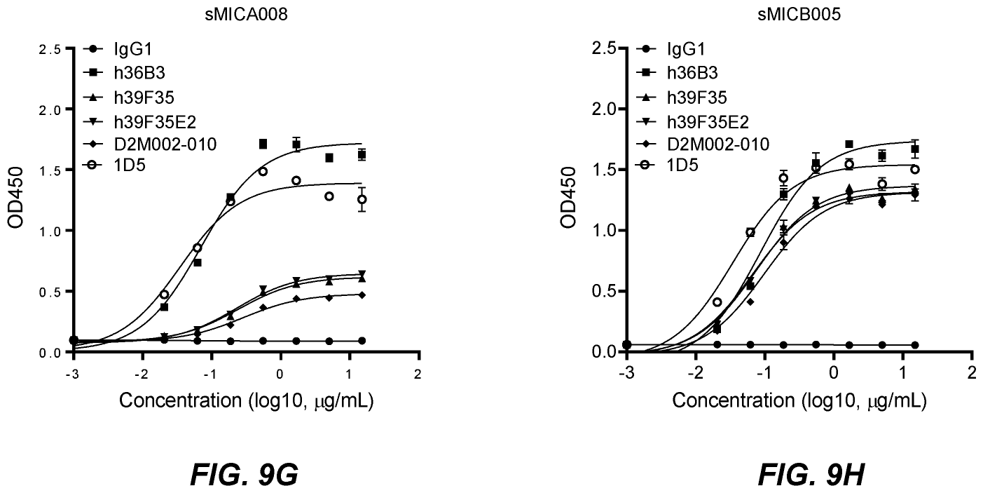

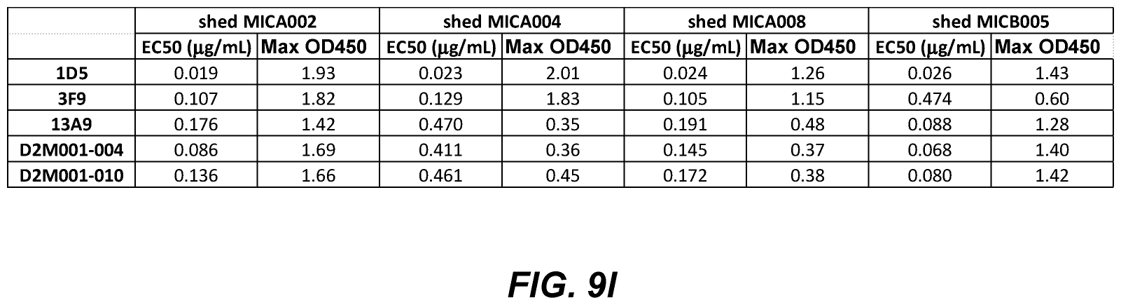

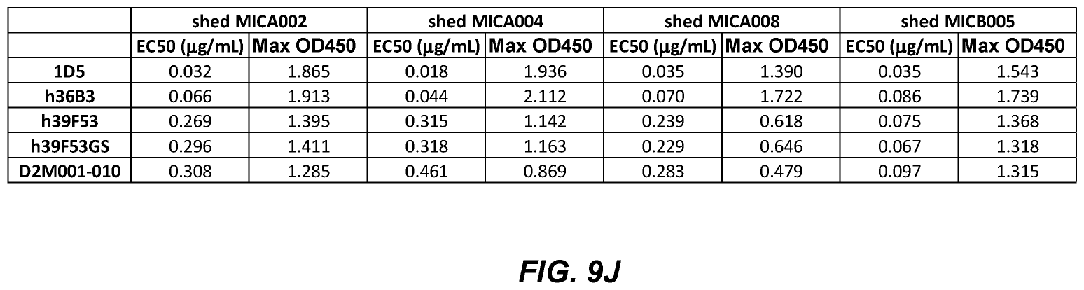

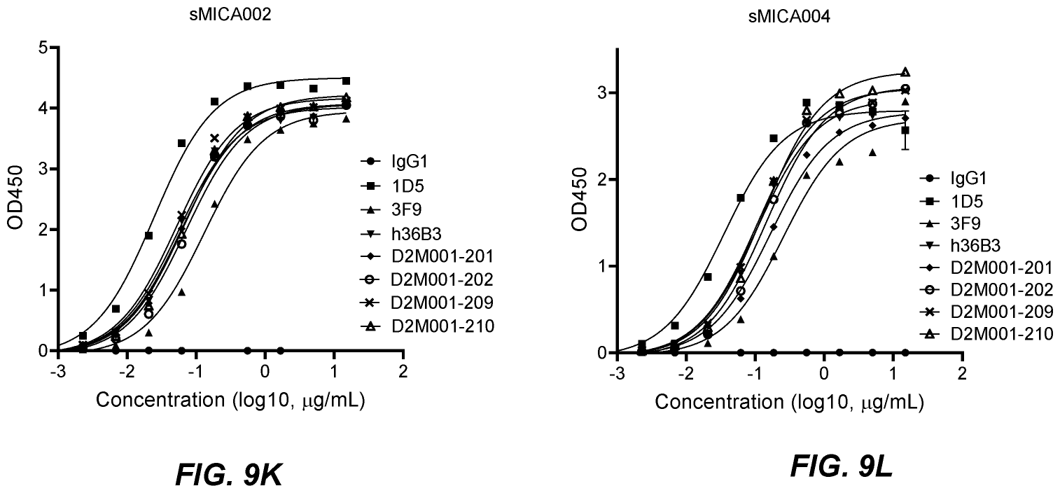

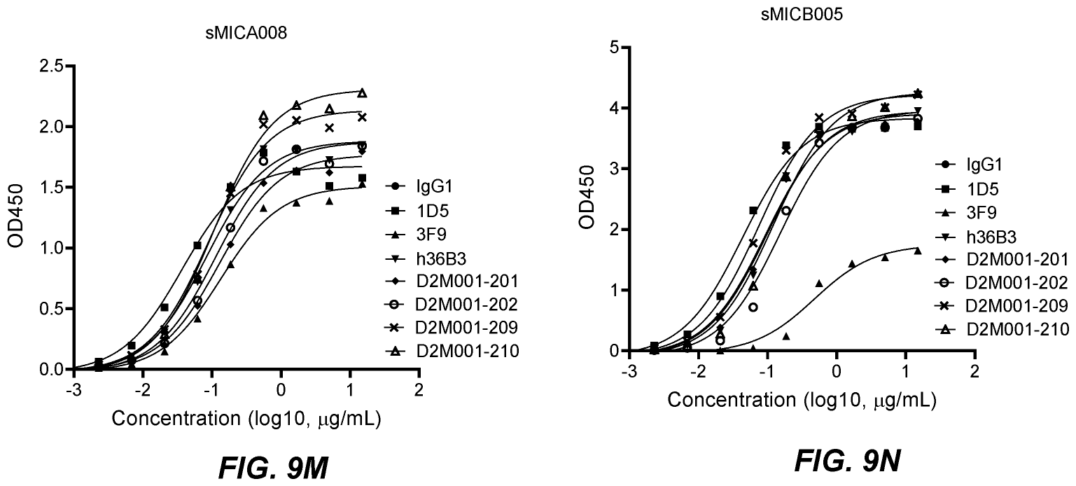

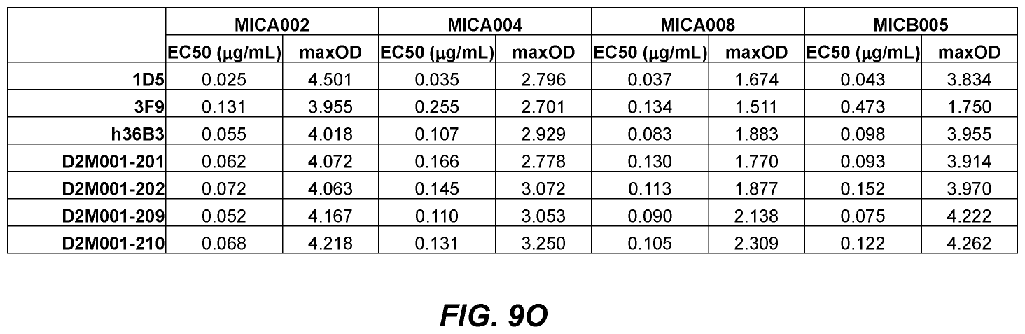

As shown in FIGS. 9A-9H and FIGS. 9K-9N, the humanized anti-MICA/B antibodies and the reference antibodies (1D5, 13A9 and 3F9) exhibited strong binding affinities to all four shed MICA/B variants released from CHO cells overexpressing MICA/B alleles while the isotype control antibody IgG1 did not. The maximum binding signals (OD450) and EC50 values of shed MICA/B binding shown in FIGS. 9I-9J and FIG. 9O were calculated based on non-linear fitting curves by GraphPad Prism software.

As shown in FIGS. 9K-9N, D2M001-209 exhibited the strong ability to capture soluble MICA/B, which is comparable to 1D5 and more potent than 3F9 for all alleles tested. In particular, D2M001-209 not only can capture MICB005 much more efficiently than 3F9, but also can capture more MICB than 3F9. Even when reached plateau, 3F9 can only capture less than half amount of MICB, compared to D2M001-209. The superior ability to capture soluble MICB is important and well differentiates D2M001-209 and 3F9, as indicated in Example 6 that MICB may play more important role in NKG2D biology.

### Example 6: Immunocomplexes of Anti-MICA/B Antibodies and Shed MICA/B Polypeptides Co-Stimulate the Activation of Human T Cells

The immunocomplex of anti-MICA/B antibodies and shed MICA/B polypeptide variants can bind to the activating receptor NKG2D as its ligand.

Firstly, to verify the interaction of NKG2D with MICA/B and prove an anti-MICA/B alpha3 domain antibody does not block the interaction, a 96-well assay plate was coated with NKG2D-His (BioLegend, Cat #: 781906)(200 ng/well) at 4° C. overnight. After two PBS washes and blocked with 1× Reagent diluent concentrate 2 (R&D systems, Cat #: 841380), titrated MICA/B-ECD solution (starting at 3 μg/mL and 3-fold dilution) was added and incubated for 2 hours at room temperature. After washes, 100 μL of 1 μg/mL reference anti-MICA/B alpha3 domain antibody (ID5) (100 ng/well) were added and incubated for 1.5 hours at room temperature. After washes, 100 μL anti-hFc-HRP (Jackson ImmunoResearch, Cat #: 209-035-098, 1:5000) was added and incubated for 1 hour at room temperature. After washes, the plate was developed with TMB and stopped with BioFX 450 nM Liquid stop solution. OD450 was measured using the Varioskan™ Lux plate reader.

Secondly, to investigate the interaction of NKG2D and the immunocomplex of anti-MICA/B antibodies and shed MICA/B polypeptides, shed MICA/B polypeptides were concentrated from conditional medium of CHO-MICA002 or CHO-MICA004 using the Spin-X concentrator (Corning, Cat #: 431489). Specifically, a 96-well assay plate was coated with 100 μL goat anti-human Fc (Jackson ImmunoResearch, Cat #: 109-005-098, 5 μg/mL in 1×PBS) overnight. After two PBS washes and blocked with 1% BSA in PBS, 100 μL each of titrated anti-MICA/B antibodies, three reference antibodies (1D5, 13A9 and 3F9), or an isotype control antibody (IgG1) were added to wells of the plate to final concentrations of 15 μg/mL, 5 μg/mL, 1.667 μg/mL, 0.556 μg/mL, 0.185 μg/mL, 0.062 μg/mL, 0.021 μg/mL, 0.007 μg/mL and 0 μg/mL, and incubated for 30 minutes at room temperature. The plate was washed with PBS twice and 100 μL shed MICA002 polypeptide (250 ng/mL) was added and incubated for 2 hours at room temperature. After two additional PBS washes, 100 μL 1 μg/mL NKG2D-His (BioLegend, Cat #: 781906) were added and incubated for 1.5 hours at room temperature. 100 μL Anti-His-HRP (BioLegend, Cat #: 652504, 1:10000) were added and incubated for 1 hour at room temperature. After washes, the plate was developed with TMB and stopped with BioFX 450 nM Liquid stop solution. OD450 was measured using the Varioskan™ Lux plate reader.

Thirdly, to demonstrate the immune complex (IC) formed by anti-MICA/B and MICA/B, Hut-78 human CD4 T-cell line which constitutively expresses NKG2D was used to develop an assay. The assay investigated whether the immunocomplexes co-stimulate the activation of Hut-78 cells through MICA/B-NKG2D under anti-CD3 (TCR signaling) stimulation by measuring the production of IL2. Specifically, each well of a 96-well assay plate was coated with 100 μL of a cocktail (5 μg/mL each) of goat anti-human Fc (Jackson ImmunoResearch, Cat #: 109-005-098) and goat anti-mouse Fc (Jackson ImmunoResearch, Cat #: 115-005-071) in 1×PBS overnight. The plate was washed with PBS twice and 100 μL of anti-CD3 (1 μg/mL) alone or cocktail of anti-CD3 (1 μg/mL) and anti-MICAB (2 μg/mL) were added to each well of the plate and incubated for 1 hour at room temperature. After washing with PBS twice, 50 μL shed MICA/B polypeptides were added to each well of the plates and incubated for 1 hour at room temperature. 50 μL Hut-78 cells (approximately 4.0×10⁶ cells/mL) were added to each well of the plate, mixed, and cultured in a CO₂ cell culture incubator for 16 hours. To assess the activation of Hut-78 cells, cytokine levels of IL2 in the cell culture supernatants were quantified by an ELISA kit of human IL2 (BioLegend, Cat #: 431816), according to manufacturer's instruction.

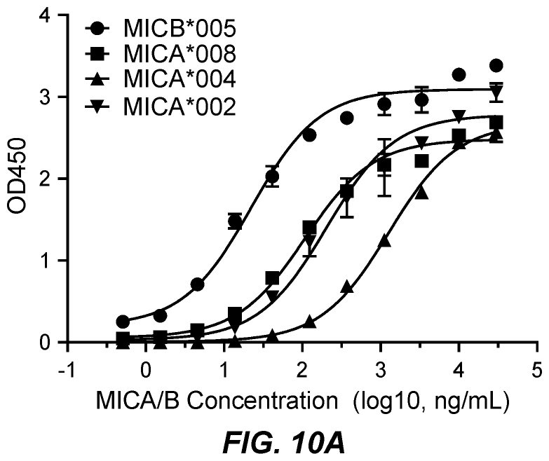

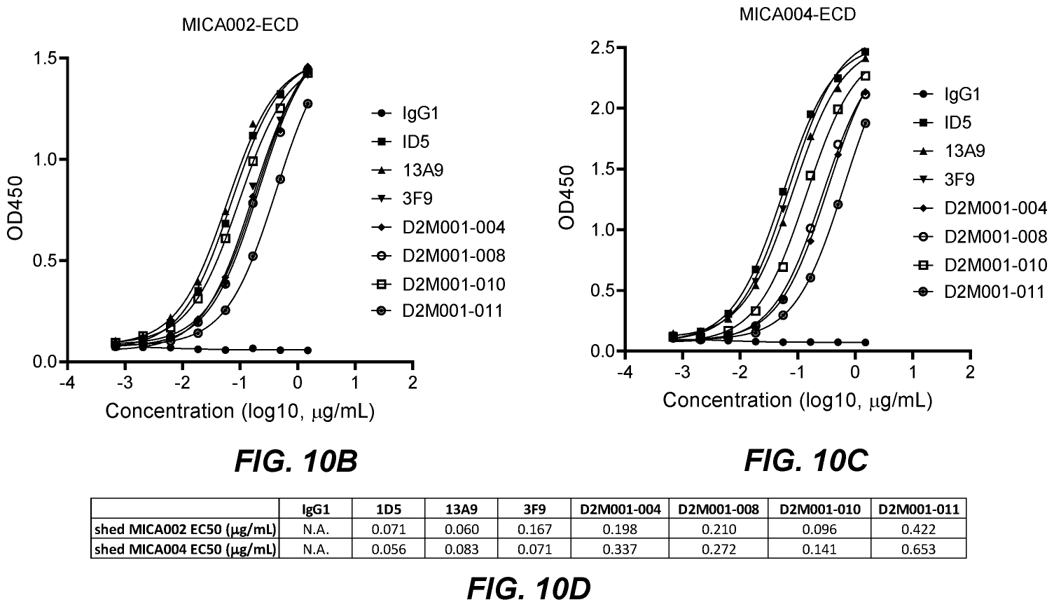

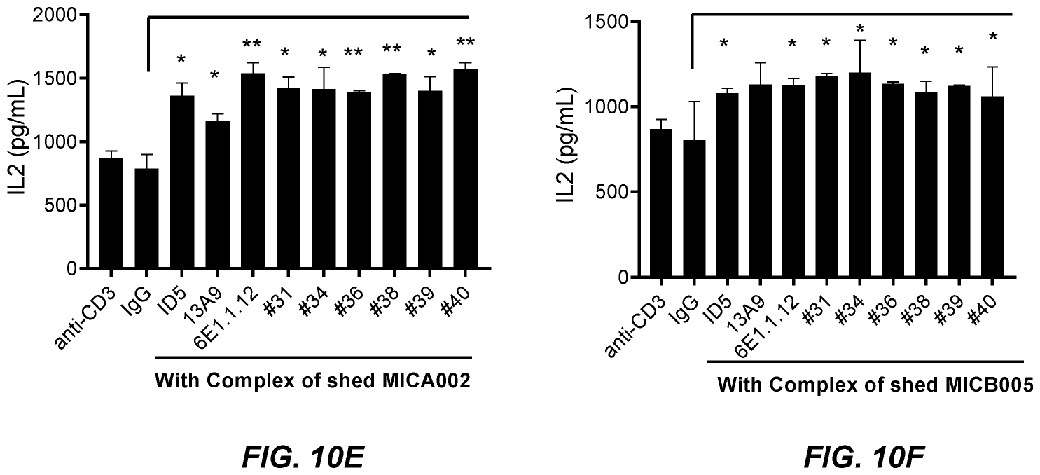

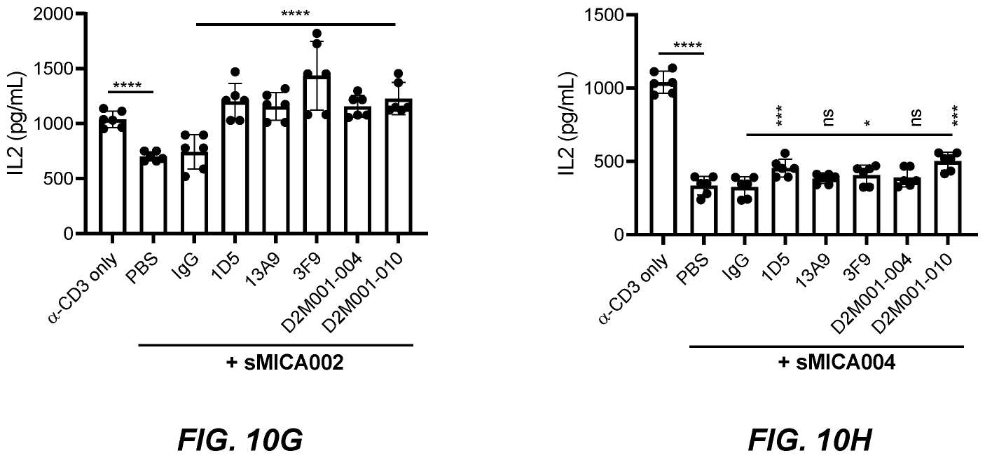

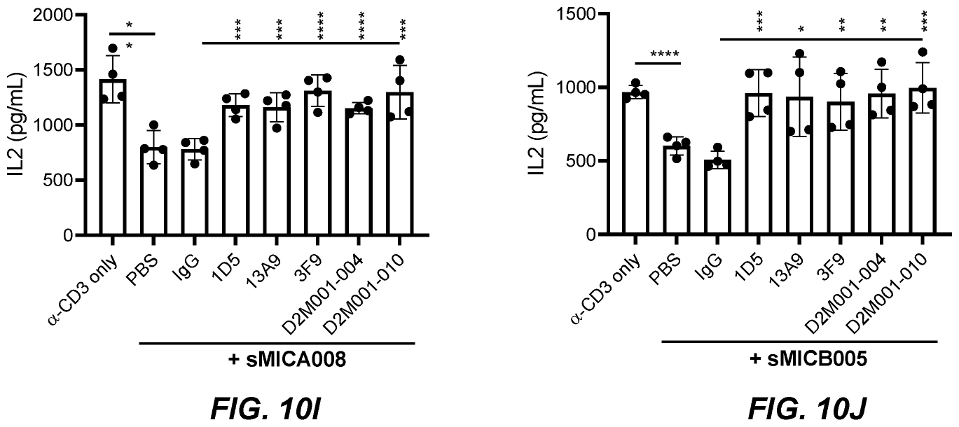

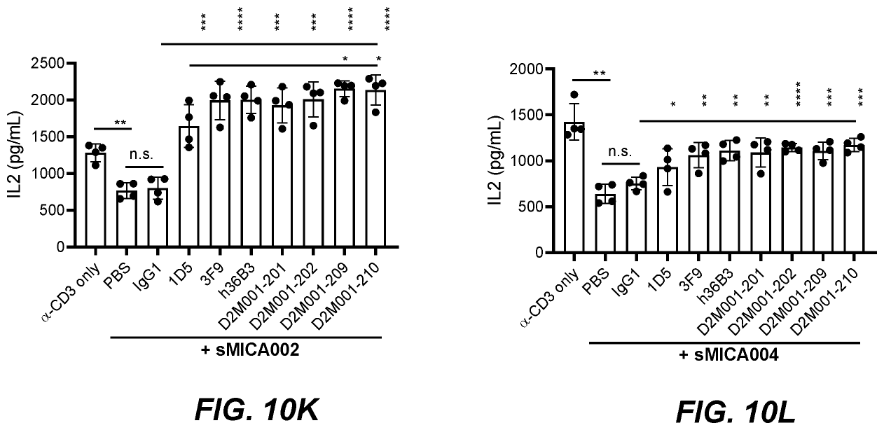

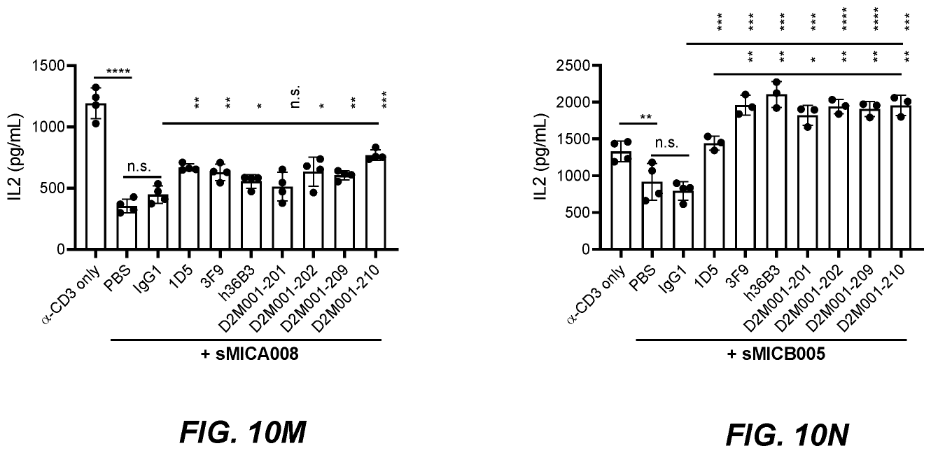

As shown in FIG. 10A, although all synthetic MICA/B polypeptides bound to NKG2D, MICB has the stronger binding affinity to NKG2D than other alleles. This observation suggests that MICB plays more important role than MICA alleles in NKG2D pathway, because with higher binding affinity to NKG2D, on one hand, MICB may agonize NKG2D more efficiently than MICA alleles do; on the other hand, once shed from surface, soluble MICB would antagonize NKG2D more severely than MICA alleles do. Therefore, higher binding affinity to MICB and higher potency in capture soluble MICB are preferable.

As shown in FIGS. 10B-10C, the immunocomplex of humanized anti-MICA/B antibody and shed MICA/B polypeptides bound to NKG2D. The EC50 values of NKG2D binding (shown in FIG. 10D) were calculated based on non-linear fitting curves by GraphPad Prism software.

As shown in FIGS. 10E-10F, the immunocomplex of chimeric anti-MICA/B antibody and shed MICA002 polypeptide (FIG. 10E) and MICB005 polypeptide (FIG. 10F) promoted the activation of Hut-78 cells by increasing the production of IL2. As shown in FIGS. 10G-10N, the immunocomplex of humanized anti-MICA/B antibody and shed MICA/B polypeptides promoted the activation of Hut-78 cells, accordingly to the measurement of increased production of IL2.

As shown in FIGS. 10K, 10L and 10N, compared to 1D5, D2M001-209 appeared to form an immune complex with MICA002, MICA004, or MICB005 that can more actively coactivate T cells through NKG2D,

### Example 7: Immunocomplexes of Anti-MICA/B Antibodies and MICA/B Polypeptides Activate Human Primary NK Cells

Like human T cells, human natural killer (NK) cells express NKG2D. Primary human NK cells were used to investigate whether the immunocomplexes of anti-MICA/B antibodies and shed MICA/B polypeptides activate human NK cells via NKG2D. Human primary NK cells were isolated from human PBMC using the MojoSort Human NK Cell Isolation Kit (BioLegend, Cat #: 480054). Shed MICA/B polypeptides were concentrated from conditional medium of CHO-MICA002, CHO-MICA004, CHO-MICA008, and CHO-MICB005 cells, respectively, by using the Spin-X concentrator (Corning, Cat #: 438419). In a 96-well V-bottom culture plate, 50 μL of the immunocomplex were added by mixing concentrated sMICA/B polypeptides (500 ng/mL) with anti-MICA/B antibodies or controls (2 μg/mL) in wells of the culture plates and incubated for 30 minutes at room temperature. 50 μL purified human NK cells (approximately 1.0×10⁴ cells) were added along with 2× Brefeldin A (BioLegend, Cat #: 420601) and mixed well. The plate was centrifuged for 1 minute at 800 g and cultured for 6 hours in a CO₂ cell culture incubator. The cells were fixed using Intracellular Fixation Buffer (BioLegend, Cat #: 420801) and stained with PE anti-human IFNγ (BioLegend, Cat #: 502509) according to the intracellular flow cytometry staining protocol provided with the Intracellular Staining Perm Wash Buffer (BioLegend, Cat #: 421002).

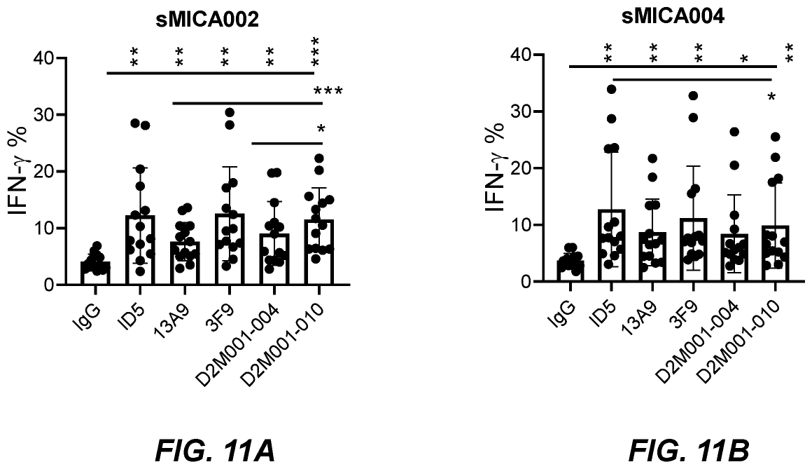

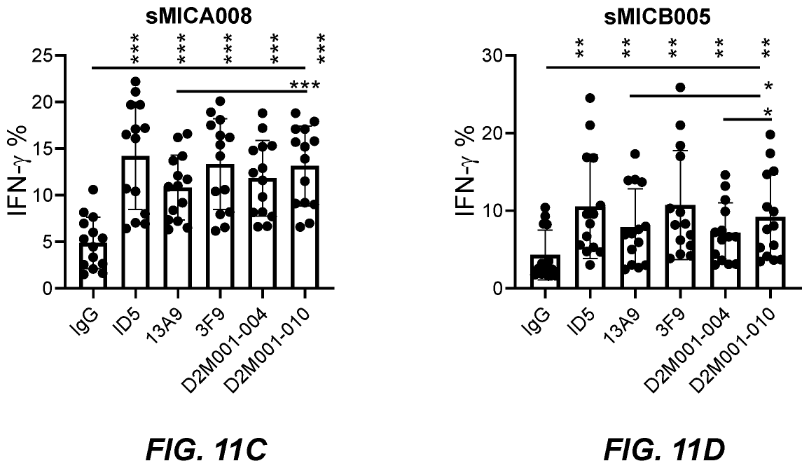

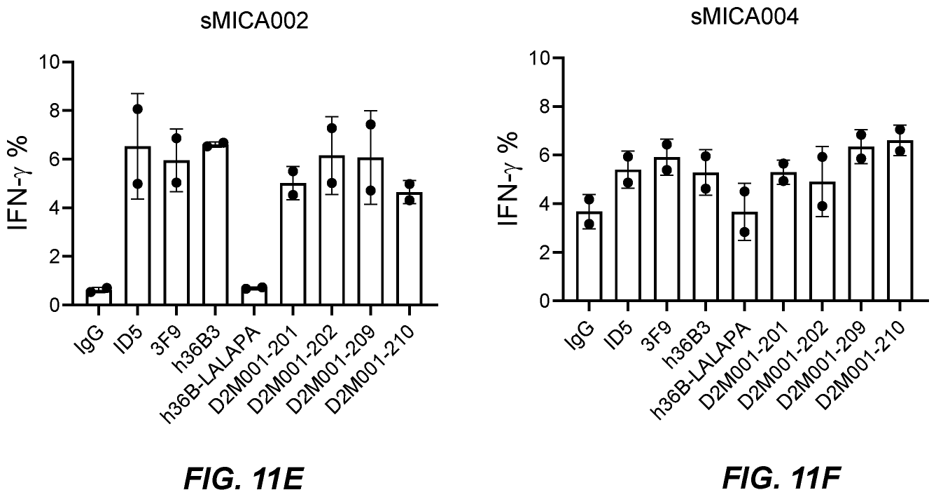

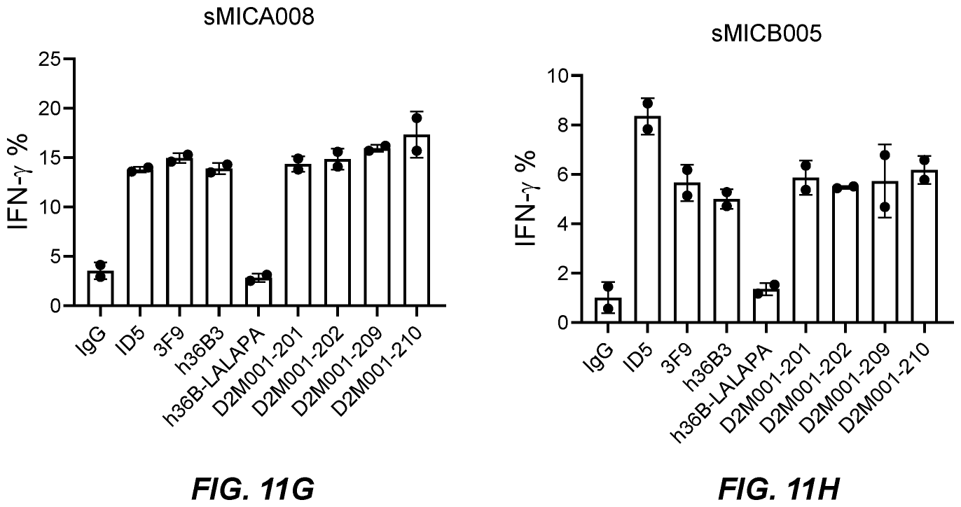

As shown in FIGS. 11A-11H, the immunocomplex of humanized anti-MICA/B antibody and shed MICA/B polypeptides can promote the activation of human NK cells by increasing the expression of IFNγ in multiple donors. As shown in FIGS. 11E-11H, LALAPA mutation abolished the activity to activate NKG2D, indicating that the activity is FcγR dependent.

### Example 8: Anti-MICA/B Antibodies Enhance Primary NK Cell Activation Through NKG2D

It has been hypothesized that the overexpressed MICA/B proteins on the surfaces of tumor cells bound by anti-MICA/B antibodies can promote the activation of NK cells through NKG2D. NK activation assays were performed with human primary NK cells isolated from PBMC using the MojoSort Human NK Cell Isolation Kit (BioLegend, Cat #: 480054). Specifically, C1R-MICA/B tumor cells were labeled with 1 mM CSFE (BioLegend Cat #: 423801) for 10 minutes at 37° C. C1R-MICA/B cells were treated with 10 mg/mL anti-MICA/B antibodies or IgG1 for 24 hours at room temperature. In a 96-well V-bottom culture plate, 50 μL (approximately 1.0×10⁶/mL) pretreated tumor cells and 50 μL (approximately 2.0×10⁶/mL) NK cells in the presence of 2× Brefeldin A (BioLegend, Cat #: 420601) were added to each well of the plate and mixed well. The plate was centrifuged for 3 minutes at 800 g and cultured for 5 hours in a CO₂ cell culture incubator. The cells were stained with the surface CD107a with Alexa Fluor® 647 anti-human CD107a (LAMP-1) Antibody (BioLegend, Cat #: 328612) and then intracellularly stained with PE anti-human IFNγ (BioLegend, Cat #: 502509) according to the intracellular flow cytometry staining protocol provided with the Intracellular Staining Perm Wash Buffer (BioLegend, Cat #: 421002). PE signals of IFNγ and APC signals of CD107a were quantified by flow cytometry gated with CSFE negative NK cells.

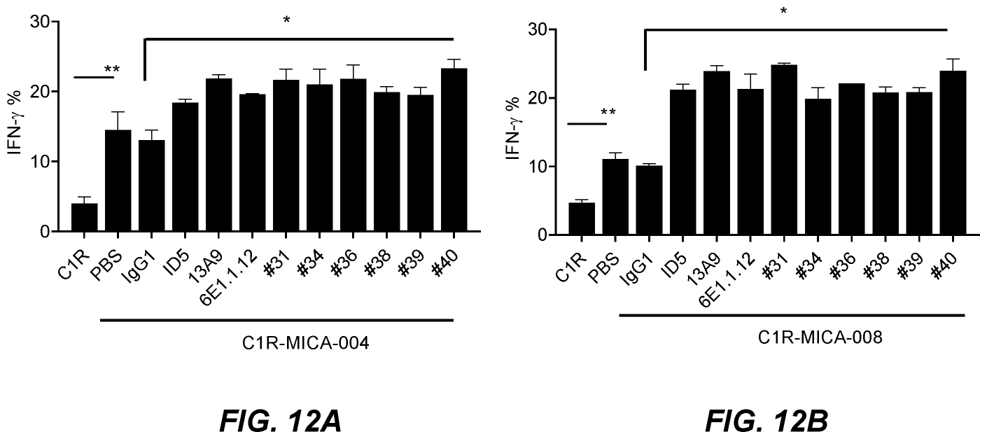

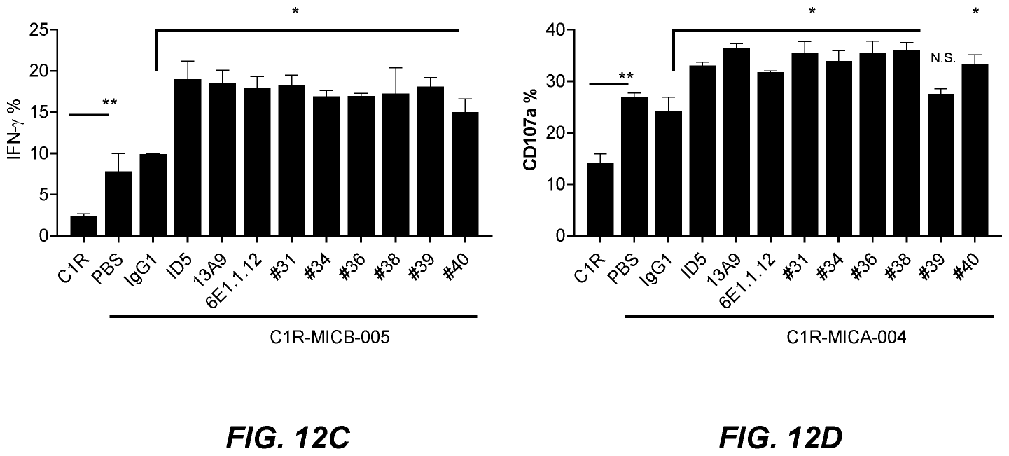

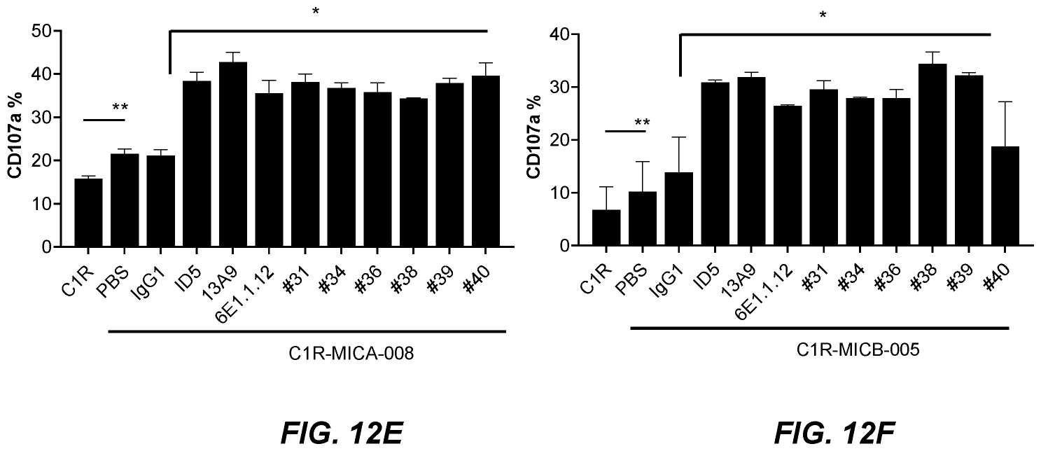

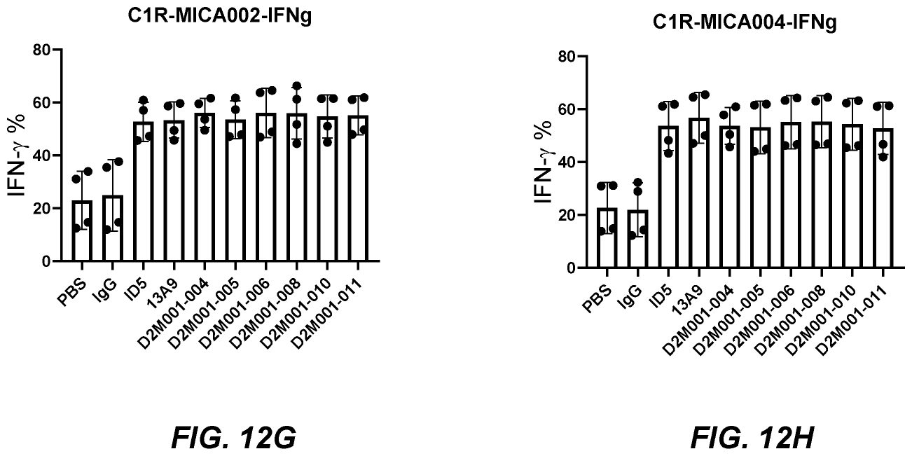

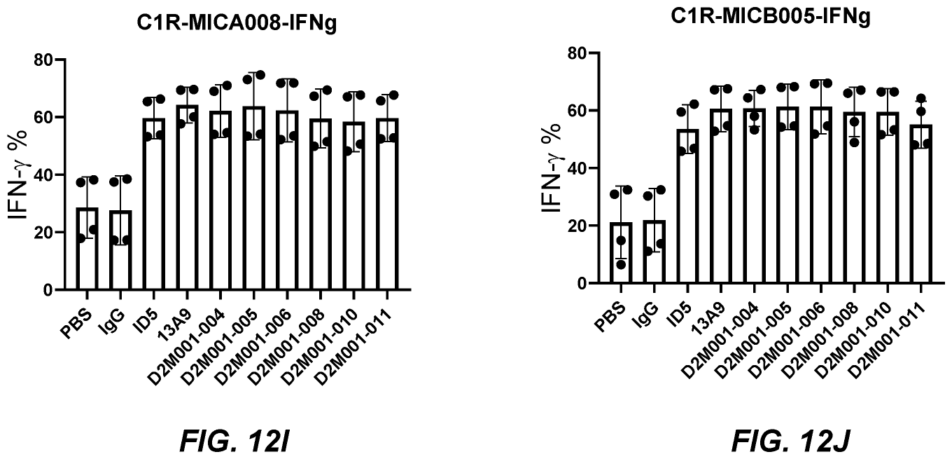

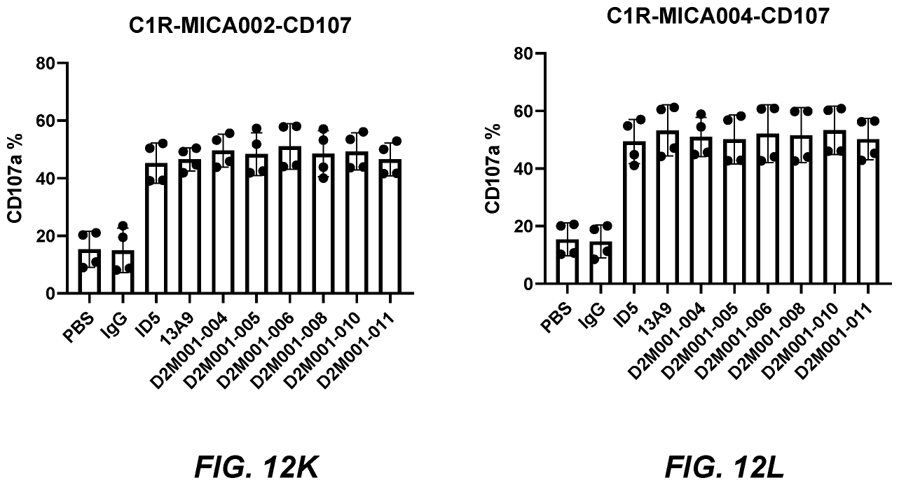

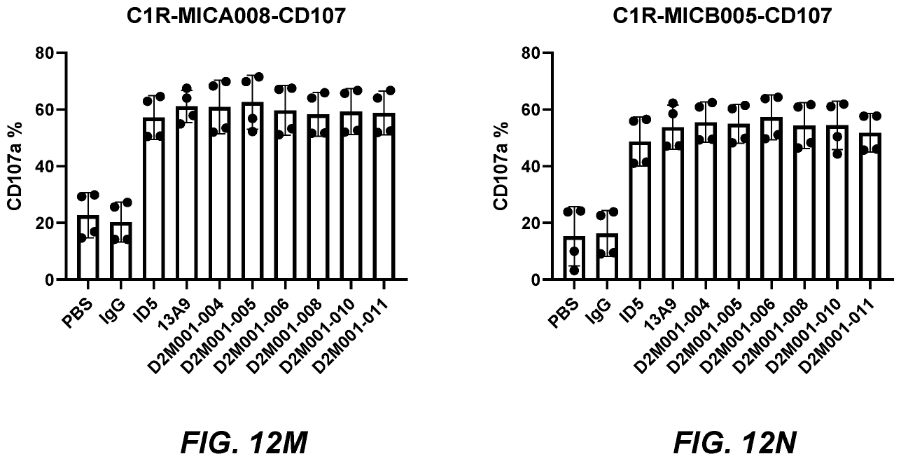

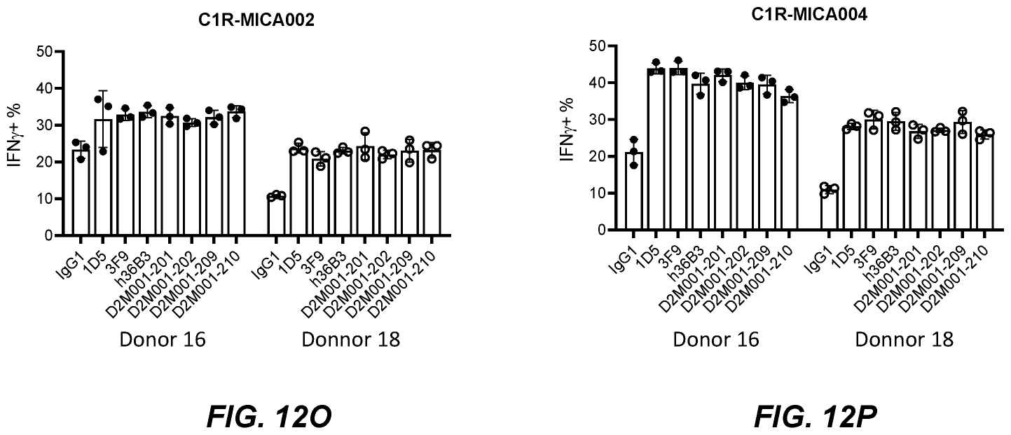

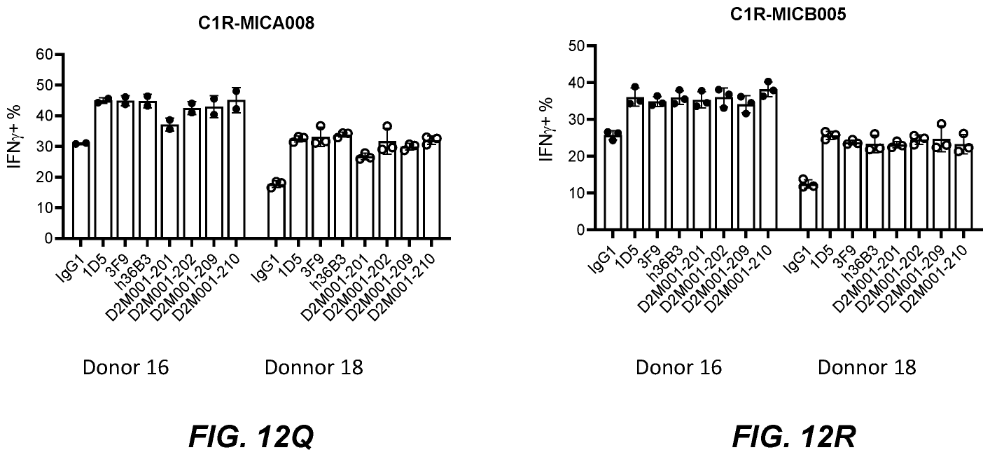

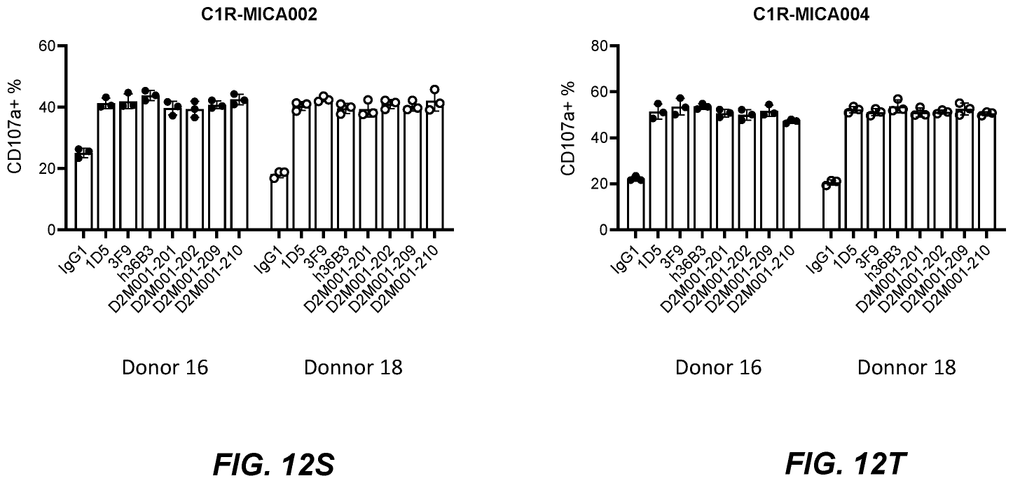

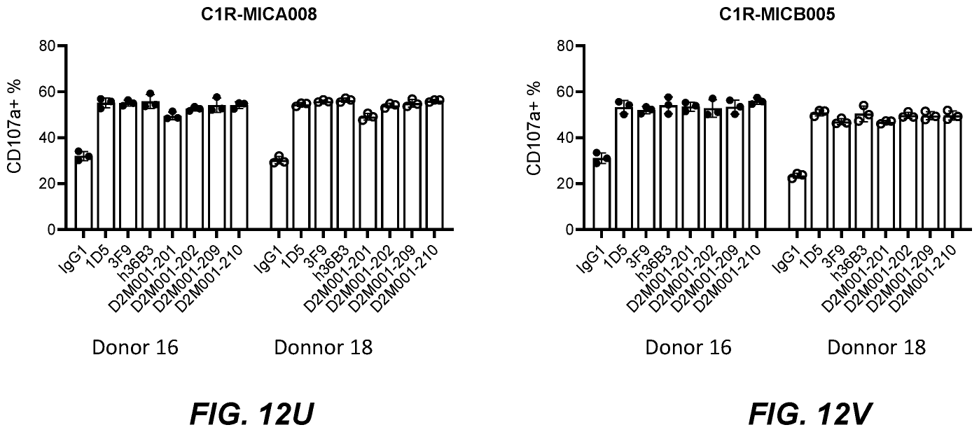

As shown in FIGS. 12A-12F, CIR cells expressing MICA/B polypeptides alone enhanced the activation and degranulation of NK cells compared to parental CIR cells that do not express MICA/B, pretreated tumor cells expressing MICA/B polypeptides treated with chimeric anti-MICA/B antibodies further enhanced the activation and degranulation of NK cells by elevating the expression of IFNγ (FIGS. 12A-12C) and CD107a (FIGS. 12D-12F) compared to the IgG1 controls or without antibody treatment (PBS alone). The assays were performed with NK cells from multiple donors.

As shown in FIGS. 12G-12N and FIGS. 120-12V, pretreated tumor cells expressing MICA/B polypeptides by humanized anti-MICA/B antibodies enhanced the activation and degranulation of NK cells by elevating the expression of IFNγ (FIGS. 12G-12J and FIGS. 120-12R) and CD107a (FIGS. 12K-12N and FIGS. 12S-12V) compared to the IgG1 controls or without antibody treatment (PBS alone).

### Example 9: Anti-MICA/B Antibodies Enhance Killing of Tumor Cells by Primary NK Cells Through Both ADCC and NKG2D Activation

To assess chimeric anti-MICA/B antibodies and #39 humanized variants, cytotoxicity assays were performed with human primary NK cells isolated from PBMC using the MojoSort Human NK Cell Isolation Kit (BioLegend, Cat #: 480054). C1R-MICA/B tumor cells were label with 1 mM CFSE (BioLegend, Cat #: 423821) for 10 minutes at 37° C. C1R-MICA/B cells were treated with 10 μg/mL anti-MICA/B antibodies or IgG1 control for 24 hours. In a 96-well V-bottom culture plate, 50 μL (approximately 1.0×10⁶ cells/mL) pretreated tumor cells and 50 μL (approximately 2.0×10⁶ cells/mL) NK cells were added to each well of the plate and mixed well. The plate was centrifuged for 3 minutes at 800 g and cultured for 4 hours in a CO₂ cell culture incubator. Afterwards, the plate was centrifuged for 3 minutes at 800 g and cells were resuspended in 100 μL FACS with 1×DAPI (Invitrogen, Cat #: 62248). The cytotoxicity of NK cells was quantified by flow cytometry by gating the DAPI+ tumor cells in total CSFE+ tumor cells.

To assess humanized and optimized #36 variants, cytotoxicity assays were performed with purified hNK cultured in cRMPI with 500 IU/mL IL2 overnight. C1R-MICA002, C1R-MICA004, C1R-MICA008, and C1R-MICB005 cells were stably transfected to express GFP The cells were named C1R-MICA004-GFP, C1R-MICA008-GFP, C1R-MICB005-GFP, and C1R-MICB005-GFP, respectively. C1R-MICA004-GFP, C1R-MICA008-GFP, C1R-MICB005-GFP, and C1R-MICB005-GFP were pretreated with 5 mg/mL anti-MICAB for 24 hours. C1R-MICA002/4/8/B005-GFP were cocultured with, at an E:T (effector:target) ratio of 1:1, 5,000 cells each in the 96-well V plate for 4 hours. The cytotoxicity of NK cells was quantified by flow cytometry by gating the DAPI+ tumor cells in total tumor cells (GFP+).

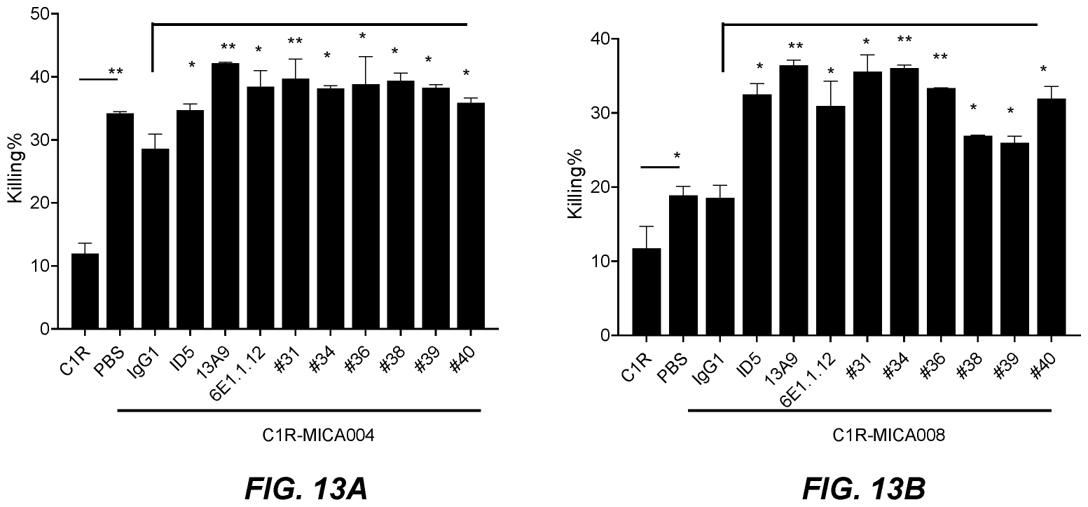

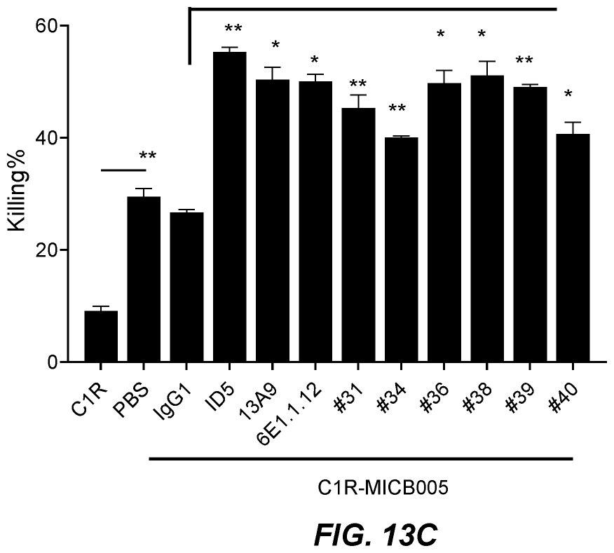

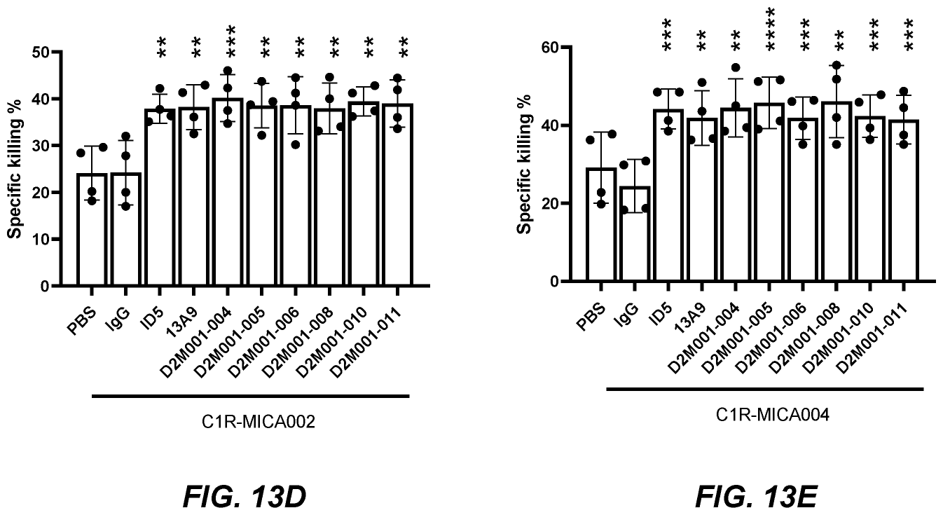

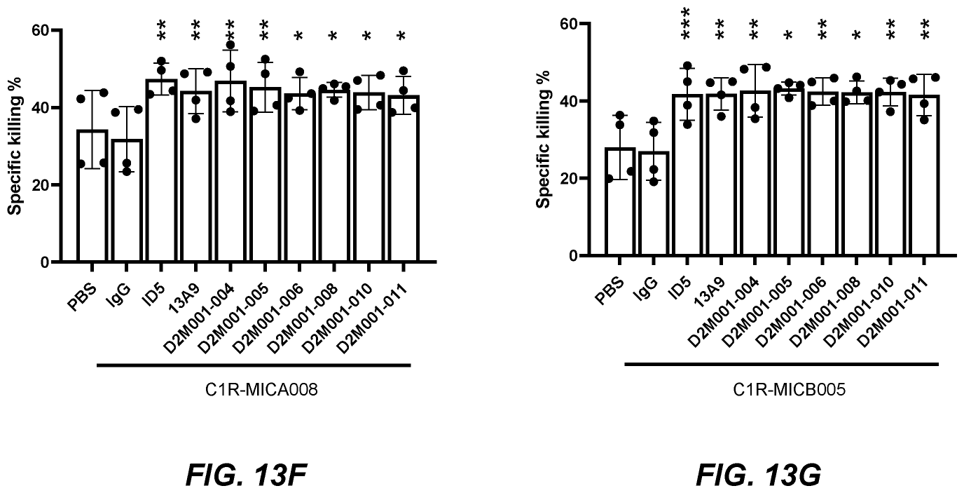

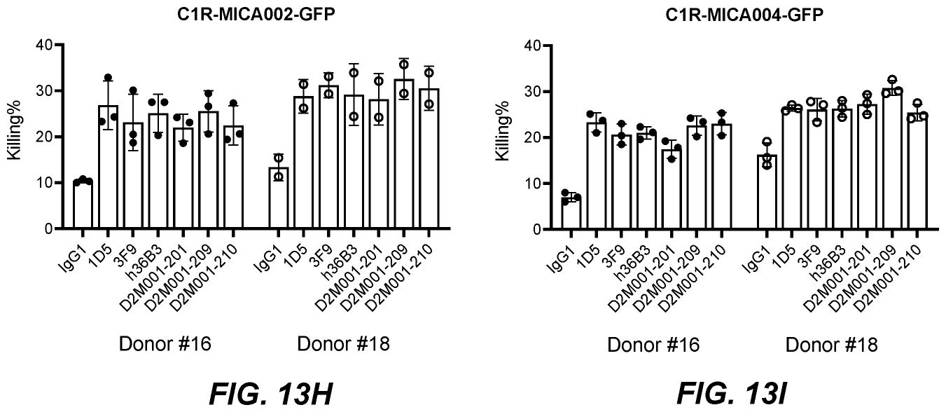

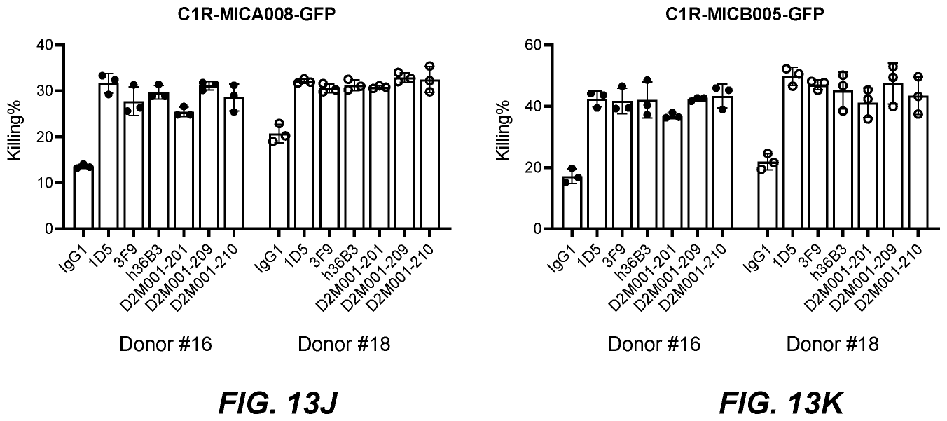

As shown in FIGS. 13A-13C, C1R cells expressing MICA/B polypeptides alone enhanced NK killing compared to parental CIR cells that do not express MICA/B. Pretreated tumor cells expressing MICA/B polypeptides treated with chimeric anti-MICA/B antibodies further enhanced the NK killing of tumor cells compared to the IgG1 controls or the no-treatment control (PBS alone). As shown in FIGS. 13D-13G, pretreated tumor cells expressing MICA/B polypeptides with humanized anti-MICA/B antibodies #39 humanized variants enhanced the NK killing of tumor cells compared to the IgG1 controls or the no-treatment control (PBS alone). C1R cells expressing MICA/B polypeptides alone also enhanced the NK cytotoxicity comparing to parental C1R cells without expressing MICA/B. The assays were performed with NKs from multiple donors. As shown in FIGS. 13H-13K, pretreated tumor cells expressing MICA/B polypeptides with humanized anti-MICA/B antibodies humanized #36 variants enhanced the NK killing of tumor cells compared to the IgG1 controls. D2M001-209 appeared to be more active than other #36 variants. D2M001-209 has exhibited the activity comparable to benchmarks. However, D2M001-209 appeared to promote more NK killing than benchmark 1D5 and 3F9 in FIG. 13H and FIG. 13J and promote more NK killing than 3F9 in FIG. 13K.

### Example 10: Humanized Anti-MICA/B Antibodies Exhibit Superior Thermostability

The melting points of full IgG and F(ab′)₂ of humanized anti-MICA/B antibodies, reference antibodies, and IgG1 were measured by the protein thermal shift dye kit (ThermoFisher, Cat #: 4461146) using a standard qPCR machine. To prepare the F(ab′)₂, 17.25 μL (1 mg/mL) full IgG antibody was digested with 0.25 μL IdeZ together with 2 μL 10× GlycoBuffer 2 (NEB, Cat #: P0770S) in 37° C. a water bath for 2 hours. 10 μL CaptureSelect™ IgG-Fc Magnetic Agarose beads (ThermoFisher, Cat #: 2882852001) were washed with PBS and added to the digestion reaction and incubated for 15 minutes at room temperature. The beads were separated by centrifugation for 2 minutes at 300 g to deplete the undigested antibody and Fc fragments. The supernatant with F(ab)₂ or full IgG antibodies (12.5 μL aliquots) was added to the wells of a PCR plate. 5 μL buffer and 2.5 μL diluted dye (8×) from the protein thermal shift dye kit (ThermoFisher, Cat #: 4461146) were added to each of the wells and mixed well. The plate was heated from 25° C. to 99° C. at a rate of 0.05° C./second and fluorescence was detected for each temperature using QuantStudio™ 6 software (ThermoFisher). The fluorescence intensity was subtracted by the lowest intensity (bottom) before the peak and normalized to the amplitude of peak to bottom. The Tm for each antibody was calculated based on the temperature of 50% of amplitude from bottom to peak.

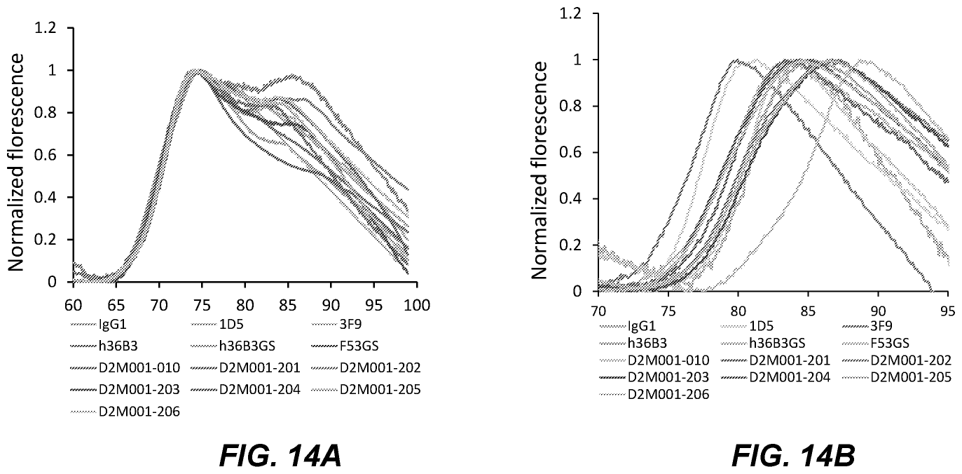

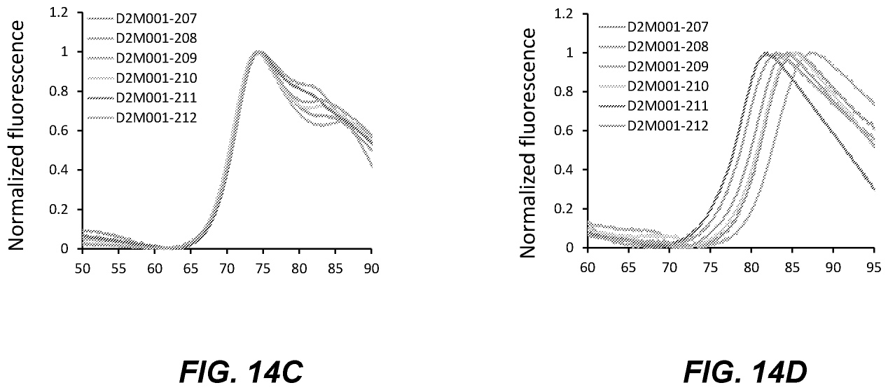

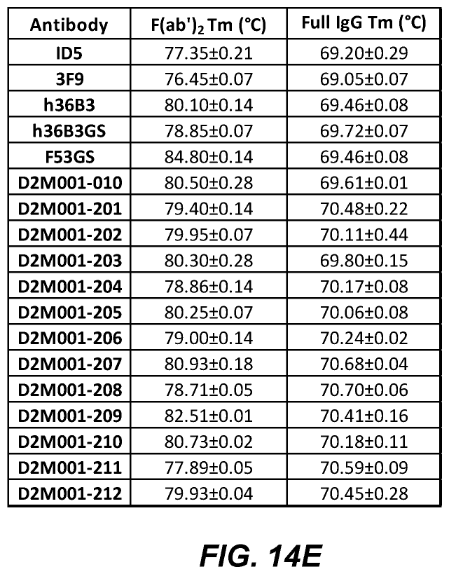

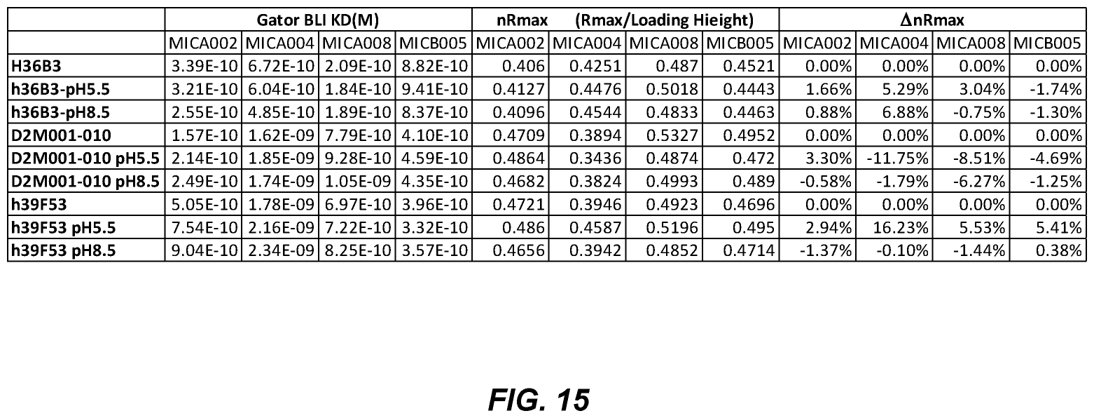

As shown in FIGS. 14A-14E, humanized and optimized anti-MICA/B antibodies demonstrated superior thermostability compared to references. D2M001-209 exhibits much superior thermostability to benchmarks. Specifically, D2M001-209's Tm is 5° C. higher than Tm of 1D5 and 6° C. higher than Tm of 3F9.

### Example 11: Humanized Anti MICA/B Antibodies Exhibited Stability Under Stress Conditions

Common chemical modifications of antibodies include deamidation and isomerization. Asparagine deamidation and aspartic acid isomerization may be induced in vitro in high pH and low pH conditions, respectively. To investigate the antibody stabilities of humanized anti-MICA/B antibodies under stress conditions, 100 μL antibody (1 mg/mL) was exchanged with pH 5.5 buffer (50 mM Sodium Acetate) or pH 8.5 buffer (20 mM Tris and 10 mM EDTA) using Zebra Spin Desalting columns (40K MWCO, ThermoFisher, Cat #: 87767), respectively. The antibodies were incubated in a 40° C. water bath for 2 weeks. The binding affinities of antibodies under stress conditions and unstressed controls were measured against MICA/B polypeptides by GatorPrime (Gator Bio, USA). Affinity and normalized Rₘₐₓ were used to evaluate functional changes.

As shown in FIG. 15, the stressed humanized anti-MICA/B antibodies had similar binding affinities comparing to unstressed controls, indicating humanized anti-MICA/B antibodies D2M001-010 and its variant and humanized #36, h36B3, are stable under stressed conditions.

### Example 12: Humanized Anti-MICA/B Antibodies Exhibit No Self-Binding

Self-interaction of a therapeutic monoclonal antibody may cause aggregation, high viscosity, or low solubility, which may limit its therapeutic usefulness. Self-interaction of the humanized and optimized anti-MICA/B antibodies were measured by clone self-interaction (CSI) using bio-layer interferometry (BLI) using GatorPrime (Gator Bio, USA). Briefly, the anti-MICA/B antibodies were loaded with HFC (Anti-hIgG Fc) (Gator Bio, Cat #: 160003). Probes were then incubated with antibodies and the Rₘₐₓ of the interaction was calculated to evaluate the binding.

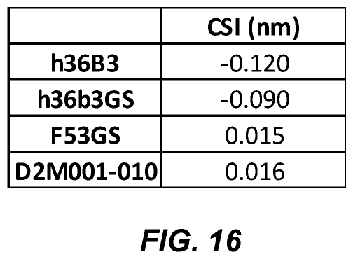

As shown in FIG. 16, the CSI scores of humanized and optimized anti-MICA/B antibodies are very low, indicating humanized anti-MICA/B antibodies D2M001-010 and its variant and humanized #36 and its variant have no self-binding.

### Example 13: Humanized Anti-MICA/B Antibodies Exhibit No Non-Specific Binding in CHO and C1R Cell Lysates

To investigate the non-specific binding of humanized and optimized anti-MICA/B antibodies, total cell lysates were prepared from CHO cells and C1R parental cells using NP-40 Cell Lysis Buffer (ThermoFisher, Cat #: AAJ60766AK). The protein concentration was measured by a BCA Protein Assay (ThermoFisher, Cat #: 23225) according to the manufacture's manual. Anti-MICA/B antibodies or IgG1 control were biotinylated using the EZ-Link Sulfo-NHS-LC-Biotinylation Kit (ThermoFisher, Cat #: 21435) according to the manufacture's manual. A 96-well assay plate was coated with 100 μl lysate with 50 g total protein per well overnight. The plate was washed twice with PBS and was then blocked with 1% BSA for 2 hours at room temperature. After two additional PBS washes, 100 μL titrated anti-MICA/B antibodies or an isotype control antibody (IgG1) were added to two test concentrations of 15 μg/mL and 1.5 μg/mL and incubated for 1 hour at room temperature. The plate was washed with PBS twice and 100 μL HRP Streptavidin (BioLegend, Cat #: 405210, 1:10000) was added and developed with TMB to detect the binding signals.

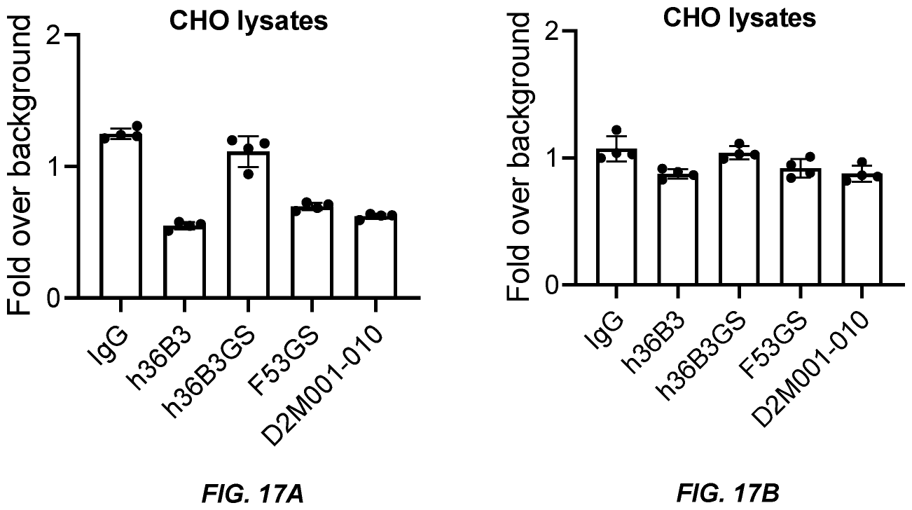

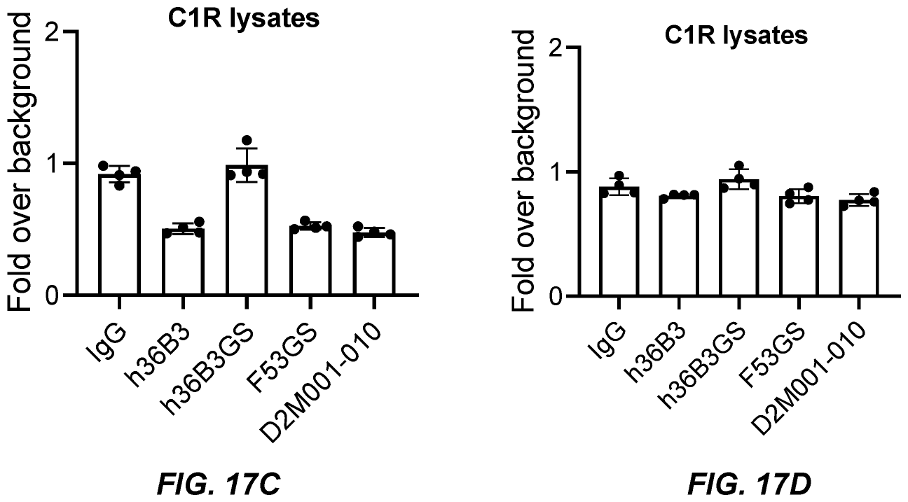

As shown in FIGS. 17A-17D, anti-MICA/B and IgG1 isotype control had approximately equal binding signals to background without antibodies (fold over-background signals were about 1), indicating humanized and optimized anti-MICA/B antibodies had very low levels of non-specific binding against proteins in CHO or human CIR tumor cells.

### Example 14: Humanized Anti-MICA/B Antibodies Exhibit Stability in Human Serum In Vitro

To further investigate the antibody stabilities of humanized anti-MICA/B antibodies, 50 μg anti-MICA/B antibody (˜1 mg/mL) was mixed with equal volume of PBS or human fresh serum or plasma sampled from healthy volunteers and incubated in a 37° C. water bath for one or two weeks. Cell-based antibody binding affinities were measured by incubating titrated antibodies from three treatment groups of anti-MICA/B antibodies (0 week, 1 week, and 2 weeks of incubation with human serum) and two reference antibodies with engineered CHO-MICB005 cells. 50 μL CHO-MICB005 or CHO-MICA (approximately 4.0×10⁴ cells) were added to each well of a 96-well U-bottom culture plate. 50 μL titrated anti-MICA/B antibodies were added to final concentrations of 10 μg/mL, 3.33 μg/mL, 1.11 μg/mL, 0.370 μg/mL, 0.123 μg/mL, 0.041 μg/mL, 0.0137 μg/mL, and 0 μg/mL, respectively, and incubated for 30 minutes at room temperature. The plate was centrifuged, and cells were washed with PBS three times. 50 μL Alexa Fluor® 647 Affini Pure F(ab′)₂ Fragment Goat Anti-Human IgG, Fcγ fragment specific (Jackson ImmunoResearch Laboratories Inc., Cat #: 109-606-098) was added to each well of the plate. After incubation for 30 minutes at room temperature, cells were washed with PBS twice. Signals of APC were determined by flow cytometry (Attune™ CytPix™, ThermoFisher) within the population of live cells (viability dye PI negative).

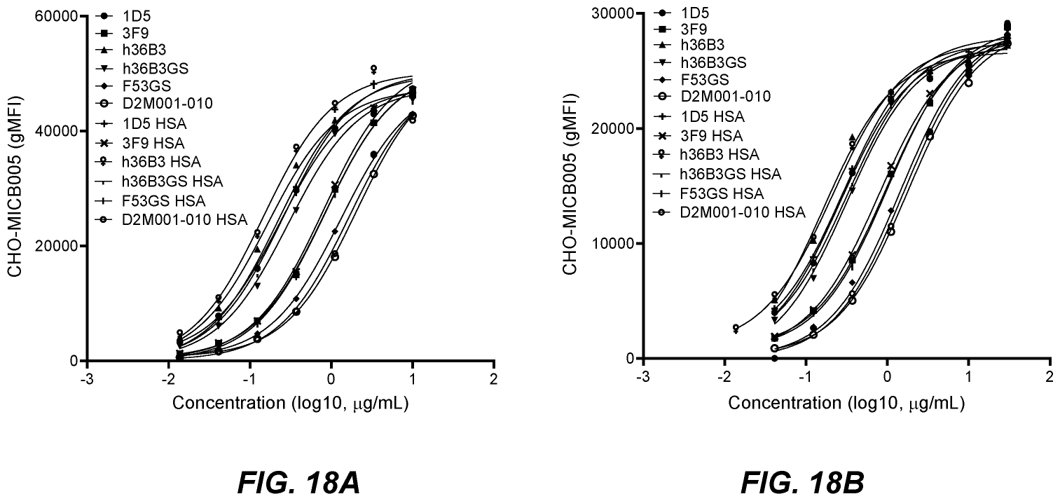

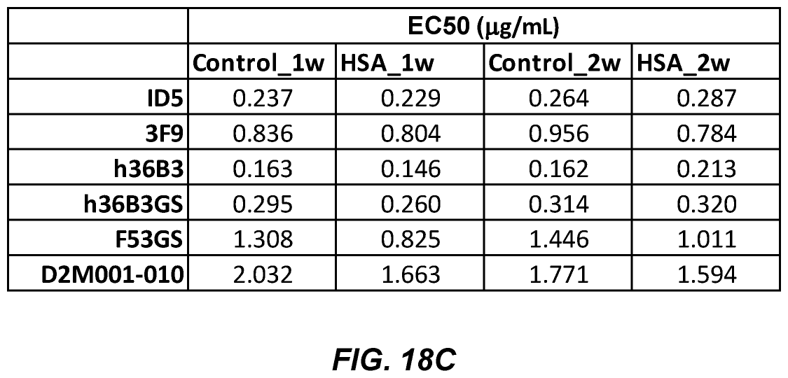

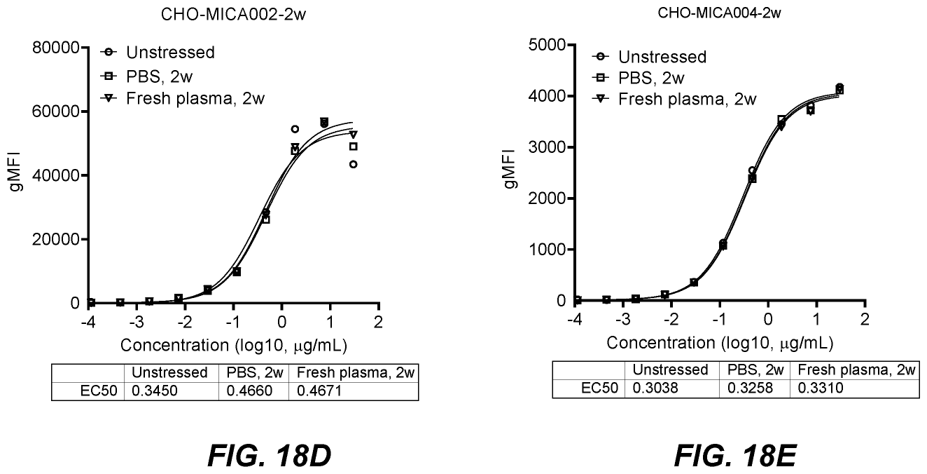

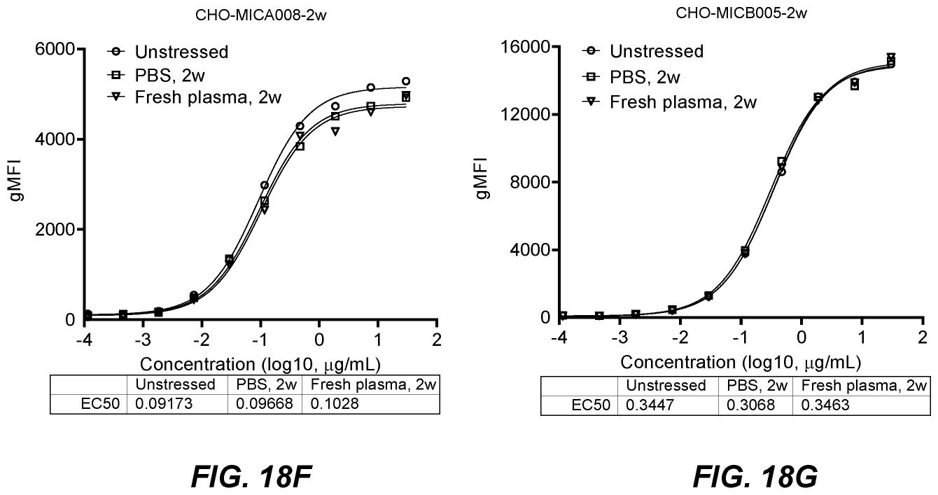

As shown in FIGS. 18A-18C, humanized anti-MICA/B antibodies D2M001-010 and it variants and humanized #36 and its variants were incubated with human serum for 1 or 2 weeks exhibited strong binding affinities to CHO-MICB005 with similar EC50 values before and post human serum incubation. The EC50 values of MICB005 binding (shown in FIG. 18C) were calculated based on non-linear fitting curves by GraphPad Prism software. As shown in FIGS. 18D-18E, D2M001-209 antibody incubated with human plasma or PBS for 2 weeks exhibited strong binding affinities to CHO-MICA002 (FIG. 18D), CHO-MICA004 (FIG. 18E), CHO-MICA008 (FIG. 18F), or CHO-MICA004 (FIG. 18E) with very similar EC50 values before and post human plasma or PBS incubation. The EC50 values were calculated based on non-linear fitting curves by GraphPad Prism software. In conclusion, D2M001-209 exhibited desirable stability in human serum in vitro.

### Example 15: Humanized Anti-MICA/B Antibodies Exhibit Long-Term Stability in Mice

To further investigate the stability of humanized anti-MICA/B antibodies in vivo, 200 μg anti-MICAB antibody (10 mg/kg) was injected retro-orbitally into B6 mice. For each animal, 50 μL of blood was collected at timepoints of 2 hours, 5 hours, 1 day, 2 days, 3 days, 6 days, 8 days and 10 days post-injection by retro-orbital bleeding. To detect the concentration of anti-MICA/B in the plasma, two methods of sandwich ELISA were developed.

For the first method (“Coated MICB”), a 96-well assay plate was coated with 100 μl MICB005-ECD-His (1 μg/mL, Sino Biological, Cat #: 10759-H08H) overnight at 4° C. After two washes with PBS, the plate was blocked with 1% BSA for 2 hours at room temperature. After two additional PBS washes, 100 μL diluted plasma samples (30,000× dilutions) and titrated standards (in vivo anti-MICA/B) were added and incubated for 1 hour at room temperature. Afterward, the plate was washed with PBS twice and 100 μL HRP anti-human hFec (1:10000, Jackson ImmunoResearch, Cat #: 109-035-008) were added and developed with TMB to detect the binding signals.

For the second method (“Coated anti-hFab”), a 96-well assay plate was coated with 100 μl goat anti-human IgG, F(ab′)₂ specific (1 μg/mL, Jackson ImmunoResearch, Cat #: 109-005-097) overnight at 4° C. After two washes with PBS, the plate was blocked with 1% BSA for 2 hours at room temperature. After two additional PBS washes, 100 μL diluted plasma samples (30,000× dilutions) and titrated standards (in vivo anti-MICA/B) were added and incubated for 1 hour at room temperature. Afterward, the plate was washed with PBS twice and 100 μL HRP anti-human hFec (1:10000, Jackson ImmunoResearch, Cat #: 109-035-008) were added and developed with TMB to detect the binding signals.

Because mouse does not have MICA/B homologues, B16F10-MICAB mouse melanoma B16F10 cells were engineered to express MICB005 and MICA004 by lentiviral transduction. Single clones were screened by MICA/B expression and shedding inhibition by anti-MICA/B antibodies as described in Example 4. To further investigate the stability of humanized anti-MICA/B antibodies in tumor-bearing in vivo, approximately 1.0×10⁶ B16F10-MICA/B (clone #57) cells were implanted subcutaneously in the left flank of 8-week-old B6 mice. On day 8, after the tumor mass reached approximately 80-100 mm³, mice were treated by intraperitoneal (i.p.) administration with 10 mg/kg or 30 mg/kg anti-MICAB antibody D2M001-209 or PBS control once. For each animal, 50 μL of blood was collected at 2 hours, 1 day, 3 days, 5 days, 7 days, and 9 days post treatment. To measure the pharmacodynamic (PD) effects, the concentrations of plasma anti-MICAB antibody D2M001-209 were measured as described above with the second method (“Coated anti-hFab”). The soluble MICA and MICB polypeptides in plasma were measured by ELISA assays as described in Example 4.

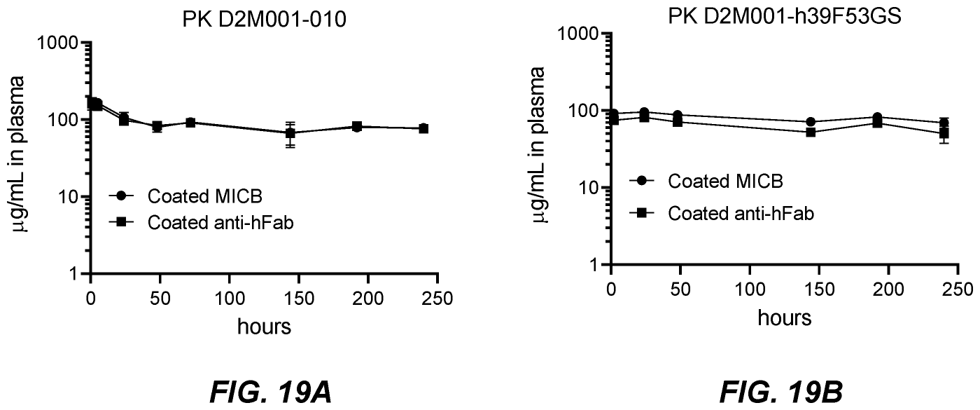

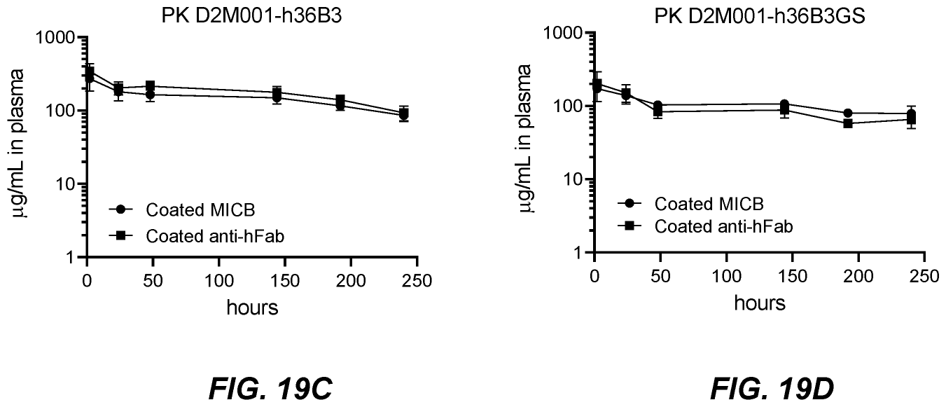

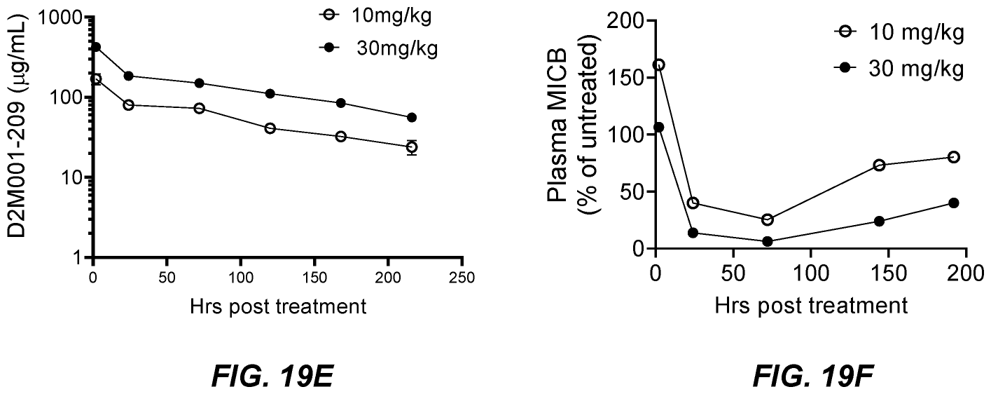

As shown in FIGS. 19A-19D, humanized anti-MICA/B antibodies D2M001-010 and h36B3 and their variants persisted in vivo through 10 days post-injection, demonstrating that both of these humanized and antibodies have long-term stability in mice in vivo.

As shown in FIGS. 19E-19F, D2M001-209 demonstrated excellent in vivo stability and pharmacodynamics effects in B16F10-MICAB tumor-bearing mice. Although as shown in FIG. 19E, D2M001-209 showed faster elimination rate, due to the presence of tumor cells expressing MICA/B, compared to its parental h36B3 in FIG. 19C, D2M001-209 exhibited stability in vivo. As shown in FIG. 19F, soluble MICB dropped off immediately post D2M001-209 administration and gradually increased over time when the plasma concentration of D2M001-209 decreased, indicating plasma concentration of soluble MICB correlates with plasma concentration of D2M001-209. Furthermore, the results suggest that soluble MICB may potentially serve as a clinic PD biomarker. In conclusion, D2M001-209 exhibited in vivo stability and PK/PD correlation.

### Example 16: Humanized Anti-MICA/B Antibodies Inhibit Shedding of MICA/B Polypeptides in Mice

To further investigate whether anti-MICA/B antibodies can inhibit the shedding of MICA/B alleles in vivo, B16F10 mouse melanoma cells were engineered to express MICB005 and MICA004 by lentiviral transduction. Single clones were screened by MICA/B expression and shedding inhibition by anti-MICA/B antibodies as described in Example 4. The two best performing clones were tested further in vivo. Approximately 1.0×10⁶ B16F10-MICAB (clone #57) cells were implanted subcutaneously in the left flank of 8-week old B6 mice. On day 14 after the tumor mass reached approximately 100-150 mm³, mice were dosed with 10 mg/kg isotype IgG/PBS (control) or h36B3 antibody (treatment). Mice were bled retro-orbitally before treatment and 24 hours after treatment. The shed MICA and MICB polypeptides in plasma were measured by ELISA assays as described in Example 4.

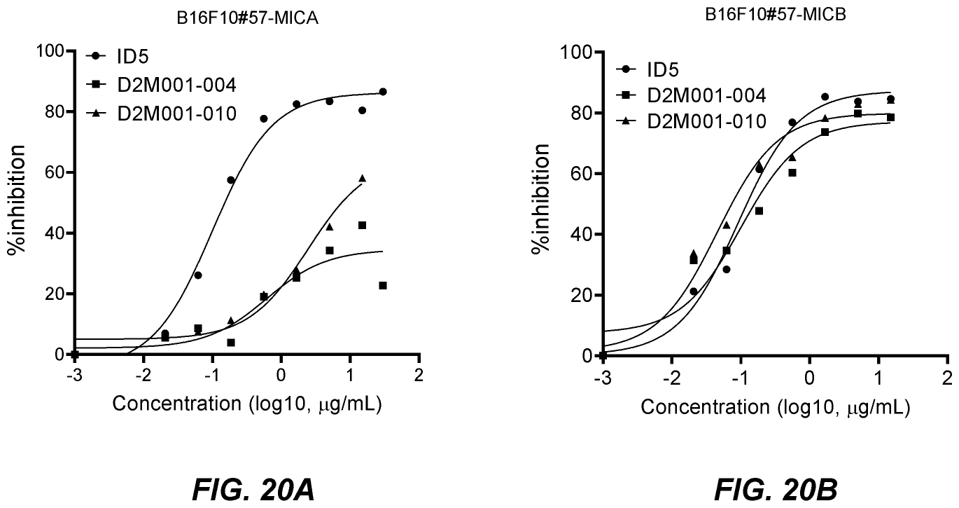

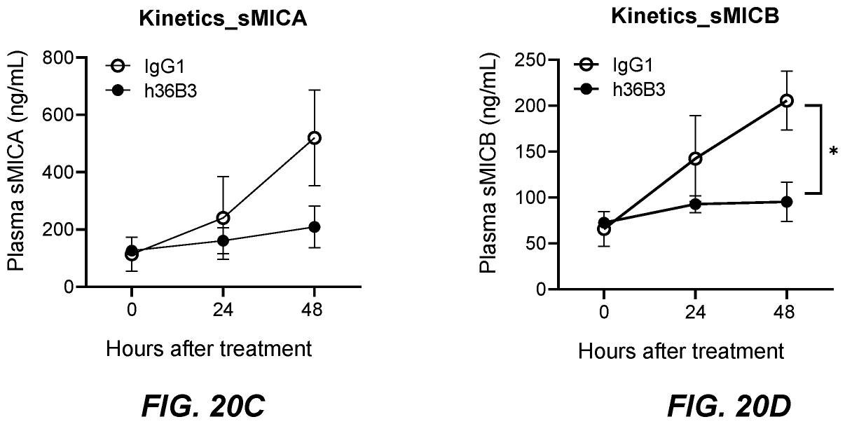

As shown in FIGS. 20A-20B, transgenic mouse tumor cells expressing human MICA/B alleles shed soluble MICA and MICB polypeptides. Humanized and optimized anti-MICA/B antibodies were able to inhibit the shedding of MICA/B polypeptides from mouse tumor cells expressing human MICA/B alleles in vitro. After treatment with humanized anti-MICA/B antibodies, the shed MICA/B polypeptides decreased significantly in the cell culture medium, indicating the humanized anti-MICA/B antibodies and the reference antibody (1D5) exhibited strong inhibition of the shedding of MICA004 and MICB005 polypeptides expressed on the surface of mouse B16F10 tumor cells.

As shown in FIGS. 20C-20D, plasma soluble MICA and MICB in animals treated with IgG1 control increased over 3 days post administration, while plasma soluble MICA and MICB in animals treated humanized and optimized anti-MICA/B antibody h36B3 did not increase over 3 days post administration, demonstrating h36B3 administration inhibited the shedding of MICA/B polypeptides in mouse tumor cells expressing human MICA/B alleles in vivo. Further confirmed that soluble IC/AB may be a good clinic PD biomarker.

### Example 17: Humanized Anti-MICA/B Antibodies Inhibit Tumor Growth and Synergize with Anti-PD-1 in Mouse Tumor Models

To investigate the efficacy of humanized anti-MICA/B antibodies for inhibiting tumor growth as well as whether anti-MICA/B antibodies have synergistic effects with anti-PD1, approximately 1.0×10⁶ B16F110-MICA/B (clone #57) cells were implanted subcutaneously in the left flank of 8-week old B6 mice. On day 6, after the tumor volume reached approximately either 20-30 mm² or 30-50 mm³, mice were dosed with 10 mg/kg isotype IgG (control); anti-PD1 (2 mg/kg, BioIntron, clone RMP1-14); D2M001-010, a combination of D2M001-010 and anti-PD1; h36B3, a combination of h36B3 and anti-PD1; h36B3-LALAPA (effector-null), a combination of h36B-LALAPA and anti-PD1; or D2M001-209, 3F9, or a combination of D2M001-209 and anti-PD1; every 2 days consecutively for a total of three serial doses. Tumor sizes were measured on day 6, day 8, day 10, day 14, and/or day 18 in some experiments by a digital caliper. Mice were bled retro-orbitally on day 14 or day 18 to measure the concentration of shed MICA and MICB polypeptides in plasma by ELISA assays as described in Example 4. Statistical analysis was performed using t-test. P-values were calculated by t-test. * P<0.05, ** P<0.01, *** P<0.001, **** P<0.0001, n.s. not significant.

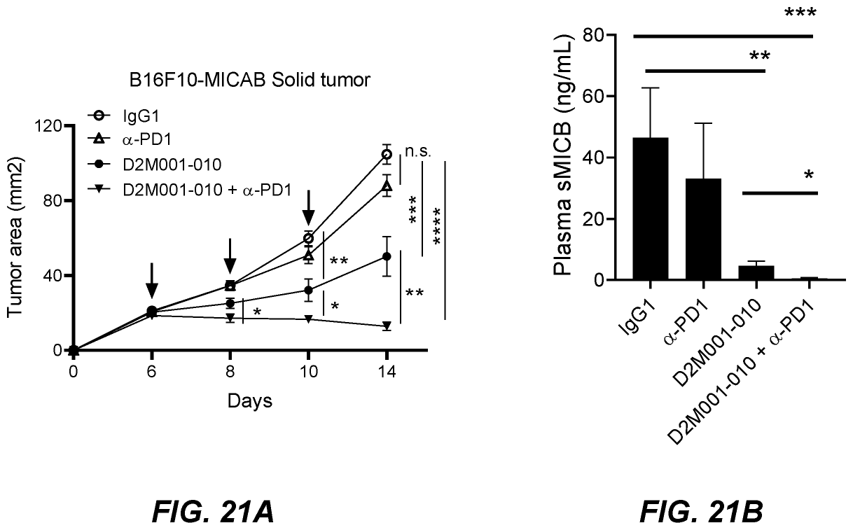

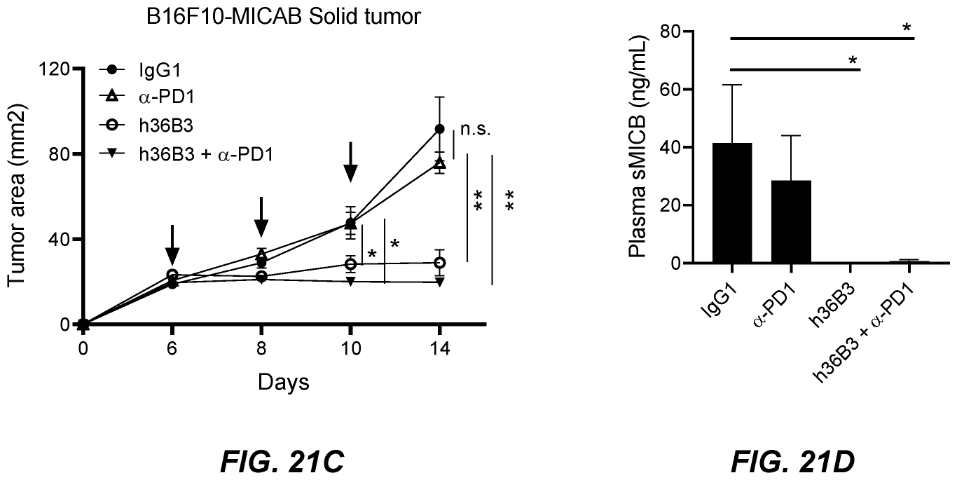

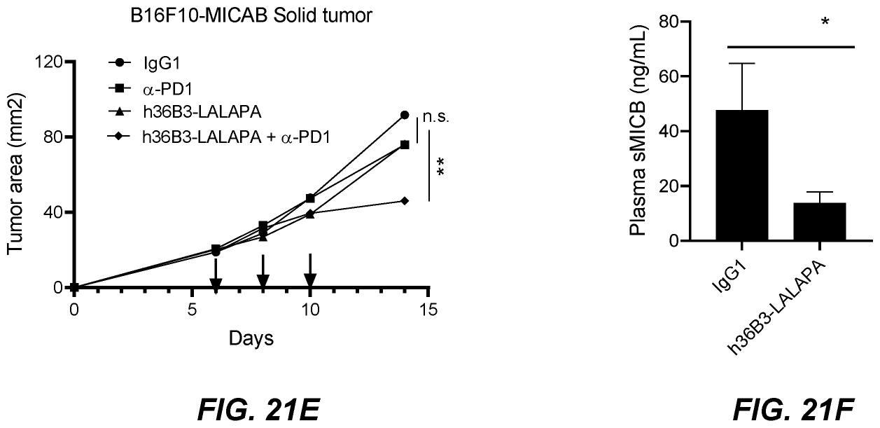

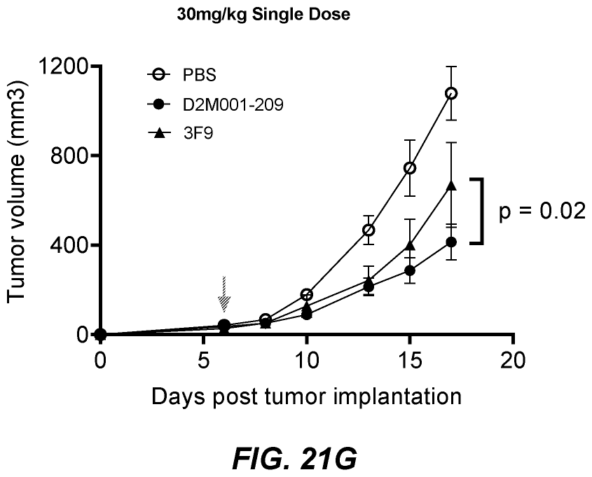

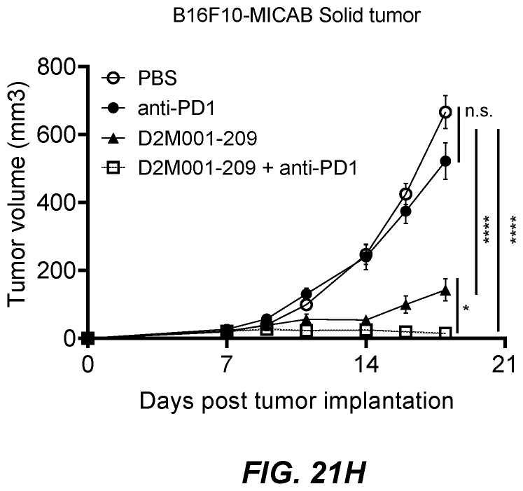

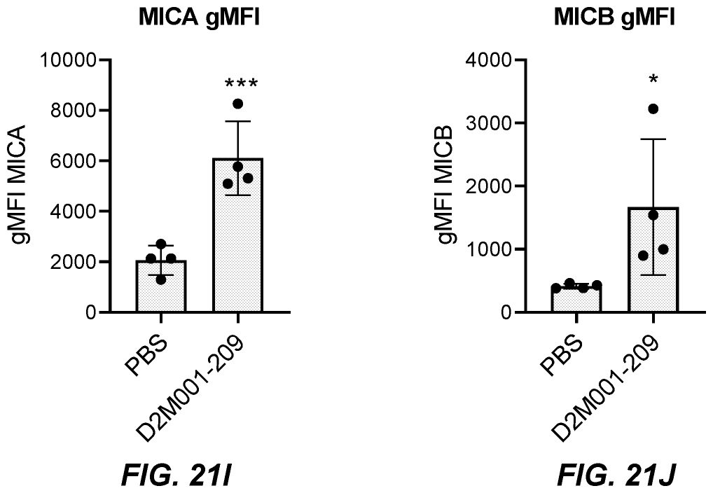

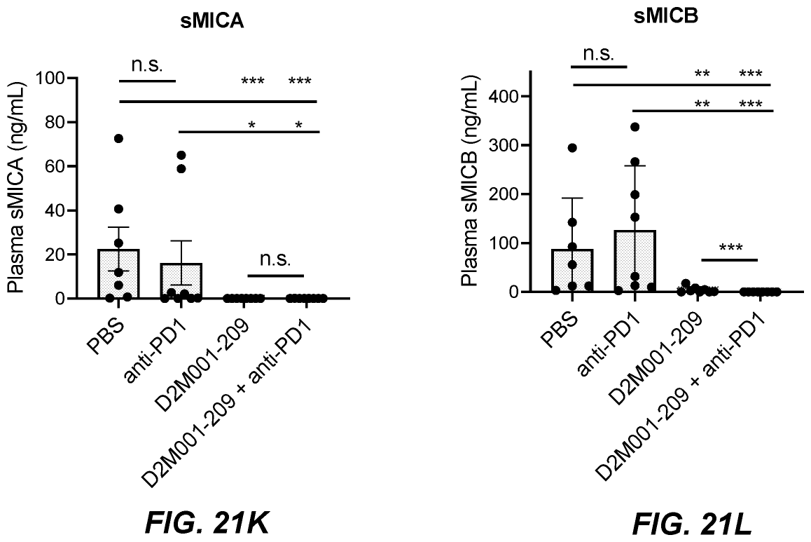

As shown in FIG. 21A, humanized anti-MICA/B antibody D2M001-010 significantly inhibited the tumor growth of B16F10-MICAB (˜52% TGI) in mice (8-12 mice per group) relative to isotype IgG control group or anti-PD1 treatment group. Anti-MICA/B antibody D2M001-010 synergistically inhibited the tumor growth (˜88% TGI).

As shown in FIG. 21B, the concentrations of shed MICB polypeptides decreased significantly in plasma in the anti-MICA/B antibody D2M001-010 treatment group and the combination treatment group relative to either the isotype IgG1 control group or the anti-PD1 treatment group, further demonstrating that humanized and optimized anti-MICA/B antibodies can inhibit tumor growth and inhibit the shedding of MICA/B polypeptides from tumor cells.

As shown in FIG. 21C, humanized anti-MICA/B antibody h36B3 significantly inhibited the tumor growth of B16F10-MICAB (˜%80 TGI) in mice (8-12 mice per group) relative to isotype IgG control group or anti-PD1 treatment group. Anti-MICA/B antibody h36B3 synergistically inhibited the tumor growth (˜88% TGI). As shown in FIG. 21D, the concentrations of shed MICB polypeptides decreased significantly in plasma in the anti-MICA/B antibody h36B3 treatment group and the combination treatment group relative to either the isotype IgG1 control group or the anti-PD1 treatment group, further demonstrating that humanized and optimized anti-MICA/B antibodies can inhibit tumor growth and inhibit the shedding of MICA/B polypeptides from tumor cells.

As shown in FIG. 21E, neither effector-null h36B3 (h36B3-LALAPA) nor anti-PD1 treatment significantly inhibited the tumor growth of B16F10-MICAB in mice (8-12 mice per group) relative to isotype IgG control group. However, the combination of h36B3-LALAPA synergistically inhibited the tumor growth (˜50% TGI). Moreover, as shown in FIG. 21F, h36B3-LALAPA treatment alone inhibited the shedding of MICA/B polypeptides from tumor cells, demonstrating effector-null h36B has biological function to inhibit shedding of MICA/B. Although the majority of anti-MICA/B anti-tumor activity is Fc receptor dependent, effector-null h36B3 has ability to inhibit MICA/B shedding and therefore induced anti-tumor activity and synergize with anti-PD-1 treatment.

As shown in FIG. 21G, humanized anti-MICA/B antibody D2M001-209 and reference antibody 3F9 both significantly inhibited the tumor growth of B16F10-MICAB in mice (8-12 mice per group) relative to isotype IgG control group. However, D2M001-209 had superior anti-tumor activity comparing to 3F9.

As shown in FIG. 21H, humanized anti-MICA/B antibody D2M001-209 significantly inhibited the tumor growth of B16F10-MICAB (˜%85 TGI) in mice (8-12 mice per group) relative to isotype IgG control group or anti-PD1 treatment group. Anti-MICA/B antibody D2M001-209 synergistically inhibited the tumor growth (˜90% TGI).

As shown in FIGS. 21I-21J, the surface expression of MICA/B in tumor cells increased significantly in the anti-MICA/B antibody D2M001-209 treatment group relative to the isotype IgG1 control group.

As shown in FIGS. 21K-21L, the concentrations of shed MICA/B polypeptides decreased significantly in plasma in the anti-MICA/B antibody D2M001-209 treatment group and the combination treatment group relative to either the isotype IgG1 control group or the anti-PD1 treatment group, further demonstrating that humanized and optimized anti-MICA/B antibodies can inhibit tumor growth and inhibit the shedding of MICA/B polypeptides from tumor cells.

Overall, D2M001-209 has demonstrated strong ability to inhibit MICA/B shedding and stabilize surface expression of MICA/B in vivo, and more importantly exhibited significant antitumor activity as single agent and synergistic effects in the combination with anti-PD-1 treatment. Furthermore, D2M001-209 exhibited superior in vivo antitumor activity as compared to 3F9.

### Example 18: Humanized Anti-MICA/B Antibodies Inhibit Tumor Metastasis

To further investigate the efficacy of humanized anti-MICA/B antibodies for inhibiting tumor growth, approximately 5.0×10⁵ B16F10-MICAB (clone #57) cells were injected intravenously (tail vein) in 8-week old B6 mice. The mice were grouped randomly and treated intraperitoneally with 10 mg/kg isotype IgG (control), or antibody D2M001-010, or antibody h36B3 on day 1, day 2, day 6, and day 10 after tumor cell injection. On day 14, mice were sacrificed and 100 μL blood was collected from each animal. Lungs were collected from each animal and perfused with PBS and 2 mM EDTA and fixed in 10% formalin. The numbers of metastasis tumor in the lungs were counted under a stereotype microscope. The concentration of shed MICA and MICB polypeptides in plasma were measured by ELISA assays as described in Example 4.

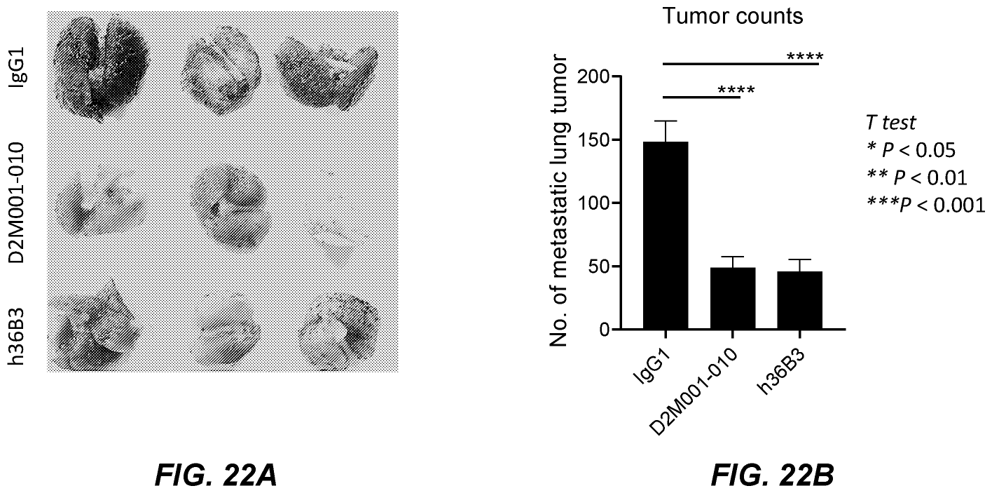

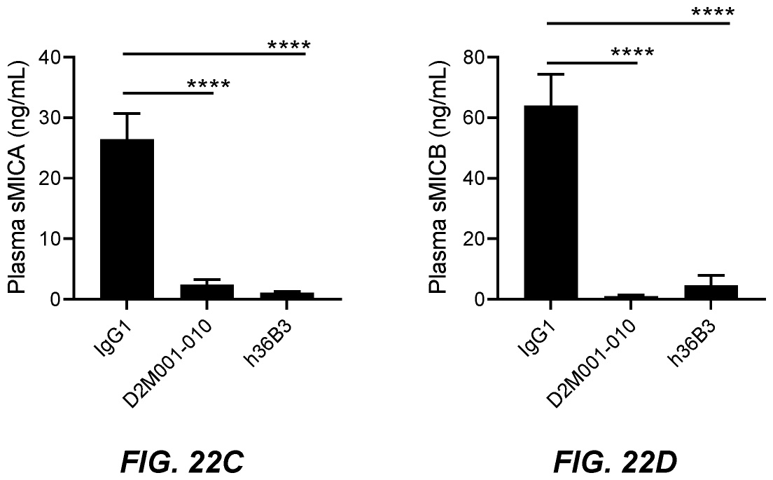

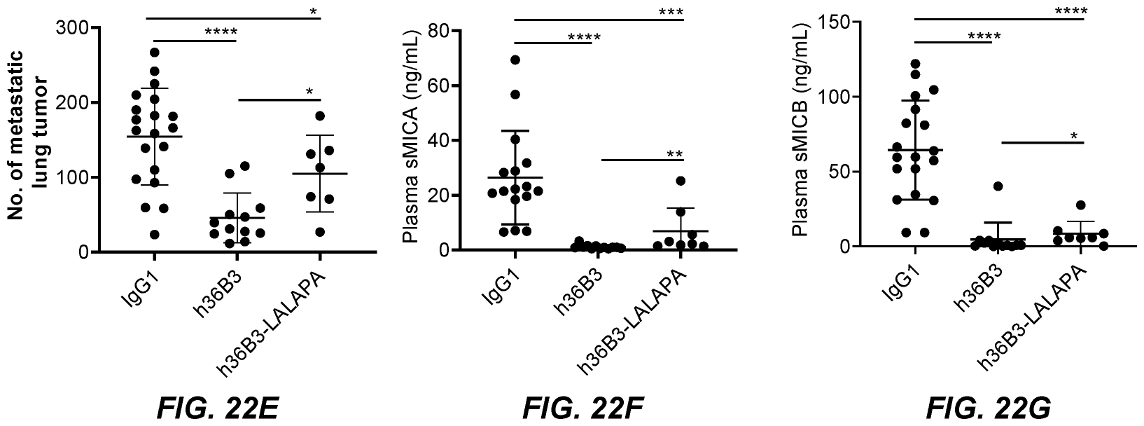

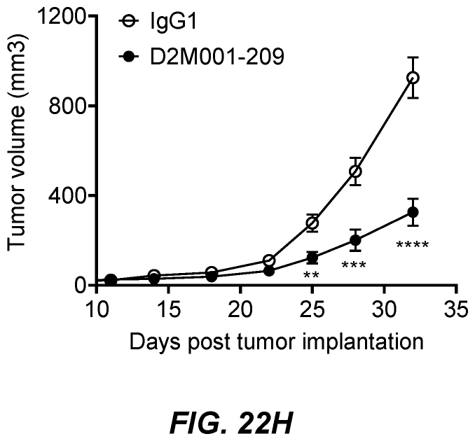

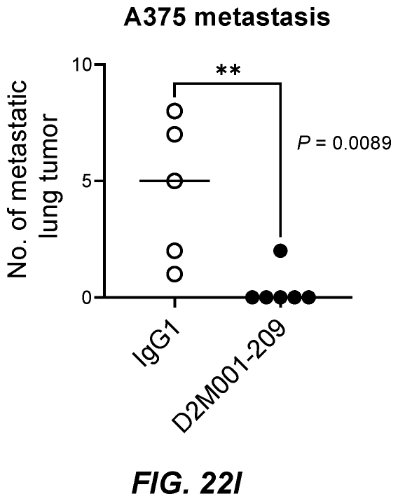

As shown in FIGS. 22A-22B, both humanized and optimized anti-MICA/B antibody D2M001-010 and h36B3 significantly reduced metastasis of lung tumors of B16F10-MICAB in mice (10-12 mice per group) relative to isotype IgG control. As shown in FIGS. 22C-22D, the concentrations of shed MICA and MICB polypeptides decreased significantly in plasma in animals treated with anti-MICA/B antibody D2M001-010 or h36B3 relative to isotype IgG1 controls, further demonstrating that humanized and optimized anti-MICA/B antibodies can inhibit tumor growth in lungs and inhibit the shedding of MICA/B polypeptides from tumor cells.

As shown in FIG. 22E, both humanized and optimized anti-MICA/B antibody h36B3 and its effector-null format (h36B3-LALAPA) significantly reduced metastasis of lung tumors of B16F10-MICAB in mice (10-12 mice per group) relative to the isotype IgG control group. h36B3-LALAPA had less anti-tumor activity relative to h36B3. As shown in FIGS. 22F-22G, the concentrations of shed MICA and MICB polypeptides decreased significantly in plasma in animals treated with anti-MICA/B antibody h36B3 and h36B3-LALAPA relative to isotype IgG1 controls, further demonstrating that humanized and optimized anti-MICA/B antibodies can inhibit tumor growth in lungs and inhibit the shedding of MICA/B polypeptides from tumor cells. In comparison of h36B3, h36B3-LALAPA had reduced function to inhibit the shedding of MICA/B polypeptides in plasma from tumor cells.

Overall, h36B3 demonstrated strong ability to inhibit MICA/B shedding from metastatic tumor cells in vivo, and more importantly exhibited significant anti-tumor activity to inhibit metastatic tumor growth. Although Fc effector function contributed significantly to anti-tumor activity, FcγR independent ability to inhibit MICA/B shedding plays an important role in the mode of action of anti-MICA/B antibody.

### Example 19: Humanized Anti-MICA/B Antibody Inhibit Tumor Growth and Metastasis in SCID Mice

To further investigate the efficacy of humanized anti-MICA/B antibodies for inhibiting the growth of xenograft human tumors, approximately 5.0×10⁶ A375 human melanoma cells were implanted subcutaneously in the left flank of 8-week-old SCID mice. On day 11, after the tumor volume reached approximately 30-50 mm³, mice were randomly grouped and dosed with 10 mg/kg isotype IgG (control), or anti-MICA/B D2M001-209 with 2 doses weekly consecutively for a total of five doses. Tumor sizes were measured on day 11, day 14, day 18, day 22, day 25, day 28, and day 32 by a digital caliper. Statistical analysis was performed using t-test. ** P<0.01, *** P<0.001, **** P<0.0001.

To further investigate the efficacy of humanized anti-MICA/B antibodies for inhibiting the metastasis of xenograft human tumors, approximately 5.0×10⁶ A375 human melanoma cells were injected intravenously (tail vein) in 8-week-old SCID mice. Mice were randomly grouped and dosed with 10 mg/kg isotype IgG (control), or anti-MICA/B D2M001-209 on day 1, day 2, day 7, day 14, and day 21. On day 30, mice were euthanized, and lungs were perfused by intratracheal injection with 1 mL 30% Indian ink (StatLab, Cat #: STIIN25) in PBS and fixed in Fekete's fixative buffer. Metastatic lung tumors were counted under microscopy with white tumor and dark lung tissue. Statistical analysis was performed using t-test. ** P<0.01.

As shown in FIG. 22H, D2M001-209 significantly inhibited the tumor growth of A375 (˜%70 TGI) in SCID mice (8-12 mice per group) relative to isotype IgG control group. As shown in FIG. 22I, D2M001-209 significantly reduced metastasis of lung tumors of A375 in SCID mice (10-12 mice per group) relative to isotype IgG control.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An antibody or antigen-binding fragment thereof that binds to MICA (major histocompatibility complex class I chain related A) and/or MICB (major histocompatibility complex class I chain related B), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are selected from VH CDRS 1, 2, 3 and VL CDRS 1, 2, 3 listed in FIGS. 24A, 24B, 24C, 24D, 24E, 28, 29, 30A, 30B, 30C, 30D, and 30E.

2. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 3, 4, 5, respectively, and the selected VL CDRs 1, 2, 3 amino acid sequences are set forth in SEQ ID NOs: 6, 7, 8, respectively;
(2) the selected VH CDRs 1, 2, 3 amino acid sequences are set forth in SEQ ID NOs: 11, 12, 13, respectively, and the selected VL CDRs 1, 2, 3 amino acid sequences are set forth in SEQ ID NOs: 14, 15, 16, respectively;
(3) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(4) the selected VH CDRs 1, 2, 3 amino acid sequences are set forth in SEQ ID NOs: 27, 28, 29, respectively, and the selected VL CDRs 1, 2, 3 amino acid sequences are set forth in SEQ ID NOs: 30, 31, 32, respectively;
(5) the selected VH CDRs 1, 2, 3 amino acid sequences are set forth in SEQ ID NOs: 35, 36, 37, respectively, and the selected VL CDRs 1, 2, 3 amino acid sequences are set forth in SEQ ID NOs: 38, 39, 40, respectively;
(6) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively;
(7) the selected VH CDRs 1, 2, 3 amino acid sequences are set forth in SEQ ID NOs: 51, 52, 53, respectively, and the selected VL CDRs 1, 2, 3 amino acid sequences are set forth in SEQ ID NOs: 54, 55, 56, respectively;
(8) the selected VH CDRs 1, 2, 3 amino acid sequences are set forth in SEQ ID NOs: 59, 60, 61, respectively, and the selected VL CDRs 1, 2, 3 amino acid sequences are set forth in SEQ ID NOs: 62, 63, 64, respectively;
(9) the selected VH CDRs 1, 2, 3 amino acid sequences are set forth in SEQ ID NOs: 67, 68, 69, respectively, and the selected VL CDRs 1, 2, 3 amino acid sequences are set forth in SEQ ID NOs: 70, 71, 72, respectively;
(10) the selected VH CDRs 1, 2, 3 amino acid sequences are set forth in SEQ ID NOs: 75, 76, 77, respectively, and the selected VL CDRs 1, 2, 3 amino acid sequences are set forth in SEQ ID NOs: 78, 79, 80, respectively;
(11) the selected VH CDRs 1, 2, 3 amino acid sequences are set forth in SEQ ID NOs: 83, 84, 85, respectively, and the selected VL CDRs 1, 2, 3 amino acid sequences are set forth in SEQ ID NOs: 86, 87, 88, respectively;
(12) the selected VH CDRs 1, 2, 3 amino acid sequences are set forth in SEQ ID NOs: 91, 92, 93, respectively, and the selected VL CDRs 1, 2, 3 amino acid sequences are set forth in SEQ ID NOs: 94, 95, 96, respectively;
(13) the selected VH CDRs 1, 2, 3 amino acid sequences are set forth in SEQ ID NOs: 99, 100, 101, respectively, and the selected VL CDRs 1, 2, 3 amino acid sequences are set forth in SEQ ID NOs: 102, 103, 104, respectively;
(14) the selected VH CDRs 1, 2, 3 amino acid sequences are set forth in SEQ ID NOs: 107, 108, 109, respectively, and the selected VL CDRs 1, 2, 3 amino acid sequences are set forth in SEQ ID NOs: 110, 111, 112, respectively;
(15) the selected VH CDRs 1, 2, 3 amino acid sequences are set forth in SEQ ID NOs: 115, 116, 117, respectively, and the selected VL CDRs 1, 2, 3 amino acid sequences are set forth in SEQ ID NOs: 118, 119, 120, respectively;
(16) the selected VH CDRs 1, 2, 3 amino acid sequences are set forth in SEQ ID NOs: 123, 124, 125, respectively, and the selected VL CDRs 1, 2, 3 amino acid sequences are set forth in SEQ ID NOs: 126, 127, 128, respectively;
(17) the selected VH CDRs 1, 2, 3 amino acid sequences are set forth in SEQ ID NOs: 131, 132, 133, respectively, and the selected VL CDRs 1, 2, 3 amino acid sequences are set forth in SEQ ID NOs: 134, 135, 136, respectively;
(18) the selected VH CDRs 1, 2, 3 amino acid sequences are set forth in SEQ ID NOs: 139, 140, 141, respectively, and the selected VL CDRs 1, 2, 3 amino acid sequences are set forth in SEQ ID NOs: 142, 143, 144, respectively;
(19) the selected VH CDRs 1, 2, 3 amino acid sequences are set forth in SEQ ID NOs: 147, 148, 149, respectively, and the selected VL CDRs 1, 2, 3 amino acid sequences are set forth in SEQ ID NOs: 150, 151, 152, respectively;
(20) the selected VH CDRs 1, 2, 3 amino acid sequences are set forth in SEQ ID NOs: 155, 156, 157, respectively, and the selected VL CDRs 1, 2, 3 amino acid sequences are set forth in SEQ ID NOs: 158, 159, 160, respectively;
(21) the selected VH CDRs 1, 2, 3 amino acid sequences are set forth in SEQ ID NOs: 815, 816, 817, respectively, and the selected VL CDRs 1, 2, 3 amino acid sequences are set forth in SEQ ID NOs: 818, 819, 820, respectively;
(22) the selected VH CDRs 1, 2, 3 amino acid sequences are set forth in SEQ ID NOs: 821, 822, 823, respectively, and the selected VL CDRs 1, 2, 3 amino acid sequences are set forth in SEQ ID NOs: 824, 825, 826, respectively;
(23) the selected VH CDRs 1, 2, 3 amino acid sequences are set forth in SEQ ID NOs: 827, 828, 829, respectively, and the selected VL CDRs 1, 2, 3 amino acid sequences are set forth in SEQ ID NOs: 830, 831, 832, respectively;
(24) the selected VH CDRs 1, 2, 3 amino acid sequences are set forth in SEQ ID NOs: 833, 834, 835, respectively, and the selected VL CDRs 1, 2, 3 amino acid sequences are set forth in SEQ ID NOs: 836, 837, 838, respectively;
(25) the selected VH CDRs 1, 2, 3 amino acid sequences are set forth in SEQ ID NOs: 839, 840, 841, respectively, and the selected VL CDRs 1, 2, 3 amino acid sequences are set forth in SEQ ID NOs: 842, 843, 844, respectively;
(26) the selected VH CDRs 1, 2, 3 amino acid sequences are set forth in SEQ ID NOs: 845, 846, 847, respectively, and the selected VL CDRs 1, 2, 3 amino acid sequences are set forth in SEQ ID NOs: 848, 849, 850, respectively;
(27) the selected VH CDRs 1, 2, 3 amino acid sequences are set forth in SEQ ID NOs: 851, 852, 853, respectively, and the selected VL CDRs 1, 2, 3 amino acid sequences are set forth in SEQ ID NOs: 854, 855, 856, respectively;
(28) the selected VH CDRs 1, 2, 3 amino acid sequences are set forth in SEQ ID NOs: 857, 858, 859, respectively, and the selected VL CDRs 1, 2, 3 amino acid sequences are set forth in SEQ ID NOs: 860, 861, 862, respectively;
(29) the selected VH CDRs 1, 2, 3 amino acid sequences are set forth in SEQ ID NOs: 863, 864, 865, respectively, and the selected VL CDRs 1, 2, 3 amino acid sequences are set forth in SEQ ID NOs: 866, 867, 868, respectively;
(30) the selected VH CDRs 1, 2, 3 amino acid sequences are set forth in SEQ ID NOs: 869, 870, 871, respectively, and the selected VL CDRs 1, 2, 3 amino acid sequences are set forth in SEQ ID NOs: 872, 873, 874, respectively;
(31) the selected VH CDRs 1, 2, 3 amino acid sequences are set forth in SEQ ID NOs: 875, 876, 877, respectively, and the selected VL CDRs 1, 2, 3 amino acid sequences are set forth in SEQ ID NOs: 878, 879, 880, respectively;
(32) the selected VH CDRs 1, 2, 3 amino acid sequences are set forth in SEQ ID NOs: 881, 882, 883, respectively, and the selected VL CDRs 1, 2, 3 amino acid sequences are set forth in SEQ ID NOs: 884, 885, 886, respectively;
(33) the selected VH CDRs 1, 2, 3 amino acid sequences are set forth in SEQ ID NOs: 887, 888, 889, respectively, and the selected VL CDRs 1, 2, 3 amino acid sequences are set forth in SEQ ID NOs: 890, 891, 892, respectively;
(34) the selected VH CDRs 1, 2, 3 amino acid sequences are set forth in SEQ ID NOs: 893, 894, 895, respectively, and the selected VL CDRs 1, 2, 3 amino acid sequences are set forth in SEQ ID NOs: 896, 897, 898, respectively;
(35) the selected VH CDRs 1, 2, 3 amino acid sequences are set forth in SEQ ID NOs: 899, 900, 901, respectively, and the selected VL CDRs 1, 2, 3 amino acid sequences are set forth in SEQ ID NOs: 902, 903, 904, respectively;
(36) the selected VH CDRs 1, 2, 3 amino acid sequences are set forth in SEQ ID NOs: 905, 906, 907, respectively, and the selected VL CDRs 1, 2, 3 amino acid sequences are set forth in SEQ ID NOs: 908, 909, 910, respectively;
(37) the selected VH CDRs 1, 2, 3 amino acid sequences are set forth in SEQ ID NOs: 911, 912, 913, respectively, and the selected VL CDRs 1, 2, 3 amino acid sequences are set forth in SEQ ID NOs: 914, 915, 916, respectively;
(38) the selected VH CDRs 1, 2, 3 amino acid sequences are set forth in SEQ ID NOs: 917, 918, 919, respectively, and the selected VL CDRs 1, 2, 3 amino acid sequences are set forth in SEQ ID NOs: 920, 921, 922, respectively;
(39) the selected VH CDRs 1, 2, 3 amino acid sequences are set forth in SEQ ID NOs: 923, 924 925, respectively, and the selected VL CDRs 1, 2, 3 amino acid sequences are set forth in SEQ ID NOs: 926, 927, 928, respectively; and
(40) the selected VH CDRs 1, 2, 3 amino acid sequences are set forth in SEQ ID NOs: 929, 930 931, respectively, and the selected VL CDRs 1, 2, 3 amino acid sequences are set forth in SEQ ID NOs: 932, 933, 934, respectively.

3. (canceled)

4. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 161, 162, 163, respectively, and the selected VL CDRs 1, 2, 3 amino acid sequences are set forth in SEQ ID NOs: 164, 165, 166, respectively;
(2) the selected VH CDRs 1, 2, 3 amino acid sequences are set forth in SEQ ID NOs: 167, 168, 169, respectively, and the selected VL CDRs 1, 2, 3 amino acid sequences are set forth in SEQ ID NOs: 170, 171, 172, respectively;
(3) the selected VH CDRs 1, 2, 3 amino acid sequences are set forth in SEQ ID NOs: 173, 174, 175, respectively, and the selected VL CDRs 1, 2, 3 amino acid sequences are set forth in SEQ ID NOs: 176, 177, 178, respectively;
(4) the selected VH CDRs 1, 2, 3 amino acid sequences are set forth in SEQ ID NOs: 179, 180, 181, respectively, and the selected VL CDRs 1, 2, 3 amino acid sequences are set forth in SEQ ID NOs: 182, 183, 184, respectively;
(5) the selected VH CDRs 1, 2, 3 amino acid sequences are set forth in SEQ ID NOs: 185, 186, 187, respectively, and the selected VL CDRs 1, 2, 3 amino acid sequences are set forth in SEQ ID NOs: 188, 189, 190, respectively;
(6) the selected VH CDRs 1, 2, 3 amino acid sequences are set forth in SEQ ID NOs: 191, 192, 193, respectively, and the selected VL CDRs 1, 2, 3 amino acid sequences are set forth in SEQ ID NOs: 194, 195, 196, respectively;
(7) the selected VH CDRs 1, 2, 3 amino acid sequences are set forth in SEQ ID NOs: 197, 198, 199, respectively, and the selected VL CDRs 1, 2, 3 amino acid sequences are set forth in SEQ ID NOs: 200, 201, 202, respectively;
(8) the selected VH CDRs 1, 2, 3 amino acid sequences are set forth in SEQ ID NOs: 203, 204, 205, respectively, and the selected VL CDRs 1, 2, 3 amino acid sequences are set forth in SEQ ID NOs: 206, 207, 208, respectively;
(9) the selected VH CDRs 1, 2, 3 amino acid sequences are set forth in SEQ ID NOs: 209, 210, 211, respectively, and the selected VL CDRs 1, 2, 3 amino acid sequences are set forth in SEQ ID NOs: 212, 213, 214, respectively;
(10) the selected VH CDRs 1, 2, 3 amino acid sequences are set forth in SEQ ID NOs: 215, 216, 217, respectively, and the selected VL CDRs 1, 2, 3 amino acid sequences are set forth in SEQ ID NOs: 218, 219, 220, respectively;
(11) the selected VH CDRs 1, 2, 3 amino acid sequences are set forth in SEQ ID NOs: 221, 222, 223, respectively, and the selected VL CDRs 1, 2, 3 amino acid sequences are set forth in SEQ ID NOs: 224, 225, 226, respectively;
(12) the selected VH CDRs 1, 2, 3 amino acid sequences are set forth in SEQ ID NOs: 227, 228, 229, respectively, and the selected VL CDRs 1, 2, 3 amino acid sequences are set forth in SEQ ID NOs: 230, 231, 232, respectively;
(13) the selected VH CDRs 1, 2, 3 amino acid sequences are set forth in SEQ ID NOs: 233, 234, 235, respectively, and the selected VL CDRs 1, 2, 3 amino acid sequences are set forth in SEQ ID NOs: 236, 237, 238, respectively;
(14) the selected VH CDRs 1, 2, 3 amino acid sequences are set forth in SEQ ID NOs: 239, 240, 241, respectively, and the selected VL CDRs 1, 2, 3 amino acid sequences are set forth in SEQ ID NOs: 242, 243, 244, respectively;
(15) the selected VH CDRs 1, 2, 3 amino acid sequences are set forth in SEQ ID NOs: 245, 246, 247, respectively, and the selected VL CDRs 1, 2, 3 amino acid sequences are set forth in SEQ ID NOs: 248, 249, 250, respectively;
(16) the selected VH CDRs 1, 2, 3 amino acid sequences are set forth in SEQ ID NOs: 251, 252, 253, respectively, and the selected VL CDRs 1, 2, 3 amino acid sequences are set forth in SEQ ID NOs: 254, 255, 256, respectively;
(17) the selected VH CDRs 1, 2, 3 amino acid sequences are set forth in SEQ ID NOs: 257, 258, 259, respectively, and the selected VL CDRs 1, 2, 3 amino acid sequences are set forth in SEQ ID NOs: 260, 261, 262, respectively;
(18) the selected VH CDRs 1, 2, 3 amino acid sequences are set forth in SEQ ID NOs: 263, 264, 265, respectively, and the selected VL CDRs 1, 2, 3 amino acid sequences are set forth in SEQ ID NOs: 266, 267, 268, respectively;
(19) the selected VH CDRs 1, 2, 3 amino acid sequences are set forth in SEQ ID NOs: 269, 270, 271, respectively, and the selected VL CDRs 1, 2, 3 amino acid sequences are set forth in SEQ ID NOs: 272, 273, 274, respectively;
(20) the selected VH CDRs 1, 2, 3 amino acid sequences are set forth in SEQ ID NOs: 275, 276, 277, respectively, and the selected VL CDRs 1, 2, 3 amino acid sequences are set forth in SEQ ID NOs: 278, 279, 280, respectively;
(21) the selected VH CDRs 1, 2, 3 amino acid sequences are set forth in SEQ ID NOs: 935, 936, 937, respectively, and the selected VL CDRs 1, 2, 3 amino acid sequences are set forth in SEQ ID NOs: 938, 939, 940, respectively;
(22) the selected VH CDRs 1, 2, 3 amino acid sequences are set forth in SEQ ID NOs: 941, 942, 943, respectively, and the selected VL CDRs 1, 2, 3 amino acid sequences are set forth in SEQ ID NOs: 944, 945, 946, respectively;
(23) the selected VH CDRs 1, 2, 3 amino acid sequences are set forth in SEQ ID NOs: 947, 948, 949, respectively, and the selected VL CDRs 1, 2, 3 amino acid sequences are set forth in SEQ ID NOs: 950, 951, 952, respectively;
(24) the selected VH CDRs 1, 2, 3 amino acid sequences are set forth in SEQ ID NOs: 953, 954, 955, respectively, and the selected VL CDRs 1, 2, 3 amino acid sequences are set forth in SEQ ID NOs: 956, 957, 958, respectively;
(25) the selected VH CDRs 1, 2, 3 amino acid sequences are set forth in SEQ ID NOs: 959, 960, 961, respectively, and the selected VL CDRs 1, 2, 3 amino acid sequences are set forth in SEQ ID NOs: 962, 963, 964, respectively;
(26) the selected VH CDRs 1, 2, 3 amino acid sequences are set forth in SEQ ID NOs: 965, 966, 967, respectively, and the selected VL CDRs 1, 2, 3 amino acid sequences are set forth in SEQ ID NOs: 968, 969, 970, respectively;
(27) the selected VH CDRs 1, 2, 3 amino acid sequences are set forth in SEQ ID NOs: 971, 972, 973, respectively, and the selected VL CDRs 1, 2, 3 amino acid sequences are set forth in SEQ ID NOs: 974, 975, 976, respectively;
(28) the selected VH CDRs 1, 2, 3 amino acid sequences are set forth in SEQ ID NOs: 977, 978, 979, respectively, and the selected VL CDRs 1, 2, 3 amino acid sequences are set forth in SEQ ID NOs: 980, 981, 982, respectively;
(29) the selected VH CDRs 1, 2, 3 amino acid sequences are set forth in SEQ ID NOs: 983, 984, 985, respectively, and the selected VL CDRs 1, 2, 3 amino acid sequences are set forth in SEQ ID NOs: 986, 987, 988, respectively;
(30) the selected VH CDRs 1, 2, 3 amino acid sequences are set forth in SEQ ID NOs: 989, 990, 991, respectively, and the selected VL CDRs 1, 2, 3 amino acid sequences are set forth in SEQ ID NOs: 992, 993, 994, respectively;
(31) the selected VH CDRs 1, 2, 3 amino acid sequences are set forth in SEQ ID NOs: 995, 996, 997, respectively, and the selected VL CDRs 1, 2, 3 amino acid sequences are set forth in SEQ ID NOs: 998, 999, 1000, respectively;
(32) the selected VH CDRs 1, 2, 3 amino acid sequences are set forth in SEQ ID NOs: 1001, 1002, 1003, respectively, and the selected VL CDRs 1, 2, 3 amino acid sequences are set forth in SEQ ID NOs: 1004, 1005, 1006, respectively;
(33) the selected VH CDRs 1, 2, 3 amino acid sequences are set forth in SEQ ID NOs: 1007, 1008, 1009, respectively, and the selected VL CDRs 1, 2, 3 amino acid sequences are set forth in SEQ ID NOs: 1010, 1011, 1012, respectively;
(34) the selected VH CDRs 1, 2, 3 amino acid sequences are set forth in SEQ ID NOs: 1013, 1014, 1015, respectively, and the selected VL CDRs 1, 2, 3 amino acid sequences are set forth in SEQ ID NOs: 1016, 1017, 1018, respectively;
(35) the selected VH CDRs 1, 2, 3 amino acid sequences are set forth in SEQ ID NOs: 1019, 1020, 1021, respectively, and the selected VL CDRs 1, 2, 3 amino acid sequences are set forth in SEQ ID NOs: 1022, 1023, 1024, respectively;
(36) the selected VH CDRs 1, 2, 3 amino acid sequences are set forth in SEQ ID NOs: 1025, 1026, 1027, respectively, and the selected VL CDRs 1, 2, 3 amino acid sequences are set forth in SEQ ID NOs: 1028, 1029, 1030, respectively;
(37) the selected VH CDRs 1, 2, 3 amino acid sequences are set forth in SEQ ID NOs: 1031, 1032, 1033, respectively, and the selected VL CDRs 1, 2, 3 amino acid sequences are set forth in SEQ ID NOs: 1034, 1035, 1036, respectively;
(38) the selected VH CDRs 1, 2, 3 amino acid sequences are set forth in SEQ ID NOs: 1037, 1038, 1039, respectively, and the selected VL CDRs 1, 2, 3 amino acid sequences are set forth in SEQ ID NOs: 1040, 1041, 1042, respectively;
(39) the selected VH CDRs 1, 2, 3 amino acid sequences are set forth in SEQ ID NOs: 1043, 1044, 1045, respectively, and the selected VL CDRs 1, 2, 3 amino acid sequences are set forth in SEQ ID NOs: 1046, 1047, 1048, respectively; and
(40) the selected VH CDRs 1, 2, 3 amino acid sequences are set forth in SEQ ID NOs: 1049, 1050, 1051, respectively, and the selected VL CDRs 1, 2, 3 amino acid sequences are set forth in SEQ ID NOs: 1052, 1053, 1054, respectively.

5. (canceled)

6. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 281, 282, 283, respectively, and the selected VL CDRs 1, 2, 3 amino acid sequences are set forth in SEQ ID NOs: 284, 285, 286, respectively;
(2) the selected VH CDRs 1, 2, 3 amino acid sequences are set forth in SEQ ID NOs: 287, 288, 289, respectively, and the selected VL CDRs 1, 2, 3 amino acid sequences are set forth in SEQ ID NOs: 290, 291, 292, respectively;
(3) the selected VH CDRs 1, 2, 3 amino acid sequences are set forth in SEQ ID NOs: 293, 294, 295, respectively, and the selected VL CDRs 1, 2, 3 amino acid sequences are set forth in SEQ ID NOs: 296, 297, 298, respectively;
(4) the selected VH CDRs 1, 2, 3 amino acid sequences are set forth in SEQ ID NOs: 299, 300, 301, respectively, and the selected VL CDRs 1, 2, 3 amino acid sequences are set forth in SEQ ID NOs: 302, 303, 304, respectively;
(5) the selected VH CDRs 1, 2, 3 amino acid sequences are set forth in SEQ ID NOs: 305, 306, 307, respectively, and the selected VL CDRs 1, 2, 3 amino acid sequences are set forth in SEQ ID NOs: 308, 309, 310, respectively;
(6) the selected VH CDRs 1, 2, 3 amino acid sequences are set forth in SEQ ID NOs: 311, 312, 313, respectively, and the selected VL CDRs 1, 2, 3 amino acid sequences are set forth in SEQ ID NOs: 314, 315, 316, respectively;
(7) the selected VH CDRs 1, 2, 3 amino acid sequences are set forth in SEQ ID NOs: 317, 318, 319, respectively, and the selected VL CDRs 1, 2, 3 amino acid sequences are set forth in SEQ ID NOs: 320, 321, 322, respectively;
(8) the selected VH CDRs 1, 2, 3 amino acid sequences are set forth in SEQ ID NOs: 323, 324, 325, respectively, and the selected VL CDRs 1, 2, 3 amino acid sequences are set forth in SEQ ID NOs: 326, 327, 328, respectively;
(9) the selected VH CDRs 1, 2, 3 amino acid sequences are set forth in SEQ ID NOs: 329, 330, 331, respectively, and the selected VL CDRs 1, 2, 3 amino acid sequences are set forth in SEQ ID NOs: 332, 333, 334, respectively;
(10) the selected VH CDRs 1, 2, 3 amino acid sequences are set forth in SEQ ID NOs: 335, 336, 337, respectively, and the selected VL CDRs 1, 2, 3 amino acid sequences are set forth in SEQ ID NOs: 338, 339, 340, respectively;
(11) the selected VH CDRs 1, 2, 3 amino acid sequences are set forth in SEQ ID NOs: 341, 342, 343, respectively, and the selected VL CDRs 1, 2, 3 amino acid sequences are set forth in SEQ ID NOs: 344, 345, 346, respectively;
(12) the selected VH CDRs 1, 2, 3 amino acid sequences are set forth in SEQ ID NOs: 347, 348, 349, respectively, and the selected VL CDRs 1, 2, 3 amino acid sequences are set forth in SEQ ID NOs: 350, 351, 352, respectively;
(13) the selected VH CDRs 1, 2, 3 amino acid sequences are set forth in SEQ ID NOs: 353, 354, 355, respectively, and the selected VL CDRs 1, 2, 3 amino acid sequences are set forth in SEQ ID NOs: 356, 357, 358, respectively;
(14) the selected VH CDRs 1, 2, 3 amino acid sequences are set forth in SEQ ID NOs: 359, 360, 361, respectively, and the selected VL CDRs 1, 2, 3 amino acid sequences are set forth in SEQ ID NOs: 362, 363, 364, respectively;
(15) the selected VH CDRs 1, 2, 3 amino acid sequences are set forth in SEQ ID NOs: 365, 366, 367, respectively, and the selected VL CDRs 1, 2, 3 amino acid sequences are set forth in SEQ ID NOs: 368, 369, 370, respectively;
(16) the selected VH CDRs 1, 2, 3 amino acid sequences are set forth in SEQ ID NOs: 371, 372, 373, respectively, and the selected VL CDRs 1, 2, 3 amino acid sequences are set forth in SEQ ID NOs: 374, 375, 376, respectively;
(17) the selected VH CDRs 1, 2, 3 amino acid sequences are set forth in SEQ ID NOs: 377, 378, 379, respectively, and the selected VL CDRs 1, 2, 3 amino acid sequences are set forth in SEQ ID NOs: 380, 381, 382, respectively;
(18) the selected VH CDRs 1, 2, 3 amino acid sequences are set forth in SEQ ID NOs: 383, 384, 385, respectively, and the selected VL CDRs 1, 2, 3 amino acid sequences are set forth in SEQ ID NOs: 386, 387, 388, respectively;
(19) the selected VH CDRs 1, 2, 3 amino acid sequences are set forth in SEQ ID NOs: 389, 390, 391, respectively, and the selected VL CDRs 1, 2, 3 amino acid sequences are set forth in SEQ ID NOs: 392, 393, 394, respectively;
(20) the selected VH CDRs 1, 2, 3 amino acid sequences are set forth in SEQ ID NOs: 395, 396, 397, respectively, and the selected VL CDRs 1, 2, 3 amino acid sequences are set forth in SEQ ID NOs: 398, 399, 400, respectively;
(21) the selected VH CDRs 1, 2, 3 amino acid sequences are set forth in SEQ ID NOs: 1055, 1056, 1057, respectively, and the selected VL CDRs 1, 2, 3 amino acid sequences are set forth in SEQ ID NOs: 1058, 1059, 1060, respectively;
(22) the selected VH CDRs 1, 2, 3 amino acid sequences are set forth in SEQ ID NOs: 1061, 1062, 1063, respectively, and the selected VL CDRs 1, 2, 3 amino acid sequences are set forth in SEQ ID NOs: 1064, 1065, 1066, respectively;
(23) the selected VH CDRs 1, 2, 3 amino acid sequences are set forth in SEQ ID NOs: 1067, 1068, 1069, respectively, and the selected VL CDRs 1, 2, 3 amino acid sequences are set forth in SEQ ID NOs: 1070, 1071, 1072, respectively;
(24) the selected VH CDRs 1, 2, 3 amino acid sequences are set forth in SEQ ID NOs: 1073, 1074, 1075, respectively, and the selected VL CDRs 1, 2, 3 amino acid sequences are set forth in SEQ ID NOs: 1076, 1077, 1078, respectively;
(25) the selected VH CDRs 1, 2, 3 amino acid sequences are set forth in SEQ ID NOs: 1079, 1080, 1081, respectively, and the selected VL CDRs 1, 2, 3 amino acid sequences are set forth in SEQ ID NOs: 1082, 1083, 1084, respectively;
(26) the selected VH CDRs 1, 2, 3 amino acid sequences are set forth in SEQ ID NOs: 1085, 1086, 1087, respectively, and the selected VL CDRs 1, 2, 3 amino acid sequences are set forth in SEQ ID NOs: 1088, 1089, 1090, respectively;
(27) the selected VH CDRs 1, 2, 3 amino acid sequences are set forth in SEQ ID NOs: 1091, 1092, 1093, respectively, and the selected VL CDRs 1, 2, 3 amino acid sequences are set forth in SEQ ID NOs: 1094, 1095, 1096, respectively;
(28) the selected VH CDRs 1, 2, 3 amino acid sequences are set forth in SEQ ID NOs: 1097, 1098, 1099, respectively, and the selected VL CDRs 1, 2, 3 amino acid sequences are set forth in SEQ ID NOs: 1100, 1101, 1102, respectively;
(29) the selected VH CDRs 1, 2, 3 amino acid sequences are set forth in SEQ ID NOs: 1103, 1104, 1105, respectively, and the selected VL CDRs 1, 2, 3 amino acid sequences are set forth in SEQ ID NOs: 1106, 1107, 1108, respectively;
(30) the selected VH CDRs 1, 2, 3 amino acid sequences are set forth in SEQ ID NOs: 1109, 1110, 1111, respectively, and the selected VL CDRs 1, 2, 3 amino acid sequences are set forth in SEQ ID NOs: 1112, 1113, 1114, respectively;
(31) the selected VH CDRs 1, 2, 3 amino acid sequences are set forth in SEQ ID NOs: 1115, 1116, 1117, respectively, and the selected VL CDRs 1, 2, 3 amino acid sequences are set forth in SEQ ID NOs: 1118, 1119, 1120, respectively;
(32) the selected VH CDRs 1, 2, 3 amino acid sequences are set forth in SEQ ID NOs: 1121, 1122, 1123, respectively, and the selected VL CDRs 1, 2, 3 amino acid sequences are set forth in SEQ ID NOs: 1124, 1125, 1126, respectively;
(33) the selected VH CDRs 1, 2, 3 amino acid sequences are set forth in SEQ ID NOs: 1127, 1128, 1129, respectively, and the selected VL CDRs 1, 2, 3 amino acid sequences are set forth in SEQ ID NOs: 1130, 1131, 1132, respectively;
(34) the selected VH CDRs 1, 2, 3 amino acid sequences are set forth in SEQ ID NOs: 1133, 1134, 1135, respectively, and the selected VL CDRs 1, 2, 3 amino acid sequences are set forth in SEQ ID NOs: 1136, 1137, 1138, respectively;
(35) the selected VH CDRs 1, 2, 3 amino acid sequences are set forth in SEQ ID NOs: 1139, 1140, 1141, respectively, and the selected VL CDRs 1, 2, 3 amino acid sequences are set forth in SEQ ID NOs: 1142, 1143, 1144, respectively;
(36) the selected VH CDRs 1, 2, 3 amino acid sequences are set forth in SEQ ID NOs: 1145, 1146, 1147, respectively, and the selected VL CDRs 1, 2, 3 amino acid sequences are set forth in SEQ ID NOs: 1148, 1149, 1150, respectively;
(37) the selected VH CDRs 1, 2, 3 amino acid sequences are set forth in SEQ ID NOs: 1151, 1152, 1153, respectively, and the selected VL CDRs 1, 2, 3 amino acid sequences are set forth in SEQ ID NOs: 1154, 1155, 1156, respectively;
(38) the selected VH CDRs 1, 2, 3 amino acid sequences are set forth in SEQ ID NOs: 1157, 1158, 1159, respectively, and the selected VL CDRs 1, 2, 3 amino acid sequences are set forth in SEQ ID NOs: 1160, 1161, 1162, respectively;
(39) the selected VH CDRs 1, 2, 3 amino acid sequences are set forth in SEQ ID NOs: 1163, 1164, 1165, respectively, and the selected VL CDRs 1, 2, 3 amino acid sequences are set forth in SEQ ID NOs: 1166, 1167, 1168, respectively; and
(40) the selected VH CDRs 1, 2, 3 amino acid sequences are set forth in SEQ ID NOs: 1169, 1170, 1171, respectively, and the selected VL CDRs 1, 2, 3 amino acid sequences are set forth in SEQ ID NOs: 1172, 1173, 1174, respectively.

7. (canceled)

8. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 401, 402, 403, respectively, and the selected VL CDRs 1, 2, 3 amino acid sequences are set forth in SEQ ID NOs: 404, 405, 406, respectively;
(2) the selected VH CDRs 1, 2, 3 amino acid sequences are set forth in SEQ ID NOs: 407, 408, 409, respectively, and the selected VL CDRs 1, 2, 3 amino acid sequences are set forth in SEQ ID NOs: 410, 411, 412, respectively;
(3) the selected VH CDRs 1, 2, 3 amino acid sequences are set forth in SEQ ID NOs: 413, 414, 415, respectively, and the selected VL CDRs 1, 2, 3 amino acid sequences are set forth in SEQ ID NOs: 416, 417, 418, respectively;
(4) the selected VH CDRs 1, 2, 3 amino acid sequences are set forth in SEQ ID NOs: 419, 420, 421, respectively, and the selected VL CDRs 1, 2, 3 amino acid sequences are set forth in SEQ ID NOs: 422, 423, 424, respectively;
(5) the selected VH CDRs 1, 2, 3 amino acid sequences are set forth in SEQ ID NOs: 425, 426, 427, respectively, and the selected VL CDRs 1, 2, 3 amino acid sequences are set forth in SEQ ID NOs: 428, 429, 430, respectively;
(6) the selected VH CDRs 1, 2, 3 amino acid sequences are set forth in SEQ ID NOs: 431, 432, 433, respectively, and the selected VL CDRs 1, 2, 3 amino acid sequences are set forth in SEQ ID NOs: 434, 435, 436, respectively;
(7) the selected VH CDRs 1, 2, 3 amino acid sequences are set forth in SEQ ID NOs: 437, 438, 439, respectively, and the selected VL CDRs 1, 2, 3 amino acid sequences are set forth in SEQ ID NOs: 440, 441, 442, respectively;
(8) the selected VH CDRs 1, 2, 3 amino acid sequences are set forth in SEQ ID NOs: 443, 444, 445, respectively, and the selected VL CDRs 1, 2, 3 amino acid sequences are set forth in SEQ ID NOs: 446, 447, 448, respectively;
(9) the selected VH CDRs 1, 2, 3 amino acid sequences are set forth in SEQ ID NOs: 449, 450, 451, respectively, and the selected VL CDRs 1, 2, 3 amino acid sequences are set forth in SEQ ID NOs: 452, 453, 454, respectively;
(10) the selected VH CDRs 1, 2, 3 amino acid sequences are set forth in SEQ ID NOs: 455, 456, 457, respectively, and the selected VL CDRs 1, 2, 3 amino acid sequences are set forth in SEQ ID NOs: 458, 459, 460, respectively;
(11) the selected VH CDRs 1, 2, 3 amino acid sequences are set forth in SEQ ID NOs: 461, 462, 463, respectively, and the selected VL CDRs 1, 2, 3 amino acid sequences are set forth in SEQ ID NOs: 464, 465, 466, respectively;
(12) the selected VH CDRs 1, 2, 3 amino acid sequences are set forth in SEQ ID NOs: 467, 468, 469, respectively, and the selected VL CDRs 1, 2, 3 amino acid sequences are set forth in SEQ ID NOs: 470, 471, 472, respectively;
(13) the selected VH CDRs 1, 2, 3 amino acid sequences are set forth in SEQ ID NOs: 473, 474, 475, respectively, and the selected VL CDRs 1, 2, 3 amino acid sequences are set forth in SEQ ID NOs: 476, 477, 478, respectively;
(14) the selected VH CDRs 1, 2, 3 amino acid sequences are set forth in SEQ ID NOs: 479, 480, 481, respectively, and the selected VL CDRs 1, 2, 3 amino acid sequences are set forth in SEQ ID NOs: 482, 483, 484, respectively;
(15) the selected VH CDRs 1, 2, 3 amino acid sequences are set forth in SEQ ID NOs: 485, 486, 487, respectively, and the selected VL CDRs 1, 2, 3 amino acid sequences are set forth in SEQ ID NOs: 488, 489, 490, respectively;
(16) the selected VH CDRs 1, 2, 3 amino acid sequences are set forth in SEQ ID NOs: 491, 492, 493, respectively, and the selected VL CDRs 1, 2, 3 amino acid sequences are set forth in SEQ ID NOs: 494, 495, 496, respectively;
(17) the selected VH CDRs 1, 2, 3 amino acid sequences are set forth in SEQ ID NOs: 497, 498, 499, respectively, and the selected VL CDRs 1, 2, 3 amino acid sequences are set forth in SEQ ID NOs: 500, 501, 502, respectively;
(18) the selected VH CDRs 1, 2, 3 amino acid sequences are set forth in SEQ ID NOs: 503, 504, 505, respectively, and the selected VL CDRs 1, 2, 3 amino acid sequences are set forth in SEQ ID NOs: 506, 507, 508, respectively;
(19) the selected VH CDRs 1, 2, 3 amino acid sequences are set forth in SEQ ID NOs: 509, 510, 511, respectively, and the selected VL CDRs 1, 2, 3 amino acid sequences are set forth in SEQ ID NOs: 512, 513, 514, respectively;
(20) the selected VH CDRs 1, 2, 3 amino acid sequences are set forth in SEQ ID NOs: 515, 516, 517, respectively, and the selected VL CDRs 1, 2, 3 amino acid sequences are set forth in SEQ ID NOs: 518, 519, 520, respectively;
(21) the selected VH CDRs 1, 2, 3 amino acid sequences are set forth in SEQ ID NOs: 1175, 1176, 1177, respectively, and the selected VL CDRs 1, 2, 3 amino acid sequences are set forth in SEQ ID NOs: 1178, 1179, 1180, respectively;
(22) the selected VH CDRs 1, 2, 3 amino acid sequences are set forth in SEQ ID NOs: 1181, 1182, 1183, respectively, and the selected VL CDRs 1, 2, 3 amino acid sequences are set forth in SEQ ID NOs: 1184, 1185, 1186, respectively;
(23) the selected VH CDRs 1, 2, 3 amino acid sequences are set forth in SEQ ID NOs: 1187, 1188, 1189, respectively, and the selected VL CDRs 1, 2, 3 amino acid sequences are set forth in SEQ ID NOs: 1190, 1191, 1192, respectively;
(24) the selected VH CDRs 1, 2, 3 amino acid sequences are set forth in SEQ ID NOs: 1193, 1194, 1195, respectively, and the selected VL CDRs 1, 2, 3 amino acid sequences are set forth in SEQ ID NOs: 1196, 1197, 1198, respectively;
(25) the selected VH CDRs 1, 2, 3 amino acid sequences are set forth in SEQ ID NOs: 1199, 1200, 1201, respectively, and the selected VL CDRs 1, 2, 3 amino acid sequences are set forth in SEQ ID NOs: 1202, 1203, 1204, respectively;
(26) the selected VH CDRs 1, 2, 3 amino acid sequences are set forth in SEQ ID NOs: 1205, 1206, 1207, respectively, and the selected VL CDRs 1, 2, 3 amino acid sequences are set forth in SEQ ID NOs: 1208, 1209, 1210, respectively;
(27) the selected VH CDRs 1, 2, 3 amino acid sequences are set forth in SEQ ID NOs: 1211, 1212, 1213, respectively, and the selected VL CDRs 1, 2, 3 amino acid sequences are set forth in SEQ ID NOs: 1214, 1215, 1216, respectively;
(28) the selected VH CDRs 1, 2, 3 amino acid sequences are set forth in SEQ ID NOs: 1217, 1218, 1219, respectively, and the selected VL CDRs 1, 2, 3 amino acid sequences are set forth in SEQ ID NOs: 1220, 1221, 1222, respectively;
(29) the selected VH CDRs 1, 2, 3 amino acid sequences are set forth in SEQ ID NOs: 1223, 1224, 1225, respectively, and the selected VL CDRs 1, 2, 3 amino acid sequences are set forth in SEQ ID NOs: 1226, 1227, 1228, respectively;
(30) the selected VH CDRs 1, 2, 3 amino acid sequences are set forth in SEQ ID NOs: 1229, 1230, 1231, respectively, and the selected VL CDRs 1, 2, 3 amino acid sequences are set forth in SEQ ID NOs: 1232, 1233, 1234, respectively;
(31) the selected VH CDRs 1, 2, 3 amino acid sequences are set forth in SEQ ID NOs: 1235, 1236, 1237, respectively, and the selected VL CDRs 1, 2, 3 amino acid sequences are set forth in SEQ ID NOs: 1238, 1239, 1240, respectively;
(32) the selected VH CDRs 1, 2, 3 amino acid sequences are set forth in SEQ ID NOs: 1241, 1242, 1243, respectively, and the selected VL CDRs 1, 2, 3 amino acid sequences are set forth in SEQ ID NOs: 1244, 1245, 1246, respectively;
(33) the selected VH CDRs 1, 2, 3 amino acid sequences are set forth in SEQ ID NOs: 1247, 1248, 1249, respectively, and the selected VL CDRs 1, 2, 3 amino acid sequences are set forth in SEQ ID NOs: 1250, 1251, 1252, respectively;
(34) the selected VH CDRs 1, 2, 3 amino acid sequences are set forth in SEQ ID NOs: 1253, 1254, 1255, respectively, and the selected VL CDRs 1, 2, 3 amino acid sequences are set forth in SEQ ID NOs: 1256, 1257, 1258, respectively;
(35) the selected VH CDRs 1, 2, 3 amino acid sequences are set forth in SEQ ID NOs: 1259, 1260, 1261, respectively, and the selected VL CDRs 1, 2, 3 amino acid sequences are set forth in SEQ ID NOs: 1262, 1263, 1264, respectively;
(36) the selected VH CDRs 1, 2, 3 amino acid sequences are set forth in SEQ ID NOs: 1265, 1266, 1267, respectively, and the selected VL CDRs 1, 2, 3 amino acid sequences are set forth in SEQ ID NOs: 1268, 1269, 1270, respectively;
(37) the selected VH CDRs 1, 2, 3 amino acid sequences are set forth in SEQ ID NOs: 1271, 1272, 1273, respectively, and the selected VL CDRs 1, 2, 3 amino acid sequences are set forth in SEQ ID NOs: 1274, 1275, 1276, respectively;
(38) the selected VH CDRs 1, 2, 3 amino acid sequences are set forth in SEQ ID NOs: 1277, 1278, 1279, respectively, and the selected VL CDRs 1, 2, 3 amino acid sequences are set forth in SEQ ID NOs: 1280, 1281, 1282, respectively;
(39) the selected VH CDRs 1, 2, 3 amino acid sequences are set forth in SEQ ID NOs: 1283, 1284, 1285, respectively, and the selected VL CDRs 1, 2, 3 amino acid sequences are set forth in SEQ ID NOs: 1286, 1287, 1288, respectively; and
(40) the selected VH CDRs 1, 2, 3 amino acid sequences are set forth in SEQ ID NOs: 1289, 1290, 1291, respectively, and the selected VL CDRs 1, 2, 3 amino acid sequences are set forth in SEQ ID NOs: 1292, 1293, 1294, respectively.

9. (canceled)

10. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 521, 522, 523, respectively, and the selected VL CDRs 1, 2, 3 amino acid sequences are set forth in SEQ ID NOs: 524, 525, 526, respectively;
(2) the selected VH CDRs 1, 2, 3 amino acid sequences are set forth in SEQ ID NOs: 527, 528, 529, respectively, and the selected VL CDRs 1, 2, 3 amino acid sequences are set forth in SEQ ID NOs: 530, 531, 532, respectively;
(3) the selected VH CDRs 1, 2, 3 amino acid sequences are set forth in SEQ ID NOs: 533, 534, 535, respectively, and the selected VL CDRs 1, 2, 3 amino acid sequences are set forth in SEQ ID NOs: 536, 537, 538, respectively;
(4) the selected VH CDRs 1, 2, 3 amino acid sequences are set forth in SEQ ID NOs: 539, 540, 541, respectively, and the selected VL CDRs 1, 2, 3 amino acid sequences are set forth in SEQ ID NOs: 542, 543, 544, respectively;
(5) the selected VH CDRs 1, 2, 3 amino acid sequences are set forth in SEQ ID NOs: 545, 546, 547, respectively, and the selected VL CDRs 1, 2, 3 amino acid sequences are set forth in SEQ ID NOs: 548, 549, 550, respectively;
(6) the selected VH CDRs 1, 2, 3 amino acid sequences are set forth in SEQ ID NOs: 551, 552, 553, respectively, and the selected VL CDRs 1, 2, 3 amino acid sequences are set forth in SEQ ID NOs: 554, 555, 556, respectively;
(7) the selected VH CDRs 1, 2, 3 amino acid sequences are set forth in SEQ ID NOs: 557, 558, 559, respectively, and the selected VL CDRs 1, 2, 3 amino acid sequences are set forth in SEQ ID NOs: 560, 561, 562, respectively;
(8) the selected VH CDRs 1, 2, 3 amino acid sequences are set forth in SEQ ID NOs: 563, 564, 565, respectively, and the selected VL CDRs 1, 2, 3 amino acid sequences are set forth in SEQ ID NOs: 566, 567, 568, respectively;
(9) the selected VH CDRs 1, 2, 3 amino acid sequences are set forth in SEQ ID NOs: 569, 570, 571, respectively, and the selected VL CDRs 1, 2, 3 amino acid sequences are set forth in SEQ ID NOs: 572, 573, 574, respectively;
(10) the selected VH CDRs 1, 2, 3 amino acid sequences are set forth in SEQ ID NOs: 575, 576, 577, respectively, and the selected VL CDRs 1, 2, 3 amino acid sequences are set forth in SEQ ID NOs: 578, 579, 580, respectively;
(11) the selected VH CDRs 1, 2, 3 amino acid sequences are set forth in SEQ ID NOs: 581, 582, 583, respectively, and the selected VL CDRs 1, 2, 3 amino acid sequences are set forth in SEQ ID NOs: 584, 585, 586, respectively;
(12) the selected VH CDRs 1, 2, 3 amino acid sequences are set forth in SEQ ID NOs: 587, 588, 589, respectively, and the selected VL CDRs 1, 2, 3 amino acid sequences are set forth in SEQ ID NOs: 590, 591, 592, respectively;
(13) the selected VH CDRs 1, 2, 3 amino acid sequences are set forth in SEQ ID NOs: 593, 594, 595, respectively, and the selected VL CDRs 1, 2, 3 amino acid sequences are set forth in SEQ ID NOs: 596, 597, 598, respectively;
(14) the selected VH CDRs 1, 2, 3 amino acid sequences are set forth in SEQ ID NOs: 599, 600, 601, respectively, and the selected VL CDRs 1, 2, 3 amino acid sequences are set forth in SEQ ID NOs: 602, 603, 604, respectively;
(15) the selected VH CDRs 1, 2, 3 amino acid sequences are set forth in SEQ ID NOs: 605, 606, 607, respectively, and the selected VL CDRs 1, 2, 3 amino acid sequences are set forth in SEQ ID NOs: 608, 609, 610, respectively;
(16) the selected VH CDRs 1, 2, 3 amino acid sequences are set forth in SEQ ID NOs: 611, 612, 613, respectively, and the selected VL CDRs 1, 2, 3 amino acid sequences are set forth in SEQ ID NOs: 614, 615, 616, respectively;
(17) the selected VH CDRs 1, 2, 3 amino acid sequences are set forth in SEQ ID NOs: 617, 618, 619, respectively, and the selected VL CDRs 1, 2, 3 amino acid sequences are set forth in SEQ ID NOs: 620, 621, 622, respectively;
(18) the selected VH CDRs 1, 2, 3 amino acid sequences are set forth in SEQ ID NOs: 623, 624, 625, respectively, and the selected VL CDRs 1, 2, 3 amino acid sequences are set forth in SEQ ID NOs: 626, 627, 628, respectively;
(19) the selected VH CDRs 1, 2, 3 amino acid sequences are set forth in SEQ ID NOs: 629, 630, 631, respectively, and the selected VL CDRs 1, 2, 3 amino acid sequences are set forth in SEQ ID NOs: 632, 633, 634, respectively;
(20) the selected VH CDRs 1, 2, 3 amino acid sequences are set forth in SEQ ID NOs: 635, 636, 637, respectively, and the selected VL CDRs 1, 2, 3 amino acid sequences are set forth in SEQ ID NOs: 638, 639, 640, respectively;
(21) the selected VH CDRs 1, 2, 3 amino acid sequences are set forth in SEQ ID NOs: 1295, 1296, 1297, respectively, and the selected VL CDRs 1, 2, 3 amino acid sequences are set forth in SEQ ID NOs: 1298, 1299, 1300, respectively;
(22) the selected VH CDRs 1, 2, 3 amino acid sequences are set forth in SEQ ID NOs: 1301, 1302, 1303, respectively, and the selected VL CDRs 1, 2, 3 amino acid sequences are set forth in SEQ ID NOs: 1304, 1305, 1306, respectively;
(23) the selected VH CDRs 1, 2, 3 amino acid sequences are set forth in SEQ ID NOs: 1307, 1308, 1309, respectively, and the selected VL CDRs 1, 2, 3 amino acid sequences are set forth in SEQ ID NOs: 1310, 1311, 1312, respectively;
(24) the selected VH CDRs 1, 2, 3 amino acid sequences are set forth in SEQ ID NOs: 1313, 1314, 1315, respectively, and the selected VL CDRs 1, 2, 3 amino acid sequences are set forth in SEQ ID NOs: 1316, 1317, 1318, respectively;
(25) the selected VH CDRs 1, 2, 3 amino acid sequences are set forth in SEQ ID NOs: 1319, 1320, 1321, respectively, and the selected VL CDRs 1, 2, 3 amino acid sequences are set forth in SEQ ID NOs: 1322, 1323, 1324, respectively;
(26) the selected VH CDRs 1, 2, 3 amino acid sequences are set forth in SEQ ID NOs: 1325, 1326, 1327, respectively, and the selected VL CDRs 1, 2, 3 amino acid sequences are set forth in SEQ ID NOs: 1328, 1329, 1330, respectively;
(27) the selected VH CDRs 1, 2, 3 amino acid sequences are set forth in SEQ ID NOs: 1331, 1332, 1333, respectively, and the selected VL CDRs 1, 2, 3 amino acid sequences are set forth in SEQ ID NOs: 1334, 1335, 1336, respectively;
(28) the selected VH CDRs 1, 2, 3 amino acid sequences are set forth in SEQ ID NOs: 1337, 1338, 1339, respectively, and the selected VL CDRs 1, 2, 3 amino acid sequences are set forth in SEQ ID NOs: 1340, 1341, 1342, respectively;
(29) the selected VH CDRs 1, 2, 3 amino acid sequences are set forth in SEQ ID NOs: 1343, 1344, 1345, respectively, and the selected VL CDRs 1, 2, 3 amino acid sequences are set forth in SEQ ID NOs: 1346, 1347, 1348, respectively;
(30) the selected VH CDRs 1, 2, 3 amino acid sequences are set forth in SEQ ID NOs: 1349, 1350, 1351, respectively, and the selected VL CDRs 1, 2, 3 amino acid sequences are set forth in SEQ ID NOs: 1352, 1353, 1354, respectively;
(31) the selected VH CDRs 1, 2, 3 amino acid sequences are set forth in SEQ ID NOs: 1355, 1356, 1357, respectively, and the selected VL CDRs 1, 2, 3 amino acid sequences are set forth in SEQ ID NOs: 1358, 1359, 1360, respectively;
(32) the selected VH CDRs 1, 2, 3 amino acid sequences are set forth in SEQ ID NOs: 1361, 1362, 1363, respectively, and the selected VL CDRs 1, 2, 3 amino acid sequences are set forth in SEQ ID NOs: 1364, 1365, 1366, respectively;
(33) the selected VH CDRs 1, 2, 3 amino acid sequences are set forth in SEQ ID NOs: 1367, 1368, 1369, respectively, and the selected VL CDRs 1, 2, 3 amino acid sequences are set forth in SEQ ID NOs: 1370, 1371, 1372, respectively;
(34) the selected VH CDRs 1, 2, 3 amino acid sequences are set forth in SEQ ID NOs: 1373, 1374, 1375, respectively, and the selected VL CDRs 1, 2, 3 amino acid sequences are set forth in SEQ ID NOs: 1376, 1377, 1378, respectively;
(35) the selected VH CDRs 1, 2, 3 amino acid sequences are set forth in SEQ ID NOs: 1379, 1380, 1381, respectively, and the selected VL CDRs 1, 2, 3 amino acid sequences are set forth in SEQ ID NOs: 1382, 1383, 1384, respectively;
(36) the selected VH CDRs 1, 2, 3 amino acid sequences are set forth in SEQ ID NOs: 1385, 1386, 1387, respectively, and the selected VL CDRs 1, 2, 3 amino acid sequences are set forth in SEQ ID NOs: 1388, 1389, 1390, respectively;
(37) the selected VH CDRs 1, 2, 3 amino acid sequences are set forth in SEQ ID NOs: 1391, 1392, 1393, respectively, and the selected VL CDRs 1, 2, 3 amino acid sequences are set forth in SEQ ID NOs: 1394, 1395, 1396, respectively;
(38) the selected VH CDRs 1, 2, 3 amino acid sequences are set forth in SEQ ID NOs: 1397, 1398, 1399, respectively, and the selected VL CDRs 1, 2, 3 amino acid sequences are set forth in SEQ ID NOs: 1400, 1401, 1402, respectively;
(39) the selected VH CDRs 1, 2, 3 amino acid sequences are set forth in SEQ ID NOs: 1403, 1404, 1405, respectively, and the selected VL CDRs 1, 2, 3 amino acid sequences are set forth in SEQ ID NOs: 1406, 1407, 1408, respectively; and
(40) the selected VH CDRs 1, 2, 3 amino acid sequences are set forth in SEQ ID NOs: 1409, 1410, 1411, respectively, and the selected VL CDRs 1, 2, 3 amino acid sequences are set forth in SEQ ID NOs: 1412, 1413, 1414, respectively.

11. (canceled)

12. The antibody or antigen-binding fragment thereof of claim 1, wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 708, 709, 710, respectively, and the selected VL CDRs 1, 2, 3 amino acid sequences are set forth in SEQ ID NOs: 711, 712, 713, respectively;
(2) the selected VH CDRs 1, 2, 3 amino acid sequences are set forth in SEQ ID NOs: 714, 715, 716, respectively, and the selected VL CDRs 1, 2, 3 amino acid sequences are set forth in SEQ ID NOs: 717, 718, 719, respectively;
(3) the selected VH CDRs 1, 2, 3 amino acid sequences are set forth in SEQ ID NOs: 720, 721, 722, respectively, and the selected VL CDRs 1, 2, 3 amino acid sequences are set forth in SEQ ID NOs: 723, 724, 725, respectively;
(4) the selected VH CDRs 1, 2, 3 amino acid sequences are set forth in SEQ ID NOs: 726, 727, 728, respectively, and the selected VL CDRs 1, 2, 3 amino acid sequences are set forth in SEQ ID NOs: 729, 730, 731, respectively;
(5) the selected VH CDRs 1, 2, 3 amino acid sequences are set forth in SEQ ID NOs: 732, 733, 734, respectively, and the selected VL CDRs 1, 2, 3 amino acid sequences are set forth in SEQ ID NOs: 735, 736, 737, respectively;
(6) the selected VH CDRs 1, 2, 3 amino acid sequences are set forth in SEQ ID NOs: 738, 739, 740, respectively, and the selected VL CDRs 1, 2, 3 amino acid sequences are set forth in SEQ ID NOs: 741, 742, 743, respectively;
(7) the selected VH CDRs 1, 2, 3 amino acid sequences are set forth in SEQ ID NOs: 744, 745, 746, respectively, and the selected VL CDRs 1, 2, 3 amino acid sequences are set forth in SEQ ID NOs: 747, 748, 749, respectively;
(8) the selected VH CDRs 1, 2, 3 amino acid sequences are set forth in SEQ ID NOs: 750, 751, 752, respectively, and the selected VL CDRs 1, 2, 3 amino acid sequences are set forth in SEQ ID NOs: 753, 754, 755, respectively;
(9) the selected VH CDRs 1, 2, 3 amino acid sequences are set forth in SEQ ID NOs: 756, 757, 758, respectively, and the selected VL CDRs 1, 2, 3 amino acid sequences are set forth in SEQ ID NOs: 759, 760, 761, respectively;
(10) the selected VH CDRs 1, 2, 3 amino acid sequences are set forth in SEQ ID NOs: 762, 763, 764, respectively, and the selected VL CDRs 1, 2, 3 amino acid sequences are set forth in SEQ ID NOs: 765, 766, 767, respectively;
(11) the selected VH CDRs 1, 2, 3 amino acid sequences are set forth in SEQ ID NOs: 768, 769, 770, respectively, and the selected VL CDRs 1, 2, 3 amino acid sequences are set forth in SEQ ID NOs: 771, 772, 773, respectively;
(12) the selected VH CDRs 1, 2, 3 amino acid sequences are set forth in SEQ ID NOs: 774, 775, 776, respectively, and the selected VL CDRs 1, 2, 3 amino acid sequences are set forth in SEQ ID NOs: 777, 778, 779, respectively;
(13) the selected VH CDRs 1, 2, 3 amino acid sequences are set forth in SEQ ID NOs: 780, 781, 782, respectively, and the selected VL CDRs 1, 2, 3 amino acid sequences are set forth in SEQ ID NOs: 783, 784, 785, respectively;
(14) the selected VH CDRs 1, 2, 3 amino acid sequences are set forth in SEQ ID NOs: 786, 787, 788, respectively, and the selected VL CDRs 1, 2, 3 amino acid sequences are set forth in SEQ ID NOs: 789, 790, 791, respectively;
(15) the selected VH CDRs 1, 2, 3 amino acid sequences are set forth in SEQ ID NOs: 792, 793, 794, respectively, and the selected VL CDRs 1, 2, 3 amino acid sequences are set forth in SEQ ID NOs: 795, 796, 797, respectively;
(16) the selected VH CDRs 1, 2, 3 amino acid sequences are set forth in SEQ ID NOs: 798, 799, 800, respectively, and the selected VL CDRs 1, 2, 3 amino acid sequences are set forth in SEQ ID NOs: 801, 802, 803, respectively;
(17) the selected VH CDRs 1, 2, 3 amino acid sequences are set forth in SEQ ID NOs: 804, 805, 806, respectively, and the selected VL CDRs 1, 2, 3 amino acid sequences are set forth in SEQ ID NOs: 807, 808, 809, respectively; and
(18) the selected VH CDRs 1, 2, 3 amino acid sequences are set forth in SEQ ID NOs: 810, 811, 812, respectively, and the selected VL CDRs 1, 2, 3 amino acid sequences are set forth in SEQ ID NOs: 813, 814, 815, respectively.

13. (canceled)

14. (canceled)

15. (canceled)

16. (canceled)

17. (canceled)

18. An antibody or antigen-binding fragment thereof that binds to MICA and/or MICB comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are selected from FIG. 23, 25, 26, 27, 28, or 29.

19.-84. (canceled)

85. An antibody or antigen-binding fragment thereof comprising a heavy chain variable region (VH) comprising VH CDR1, VH CDR2, and VH CDR3, and a light chain variable region (VL) comprising VL CDR1, VL CDR2, and VL CDR3, wherein the VH CDR1, VH CDR2, VH CDR3, VL CDR1, VL CDR2, and VL CDR3 are identical to VH CDR1, VH CDR2, VH CDR3, VL CDR1, VL CDR2, and VL CDR3 of the antibody or antigen-binding fragment thereof of claim 1.

86. An antibody-drug conjugate comprising the antibody or antigen-binding fragment thereof of claim 1 covalently bound to a therapeutic agent.

87. (canceled)

88. A method of treating a subject having cancer, the method comprising administering a therapeutically effective amount of a composition comprising the antibody or antigen-binding fragment thereof of claim 1 to the subject.

89. The method of claim 88, wherein the subject has a solid tumor or hematological cancer.

90. (canceled)

91. A method of decreasing the rate of tumor growth, the method comprising contacting a tumor cell with an effective amount of a composition comprising the antibody or antigen-binding fragment thereof of claim 1.

92. A method of killing a tumor cell, the method comprising contacting a tumor cell with an effective amount of a composition comprising the antibody or antigen-binding fragment thereof of claim 1.

93. (canceled)

94. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising VH CDR 1, 2, 3 set forth in FIG. 24A, 24B, 24C, 24D, 24E, 28, 29, 30A, 30B, 30C, 30D, or 30E, and wherein the VH, when paired with a corresponding light chain variable region (VL) binds to MICA and/or MICB; or
(2) an immunoglobulin light chain or a fragment thereof comprising a VL comprising CDRs 1, 2, and 3 comprising VL CDR 1, 2, 3 set forth in FIG. 24A, 24B, 24C, 24D, 24E, 28, 29, 30A, 30B, 30C, 30D, or 30E, when paired with a corresponding VH binds to MICA and/or MICB.

95. The nucleic acid of claim 94, wherein
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 3, 4, and 5, respectively, and wherein the VH, when paired with a light chain variable region (VL) comprising the amino acid sequence set forth in SEQ ID NO: 2 binds to MICA and/or MICB;
(2) an immunoglobulin heavy chain or a fragment thereof comprising a VH comprising CDRs 1, 2, and 3 comprising the amino acid sequences set forth in SEQ ID NOs: 11, 12, and 13, respectively, and wherein the VH, when paired with a VL comprising the amino acid sequence set forth in SEQ ID NO: 10 binds to MICA and/or MICB;
(3) an immunoglobulin heavy chain or a fragment thereof comprising a VH comprising CDRs 1, 2, and 3 comprising the amino acid sequences set forth in SEQ ID NOs: 19, 20, and 21, respectively, and wherein the VH, when paired with a VL comprising the amino acid sequence set forth in SEQ ID NO: 18 binds to MICA and/or MICB;
(4) an immunoglobulin heavy chain or a fragment thereof comprising a VH comprising CDRs 1, 2, and 3 comprising the amino acid sequences set forth in SEQ ID NOs: 27, 28, and 29, respectively, and wherein the VH, when paired with a VL comprising the amino acid sequence set forth in SEQ ID NO: 26 binds to MICA and/or MICB;
(5) an immunoglobulin heavy chain or a fragment thereof comprising a VH comprising CDRs 1, 2, and 3 comprising the amino acid sequences set forth in SEQ ID NOs: 35, 36, and 37, respectively, and wherein the VH, when paired with a VL comprising the amino acid sequence set forth in SEQ ID NO: 34 binds to MICA and/or MICB;
(6) an immunoglobulin heavy chain or a fragment thereof comprising a VH comprising CDRs 1, 2, and 3 comprising the amino acid sequences set forth in SEQ ID NOs: 43, 44, and 45, respectively, and wherein the VH, when paired with a VL comprising the amino acid sequence set forth in SEQ ID NO: 42 binds to MICA and/or MICB;
(7) an immunoglobulin heavy chain or a fragment thereof comprising a VH comprising CDRs 1, 2, and 3 comprising the amino acid sequences set forth in SEQ ID NOs: 51, 52, and 53, respectively, and wherein the VH, when paired with a VL comprising the amino acid sequence set forth in SEQ ID NO: 50 binds to MICA and/or MICB;
(8) an immunoglobulin heavy chain or a fragment thereof comprising a VH comprising CDRs 1, 2, and 3 comprising the amino acid sequences set forth in SEQ ID NOs: 59, 60, and 61, respectively, and wherein the VH, when paired with a VL comprising the amino acid sequence set forth in SEQ ID NO: 58 binds to MICA and/or MICB;
(9) an immunoglobulin heavy chain or a fragment thereof comprising a VH comprising CDRs 1, 2, and 3 comprising the amino acid sequences set forth in SEQ ID NOs: 67, 68, and 69, respectively, and wherein the VH, when paired with a VL comprising the amino acid sequence set forth in SEQ ID NO: 66 binds to MICA and/or MICB;
(10) an immunoglobulin heavy chain or a fragment thereof comprising a VH comprising CDRs 1, 2, and 3 comprising the amino acid sequences set forth in SEQ ID NOs: 75, 76, and 77, respectively, and wherein the VH, when paired with a VL comprising the amino acid sequence set forth in SEQ ID NO: 74 binds to MICA and/or MICB;
(11) an immunoglobulin heavy chain or a fragment thereof comprising a VH comprising CDRs 1, 2, and 3 comprising the amino acid sequences set forth in SEQ ID NOs: 83, 84, and 85, respectively, and wherein the VH, when paired with a VL comprising the amino acid sequence set forth in SEQ ID NO: 82 binds to MICA and/or MICB;
(12) an immunoglobulin heavy chain or a fragment thereof comprising a VH comprising CDRs 1, 2, and 3 comprising the amino acid sequences set forth in SEQ ID NOs: 91, 92, and 93, respectively, and wherein the VH, when paired with a VL comprising the amino acid sequence set forth in SEQ ID NO: 90 binds to MICA and/or MICB;
(13) an immunoglobulin heavy chain or a fragment thereof comprising a VH comprising CDRs 1, 2, and 3 comprising the amino acid sequences set forth in SEQ ID NOs: 99, 100, and 101, respectively, and wherein the VH, when paired with a VL comprising the amino acid sequence set forth in SEQ ID NO: 98 binds to MICA and/or MICB;
(14) an immunoglobulin heavy chain or a fragment thereof comprising a VH comprising CDRs 1, 2, and 3 comprising the amino acid sequences set forth in SEQ ID NOs: 107, 108, and 109, respectively, and wherein the VH, when paired with a VL comprising the amino acid sequence set forth in SEQ ID NO: 106 binds to MICA and/or MICB;
(15) an immunoglobulin heavy chain or a fragment thereof comprising a VH comprising CDRs 1, 2, and 3 comprising the amino acid sequences set forth in SEQ ID NOs: 115, 116, and 117, respectively, and wherein the VH, when paired with a VL comprising the amino acid sequence set forth in SEQ ID NO: 114 binds to MICA and/or MICB;
(16) an immunoglobulin heavy chain or a fragment thereof comprising a VH comprising CDRs 1, 2, and 3 comprising the amino acid sequences set forth in SEQ ID NOs: 123, 124, and 125, respectively, and wherein the VH, when paired with a VL comprising the amino acid sequence set forth in SEQ ID NO: 122 binds to MICA and/or MICB;
(17) an immunoglobulin heavy chain or a fragment thereof comprising a VH comprising CDRs 1, 2, and 3 comprising the amino acid sequences set forth in SEQ ID NOs: 131, 132, and 133, respectively, and wherein the VH, when paired with a VL comprising the amino acid sequence set forth in SEQ ID NO: 130 binds to MICA and/or MICB;
(18) an immunoglobulin heavy chain or a fragment thereof comprising a VH comprising CDRs 1, 2, and 3 comprising the amino acid sequences set forth in SEQ ID NOs: 139, 140, and 141, respectively, and wherein the VH, when paired with a VL comprising the amino acid sequence set forth in SEQ ID NO: 138 binds to MICA and/or MICB;
(19) an immunoglobulin heavy chain or a fragment thereof comprising a VH comprising CDRs 1, 2, and 3 comprising the amino acid sequences set forth in SEQ ID NOs: 147, 148, and 149, respectively, and wherein the VH, when paired with a VL comprising the amino acid sequence set forth in SEQ ID NO: 146 binds to MICA and/or MICB;
(20) an immunoglobulin heavy chain or a fragment thereof comprising a VH comprising CDRs 1, 2, and 3 comprising the amino acid sequences set forth in SEQ ID NOs: 155, 156, and 157, respectively, and wherein the VH, when paired with a VL comprising the amino acid sequence set forth in SEQ ID NO: 154 binds to MICA and/or MICB;
(21) an immunoglobulin light chain or a fragment thereof comprising a VL comprising CDRs 1, 2, and 3 comprising the amino acid sequences set forth in SEQ ID NOs: 6, 7, and 8, respectively, and wherein the VL, when paired with a VH comprising the amino acid sequence set forth in SEQ ID NO: 1 binds to MICA and/or MICB;
(22) an immunoglobulin light chain or a fragment thereof comprising a VL comprising CDRs 1, 2, and 3 comprising the amino acid sequences set forth in SEQ ID NOs: 14, 15, and 16, respectively, and wherein the VL, when paired with a VH comprising the amino acid sequence set forth in SEQ ID NO: 9 binds to MICA and/or MICB;
(23) an immunoglobulin light chain or a fragment thereof comprising a VL comprising CDRs 1, 2, and 3 comprising the amino acid sequences set forth in SEQ ID NOs: 22, 23, and 24, respectively, and wherein the VL, when paired with a VH comprising the amino acid sequence set forth in SEQ ID NO: 17 binds to MICA and/or MICB;
(24) an immunoglobulin light chain or a fragment thereof comprising a VL comprising CDRs 1, 2, and 3 comprising the amino acid sequences set forth in SEQ ID NOs: 30, 31, and 32, respectively, and wherein the VL, when paired with a VH comprising the amino acid sequence set forth in SEQ ID NO: 25 binds to MICA and/or MICB;
(25) an immunoglobulin light chain or a fragment thereof comprising a VL comprising CDRs 1, 2, and 3 comprising the amino acid sequences set forth in SEQ ID NOs: 38, 39, and 40, respectively, and wherein the VL, when paired with a VH comprising the amino acid sequence set forth in SEQ ID NO: 33 binds to MICA and/or MICB;
(26) an immunoglobulin light chain or a fragment thereof comprising a VL comprising CDRs 1, 2, and 3 comprising the amino acid sequences set forth in SEQ ID NOs: 46, 47, and 48, respectively, and wherein the VL, when paired with a VH comprising the amino acid sequence set forth in SEQ ID NO: 41 binds to MICA and/or MICB;
(27) an immunoglobulin light chain or a fragment thereof comprising a VL comprising CDRs 1, 2, and 3 comprising the amino acid sequences set forth in SEQ ID NOs: 54, 55, and 56, respectively, and wherein the VL, when paired with a VH comprising the amino acid sequence set forth in SEQ ID NO: 49 binds to MICA and/or MICB;
(28) an immunoglobulin light chain or a fragment thereof comprising a VL comprising CDRs 1, 2, and 3 comprising the amino acid sequences set forth in SEQ ID NOs: 62, 63, and 64, respectively, and wherein the VL, when paired with a VH comprising the amino acid sequence set forth in SEQ ID NO: 57 binds to MICA and/or MICB;
(29) an immunoglobulin light chain or a fragment thereof comprising a VL comprising CDRs 1, 2, and 3 comprising the amino acid sequences set forth in SEQ ID NOs: 70, 71, and 72, respectively, and wherein the VL, when paired with a VH comprising the amino acid sequence set forth in SEQ ID NO: 65 binds to MICA and/or MICB;
(30) an immunoglobulin light chain or a fragment thereof comprising a VL comprising CDRs 1, 2, and 3 comprising the amino acid sequences set forth in SEQ ID NOs: 78, 79, and 80, respectively, and wherein the VL, when paired with a VH comprising the amino acid sequence set forth in SEQ ID NO: 73 binds to MICA and/or MICB;
(31) an immunoglobulin light chain or a fragment thereof comprising a VL comprising CDRs 1, 2, and 3 comprising the amino acid sequences set forth in SEQ ID NOs: 86, 87, and 88, respectively, and wherein the VL, when paired with a VH comprising the amino acid sequence set forth in SEQ ID NO: 81 binds to MICA and/or MICB;
(32) an immunoglobulin light chain or a fragment thereof comprising a VL comprising CDRs 1, 2, and 3 comprising the amino acid sequences set forth in SEQ ID NOs: 94, 95, and 96, respectively, and wherein the VL, when paired with a VH comprising the amino acid sequence set forth in SEQ ID NO: 89 binds to MICA and/or MICB;
(33) an immunoglobulin light chain or a fragment thereof comprising a VL comprising CDRs 1, 2, and 3 comprising the amino acid sequences set forth in SEQ ID NOs: 102, 103, and 104, respectively, and wherein the VL, when paired with a VH comprising the amino acid sequence set forth in SEQ ID NO: 97 binds to MICA and/or MICB;
(34) an immunoglobulin light chain or a fragment thereof comprising a VL comprising CDRs 1, 2, and 3 comprising the amino acid sequences set forth in SEQ ID NOs: 110, 111, and 112, respectively, and wherein the VL, when paired with a VH comprising the amino acid sequence set forth in SEQ ID NO: 105 binds to MICA and/or MICB;
(35) an immunoglobulin light chain or a fragment thereof comprising a VL comprising CDRs 1, 2, and 3 comprising the amino acid sequences set forth in SEQ ID NOs: 118, 119, and 120, respectively, and wherein the VL, when paired with a VH comprising the amino acid sequence set forth in SEQ ID NO: 113 binds to MICA and/or MICB;
(36) an immunoglobulin light chain or a fragment thereof comprising a VL comprising CDRs 1, 2, and 3 comprising the amino acid sequences set forth in SEQ ID NOs: 126, 127, and 128, respectively, and wherein the VL, when paired with a VH comprising the amino acid sequence set forth in SEQ ID NO: 121 binds to MICA and/or MICB;
(37) an immunoglobulin light chain or a fragment thereof comprising a VL comprising CDRs 1, 2, and 3 comprising the amino acid sequences set forth in SEQ ID NOs: 134, 135, and 136, respectively, and wherein the VL, when paired with a VH comprising the amino acid sequence set forth in SEQ ID NO: 129 binds to MICA and/or MICB;
(38) an immunoglobulin light chain or a fragment thereof comprising a VL comprising CDRs 1, 2, and 3 comprising the amino acid sequences set forth in SEQ ID NOs: 142, 143, and 144, respectively, and wherein the VL, when paired with a VH comprising the amino acid sequence set forth in SEQ ID NO: 137 binds to MICA and/or MICB;
(39) an immunoglobulin light chain or a fragment thereof comprising a VL comprising CDRs 1, 2, and 3 comprising the amino acid sequences set forth in SEQ ID NOs: 150, 151, and 152, respectively, and wherein the VL, when paired with a VH comprising the amino acid sequence set forth in SEQ ID NO: 145 binds to MICA and/or MICB;
(40) an immunoglobulin light chain or a fragment thereof comprising a VL comprising CDRs 1, 2, and 3 comprising the amino acid sequences set forth in SEQ ID NOs: 158, 159, and 160, respectively, and wherein the VL, when paired with a VH comprising the amino acid sequence set forth in SEQ ID NO: 153 binds to MICA and/or MICB;
(41) an immunoglobulin heavy chain or a fragment thereof comprising a VH comprising CDRs 1, 2, and 3 comprising the amino acid sequences set forth in SEQ ID NOs: 815, 816, and 817, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(42) an immunoglobulin heavy chain or a fragment thereof comprising a VH comprising CDRs 1, 2, and 3 comprising the amino acid sequences set forth in SEQ ID NOs: 821, 822, and 823, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(43) an immunoglobulin heavy chain or a fragment thereof comprising a VH comprising CDRs 1, 2, and 3 comprising the amino acid sequences set forth in SEQ ID NOs: 827, 828, and 829, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(44) an immunoglobulin heavy chain or a fragment thereof comprising a VH comprising CDRs 1, 2, and 3 comprising the amino acid sequences set forth in SEQ ID NOs: 833, 834, and 835, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(45) an immunoglobulin heavy chain or a fragment thereof comprising a VH comprising CDRs 1, 2, and 3 comprising the amino acid sequences set forth in SEQ ID NOs: 839, 840, and 841, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(46) an immunoglobulin heavy chain or a fragment thereof comprising a VH comprising CDRs 1, 2, and 3 comprising the amino acid sequences set forth in SEQ ID NOs: 845, 846, and 847, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(47) an immunoglobulin heavy chain or a fragment thereof comprising a VH comprising CDRs 1, 2, and 3 comprising the amino acid sequences set forth in SEQ ID NOs: 851, 852, and 853, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(48) an immunoglobulin heavy chain or a fragment thereof comprising a VH comprising CDRs 1, 2, and 3 comprising the amino acid sequences set forth in SEQ ID NOs: 857, 858, and 859, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(49) an immunoglobulin heavy chain or a fragment thereof comprising a VH comprising CDRs 1, 2, and 3 comprising the amino acid sequences set forth in SEQ ID NOs: 863, 864, and 865, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(50) an immunoglobulin heavy chain or a fragment thereof comprising a VH comprising CDRs 1, 2, and 3 comprising the amino acid sequences set forth in SEQ ID NOs: 869, 870, and 871, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(51) an immunoglobulin heavy chain or a fragment thereof comprising a VH comprising CDRs 1, 2, and 3 comprising the amino acid sequences set forth in SEQ ID NOs: 875, 876, and 877, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(52) an immunoglobulin heavy chain or a fragment thereof comprising a VH comprising CDRs 1, 2, and 3 comprising the amino acid sequences set forth in SEQ ID NOs: 881, 882, and 883, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(53) an immunoglobulin heavy chain or a fragment thereof comprising a VH comprising CDRs 1, 2, and 3 comprising the amino acid sequences set forth in SEQ ID NOs: 887, 888, and 889, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(54) an immunoglobulin heavy chain or a fragment thereof comprising a VH comprising CDRs 1, 2, and 3 comprising the amino acid sequences set forth in SEQ ID NOs: 893, 894, and 895, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(55) an immunoglobulin heavy chain or a fragment thereof comprising a VH comprising CDRs 1, 2, and 3 comprising the amino acid sequences set forth in SEQ ID NOs: 899, 900, and 901, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(56) an immunoglobulin heavy chain or a fragment thereof comprising a VH comprising CDRs 1, 2, and 3 comprising the amino acid sequences set forth in SEQ ID NOs: 905, 906, and 907, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(57) an immunoglobulin heavy chain or a fragment thereof comprising a VH comprising CDRs 1, 2, and 3 comprising the amino acid sequences set forth in SEQ ID NOs: 911, 912, and 913, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(58) an immunoglobulin heavy chain or a fragment thereof comprising a VH comprising CDRs 1, 2, and 3 comprising the amino acid sequences set forth in SEQ ID NOs: 917, 918, and 919, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(59) an immunoglobulin heavy chain or a fragment thereof comprising a VH comprising CDRs 1, 2, and 3 comprising the amino acid sequences set forth in SEQ ID NOs: 923, 924, and 925, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(60) an immunoglobulin heavy chain or a fragment thereof comprising a VH comprising CDRs 1, 2, and 3 comprising the amino acid sequences set forth in SEQ ID NOs: 929, 930, and 931, respectively, and wherein the VH, when paired with a VL comprising the amino acid sequence set forth in SEQ ID NO: 653, 654, 655, 656, 657, 658, 691, 693, 695, 697, 699, 701, 703, 705, or 707 binds to MICA and/or MICB;
(61) an immunoglobulin light chain or a fragment thereof comprising a VL comprising CDRs 1, 2, and 3 comprising the amino acid sequences set forth in SEQ ID NOs: 818, 819, and 820, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(62) an immunoglobulin light chain or a fragment thereof comprising a VL comprising CDRs 1, 2, and 3 comprising the amino acid sequences set forth in SEQ ID NOs: 824, 825, and 826, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(63) an immunoglobulin light chain or a fragment thereof comprising a VL comprising CDRs 1, 2, and 3 comprising the amino acid sequences set forth in SEQ ID NOs: 830, 831, and 832, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(64) an immunoglobulin light chain or a fragment thereof comprising a VL comprising CDRs 1, 2, and 3 comprising the amino acid sequences set forth in SEQ ID NOs: 836, 837, and 838, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(65) an immunoglobulin light chain or a fragment thereof comprising a VL comprising CDRs 1, 2, and 3 comprising the amino acid sequences set forth in SEQ ID NOs: 842, 843, and 844, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(66) an immunoglobulin light chain or a fragment thereof comprising a VL comprising CDRs 1, 2, and 3 comprising the amino acid sequences set forth in SEQ ID NOs: 848, 849, and 850, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(67) an immunoglobulin light chain or a fragment thereof comprising a VL comprising CDRs 1, 2, and 3 comprising the amino acid sequences set forth in SEQ ID NOs: 854, 855, and 856, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(68) an immunoglobulin light chain or a fragment thereof comprising a VL comprising CDRs 1, 2, and 3 comprising the amino acid sequences set forth in SEQ ID NOs: 860, 861, and 862, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(69) an immunoglobulin light chain or a fragment thereof comprising a VL comprising CDRs 1, 2, and 3 comprising the amino acid sequences set forth in SEQ ID NOs: 866, 867, and 868, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(70) an immunoglobulin light chain or a fragment thereof comprising a VL comprising CDRs 1, 2, and 3 comprising the amino acid sequences set forth in SEQ ID NOs: 872, 873, and 874, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(71) an immunoglobulin light chain or a fragment thereof comprising a VL comprising CDRs 1, 2, and 3 comprising the amino acid sequences set forth in SEQ ID NOs: 878, 879, and 880, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(72) an immunoglobulin light chain or a fragment thereof comprising a VL comprising CDRs 1, 2, and 3 comprising the amino acid sequences set forth in SEQ ID NOs: 884, 885, and 886, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(73) an immunoglobulin light chain or a fragment thereof comprising a VL comprising CDRs 1, 2, and 3 comprising the amino acid sequences set forth in SEQ ID NOs: 890, 891, and 892, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(74) an immunoglobulin light chain or a fragment thereof comprising a VL comprising CDRs 1, 2, and 3 comprising the amino acid sequences set forth in SEQ ID NOs: 896, 897, and 898, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(75) an immunoglobulin light chain or a fragment thereof comprising a VL comprising CDRs 1, 2, and 3 comprising the amino acid sequences set forth in SEQ ID NOs: 902, 903, and 904, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(76) an immunoglobulin light chain or a fragment thereof comprising a VL comprising CDRs 1, 2, and 3 comprising the amino acid sequences set forth in SEQ ID NOs: 908, 909, and 910, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(77) an immunoglobulin light chain or a fragment thereof comprising a VL comprising CDRs 1, 2, and 3 comprising the amino acid sequences set forth in SEQ ID NOs: 914, 915, and 916, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(78) an immunoglobulin light chain or a fragment thereof comprising a VL comprising CDRs 1, 2, and 3 comprising the amino acid sequences set forth in SEQ ID NOs: 920, 921, and 922, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB;
(79) an immunoglobulin light chain or a fragment thereof comprising a VL comprising CDRs 1, 2, and 3 comprising the amino acid sequences set forth in SEQ ID NOs: 926, 927, and 928, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB; or
(80) an immunoglobulin light chain or a fragment thereof comprising a VL comprising CDRs 1, 2, and 3 comprising the amino acid sequences set forth in SEQ ID NOs: 932, 933, and 934, respectively, and wherein the VL, when paired with a VH comprising the amino acid sequence set forth in SEQ ID NO: 641, 642, 643, 644, 645, 646, 647, 648, 649, 650, 651, 652, 690, 692, 694, 696, 698, 700, 702, 704, or 706 binds to MICA and/or MICB.

96.-99. (canceled)

100. A vector comprising one or more of the nucleic acids of claim 94.

101. (canceled)

102. (canceled)

103. A cell comprising the vector of claim 100.

104. (canceled)

105. A cell comprising one or more of the nucleic acids of claim 94.

106. (canceled)

107. (canceled)

108. A method of producing an antibody or an antigen-binding fragment thereof, the method comprising
(a) culturing the cell of claim 105 under conditions sufficient for the cell to produce the antibody or the antigen-binding fragment; and
(b) collecting the antibody or the antigen-binding fragment produced by the cell.


SEQUENCE LISTING (1445 sequences)

SEQ ID NO: 1 - protein, 124 aa, synthetic construct
QIQLLQSGPELKKPGETVKISCKASGYTFTNYGMNWVKQAPGKGLKWMGWINTYTGEPTYADDFKGRFAFSLETSASTAYLQINNLKNEDMATYFCAREINDYVSGRYYAMDYWGQGTSVTVSS

SEQ ID NO: 2 - protein, 107 aa, synthetic construct
DIVLTQSPASLSASVGETVTITCRASENIYSYLAWYQQKQGKSPQLLVYNAKTLAEGVPSRFSGSGSGTQFSLKINSLQPEDFGSYYCQHHYNTPLTFGAGTKLEIK

SEQ ID NO: 3 - protein, 8 aa, synthetic construct
GYTFTNYG

SEQ ID NO: 4 - protein, 8 aa, synthetic construct
INTYTGEP

SEQ ID NO: 5 - protein, 17 aa, synthetic construct
AREINDYVSGRYYAMDY

SEQ ID NO: 6 - protein, 6 aa, synthetic construct
ENIYSY

SEQ ID NO: 7 - skipped

SEQ ID NO: 8 - protein, 9 aa, synthetic construct
QHHYNTPLT

SEQ ID NO: 9 - protein, 118 aa, synthetic construct
EVQLQQSGAELVKPGASVKLSCTASGFNITDTYMHWVKQRPEQGLEWIGRIDPANGNTKYDPKFQGKATITADTSSNTAYLQLSSLTSEDTAVYYCAREYYGSSLFDYWGQGTTLTVS

SEQ ID NO: 10 - protein, 108 aa, synthetic construct
DILLTQSPAFMAASPGEKVTITCSVSSSISSSYLHWYQQKSETSPKYWIYGTSNLASGVPVRFSGSGSGTSYSLTISSMEAEDAATYYCQQWSGYPFTFGGGTKLEIK

SEQ ID NO: 11 - protein, 8 aa, synthetic construct
GFNITDTY

SEQ ID NO: 12 - protein, 8 aa, synthetic construct
IDPANGNT

SEQ ID NO: 13 - protein, 12 aa, synthetic construct
AREYYGSSLFDY

SEQ ID NO: 14 - protein, 7 aa, synthetic construct
SSISSSY

SEQ ID NO: 15 - skipped

SEQ ID NO: 16 - protein, 9 aa, synthetic construct
QQWSGYPFT

SEQ ID NO: 17 - protein, 117 aa, synthetic construct
EVQLQQSGAELVRPGTSVKVSCKASGYAFTDYLIEWVKQRPGQGLEWIGGINPGSGSTNYNEKFKGKAILTADKSSSTAYMQLSSLTSDDSAVYFCARLDGNYVAYWGQGTTLTVSS

SEQ ID NO: 18 - protein, 107 aa, synthetic construct
DIVMTQSPASLSASVGETVTITCRASGNIHNYLAWYQQNQGKSPRLLVYHAKTLADGVPSRFSGTGSGTQYSLKINSLQPGDFGSYYCQHFWSTPWTFGGGTKLEIK

SEQ ID NO: 19 - protein, 8 aa, synthetic construct
GYAFTDYL

SEQ ID NO: 20 - protein, 8 aa, synthetic construct
INPGSGST

SEQ ID NO: 21 - protein, 10 aa, synthetic construct
ARLDGNYVAY

SEQ ID NO: 22 - protein, 6 aa, synthetic construct
GNIHNY

SEQ ID NO: 23 - skipped

SEQ ID NO: 24 - protein, 9 aa, synthetic construct
QHFWSTPWT

SEQ ID NO: 25 - protein, 117 aa, synthetic construct
QIQLLQSGAELVRPGTSVKVSCKASGYAFTDYLIEWVKQRPGQGLEWIGGINPGSGSTNYNEKFKGKAILTADKSSSTAYMQLSSLTSDDSAVYFCARLDGNYVGYWGQGTTLTVSS

SEQ ID NO: 26 - protein, 107 aa, synthetic construct
DIVMTQSPASLSASVGETVTITCRASGNIHNYLAWYQQKQGKSPQLLVYHAKTLADGVPSRFSGSGSGTQYSLKIDSLQPEDFGTYYCQHFWSTPWTFGGGTKLEIK

SEQ ID NO: 27 - protein, 8 aa, synthetic construct
GYAFTDYL

SEQ ID NO: 28 - protein, 8 aa, synthetic construct
INPGSGST

SEQ ID NO: 29 - protein, 10 aa, synthetic construct
ARLDGNYVGY

SEQ ID NO: 30 - protein, 6 aa, synthetic construct
GNIHNY

SEQ ID NO: 31 - skipped

SEQ ID NO: 32 - protein, 9 aa, synthetic construct
QHFWSTPWT

SEQ ID NO: 33 - protein, 117 aa, synthetic construct; identical to SEQ ID NO: 25

SEQ ID NO: 34 - protein, 107 aa, synthetic construct
DIVMTQSPASLSASVGETVTITCRASGNIHNYLAWYQQKQGKSPQLLVYHAKTLADGVPSRFSGSGSGTQYSLKINSLQPEDFGSYYCQHFWSTPWTFGGGTKLEIK

SEQ ID NO: 35 - protein, 8 aa, synthetic construct
GYAFTDYL

SEQ ID NO: 36 - protein, 8 aa, synthetic construct
INPGSGST

SEQ ID NO: 37 - protein, 10 aa, synthetic construct
ARLDGNYVGY

SEQ ID NO: 38 - protein, 6 aa, synthetic construct
GNIHNY

SEQ ID NO: 39 - skipped

SEQ ID NO: 40 - protein, 9 aa, synthetic construct
QHFWSTPWT

SEQ ID NO: 41 - protein, 117 aa, synthetic construct
QIQLLQSGAELVRPGTSVKVTCKASGYAFIDYLIEWVKQGPGQGLEWIGGINPGSGSTNYNEKFKGKAILTADKSSSTAYMQLSRLTSDDSAVYFCARLDGNYVGYWGQGTTLTVSS

SEQ ID NO: 42 - protein, 107 aa, synthetic construct
DIVLTQSPASLSASVGETVTITCRASGNIHNYLTWYQQKQGKSPQLLVYHAKTLADGVPSRFSGSGSGTQYSLKINNLQPEDFGSYYCQHFWSTPWTFGGGTKLEIK

SEQ ID NO: 43 - protein, 8 aa, synthetic construct
GYAFIDYL

SEQ ID NO: 44 - protein, 8 aa, synthetic construct
INPGSGST

SEQ ID NO: 45 - protein, 10 aa, synthetic construct
ARLDGNYVGY

SEQ ID NO: 46 - protein, 6 aa, synthetic construct
GNIHNY

SEQ ID NO: 47 - skipped

SEQ ID NO: 48 - protein, 9 aa, synthetic construct
QHFWSTPWT

SEQ ID NO: 49 - protein, 117 aa, synthetic construct
QVQLQQSGAELVRPGTSVKVSCKASGYAFTDYLIEWVKQRPGQGLEWIGGINPGSGSTNYNEKFKGKAILTADKSSSTAYMQLSSLTSDDSAVYFCARLDGNYVGYWGQGTTLTVSS

SEQ ID NO: 50 - protein, 107 aa, synthetic construct
DILLTQSPASLSASVGETVTITCRASENIHNYLAWFQLKQGKSPQLLVYYAKTLADGVPSRFSGSGSGTQYSLKINSLQPEDFGSYYCQHFWSTPRTFGGGTKLEIK

SEQ ID NO: 51 - protein, 8 aa, synthetic construct
GYAFTDYL

SEQ ID NO: 52 - protein, 8 aa, synthetic construct
INPGSGST

SEQ ID NO: 53 - protein, 10 aa, synthetic construct
ARLDGNYVGY

SEQ ID NO: 54 - protein, 6 aa, synthetic construct
ENIHNY

SEQ ID NO: 55 - skipped

SEQ ID NO: 56 - protein, 9 aa, synthetic construct
QHFWSTPRT

SEQ ID NO: 57 - protein, 117 aa, synthetic construct; identical to SEQ ID NO: 49

SEQ ID NO: 58 - protein, 107 aa, synthetic construct
DIVLTQSPASLSASVGETVTITCRASENIHNYLAWFQLKQGKSPQLLVYYAKTLADGVPSRFSGSGSGTQYSLKINSLQPEDFGSYYCQHFWSTPRTFGGGTKLEIK

SEQ ID NO: 59 - protein, 8 aa, synthetic construct
GYAFTDYL

SEQ ID NO: 60 - protein, 8 aa, synthetic construct
INPGSGST

SEQ ID NO: 61 - protein, 10 aa, synthetic construct
ARLDGNYVGY

SEQ ID NO: 62 - protein, 6 aa, synthetic construct
ENIHNY

SEQ ID NO: 63 - skipped

SEQ ID NO: 64 - protein, 9 aa, synthetic construct
QHFWSTPRT

SEQ ID NO: 65 - protein, 117 aa, synthetic construct; identical to SEQ ID NO: 49

SEQ ID NO: 66 - protein, 107 aa, synthetic construct
DIVMTQSPASLSASVGETVTITCRASGNIHNYLAWYQQKQGKSPQLLVYHAKTLADGVPSRFSGSGSGTQYSLKINSLQPEDFGSYYCQHFWNTPWTFGGGTKLEIK

SEQ ID NO: 67 - protein, 8 aa, synthetic construct
GYAFTDYL

SEQ ID NO: 68 - protein, 8 aa, synthetic construct
INPGSGST

SEQ ID NO: 69 - protein, 10 aa, synthetic construct
ARLDGNYVGY

SEQ ID NO: 70 - protein, 6 aa, synthetic construct
GNIHNY

SEQ ID NO: 71 - skipped

SEQ ID NO: 72 - protein, 9 aa, synthetic construct
QHFWNTPWT

SEQ ID NO: 73 - protein, 117 aa, synthetic construct
EVQLQQSGPELVKPGASVKMSCKASGYTFTDYYMKWVKQSHGKSLEWIGDINPNNGDTFYNQKFKGKATLTVDKSSSTAYMHLNSLTSEDSAVYYCATGGYDGFAYWGQGTLVTVSA

SEQ ID NO: 74 - protein, 111 aa, synthetic construct
DIVLTQSPASLAVSLGQRATISCKASQSVDYDGDSYMNWYQQKPGQPPKLLIYAASNLESGIPARFSGSGSGTDFTLNIHPVEEEDAATYYCQESNEDPYTFGGGTKLEIK

SEQ ID NO: 75 - protein, 8 aa, synthetic construct
GYTFTDYY

SEQ ID NO: 76 - protein, 8 aa, synthetic construct
INPNNGDT

SEQ ID NO: 77 - protein, 10 aa, synthetic construct
ATGGYDGFAY

SEQ ID NO: 78 - protein, 10 aa, synthetic construct
QSVDYDGDSY

SEQ ID NO: 79 - skipped

SEQ ID NO: 80 - protein, 9 aa, synthetic construct
QESNEDPYT

SEQ ID NO: 81 - protein, 118 aa, synthetic construct
EVQLQQSGPELVKPGASVKVSCKASGYAFTKYNIYWVKQSHGKSLEWIGYIDPYNGGTVQNQKFKGKATLTVDKSSSTAYMHLNSLTSEDSAVYYCATTDGYSLFTYWGQGTLVTVSS

SEQ ID NO: 82 - protein, 107 aa, synthetic construct
DIQMTQTTSSLSASLGDRVTISCRASQDIGNYLNWYQQKPDGTVKLLIYYTSRLHSGVPSRFSGSGSGTDYSLTISNLEQEDIATYFCQQGNTLPFTFGSGTKLEIK

SEQ ID NO: 83 - protein, 8 aa, synthetic construct
GYAFTKYN

SEQ ID NO: 84 - protein, 8 aa, synthetic construct
IDPYNGGT

SEQ ID NO: 85 - protein, 11 aa, synthetic construct
ATTDGYSLFTY

SEQ ID NO: 86 - protein, 6 aa, synthetic construct
QDIGNY

SEQ ID NO: 87 - skipped

SEQ ID NO: 88 - protein, 9 aa, synthetic construct
QQGNTLPFT

SEQ ID NO: 89 - protein, 118 aa, synthetic construct
EVQLQQSGPELVKPGASVKVSCKASGYAFTKYNIYWVKQSHGKSLEWIGYIDPYNGGTVYNQKFEGKATLTVDKSSSTAYMHLNSLTSEDSAVYYCATTDGYSLFTYWGQGTLVTVSA

SEQ ID NO: 90 - protein, 107 aa, synthetic construct
DIQMTQTTSSLSASLGDRVTISCSAGQGINNYLNWYQQKPDGTVKLLIYYTSSLQSGVPSRFSGSGSGTDYSLTISNLEPEDIATYYCQQYSKLPLTFGAGTKLELK

SEQ ID NO: 91 - protein, 8 aa, synthetic construct
GYAFTKYN

SEQ ID NO: 92 - protein, 8 aa, synthetic construct
IDPYNGGT

SEQ ID NO: 93 - protein, 11 aa, synthetic construct
ATTDGYSLFTY

SEQ ID NO: 94 - protein, 6 aa, synthetic construct
QGINNY

SEQ ID NO: 95 - skipped

SEQ ID NO: 96 - protein, 9 aa, synthetic construct
QQYSKLPLT

SEQ ID NO: 97 - protein, 118 aa, synthetic construct
EVQLQQSGPELVKPGASVKVSCKASGYAFTKYNIYWVKQSHGKSLEWIGYIDPYNGGTVYNQKFKGKATLTVDKSSSTAYMHLNSLTSEDSAVYYCATTDGYSLFTYWGQGTLVTVSA

SEQ ID NO: 98 - protein, 107 aa, synthetic construct
DIQMTQSSSSLSASLGDRVTITCSAGQGINNYLNWYQQRPDGTVKLLIFYTSSLQSGVPSRFSGSGSGADYSLTISNLEPEDIATYYCQQYSKLPLTFGAGTKLELK

SEQ ID NO: 99 - protein, 8 aa, synthetic construct
GYAFTKYN

SEQ ID NO: 100 - protein, 8 aa, synthetic construct
IDPYNGGT

SEQ ID NO: 101 - protein, 11 aa, synthetic construct
ATTDGYSLFTY

SEQ ID NO: 102 - protein, 6 aa, synthetic construct
QGINNY

SEQ ID NO: 103 - skipped

SEQ ID NO: 104 - protein, 9 aa, synthetic construct
QQYSKLPLT

SEQ ID NO: 105 - protein, 118 aa, synthetic construct
QVQLKQSGPELVKPGASVKVSCKASGYAFTKYNIYWVKQSHGKSLEWIGYIDPYNGGTVQNQKFKGKATLTVDKSSSTAYMHLNSLTSEDSAVYYCATTDGYSLFTYWGQGTLVTVSS

SEQ ID NO: 106 - protein, 108 aa, synthetic construct
DIVLTQSPTTMAASPGEKITITCSASSSISSNYLHWYQQKPGFSPKLLIYRTSNLASGVPARFSGSGSGTSYSLTIGTMEAEDVATYYCQQGSSIPFTFGSGTKLEIK

SEQ ID NO: 107 - protein, 8 aa, synthetic construct
GYAFTKYN

SEQ ID NO: 108 - protein, 8 aa, synthetic construct
IDPYNGGT

SEQ ID NO: 109 - protein, 11 aa, synthetic construct
ATTDGYSLFTY

SEQ ID NO: 110 - protein, 7 aa, synthetic construct
SSISSNY

SEQ ID NO: 111 - skipped

SEQ ID NO: 112 - protein, 9 aa, synthetic construct
QQGSSIPFT

SEQ ID NO: 113 - protein, 118 aa, synthetic construct
QVQLQQSGPELVKPGASVKVSCKASGYAFTKYNIYWVKQSHGKSLEWIGYIDPYNGGTVYNQKFEGKATLTVDKSSSTAYMHLNSLTSEDSAVYYCATTDGYSLFTYWGQGTLVTVSA

SEQ ID NO: 114 - protein, 107 aa, synthetic construct; identical to SEQ ID NO: 90

SEQ ID NO: 115 - protein, 8 aa, synthetic construct
GYAFTKYN

SEQ ID NO: 116 - protein, 8 aa, synthetic construct
IDPYNGGT

SEQ ID NO: 117 - protein, 11 aa, synthetic construct
ATTDGYSLFTY

SEQ ID NO: 118 - protein, 6 aa, synthetic construct
QGINNY

SEQ ID NO: 119 - skipped

SEQ ID NO: 120 - protein, 9 aa, synthetic construct
QQYSKLPLT

SEQ ID NO: 121 - protein, 118 aa, synthetic construct
QVQLQQSGPELVKPGASVKVSCKASGYAFTKYNIYWVKQSHGKSLEWIGYIDPYNGGTVYNQKFKGKATLTVDKSSSTAYMHLNSLTSEDSAVYYCATTDGYSLFTYWGQGTLVTVSA

SEQ ID NO: 122 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 123 - protein, 8 aa, Mus musculus
GYAFTKYN

SEQ ID NO: 124 - protein, 8 aa, Mus musculus
IDPYNGGT

SEQ ID NO: 125 - protein, 11 aa, Mus musculus
ATTDGYSLFTY

SEQ ID NO: 126 - protein, 6 aa, Mus musculus
QDISNY

SEQ ID NO: 127 - skipped

SEQ ID NO: 128 - protein, 9 aa, Mus musculus
QQGNTLPFT

SEQ ID NO: 129 - protein, 118 aa, synthetic construct
QVQLQQSGPELVKPGASVKVSCKASGYAFTKYNIYWVKQSHGKSLEWIGYIDPYNGGTVYNQKFKGKATLTVDKSSSTAYMHLNSLTSEDSAVYYCATTDGYSLFTYWGQGTLVTVSS

SEQ ID NO: 130 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLELK

SEQ ID NO: 131 - protein, 8 aa, synthetic construct
GYAFTKYN

SEQ ID NO: 132 - protein, 8 aa, synthetic construct
IDPYNGGT

SEQ ID NO: 133 - protein, 11 aa, synthetic construct
ATTDGYSLFTY

SEQ ID NO: 134 - protein, 6 aa, synthetic construct
QDISNY

SEQ ID NO: 135 - skipped

SEQ ID NO: 136 - protein, 9 aa, synthetic construct
QQGNTLPLT

SEQ ID NO: 137 - protein, 117 aa, synthetic construct
QVQLQQSGAELARPGASVKLSCKASGYTFSIHWMQWVKQRPGQGLEWIGAIYPGNGDTKYTQKFKGKATLTADKSSSTAYMQLSNLASEDSAVYYCATLYGNYEAYWGQGTLVTVSA

SEQ ID NO: 138 - protein, 108 aa, synthetic construct
DIVLTQSPASLSASVGETVTITCGASENIYGALNWYQRKQGKSPQLLIYGTTNLADGMSSRFSGSGSGRQYSLKISSLHPDDVATYYCQNVLTTPPWTFGGGTKLEIK

SEQ ID NO: 139 - protein, 8 aa, synthetic construct
GYTFSIHW

SEQ ID NO: 140 - protein, 8 aa, synthetic construct
IYPGNGDT

SEQ ID NO: 141 - protein, 10 aa, synthetic construct
ATLYGNYEAY

SEQ ID NO: 142 - protein, 9 aa, synthetic construct
ENIYGALNW

SEQ ID NO: 143 - skipped

SEQ ID NO: 144 - protein, 10 aa, synthetic construct
QNVLTTPPWT

SEQ ID NO: 145 - protein, 117 aa, synthetic construct; identical to SEQ ID NO: 49

SEQ ID NO: 146 - protein, 107 aa, synthetic construct
DILLTQSPASLSASVGETVTITCRASGNIHNYLAWYQQKQGKSPQLLVYHAKTLADGVPSRFSGSGSGTQYSLKINSLQPEDFGSYYCQHFWSTPWTFGGGTKLEIK

SEQ ID NO: 147 - protein, 8 aa, synthetic construct
GYAFTDYL

SEQ ID NO: 148 - protein, 8 aa, synthetic construct
INPGSGST

SEQ ID NO: 149 - protein, 10 aa, synthetic construct
ARLDGNYVGY

SEQ ID NO: 150 - protein, 9 aa, synthetic construct
GNIHNYLAW

SEQ ID NO: 151 - skipped

SEQ ID NO: 152 - protein, 9 aa, synthetic construct
QHFWSTPWT

SEQ ID NO: 153 - protein, 118 aa, synthetic construct; identical to SEQ ID NO: 121

SEQ ID NO: 154 - protein, 107 aa, synthetic construct
DIQMTQTTSSLSASLGDRVTITCSAGQGINNYLNWYQQKPDGTVKLLIFYTSSLQSGVPSRFSGSGSGADYSLTISNLEPEDIATYYCQQYSKLPLTFGAGTKLELK

SEQ ID NO: 155 - protein, 8 aa, synthetic construct
GYAFTKYN

SEQ ID NO: 156 - protein, 8 aa, synthetic construct
IDPYNGGT

SEQ ID NO: 157 - protein, 11 aa, synthetic construct
ATTDGYSLFTY

SEQ ID NO: 158 - protein, 6 aa, synthetic construct
QGINNY

SEQ ID NO: 159 - skipped

SEQ ID NO: 160 - protein, 9 aa, synthetic construct
QQYSKLPLT

SEQ ID NO: 161 - protein, 5 aa, synthetic construct
NYGMN

SEQ ID NO: 162 - protein, 17 aa, synthetic construct
WINTYTGEPTYADDFKG

SEQ ID NO: 163 - protein, 15 aa, synthetic construct
EINDYVSGRYYAMDY

SEQ ID NO: 164 - protein, 11 aa, synthetic construct
RASENIYSYLA

SEQ ID NO: 165 - protein, 7 aa, synthetic construct
NAKTLAE

SEQ ID NO: 166 - protein, 9 aa, synthetic construct
QHHYNTPLT

SEQ ID NO: 167 - protein, 5 aa, synthetic construct
DTYMH

SEQ ID NO: 168 - protein, 17 aa, synthetic construct
RIDPANGNTKYDPKFQG

SEQ ID NO: 169 - protein, 10 aa, synthetic construct
EYYGSSLFDY

SEQ ID NO: 170 - protein, 12 aa, synthetic construct
SVSSSISSSYLH

SEQ ID NO: 171 - protein, 7 aa, synthetic construct
GTSNLAS

SEQ ID NO: 172 - protein, 9 aa, synthetic construct
QQWSGYPFT

SEQ ID NO: 173 - protein, 5 aa, synthetic construct
DYLIE

SEQ ID NO: 174 - protein, 17 aa, synthetic construct
GINPGSGSTNYNEKFKG

SEQ ID NO: 175 - protein, 8 aa, synthetic construct
LDGNYVAY

SEQ ID NO: 176 - protein, 11 aa, synthetic construct
RASGNIHNYLA

SEQ ID NO: 177 - protein, 7 aa, synthetic construct
HAKTLAD

SEQ ID NO: 178 - protein, 9 aa, synthetic construct
QHFWSTPWT

SEQ ID NO: 179 - protein, 5 aa, synthetic construct
DYLIE

SEQ ID NO: 180 - protein, 17 aa, synthetic construct
GINPGSGSTNYNEKFKG

SEQ ID NO: 181 - protein, 8 aa, synthetic construct
LDGNYVGY

SEQ ID NO: 182 - protein, 11 aa, synthetic construct
RASGNIHNYLA

SEQ ID NO: 183 - protein, 7 aa, synthetic construct
HAKTLAD

SEQ ID NO: 184 - protein, 9 aa, synthetic construct
QHFWSTPWT

SEQ ID NO: 185 - protein, 5 aa, synthetic construct
DYLIE

SEQ ID NO: 186 - protein, 17 aa, synthetic construct
GINPGSGSTNYNEKFKG

SEQ ID NO: 187 - protein, 8 aa, synthetic construct
LDGNYVGY

SEQ ID NO: 188 - protein, 11 aa, synthetic construct
RASGNIHNYLA

SEQ ID NO: 189 - protein, 7 aa, synthetic construct
HAKTLAD

SEQ ID NO: 190 - protein, 9 aa, synthetic construct
QHFWSTPWT

SEQ ID NO: 191 - protein, 5 aa, synthetic construct
DYLIE

SEQ ID NO: 192 - protein, 17 aa, synthetic construct
GINPGSGSTNYNEKFKG

SEQ ID NO: 193 - protein, 8 aa, synthetic construct
LDGNYVGY

SEQ ID NO: 194 - protein, 11 aa, synthetic construct
RASGNIHNYLT

SEQ ID NO: 195 - protein, 7 aa, synthetic construct
HAKTLAD

SEQ ID NO: 196 - protein, 9 aa, synthetic construct
QHFWSTPWT

SEQ ID NO: 197 - protein, 5 aa, synthetic construct
DYLIE

SEQ ID NO: 198 - protein, 17 aa, synthetic construct
GINPGSGSTNYNEKFKG

SEQ ID NO: 199 - protein, 8 aa, synthetic construct
LDGNYVGY

SEQ ID NO: 200 - protein, 11 aa, synthetic construct
RASENIHNYLA

SEQ ID NO: 201 - protein, 7 aa, synthetic construct
YAKTLAD

SEQ ID NO: 202 - protein, 9 aa, synthetic construct
QHFWSTPRT

SEQ ID NO: 203 - protein, 5 aa, synthetic construct
DYLIE

SEQ ID NO: 204 - protein, 17 aa, synthetic construct
GINPGSGSTNYNEKFKG

SEQ ID NO: 205 - protein, 8 aa, synthetic construct
LDGNYVGY

SEQ ID NO: 206 - protein, 11 aa, synthetic construct
RASENIHNYLA

SEQ ID NO: 207 - protein, 7 aa, synthetic construct
YAKTLAD

SEQ ID NO: 208 - protein, 9 aa, synthetic construct
QHFWSTPRT

SEQ ID NO: 209 - protein, 5 aa, synthetic construct
DYLIE

SEQ ID NO: 210 - protein, 17 aa, synthetic construct
GINPGSGSTNYNEKFKG

SEQ ID NO: 211 - protein, 8 aa, synthetic construct
LDGNYVGY

SEQ ID NO: 212 - protein, 11 aa, synthetic construct
RASGNIHNYLA

SEQ ID NO: 213 - protein, 7 aa, synthetic construct
HAKTLAD

SEQ ID NO: 214 - protein, 9 aa, synthetic construct
QHFWNTPWT

SEQ ID NO: 215 - protein, 5 aa, synthetic construct
DYYMK

SEQ ID NO: 216 - protein, 17 aa, synthetic construct
DINPNNGDTFYNQKFKG

SEQ ID NO: 217 - protein, 8 aa, synthetic construct
GGYDGFAY

SEQ ID NO: 218 - protein, 15 aa, synthetic construct
KASQSVDYDGDSYMN

SEQ ID NO: 219 - protein, 7 aa, synthetic construct
AASNLES

SEQ ID NO: 220 - protein, 9 aa, synthetic construct
QESNEDPYT

SEQ ID NO: 221 - protein, 5 aa, synthetic construct
KYNIY

SEQ ID NO: 222 - protein, 17 aa, synthetic construct
YIDPYNGGTVQNQKFKG

SEQ ID NO: 223 - protein, 9 aa, synthetic construct
TDGYSLFTY

SEQ ID NO: 224 - protein, 11 aa, synthetic construct
RASQDIGNYLN

SEQ ID NO: 225 - protein, 7 aa, synthetic construct
YTSRLHS

SEQ ID NO: 226 - protein, 9 aa, synthetic construct
QQGNTLPFT

SEQ ID NO: 227 - protein, 5 aa, synthetic construct
KYNIY

SEQ ID NO: 228 - protein, 17 aa, synthetic construct
YIDPYNGGTVYNQKFEG

SEQ ID NO: 229 - protein, 9 aa, synthetic construct
TDGYSLFTY

SEQ ID NO: 230 - protein, 11 aa, synthetic construct
SAGQGINNYLN

SEQ ID NO: 231 - protein, 7 aa, synthetic construct
YTSSLQS

SEQ ID NO: 232 - protein, 9 aa, synthetic construct
QQYSKLPLT

SEQ ID NO: 233 - protein, 5 aa, synthetic construct
KYNIY

SEQ ID NO: 234 - protein, 17 aa, synthetic construct
YIDPYNGGTVYNQKFKG

SEQ ID NO: 235 - protein, 9 aa, synthetic construct
TDGYSLFTY

SEQ ID NO: 236 - protein, 11 aa, synthetic construct
SAGQGINNYLN

SEQ ID NO: 237 - protein, 7 aa, synthetic construct
YTSSLQS

SEQ ID NO: 238 - protein, 9 aa, synthetic construct
QQYSKLPLT

SEQ ID NO: 239 - protein, 5 aa, synthetic construct
KYNIY

SEQ ID NO: 240 - protein, 17 aa, synthetic construct
YIDPYNGGTVQNQKFKG

SEQ ID NO: 241 - protein, 9 aa, synthetic construct
TDGYSLFTY

SEQ ID NO: 242 - protein, 12 aa, synthetic construct
SASSSISSNYLH

SEQ ID NO: 243 - protein, 7 aa, synthetic construct
RTSNLAS

SEQ ID NO: 244 - protein, 9 aa, synthetic construct
QQGSSIPFT

SEQ ID NO: 245 - protein, 5 aa, synthetic construct
KYNIY

SEQ ID NO: 246 - protein, 17 aa, synthetic construct
YIDPYNGGTVYNQKFEG

SEQ ID NO: 247 - protein, 9 aa, synthetic construct
TDGYSLFTY

SEQ ID NO: 248 - protein, 11 aa, synthetic construct
SAGQGINNYLN

SEQ ID NO: 249 - protein, 7 aa, synthetic construct
YTSSLQS

SEQ ID NO: 250 - protein, 9 aa, synthetic construct
QQYSKLPLT

SEQ ID NO: 251 - protein, 5 aa, Mus musculus
KYNIY

SEQ ID NO: 252 - protein, 17 aa, Mus musculus
YIDPYNGGTVYNQKFKG

SEQ ID NO: 253 - protein, 9 aa, Mus musculus
TDGYSLFTY

SEQ ID NO: 254 - protein, 11 aa, Mus musculus
RASQDISNYLN

SEQ ID NO: 255 - protein, 7 aa, Mus musculus
YTSRLHS

SEQ ID NO: 256 - protein, 9 aa, Mus musculus
QQGNTLPFT

SEQ ID NO: 257 - protein, 5 aa, synthetic construct
KYNIY

SEQ ID NO: 258 - protein, 17 aa, synthetic construct
YIDPYNGGTVYNQKFKG

SEQ ID NO: 259 - protein, 9 aa, synthetic construct
TDGYSLFTY

SEQ ID NO: 260 - protein, 11 aa, synthetic construct
RASQDISNYLN

SEQ ID NO: 261 - protein, 7 aa, synthetic construct
YTSRLHS

SEQ ID NO: 262 - protein, 9 aa, synthetic construct
QQGNTLPLT

SEQ ID NO: 263 - protein, 5 aa, synthetic construct
IHWMQ

SEQ ID NO: 264 - protein, 17 aa, synthetic construct
AIYPGNGDTKYTQKFKG

SEQ ID NO: 265 - protein, 8 aa, synthetic construct
LYGNYEAY

SEQ ID NO: 266 - protein, 11 aa, synthetic construct
GASENIYGALN

SEQ ID NO: 267 - protein, 7 aa, synthetic construct
GTTNLAD

SEQ ID NO: 268 - protein, 10 aa, synthetic construct
QNVLTTPPWT

SEQ ID NO: 269 - protein, 5 aa, synthetic construct
DYLIE

SEQ ID NO: 270 - protein, 17 aa, synthetic construct
GINPGSGSTNYNEKFKG

SEQ ID NO: 271 - protein, 8 aa, synthetic construct
LDGNYVGY

SEQ ID NO: 272 - protein, 11 aa, synthetic construct
RASGNIHNYLA

SEQ ID NO: 273 - protein, 7 aa, synthetic construct
HAKTLAD

SEQ ID NO: 274 - protein, 9 aa, synthetic construct
QHFWSTPWT

SEQ ID NO: 275 - protein, 5 aa, synthetic construct
KYNIY

SEQ ID NO: 276 - protein, 17 aa, synthetic construct
YIDPYNGGTVYNQKFKG

SEQ ID NO: 277 - protein, 9 aa, synthetic construct
TDGYSLFTY

SEQ ID NO: 278 - protein, 11 aa, synthetic construct
SAGQGINNYLN

SEQ ID NO: 279 - protein, 7 aa, synthetic construct
YTSSLQS

SEQ ID NO: 280 - protein, 9 aa, synthetic construct
QQYSKLPLT

SEQ ID NO: 281 - protein, 7 aa, synthetic construct
GYTFTNY

SEQ ID NO: 282 - protein, 6 aa, synthetic construct
NTYTGE

SEQ ID NO: 283 - protein, 15 aa, synthetic construct
EINDYVSGRYYAMDY

SEQ ID NO: 284 - protein, 11 aa, synthetic construct
RASENIYSYLA

SEQ ID NO: 285 - protein, 7 aa, synthetic construct
NAKTLAE

SEQ ID NO: 286 - protein, 9 aa, synthetic construct
QHHYNTPLT

SEQ ID NO: 287 - protein, 7 aa, synthetic construct
GFNITDT

SEQ ID NO: 288 - protein, 6 aa, synthetic construct
DPANGN

SEQ ID NO: 289 - protein, 10 aa, synthetic construct
EYYGSSLFDY

SEQ ID NO: 290 - protein, 12 aa, synthetic construct
SVSSSISSSYLH

SEQ ID NO: 291 - protein, 7 aa, synthetic construct
GTSNLAS

SEQ ID NO: 292 - protein, 9 aa, synthetic construct
QQWSGYPFT

SEQ ID NO: 293 - protein, 7 aa, synthetic construct
GYAFTDY

SEQ ID NO: 294 - protein, 6 aa, synthetic construct
NPGSGS

SEQ ID NO: 295 - protein, 8 aa, synthetic construct
LDGNYVAY

SEQ ID NO: 296 - protein, 11 aa, synthetic construct
RASGNIHNYLA

SEQ ID NO: 297 - protein, 7 aa, synthetic construct
HAKTLAD

SEQ ID NO: 298 - protein, 9 aa, synthetic construct
QHFWSTPWT

SEQ ID NO: 299 - protein, 7 aa, synthetic construct
GYAFTDY

SEQ ID NO: 300 - protein, 6 aa, synthetic construct
NPGSGS

SEQ ID NO: 301 - protein, 8 aa, synthetic construct
LDGNYVGY

SEQ ID NO: 302 - protein, 11 aa, synthetic construct
RASGNIHNYLA

SEQ ID NO: 303 - protein, 7 aa, synthetic construct
HAKTLAD

SEQ ID NO: 304 - protein, 9 aa, synthetic construct
QHFWSTPWT

SEQ ID NO: 305 - protein, 7 aa, synthetic construct
GYAFTDY

SEQ ID NO: 306 - protein, 6 aa, synthetic construct
NPGSGS

SEQ ID NO: 307 - protein, 8 aa, synthetic construct
LDGNYVGY

SEQ ID NO: 308 - protein, 11 aa, synthetic construct
RASGNIHNYLA

SEQ ID NO: 309 - protein, 7 aa, synthetic construct
HAKTLAD

SEQ ID NO: 310 - protein, 9 aa, synthetic construct
QHFWSTPWT

SEQ ID NO: 311 - protein, 7 aa, synthetic construct
GYAFIDY

SEQ ID NO: 312 - protein, 6 aa, synthetic construct
NPGSGS

SEQ ID NO: 313 - protein, 8 aa, synthetic construct
LDGNYVGY

SEQ ID NO: 314 - protein, 11 aa, synthetic construct
RASGNIHNYLT

SEQ ID NO: 315 - protein, 7 aa, synthetic construct
HAKTLAD

SEQ ID NO: 316 - protein, 9 aa, synthetic construct
QHFWSTPWT

SEQ ID NO: 317 - protein, 7 aa, synthetic construct
GYAFTDY

SEQ ID NO: 318 - protein, 6 aa, synthetic construct
NPGSGS

SEQ ID NO: 319 - protein, 8 aa, synthetic construct
LDGNYVGY

SEQ ID NO: 320 - protein, 11 aa, synthetic construct
RASENIHNYLA

SEQ ID NO: 321 - protein, 7 aa, synthetic construct
YAKTLAD

SEQ ID NO: 322 - protein, 9 aa, synthetic construct
QHFWSTPRT

SEQ ID NO: 323 - protein, 7 aa, synthetic construct
GYAFTDY

SEQ ID NO: 324 - protein, 6 aa, synthetic construct
NPGSGS

SEQ ID NO: 325 - protein, 8 aa, synthetic construct
LDGNYVGY

SEQ ID NO: 326 - protein, 11 aa, synthetic construct
RASENIHNYLA

SEQ ID NO: 327 - protein, 7 aa, synthetic construct
YAKTLAD

SEQ ID NO: 328 - protein, 9 aa, synthetic construct
QHFWSTPRT

SEQ ID NO: 329 - protein, 7 aa, synthetic construct
GYAFTDY

SEQ ID NO: 330 - protein, 6 aa, synthetic construct
NPGSGS

SEQ ID NO: 331 - protein, 8 aa, synthetic construct
LDGNYVGY

SEQ ID NO: 332 - protein, 11 aa, synthetic construct
RASGNIHNYLA

SEQ ID NO: 333 - protein, 7 aa, synthetic construct
HAKTLAD

SEQ ID NO: 334 - protein, 9 aa, synthetic construct
QHFWNTPWT

SEQ ID NO: 335 - protein, 7 aa, synthetic construct
GYTFTDY

SEQ ID NO: 336 - protein, 6 aa, synthetic construct
NPNNGD

SEQ ID NO: 337 - protein, 8 aa, synthetic construct
GGYDGFAY

SEQ ID NO: 338 - protein, 15 aa, synthetic construct
KASQSVDYDGDSYMN

SEQ ID NO: 339 - protein, 7 aa, synthetic construct
AASNLES

SEQ ID NO: 340 - protein, 9 aa, synthetic construct
QESNEDPYT

SEQ ID NO: 341 - protein, 7 aa, synthetic construct
GYAFTKY

SEQ ID NO: 342 - protein, 6 aa, synthetic construct
DPYNGG

SEQ ID NO: 343 - protein, 9 aa, synthetic construct
TDGYSLFTY

SEQ ID NO: 344 - protein, 11 aa, synthetic construct
RASQDIGNYLN

SEQ ID NO: 345 - protein, 7 aa, synthetic construct
YTSRLHS

SEQ ID NO: 346 - protein, 9 aa, synthetic construct
QQGNTLPFT

SEQ ID NO: 347 - protein, 7 aa, synthetic construct
GYAFTKY

SEQ ID NO: 348 - protein, 6 aa, synthetic construct
DPYNGG

SEQ ID NO: 349 - protein, 9 aa, synthetic construct
TDGYSLFTY

SEQ ID NO: 350 - protein, 11 aa, synthetic construct
SAGQGINNYLN

SEQ ID NO: 351 - protein, 7 aa, synthetic construct
YTSSLQS

SEQ ID NO: 352 - protein, 9 aa, synthetic construct
QQYSKLPLT

SEQ ID NO: 353 - protein, 7 aa, synthetic construct
GYAFTKY

SEQ ID NO: 354 - protein, 6 aa, synthetic construct
DPYNGG

SEQ ID NO: 355 - protein, 9 aa, synthetic construct
TDGYSLFTY

SEQ ID NO: 356 - protein, 11 aa, synthetic construct
SAGQGINNYLN

SEQ ID NO: 357 - protein, 7 aa, synthetic construct
YTSSLQS

SEQ ID NO: 358 - protein, 9 aa, synthetic construct
QQYSKLPLT

SEQ ID NO: 359 - protein, 7 aa, synthetic construct
GYAFTKY

SEQ ID NO: 360 - protein, 6 aa, synthetic construct
DPYNGG

SEQ ID NO: 361 - protein, 9 aa, synthetic construct
TDGYSLFTY

SEQ ID NO: 362 - protein, 12 aa, synthetic construct
SASSSISSNYLH

SEQ ID NO: 363 - protein, 7 aa, synthetic construct
RTSNLAS

SEQ ID NO: 364 - protein, 9 aa, synthetic construct
QQGSSIPFT

SEQ ID NO: 365 - protein, 7 aa, synthetic construct
GYAFTKY

SEQ ID NO: 366 - protein, 6 aa, synthetic construct
DPYNGG

SEQ ID NO: 367 - protein, 9 aa, synthetic construct
TDGYSLFTY

SEQ ID NO: 368 - protein, 11 aa, synthetic construct
SAGQGINNYLN

SEQ ID NO: 369 - protein, 7 aa, synthetic construct
YTSSLQS

SEQ ID NO: 370 - protein, 9 aa, synthetic construct
QQYSKLPLT

SEQ ID NO: 371 - protein, 7 aa, Mus musculus
GYAFTKY

SEQ ID NO: 372 - protein, 6 aa, Mus musculus
DPYNGG

SEQ ID NO: 373 - protein, 9 aa, Mus musculus
TDGYSLFTY

SEQ ID NO: 374 - protein, 11 aa, Mus musculus
RASQDISNYLN

SEQ ID NO: 375 - protein, 7 aa, Mus musculus
YTSRLHS

SEQ ID NO: 376 - protein, 9 aa, Mus musculus
QQGNTLPFT

SEQ ID NO: 377 - protein, 7 aa, synthetic construct
GYAFTKY

SEQ ID NO: 378 - protein, 6 aa, synthetic construct
DPYNGG

SEQ ID NO: 379 - protein, 9 aa, synthetic construct
TDGYSLFTY

SEQ ID NO: 380 - protein, 11 aa, synthetic construct
RASQDISNYLN

SEQ ID NO: 381 - protein, 7 aa, synthetic construct
YTSRLHS

SEQ ID NO: 382 - protein, 9 aa, synthetic construct
QQGNTLPLT

SEQ ID NO: 383 - protein, 7 aa, synthetic construct
GYTFSIH

SEQ ID NO: 384 - protein, 6 aa, synthetic construct
YPGNGD

SEQ ID NO: 385 - protein, 8 aa, synthetic construct
LYGNYEAY

SEQ ID NO: 386 - protein, 11 aa, synthetic construct
GASENIYGALN

SEQ ID NO: 387 - protein, 7 aa, synthetic construct
GTTNLAD

SEQ ID NO: 388 - protein, 10 aa, synthetic construct
QNVLTTPPWT

SEQ ID NO: 389 - protein, 7 aa, synthetic construct
GYAFTDY

SEQ ID NO: 390 - protein, 6 aa, synthetic construct
NPGSGS

SEQ ID NO: 391 - protein, 8 aa, synthetic construct
LDGNYVGY

SEQ ID NO: 392 - protein, 11 aa, synthetic construct
RASGNIHNYLA

SEQ ID NO: 393 - protein, 7 aa, synthetic construct
HAKTLAD

SEQ ID NO: 394 - protein, 9 aa, synthetic construct
QHFWSTPWT

SEQ ID NO: 395 - protein, 7 aa, synthetic construct
GYAFTKY

SEQ ID NO: 396 - protein, 6 aa, synthetic construct
DPYNGG

SEQ ID NO: 397 - protein, 9 aa, synthetic construct
TDGYSLFTY

SEQ ID NO: 398 - protein, 11 aa, synthetic construct
SAGQGINNYLN

SEQ ID NO: 399 - protein, 7 aa, synthetic construct
YTSSLQS

SEQ ID NO: 400 - protein, 9 aa, synthetic construct
QQYSKLPLT

SEQ ID NO: 401 - protein, 8 aa, synthetic construct
GYTFTNYG

SEQ ID NO: 402 - protein, 8 aa, synthetic construct
INTYTGEP

SEQ ID NO: 403 - protein, 17 aa, synthetic construct
AREINDYVSGRYYAMDY

SEQ ID NO: 404 - protein, 6 aa, synthetic construct
ENIYSY

SEQ ID NO: 405 - skipped

SEQ ID NO: 406 - protein, 9 aa, synthetic construct
QHHYNTPLT

SEQ ID NO: 407 - protein, 8 aa, synthetic construct
GFNITDTY

SEQ ID NO: 408 - protein, 8 aa, synthetic construct
IDPANGNT

SEQ ID NO: 409 - protein, 12 aa, synthetic construct
AREYYGSSLFDY

SEQ ID NO: 410 - protein, 7 aa, synthetic construct
SSISSSY

SEQ ID NO: 411 - skipped

SEQ ID NO: 412 - protein, 9 aa, synthetic construct
QQWSGYPFT

SEQ ID NO: 413 - protein, 8 aa, synthetic construct
GYAFTDYL

SEQ ID NO: 414 - protein, 8 aa, synthetic construct
INPGSGST

SEQ ID NO: 415 - protein, 10 aa, synthetic construct
ARLDGNYVAY

SEQ ID NO: 416 - protein, 6 aa, synthetic construct
GNIHNY

SEQ ID NO: 417 - skipped

SEQ ID NO: 418 - protein, 9 aa, synthetic construct
QHFWSTPWT

SEQ ID NO: 419 - protein, 8 aa, synthetic construct
GYAFTDYL

SEQ ID NO: 420 - protein, 8 aa, synthetic construct
INPGSGST

SEQ ID NO: 421 - protein, 10 aa, synthetic construct
ARLDGNYVGY

SEQ ID NO: 422 - protein, 6 aa, synthetic construct
GNIHNY

SEQ ID NO: 423 - skipped

SEQ ID NO: 424 - protein, 9 aa, synthetic construct
QHFWSTPWT

SEQ ID NO: 425 - protein, 8 aa, synthetic construct
GYAFTDYL

SEQ ID NO: 426 - protein, 8 aa, synthetic construct
INPGSGST

SEQ ID NO: 427 - protein, 10 aa, synthetic construct
ARLDGNYVGY

SEQ ID NO: 428 - protein, 6 aa, synthetic construct
GNIHNY

SEQ ID NO: 429 - skipped

SEQ ID NO: 430 - protein, 9 aa, synthetic construct
QHFWSTPWT

SEQ ID NO: 431 - protein, 8 aa, synthetic construct
GYAFIDYL

SEQ ID NO: 432 - protein, 8 aa, synthetic construct
INPGSGST

SEQ ID NO: 433 - protein, 10 aa, synthetic construct
ARLDGNYVGY

SEQ ID NO: 434 - protein, 6 aa, synthetic construct
GNIHNY

SEQ ID NO: 435 - skipped

SEQ ID NO: 436 - protein, 9 aa, synthetic construct
QHFWSTPWT

SEQ ID NO: 437 - protein, 8 aa, synthetic construct
GYAFTDYL

SEQ ID NO: 438 - protein, 8 aa, synthetic construct
INPGSGST

SEQ ID NO: 439 - protein, 10 aa, synthetic construct
ARLDGNYVGY

SEQ ID NO: 440 - protein, 6 aa, synthetic construct
ENIHNY

SEQ ID NO: 441 - skipped

SEQ ID NO: 442 - protein, 9 aa, synthetic construct
QHFWSTPRT

SEQ ID NO: 443 - protein, 8 aa, synthetic construct
GYAFTDYL

SEQ ID NO: 444 - protein, 8 aa, synthetic construct
INPGSGST

SEQ ID NO: 445 - protein, 10 aa, synthetic construct
ARLDGNYVGY

SEQ ID NO: 446 - protein, 6 aa, synthetic construct
ENIHNY

SEQ ID NO: 447 - skipped

SEQ ID NO: 448 - protein, 9 aa, synthetic construct
QHFWSTPRT

SEQ ID NO: 449 - protein, 8 aa, synthetic construct
GYAFTDYL

SEQ ID NO: 450 - protein, 8 aa, synthetic construct
INPGSGST

SEQ ID NO: 451 - protein, 10 aa, synthetic construct
ARLDGNYVGY

SEQ ID NO: 452 - protein, 6 aa, synthetic construct
GNIHNY

SEQ ID NO: 453 - skipped

SEQ ID NO: 454 - protein, 9 aa, synthetic construct
QHFWNTPWT

SEQ ID NO: 455 - protein, 8 aa, synthetic construct
GYTFTDYY

SEQ ID NO: 456 - protein, 8 aa, synthetic construct
INPNNGDT

SEQ ID NO: 457 - protein, 10 aa, synthetic construct
ATGGYDGFAY

SEQ ID NO: 458 - protein, 10 aa, synthetic construct
QSVDYDGDSY

SEQ ID NO: 459 - skipped

SEQ ID NO: 460 - protein, 9 aa, synthetic construct
QESNEDPYT

SEQ ID NO: 461 - protein, 8 aa, synthetic construct
GYAFTKYN

SEQ ID NO: 462 - protein, 8 aa, synthetic construct
IDPYNGGT

SEQ ID NO: 463 - protein, 11 aa, synthetic construct
ATTDGYSLFTY

SEQ ID NO: 464 - protein, 6 aa, synthetic construct
QDIGNY

SEQ ID NO: 465 - skipped

SEQ ID NO: 466 - protein, 9 aa, synthetic construct
QQGNTLPFT

SEQ ID NO: 467 - protein, 8 aa, synthetic construct
GYAFTKYN

SEQ ID NO: 468 - protein, 8 aa, synthetic construct
IDPYNGGT

SEQ ID NO: 469 - protein, 11 aa, synthetic construct
ATTDGYSLFTY

SEQ ID NO: 470 - protein, 6 aa, synthetic construct
QGINNY

SEQ ID NO: 471 - skipped

SEQ ID NO: 472 - protein, 9 aa, synthetic construct
QQYSKLPLT

SEQ ID NO: 473 - protein, 8 aa, synthetic construct
GYAFTKYN

SEQ ID NO: 474 - protein, 8 aa, synthetic construct
IDPYNGGT

SEQ ID NO: 475 - protein, 11 aa, synthetic construct
ATTDGYSLFTY

SEQ ID NO: 476 - protein, 6 aa, synthetic construct
QGINNY

SEQ ID NO: 477 - skipped

SEQ ID NO: 478 - protein, 9 aa, synthetic construct
QQYSKLPLT

SEQ ID NO: 479 - protein, 8 aa, synthetic construct
GYAFTKYN

SEQ ID NO: 480 - protein, 8 aa, synthetic construct
IDPYNGGT

SEQ ID NO: 481 - protein, 11 aa, synthetic construct
ATTDGYSLFTY

SEQ ID NO: 482 - protein, 7 aa, synthetic construct
SSISSNY

SEQ ID NO: 483 - skipped

SEQ ID NO: 484 - protein, 9 aa, synthetic construct
QQGSSIPFT

SEQ ID NO: 485 - protein, 8 aa, synthetic construct
GYAFTKYN

SEQ ID NO: 486 - protein, 8 aa, synthetic construct
IDPYNGGT

SEQ ID NO: 487 - protein, 11 aa, synthetic construct
ATTDGYSLFTY

SEQ ID NO: 488 - protein, 6 aa, synthetic construct
QGINNY

SEQ ID NO: 489 - skipped

SEQ ID NO: 490 - protein, 9 aa, synthetic construct
QQYSKLPLT

SEQ ID NO: 491 - protein, 8 aa, Mus musculus
GYAFTKYN

SEQ ID NO: 492 - protein, 8 aa, Mus musculus
IDPYNGGT

SEQ ID NO: 493 - protein, 11 aa, Mus musculus
ATTDGYSLFTY

SEQ ID NO: 494 - protein, 6 aa, Mus musculus
QDISNY

SEQ ID NO: 495 - skipped

SEQ ID NO: 496 - protein, 9 aa, Mus musculus
QQGNTLPFT

SEQ ID NO: 497 - protein, 8 aa, synthetic construct
GYAFTKYN

SEQ ID NO: 498 - protein, 8 aa, synthetic construct
IDPYNGGT

SEQ ID NO: 499 - protein, 11 aa, synthetic construct
ATTDGYSLFTY

SEQ ID NO: 500 - protein, 6 aa, synthetic construct
QDISNY

SEQ ID NO: 501 - skipped

SEQ ID NO: 502 - protein, 9 aa, synthetic construct
QQGNTLPLT

SEQ ID NO: 503 - protein, 8 aa, synthetic construct
GYTFSIHW

SEQ ID NO: 504 - protein, 8 aa, synthetic construct
IYPGNGDT

SEQ ID NO: 505 - protein, 10 aa, synthetic construct
ATLYGNYEAY

SEQ ID NO: 506 - protein, 6 aa, synthetic construct
ENIYGA

SEQ ID NO: 507 - skipped

SEQ ID NO: 508 - protein, 10 aa, synthetic construct
QNVLTTPPWT

SEQ ID NO: 509 - protein, 8 aa, synthetic construct
GYAFTDYL

SEQ ID NO: 510 - protein, 8 aa, synthetic construct
INPGSGST

SEQ ID NO: 511 - protein, 10 aa, synthetic construct
ARLDGNYVGY

SEQ ID NO: 512 - protein, 6 aa, synthetic construct
GNIHNY

SEQ ID NO: 513 - skipped

SEQ ID NO: 514 - protein, 9 aa, synthetic construct
QHFWSTPWT

SEQ ID NO: 515 - protein, 8 aa, synthetic construct
GYAFTKYN

SEQ ID NO: 516 - protein, 8 aa, synthetic construct
IDPYNGGT

SEQ ID NO: 517 - protein, 11 aa, synthetic construct
ATTDGYSLFTY

SEQ ID NO: 518 - protein, 6 aa, synthetic construct
QGINNY

SEQ ID NO: 519 - skipped

SEQ ID NO: 520 - protein, 9 aa, synthetic construct
QQYSKLPLT

SEQ ID NO: 521 - protein, 13 aa, synthetic construct
KASGYTFTNYGMN

SEQ ID NO: 522 - protein, 10 aa, synthetic construct
WINTYTGEPT

SEQ ID NO: 523 - protein, 17 aa, synthetic construct
AREINDYVSGRYYAMDY

SEQ ID NO: 524 - protein, 11 aa, synthetic construct
RASENIYSYLA

SEQ ID NO: 525 - protein, 8 aa, synthetic construct
YNAKTLAE

SEQ ID NO: 526 - protein, 9 aa, synthetic construct
QHHYNTPLT

SEQ ID NO: 527 - protein, 13 aa, synthetic construct
TASGFNITDTYMH

SEQ ID NO: 528 - protein, 10 aa, synthetic construct
RIDPANGNTK

SEQ ID NO: 529 - protein, 12 aa, synthetic construct
AREYYGSSLFDY

SEQ ID NO: 530 - protein, 12 aa, synthetic construct
SVSSSISSSYLH

SEQ ID NO: 531 - protein, 8 aa, synthetic construct
YGTSNLAS

SEQ ID NO: 532 - protein, 9 aa, synthetic construct
QQWSGYPFT

SEQ ID NO: 533 - protein, 13 aa, synthetic construct
KASGYAFTDYLIE

SEQ ID NO: 534 - protein, 10 aa, synthetic construct
GINPGSGSTN

SEQ ID NO: 535 - protein, 10 aa, synthetic construct
ARLDGNYVAY

SEQ ID NO: 536 - protein, 11 aa, synthetic construct
RASGNIHNYLA

SEQ ID NO: 537 - protein, 8 aa, synthetic construct
YHAKTLAD

SEQ ID NO: 538 - protein, 9 aa, synthetic construct
QHFWSTPWT

SEQ ID NO: 539 - protein, 13 aa, synthetic construct
KASGYAFTDYLIE

SEQ ID NO: 540 - protein, 10 aa, synthetic construct
GINPGSGSTN

SEQ ID NO: 541 - protein, 10 aa, synthetic construct
ARLDGNYVGY

SEQ ID NO: 542 - protein, 11 aa, synthetic construct
RASGNIHNYLA

SEQ ID NO: 543 - protein, 8 aa, synthetic construct
YHAKTLAD

SEQ ID NO: 544 - protein, 9 aa, synthetic construct
QHFWSTPWT

SEQ ID NO: 545 - protein, 13 aa, synthetic construct
KASGYAFTDYLIE

SEQ ID NO: 546 - protein, 10 aa, synthetic construct
GINPGSGSTN

SEQ ID NO: 547 - protein, 10 aa, synthetic construct
ARLDGNYVGY

SEQ ID NO: 548 - protein, 11 aa, synthetic construct
RASGNIHNYLA

SEQ ID NO: 549 - protein, 8 aa, synthetic construct
YHAKTLAD

SEQ ID NO: 550 - protein, 9 aa, synthetic construct
QHFWSTPWT

SEQ ID NO: 551 - protein, 13 aa, synthetic construct
KASGYAFIDYLIE

SEQ ID NO: 552 - protein, 10 aa, synthetic construct
GINPGSGSTN

SEQ ID NO: 553 - protein, 10 aa, synthetic construct
ARLDGNYVGY

SEQ ID NO: 554 - protein, 11 aa, synthetic construct
RASGNIHNYLT

SEQ ID NO: 555 - protein, 8 aa, synthetic construct
YHAKTLAD

SEQ ID NO: 556 - protein, 9 aa, synthetic construct
QHFWSTPWT

SEQ ID NO: 557 - protein, 13 aa, synthetic construct
KASGYAFTDYLIE

SEQ ID NO: 558 - protein, 10 aa, synthetic construct
GINPGSGSTN

SEQ ID NO: 559 - protein, 10 aa, synthetic construct
ARLDGNYVGY

SEQ ID NO: 560 - protein, 11 aa, synthetic construct
RASENIHNYLA

SEQ ID NO: 561 - protein, 8 aa, synthetic construct
YYAKTLAD

SEQ ID NO: 562 - protein, 9 aa, synthetic construct
QHFWSTPRT

SEQ ID NO: 563 - protein, 13 aa, synthetic construct
KASGYAFTDYLIE

SEQ ID NO: 564 - protein, 10 aa, synthetic construct
GINPGSGSTN

SEQ ID NO: 565 - protein, 10 aa, synthetic construct
ARLDGNYVGY

SEQ ID NO: 566 - protein, 11 aa, synthetic construct
RASENIHNYLA

SEQ ID NO: 567 - protein, 8 aa, synthetic construct
YYAKTLAD

SEQ ID NO: 568 - protein, 9 aa, synthetic construct
QHFWSTPRT

SEQ ID NO: 569 - protein, 13 aa, synthetic construct
KASGYAFTDYLIE

SEQ ID NO: 570 - protein, 10 aa, synthetic construct
GINPGSGSTN

SEQ ID NO: 571 - protein, 10 aa, synthetic construct
ARLDGNYVGY

SEQ ID NO: 572 - protein, 11 aa, synthetic construct
RASGNIHNYLA

SEQ ID NO: 573 - protein, 8 aa, synthetic construct
YHAKTLAD

SEQ ID NO: 574 - protein, 9 aa, synthetic construct
QHFWNTPWT

SEQ ID NO: 575 - protein, 13 aa, synthetic construct
KASGYTFTDYYMK

SEQ ID NO: 576 - protein, 10 aa, synthetic construct
DINPNNGDTF

SEQ ID NO: 577 - protein, 10 aa, synthetic construct
ATGGYDGFAY

SEQ ID NO: 578 - protein, 15 aa, synthetic construct
KASQSVDYDGDSYMN

SEQ ID NO: 579 - protein, 8 aa, synthetic construct
YAASNLES

SEQ ID NO: 580 - protein, 9 aa, synthetic construct
QESNEDPYT

SEQ ID NO: 581 - protein, 13 aa, synthetic construct
KASGYAFTKYNIY

SEQ ID NO: 582 - protein, 10 aa, synthetic construct
YIDPYNGGTV

SEQ ID NO: 583 - protein, 11 aa, synthetic construct
ATTDGYSLFTY

SEQ ID NO: 584 - protein, 11 aa, synthetic construct
RASQDIGNYLN

SEQ ID NO: 585 - protein, 8 aa, synthetic construct
YYTSRLHS

SEQ ID NO: 586 - protein, 9 aa, synthetic construct
QQGNTLPFT

SEQ ID NO: 587 - protein, 13 aa, synthetic construct
KASGYAFTKYNIY

SEQ ID NO: 588 - protein, 10 aa, synthetic construct
YIDPYNGGTV

SEQ ID NO: 589 - protein, 11 aa, synthetic construct
ATTDGYSLFTY

SEQ ID NO: 590 - protein, 11 aa, synthetic construct
SAGQGINNYLN

SEQ ID NO: 591 - protein, 8 aa, synthetic construct
YYTSSLQS

SEQ ID NO: 592 - protein, 9 aa, synthetic construct
QQYSKLPLT

SEQ ID NO: 593 - protein, 13 aa, synthetic construct
KASGYAFTKYNIY

SEQ ID NO: 594 - protein, 10 aa, synthetic construct
YIDPYNGGTV

SEQ ID NO: 595 - protein, 11 aa, synthetic construct
ATTDGYSLFTY

SEQ ID NO: 596 - protein, 11 aa, synthetic construct
SAGQGINNYLN

SEQ ID NO: 597 - protein, 8 aa, synthetic construct
FYTSSLQS

SEQ ID NO: 598 - protein, 9 aa, synthetic construct
QQYSKLPLT

SEQ ID NO: 599 - protein, 13 aa, synthetic construct
KASGYAFTKYNIY

SEQ ID NO: 600 - protein, 10 aa, synthetic construct
YIDPYNGGTV

SEQ ID NO: 601 - protein, 11 aa, synthetic construct
ATTDGYSLFTY

SEQ ID NO: 602 - protein, 12 aa, synthetic construct
SASSSISSNYLH

SEQ ID NO: 603 - protein, 8 aa, synthetic construct
YRTSNLAS

SEQ ID NO: 604 - protein, 9 aa, synthetic construct
QQGSSIPFT

SEQ ID NO: 605 - protein, 13 aa, synthetic construct
KASGYAFTKYNIY

SEQ ID NO: 606 - protein, 10 aa, synthetic construct
YIDPYNGGTV

SEQ ID NO: 607 - protein, 11 aa, synthetic construct
ATTDGYSLFTY

SEQ ID NO: 608 - protein, 11 aa, synthetic construct
SAGQGINNYLN

SEQ ID NO: 609 - protein, 8 aa, synthetic construct
YYTSSLQS

SEQ ID NO: 610 - protein, 9 aa, synthetic construct
QQYSKLPLT

SEQ ID NO: 611 - protein, 13 aa, Mus musculus
KASGYAFTKYNIY

SEQ ID NO: 612 - protein, 10 aa, Mus musculus
YIDPYNGGTV

SEQ ID NO: 613 - protein, 11 aa, Mus musculus
ATTDGYSLFTY

SEQ ID NO: 614 - protein, 11 aa, Mus musculus
RASQDISNYLN

SEQ ID NO: 615 - protein, 8 aa, Mus musculus
YYTSRLHS

SEQ ID NO: 616 - protein, 9 aa, Mus musculus
QQGNTLPFT

SEQ ID NO: 617 - protein, 13 aa, synthetic construct
KASGYAFTKYNIY

SEQ ID NO: 618 - protein, 10 aa, synthetic construct
YIDPYNGGTV

SEQ ID NO: 619 - protein, 11 aa, synthetic construct
ATTDGYSLFTY

SEQ ID NO: 620 - protein, 11 aa, synthetic construct
RASQDISNYLN

SEQ ID NO: 621 - protein, 8 aa, synthetic construct
YYTSRLHS

SEQ ID NO: 622 - protein, 9 aa, synthetic construct
QQGNTLPLT

SEQ ID NO: 623 - protein, 13 aa, synthetic construct
KASGYTFSIHWMQ

SEQ ID NO: 624 - protein, 10 aa, synthetic construct
AIYPGNGDTK

SEQ ID NO: 625 - protein, 10 aa, synthetic construct
ATLYGNYEAY

SEQ ID NO: 626 - protein, 11 aa, synthetic construct
GASENIYGALN

SEQ ID NO: 627 - protein, 8 aa, synthetic construct
YGTTNLAD

SEQ ID NO: 628 - protein, 10 aa, synthetic construct
QNVLTTPPWT

SEQ ID NO: 629 - protein, 13 aa, synthetic construct
KASGYAFTDYLIE

SEQ ID NO: 630 - protein, 10 aa, synthetic construct
GINPGSGSTN

SEQ ID NO: 631 - protein, 10 aa, synthetic construct
ARLDGNYVGY

SEQ ID NO: 632 - protein, 11 aa, synthetic construct
RASGNIHNYLA

SEQ ID NO: 633 - protein, 8 aa, synthetic construct
YHAKTLAD

SEQ ID NO: 634 - protein, 9 aa, synthetic construct
QHFWSTPWT

SEQ ID NO: 635 - protein, 13 aa, synthetic construct
KASGYAFTKYNIY

SEQ ID NO: 636 - protein, 10 aa, synthetic construct
YIDPYNGGTV

SEQ ID NO: 637 - protein, 11 aa, synthetic construct
ATTDGYSLFTY

SEQ ID NO: 638 - protein, 11 aa, synthetic construct
SAGQGINNYLN

SEQ ID NO: 639 - protein, 8 aa, synthetic construct
FYTSSLQS

SEQ ID NO: 640 - protein, 9 aa, synthetic construct
QQYSKLPLT

SEQ ID NO: 641 - protein, 118 aa, synthetic construct
QVQLVQSGAEVKKPGASVKVSCKASGYAFTKYNIYWVRQAPGQGLEWIGYIDPYNGGTVYNQKFKGRATLTVDKSTSTAYMELRSLRSDDTAVYYCATTDGYSLFTYWGQGTLVTVSS

SEQ ID NO: 642 - protein, 118 aa, synthetic construct
QVQLVQSGAEVKKPGASVKVSCKASGYAFTKYNIYWVRQAPGQGLEWIGYIDPYNGGTVYNQKFQGRATLTVDKSTSTAYMELRSLRSDDTAVYYCATTDGYSLFTYWGQGTLVTVSS

SEQ ID NO: 643 - protein, 118 aa, synthetic construct
QVQLVQSGAEVKKPGASVKVSCKASGYAFTKYNIYWVRQAPGQGLEWIGYIDPYNGGTVYNQKFKGKATLTVDKSTSTAYMELRSLRSDDTAVYYCATTDGYSLFTYWGQGTLVTVSS

SEQ ID NO: 644 - protein, 118 aa, synthetic construct
QVQLVQSGAEVKKPGASVKVSCKASGYAFTKYNIYWVRQAPGQGLEWIGYIDPYNGGTVYNQKFQGKATLTVDKSTSTAYMELRSLRSDDTAVYYCATTDGYSLFTYWGQGTLVTVSS

SEQ ID NO: 645 - protein, 118 aa, synthetic construct
QVQLVQSGAEVKKPGASVKVSCKASGYAFTKYNIYWVRQAPGQGLEWIGYIDPYFGGTVYNHKFKGKATLTVDKSTSTAYMELRSLRSDDTAVYYCATTDGYSLFTYWGQGTLVTVSS

SEQ ID NO: 646 - protein, 118 aa, synthetic construct
QVQLVQSSAEVKKPGASVKVSCKASGYAFTKYNIYWVRQAPGQGLEWIGYIDPYFGGTVYNQKFKGKATLTVDKSTSTAYMELRSLRSDDTAVYYCATTDGYSLFTYWGQGTLVTVSS

SEQ ID NO: 647 - protein, 118 aa, synthetic construct
QVQLVQSGAEVKKPGASVKVSCKASGYAFTKYNFYWVRQAPGQGLEWIGYIDPYFGGTVYNQKFKGKATLTVDKSTSTAYMELRSLRSDDTAVYYCATTDGYSLFTYWGQGTLVTVSS

SEQ ID NO: 648 - protein, 118 aa, synthetic construct
QVQLVQSGAEVKKPGASVKVSCKASGYAFTKYNFYWVRQAPGQGLEWIGYIDPYFGGTVYNQKFKGKATLTVDKSTSTAYMELRSLRSDDTAVYYCATTEGYSLFTYWGQGTLVTVSS

SEQ ID NO: 649 - protein, 118 aa, synthetic construct
QVQLVQSGAEVKKPGASVKVSCKASGYAFTKYNFYWVRQAPGQGLEWIGYIDPYFGGTVYNQKFKGKATLTVDKSTSTAYMELRSLRSDDTAVYYCATTDAYSLFTYWGQGTLVTVSS

SEQ ID NO: 650 - protein, 118 aa, synthetic construct
QVQLVQSGAEVKKPGASVKVSCKASGYAFTKYNFYWVRQAPGQGLEWIGYIDPYFGGTVYNQKFKGKATLTVDKSTSTAYMELRSLRSDDTAVYYCATTDSYSLFTYWGQGTLVTVSS

SEQ ID NO: 651 - protein, 118 aa, synthetic construct
QVQLVQSGAEVKKPGASVKVSCKASGYAFTKYNFYWVRQAPGQGLEWIGYIDPYFGGTVYNQKFKGRVTMTTDTSTSTAYMELRSLRSDDTAVYYCATTDSYSLFTYWGQGTLVTVSS

SEQ ID NO: 652 - protein, 118 aa, synthetic construct
QVQLVQSGAEVKKPGASVKVSCKASGYAFTKYNFYWVRQAPGQGLEWIGYIDPYFGGTVYNQKFKGRVTMTTDTSTSTAYMELRSLRSDDTAVYYCATTDGYSLFTYWGQGTLVTVSS

SEQ ID NO: 653 - protein, 107 aa, synthetic construct
DIQMTQSPSSLSASVGDRVTITCRASQDISNYLNWYQQKPGKAVKLLIYYTSRLHSGVPSRFSGSGSGTDYTLTISSLQPEDIATYFCQQGNTLPFTFGQGTKLEIK

SEQ ID NO: 654 - protein, 107 aa, synthetic construct
DIQMTQSPSSLSASVGDRVTITCRASQDISNYLNWYQQTPGKAVKLLIYYTSRLHSGVPSRFSGSGSGTDYTLTISSLQPEDIATYFCLQGNTLPFTFGQGTKLEIK

SEQ ID NO: 655 - protein, 107 aa, synthetic construct
DIQMTQSPSSLSASVGDRVTITCRASQDISNYLNWYQQKPGKAVKLLIYYTSRLHSGVPSRFSGSGSGTDYTLTISSLQPEDIATYFCLQGNTLPFTFGQGTKLEIK

SEQ ID NO: 656 - protein, 107 aa, synthetic construct
DIQMTQSPSSLSASVGDRVTITCRASQDISNYLNWYQQTPGKAVKLLIYYTSRLHSGVPSRFSGSGSGTDYTLTISSLQPEDIATYYCLQGNTLPFTFGQGTKLEIK

SEQ ID NO: 657 - protein, 107 aa, synthetic construct
DIQMTQSPSSLSASVGDRVTITCRASQDISNYLNWYQQKPGKAVKLLIYYTSRLHSGVPSRFSGSGSGTDFTFTISSLQPEDIATYYCLQGNTLPFTFGQGTKLEIK

SEQ ID NO: 658 - protein, 107 aa, synthetic construct
DIQMTQSPSSLSASVGDRVTITCRASQDISNYLNWYQQTPGKAVKLLIYYTSRLHSGVPSRFSGSGSGTDFTFTISSLQPEDIATYYCLQGNTLPFTFGQGTKLEIK

SEQ ID NO: 659 - protein, 117 aa, synthetic construct
QVQLVQSGAEVKKPGSSVKVSCKASGYTFSHHSISWVRQAPGQGLEWIGGIYPGKIKTNYAQKFQGRATLTADKSTSTAYMELSSLRSEDTAVYYCATLYGNYEAYWGQGTLVTVSS

SEQ ID NO: 660 - protein, 108 aa, synthetic construct
DIQMTQSPSSLSASVGDRVTITCRASENIYGALNWYQQKPGKSPKLLIYGTTSLQSGVPSRFSGSGSGRDYTLTISSLQPEDFATYYCQNVLTTPPWTFGGGTKVEIK

SEQ ID NO: 661 - protein, 117 aa, synthetic construct
QVQLVQSGAEVKKPGSSVKVSCKASGYAFHDYLIEWVRQAPGQGLEWIGGINPGSGSTNYAQKFQGRATLTADKSTSTAYMELSSLRSEDTAVYYCARLDGNYVGYWGQGTLVTVSS

SEQ ID NO: 662 - protein, 117 aa, synthetic construct
QVQLVQSGAEVKKPGSSVKVSCKASGYAFVDYLIEWVRQAPGQGLEWIGGVNPGSGSTNYAQKFQGRATLTADKSTSTAYMELSSLRSEDTAVYYCARLEGNYVGYWGQGTLVTVSS

SEQ ID NO: 663 - protein, 117 aa, synthetic construct
QVQLVQSGAEVKKPGSSVKVSCKASGYAFTDYLIEWVRQAPGQGLEWIGGINPGSGSTNYAQKFQGRATLTADKSTSTAYMELSSLRSEDTAVYYCARLEGNYVGYWGQGTLATVSS

SEQ ID NO: 664 - protein, 117 aa, synthetic construct
QVQLVQSGAEVKKPRSSVKVSCKASGYAFTDYLIEWVRQAPGQGLEWIGGINPGSGSTNYAQKFQGRATLTADKSTSTAYMELSSLRSEDTAVYYCARLEGNYVGYWGQGTLVTVSS

SEQ ID NO: 665 - protein, 117 aa, synthetic construct
QVQLVQSGAEVKKPGSSVKVSCKASGYAFTDYLIEWVRQAPGQGLEWIGGINPGSGSTNYAQKFQGRATLTADKSTSTAYMELSSLRSEDTAVYYCARLEGNYVGYWGQGTLVTVSS

SEQ ID NO: 666 - protein, 117 aa, synthetic construct
QVQLVQSGAEVKKPGSSVKVSCKASGYAFTDYLIEWFRQTPGQGLEWIGGINPGSGSTNYAQKFQGRATLTADKSTSTAYMELSSLRSEDTAVYYCARLEGNYVGYWGQGTLVTVSS

SEQ ID NO: 667 - protein, 117 aa, synthetic construct
QVQLVQSGAEVKKPGSSVKVSCKASGYAFTDYLIEWFRQTPGQGLEWIGGINPGSGSTNYAQKFQGRATLTADKSTSTAYMELSSLRSEDTAVYYCARLEGQYVGYWGQGTLVTVSS

SEQ ID NO: 668 - protein, 117 aa, synthetic construct
QVQLVQSGAEVKKPGSSVKVSCKASGYAFTDYLIEWFRQTPGQGLEWIGGINPGSGSTNYAQKFQGRATLTADKSTSTAYMELSSLRSEDTAVYYCARLEGNFVGYWGQGTLVTVSS

SEQ ID NO: 669 - protein, 117 aa, synthetic construct
QVQLVQSGAEVKKPGSSVKVSCKASGYAFTDYLIEWFRQTPGQGLEWIGGINPGSGSTNYAQKFQGRATLTADKSTSTAYMELSSLRSEDTAVYYCARLEGNFVSYWGQGTLVTVSS

SEQ ID NO: 670 - protein, 117 aa, synthetic construct
QVQLVQSGAEVKKPGSSVKVSCKASGYAFTDYLIEWFRQAPGQGLEWIGGINPGSGSTNYAQKFQGRATLTADKSTSTAYMELSSLRSEDTAVYYCARLEGNYVGYWGQGTLVTVSS

SEQ ID NO: 671 - protein, 117 aa, synthetic construct
QVQLVQSGAEVKKPGSSVKVSCKASGYAFTDYLIEWVRQAPGQGLEWIGGINPGSGSTNYAQKFQGRATLTADKSTSTAYMELSSLRSEDTAVYYCARLEGNYVAYWGQGTLVTVSS

SEQ ID NO: 672 - protein, 117 aa, synthetic construct
QVQLVQSGAEIKKPGSSVKVSCKASGYAFTDYLIEWVRQAPGQGLEWIGGINPGSGSTNYAEKFQGRVTLTADKSTSTAYMELSSLRSEDTAVYYCARLEGNYVSYWGQGTLVTVSS

SEQ ID NO: 673 - protein, 117 aa, synthetic construct
QVQLVQSGAEVKKPGSSVKVSCKASGYAFTDYLIEWVRQAPGQGLEWIGGINPGSGSTNYAQKFQGRATLTADKSTSTAYMELSSLRSEDTAVYYCARLEGNYVSYWGQGTLVTVSS

SEQ ID NO: 674 - protein, 117 aa, synthetic construct
QVQLVQSGAEVKKPGSSVKVSCKASGYAFTDYLIEWVRQAPGQGLEWIGGINPGSGSTNYNEKFKGRATLTADKSSSTAYMELSSLRSDDTAVYFCARLEGNYVGYWGQGTLVTVSS

SEQ ID NO: 675 - protein, 117 aa, synthetic construct
QVQSVQSGAEVKKPGSSVKVSCKASGYAFTDYLIEWVRQAPGQGLEWIGGINPGSGSTNYNEKFKGRVTLTADKSSSTAYMELSSLRSDDTAVYFCARLEGNYVRYWGQGTLVTVSS

SEQ ID NO: 676 - protein, 117 aa, synthetic construct
QVQLVQSGAEVKKPGSSVKVSCKASGYAFTDYLIEWFRQAPGQGLEWIGGINPGSGSTNYNEKFKGRATLTADKSSSTAYMELLSLRSDDTAVYFCARLEGNYVRYWGQGTLVTVSS

SEQ ID NO: 677 - protein, 117 aa, synthetic construct
QVQLVQSGAEVKKPGSSVKVSCKASGYAFTDYLIEWLRQAPGQGLEWIGGINPSSGSTNYNEKFKGRATLTADKSSSTAYMELSSLRSDDTAVYFCARLEGNYVRYWGHGTLVTVSS

SEQ ID NO: 678 - protein, 117 aa, synthetic construct
QVQLVQSGAEVKKPGSSVKVSCKASGYAFTDYLIEWVRQTPGQGLEWIGGINPGSGSTNYNEKFKGRATLTADKSSSTAYMELLSLRSDDTAVYFCARLEGNYVRYWGQGTLVTVSS

SEQ ID NO: 679 - protein, 117 aa, synthetic construct
QVQLVQSGAEVKKPGSSVKVSCKASGYAFTDYLIEWVRQAPGQGLEWIGGINPGSGSTNYAQKFQGRATLTADKSTSTAYMELSSLRSEDTAVYYCARLEGNYVGYWGQGTPVTVSS

SEQ ID NO: 680 - protein, 117 aa, synthetic construct
QVQLVQSGAEVKKPGSSVKVSCKASGYAFTDYLIEWVRQTPGQGLEWIGGINPGSGSTNYAQKFQGRATLTADKSTSTAYMELSSPRSEDTAVYYCARLEGNYVGYWGQGTLVTVSS

SEQ ID NO: 681 - protein, 107 aa, synthetic construct
DIQMTQSPSSLSASVGDRVTITCRASGNIHNYLAWYQQKPGKSPKLLVYHAKTLQSGVPSRFSGSGSGTDYTLTISSLQPEDVATYYCQHFWSTPWTSGGGTKLEIK

SEQ ID NO: 682 - protein, 107 aa, synthetic construct
DIQLTQSPSSLSASVGDRVTITCRASGNIHNYLAWYQQKPGKAPKLLVYHAKTLETGVPSRFSGSGSGTDYTLTISSLQPEDIGSYYCQHFWSTPWTFGQGTKLEIK

SEQ ID NO: 683 - protein, 107 aa, synthetic construct
DIQLTQSPSSLSASVGDRVTITCRASGNIHNYLAWYQQKPGKAPKLLVYHAKTLDTGVPSRFSGSGSGTDYTLTISSLQPEDIGSYYCQHFWSTPWTFGQGTKLEIK

SEQ ID NO: 684 - protein, 107 aa, synthetic construct
DIQLTQSPSSLSASVGDRVTITCRASGNIHKYLAWYQQKPGKAPKLLVYHAKTLETGVPSRFSGSGSGTDYTLTISSLQPEDIGSYYCQHFWSTPWTFGQGTKLEIK

SEQ ID NO: 685 - protein, 107 aa, synthetic construct
DIQMTQSPSSLSASVGDRVTITCRASGNIHNYLAWYQQKPGKSPKLLVYHAKTLQSGVPSRFSGSGSGTDYTLTISSLQPEDVATYYCQHFWSTPRTFGGGTKVEIK

SEQ ID NO: 686 - protein, 107 aa, synthetic construct
DIQMTQSPSSLSASVGDRVTITCRASGNIHNYLAWYQQKPGKSPKLLVYHAKTLQSGVPSRFSGSGSGTDYTLTISSLQPEDVATYYCQHFWSTPWTSGGGTKVEIK

SEQ ID NO: 687 - protein, 107 aa, synthetic construct
DIQMTQSPSSLSASVGDRVTITCRASGNIHNYLAWYQQKPGKSPKLLVYHAKTLQSGVPSRFSGSGSGTDYTLTISSLQPEDVATYYCQHFWSTPWTFGGGTKVEIK

SEQ ID NO: 688 - protein, 107 aa, synthetic construct
DIQMTQSPSSLSASVGDRVTITCRASGNIHNYLAWYQQKPGKSPKLLVYHAKTLQSGIPSRFSGSGSGTDYTLTISSLQPEDVATYYCQHFWSTPWTSGGGTKVEIK

SEQ ID NO: 689 - protein, 107 aa, synthetic construct
DIQMTQSPSSLSASVGDRVTITCRASGNIHNYLAWYQQKPGKSPKLLVYHAKTLQSGVPSRFSGSGSGTDYTLTISSLQPEDVATYYCQHFWSTPWTFGGGTEVEIK

SEQ ID NO: 690 - protein, 118 aa, synthetic construct
EVQLLQSGPELEKPGASVMISCKASGSSFTKYNIYWVRQNIGKSLEWIGYIDPYNGGTVYNQKFKGRATLTVDKSSSTAYMHLKSLTSEDSAVYYCVSTDGYSLFTYWGQGTSVTVSS

SEQ ID NO: 691 - protein, 107 aa, synthetic construct
EIVMTQSPATLSVSPGERATLSCRASQDISNYLNWYLQKPGQSPKLLIHYTSRLHSGVPDRFSGSGSGTDFTLKISRVEAEDLGVYFCQQGNTLPFTFGAGTKLELK

SEQ ID NO: 692 - protein, 118 aa, synthetic construct
EVQLVESGGGLVQPGGSLRLSCAASGYDFTKYNIYWVRQAPGKGLEWVGYIDPYNGGTVYNQKFKGRFTFSLDTSKSTAYLQMNSLRAEDTAVYYCAKTDGYSLFTYWGQGTLVTVSS

SEQ ID NO: 693 - protein, 107 aa, synthetic construct
DIQLTQSPSSLSASVGDRVTITCRASQDISNYLNWYQQKPGKAPKVLIYYTSRLHSGVPSRFSGSGSGTDFTLTISSLQPEDFATYYCQQGNTLPFTFGQGTKVEIK

SEQ ID NO: 694 - protein, 118 aa, synthetic construct
QVQLVQSGGGLVQPGGSLRLSCAASGFTFSKYNIYWVRQAPGKGLEWVSYIDPYNGGTVYNQKFKGRFTISRDNAKNTVYLQLNSLRAEDTAMYYCAKTDGYSLFTYWGQGTLVTVSS

SEQ ID NO: 695 - protein, 107 aa, synthetic construct
DIQLTQSPSSVSVSVGERVTIDCRASQDISNYLNWYQQKPGQAPQLLIYYTSRLHSGVPDRFSGSGSGTDFTLTISSLQAEDAAVYYCQQGNTLPFTFGQGTKLEIK

SEQ ID NO: 696 - protein, 118 aa, synthetic construct
EVQLVESGGGVIQPGGSLRLSCAASGFTFDKYNIYWVRQGPGKGLEWVSYIDPYNGGTVYNQKFKGRFTISRDNSKNSLYLQMNSLRAEDTAFFYCAKTDGYSLFTYWGQGTMVTVSS

SEQ ID NO: 697 - protein, 107 aa, synthetic construct
DIQMTQSPSTLSASVGDRVTITCRASQDISNYLNWYQQKPGKAPKLLIYYTSRLHSGVPSRFSGSGSGTEFTLTISSLQPDDFATYYCQQGNTLPFTFGQGTKLEIK

SEQ ID NO: 698 - protein, 118 aa, synthetic construct
EVQLLQSGPELEKPGASVMISCKASGSSFTKYNIYWVRQNIGKSLEWIGYIDPYFGGTVYNQKFKGRATLTVDKSSSTAYMHLKSLTSEDSAVYYCVSTDGYSLFTYWGQGTSVTVSS

SEQ ID NO: 699 - protein, 107 aa, synthetic construct
EIVMTQSPATLSVSPGERATLSCRASQDISNYLNWYLQKPGQSPKLLIHYTSRLHSGVPDRFSGSGSGTDFTLKISRVEAEDLGVYFCLQGNTLPFTFGAGTKLELK

SEQ ID NO: 700 - protein, 118 aa, synthetic construct
QVQLVQSGGGLVQPGGSLRLSCAASGFTFSKYNIYWVRQAPGKGLEWVSYIDPYFGGTVYNQKFKGRFTISRDNAKNTVYLQLNSLRAEDTAMYYCAKTDGYSLFTYWGQGTLVTVSS

SEQ ID NO: 701 - protein, 107 aa, synthetic construct
DIQLTQSPSSVSVSVGERVTIDCRASQDISNYLNWYQQKPGQAPQLLIYYTSRLHSGVPDRFSGSGSGTDFTLTISSLQAEDAAVYYCLQGNTLPFTFGQGTKLEIK

SEQ ID NO: 702 - protein, 118 aa, synthetic construct
QVQLQQSGAELVKPGASVKLSCKASGYTFTKYNIYWVRQRPEQGLEWIGYIDPYNGGTVYNQKFKGKATLTTDKSSSTAYMQLSRLTSEDSAVYFCARTDGYSLFTYWGQGTTLTVSS

SEQ ID NO: 703 - protein, 107 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGGGTKLEIK

SEQ ID NO: 704 - protein, 118 aa, synthetic construct
EVQLVESGGGLVQPGRSLRLSCAASRFTFDKYNIYWVRQAPGKGLEWVSYIDPYNGGTVYNQKFKGRFTISRDNAENSLFLQMNGLRAEDTALYYCAKTDGYSLFTYWGQGTMVTVSS

SEQ ID NO: 705 - protein, 107 aa, synthetic construct
DIQMTQSPSSVSASVGDRVTITCRASQDISNYLNWYQQKPGKAPKLLIYYTSRLHSGVPSRFSGSGSGTDFTLTISSLQPEDFASYYCQQGNTLPFTFGQGTKLEIK

SEQ ID NO: 706 - protein, 118 aa, synthetic construct
EVQLLESGGVLVQPGGSLRLSCAASGFTFSKYNIYWVRQAPGKGLEWVSYIDPYNGGTVYNQKFKGRFTISRDNSKNTLYLQMNSLKGEDTAVYYCVKTDGYSLFTYWGQGTLVTVSS

SEQ ID NO: 707 - protein, 107 aa, synthetic construct
DIQMTQSPSSLSASVGDSITITCRASQDISNYLNWYQQKPGKAPNLLIYYTSRLHSGVPSRFSGSGSGTDFTLTIRTLQPEDFATYYCQQGNTLPFTFGPGTVVDFR

SEQ ID NO: 708 - protein, 13 aa, synthetic construct
KASGYAFTKYNIY

SEQ ID NO: 709 - protein, 17 aa, synthetic construct
YIDPYNGGTVYNQKFKG

SEQ ID NO: 710 - protein, 10 aa, synthetic construct
TTDGYSLFTY

SEQ ID NO: 711 - protein, 11 aa, synthetic construct
RASQDISNYLN

SEQ ID NO: 712 - protein, 6 aa, synthetic construct
YTSRLH

SEQ ID NO: 713 - protein, 9 aa, synthetic construct
QQGNTLPFT

SEQ ID NO: 714 - protein, 13 aa, synthetic construct
KASGYAFTKYNFY

SEQ ID NO: 715 - protein, 17 aa, synthetic construct
YIDPYFGGTVYNQKFKG

SEQ ID NO: 716 - protein, 10 aa, synthetic construct
TTDGYSLFTY

SEQ ID NO: 717 - protein, 11 aa, synthetic construct
RASQDISNYLN

SEQ ID NO: 718 - protein, 6 aa, synthetic construct
YTSRLH

SEQ ID NO: 719 - protein, 9 aa, synthetic construct
LQGNTLPFT

SEQ ID NO: 720 - protein, 13 aa, synthetic construct
KASGYAFTKYNFY

SEQ ID NO: 721 - protein, 17 aa, synthetic construct
YIDPYFGGTVYNQKFKG

SEQ ID NO: 722 - protein, 10 aa, synthetic construct
TTDSYSLFTY

SEQ ID NO: 723 - protein, 11 aa, synthetic construct
RASQDISNYLN

SEQ ID NO: 724 - protein, 6 aa, synthetic construct
YTSRLH

SEQ ID NO: 725 - protein, 9 aa, synthetic construct
LQGNTLPFT

SEQ ID NO: 726 - protein, 13 aa, synthetic construct
KASGYAFTKYNFY

SEQ ID NO: 727 - protein, 17 aa, synthetic construct
YIDPYFGGTVYNQKFKG

SEQ ID NO: 728 - protein, 10 aa, synthetic construct
TTEGYSLFTY

SEQ ID NO: 729 - protein, 11 aa, synthetic construct
RASQDISNYLN

SEQ ID NO: 730 - protein, 6 aa, synthetic construct
YTSRLH

SEQ ID NO: 731 - protein, 9 aa, synthetic construct
LQGNTLPFT

SEQ ID NO: 732 - protein, 13 aa, synthetic construct
KASGYAFTKYNFY

SEQ ID NO: 733 - protein, 17 aa, synthetic construct
YIDPYFGGTVYNQKFKG

SEQ ID NO: 734 - protein, 10 aa, synthetic construct
TTDAYSLFTY

SEQ ID NO: 735 - protein, 11 aa, synthetic construct
RASQDISNYLN

SEQ ID NO: 736 - protein, 6 aa, synthetic construct
YTSRLH

SEQ ID NO: 737 - protein, 9 aa, synthetic construct
LQGNTLPFT

SEQ ID NO: 738 - protein, 13 aa, synthetic construct
KASGYAFTKYNIY

SEQ ID NO: 739 - protein, 17 aa, synthetic construct
YIDPYFGGTVYNHKFKG

SEQ ID NO: 740 - protein, 10 aa, synthetic construct
TTDGYSLFTY

SEQ ID NO: 741 - protein, 11 aa, synthetic construct
RASQDISNYLN

SEQ ID NO: 742 - protein, 6 aa, synthetic construct
YTSRLH

SEQ ID NO: 743 - protein, 9 aa, synthetic construct
QQGNTLPFT

SEQ ID NO: 744 - protein, 13 aa, synthetic construct
KASGYAFTKYNIY

SEQ ID NO: 745 - protein, 17 aa, synthetic construct
YIDPYFGGTVYNQKFKG

SEQ ID NO: 746 - protein, 10 aa, synthetic construct
TTDGYSLFTY

SEQ ID NO: 747 - protein, 11 aa, synthetic construct
RASQDISNYLN

SEQ ID NO: 748 - protein, 6 aa, synthetic construct
YTSRLH

SEQ ID NO: 749 - protein, 9 aa, synthetic construct
QQGNTLPFT

SEQ ID NO: 750 - protein, 8 aa, synthetic construct
GYTFSHHS

SEQ ID NO: 751 - protein, 8 aa, synthetic construct
IYPGKIKT

SEQ ID NO: 752 - protein, 10 aa, synthetic construct
ATLYGNYEAY

SEQ ID NO: 753 - protein, 6 aa, synthetic construct
ENIYGA

SEQ ID NO: 754 - skipped

SEQ ID NO: 755 - protein, 10 aa, synthetic construct
QNVLTTPPWT

SEQ ID NO: 756 - protein, 8 aa, synthetic construct
GYAFHDYL

SEQ ID NO: 757 - protein, 8 aa, synthetic construct
INPGSGST

SEQ ID NO: 758 - protein, 10 aa, synthetic construct
ARLDGNYVGY

SEQ ID NO: 759 - protein, 6 aa, synthetic construct
GNIHNY

SEQ ID NO: 760 - skipped

SEQ ID NO: 761 - protein, 9 aa, synthetic construct
QHFWSTPRT

SEQ ID NO: 762 - protein, 8 aa, synthetic construct
GYAFTDYL

SEQ ID NO: 763 - protein, 8 aa, synthetic construct
INPGSGST

SEQ ID NO: 764 - protein, 10 aa, synthetic construct
ARLEGNYVGY

SEQ ID NO: 765 - protein, 6 aa, synthetic construct
GNIHNY

SEQ ID NO: 766 - skipped

SEQ ID NO: 767 - protein, 9 aa, synthetic construct
QHFWSTPWT

SEQ ID NO: 768 - protein, 8 aa, synthetic construct
GYAFVDYL

SEQ ID NO: 769 - protein, 8 aa, synthetic construct
VNPGSGST

SEQ ID NO: 770 - protein, 10 aa, synthetic construct
ARLEGNYVGY

SEQ ID NO: 771 - protein, 6 aa, synthetic construct
GNIHNY

SEQ ID NO: 772 - skipped

SEQ ID NO: 773 - protein, 9 aa, synthetic construct
QHFWSTPRT

SEQ ID NO: 774 - protein, 8 aa, synthetic construct
GYAFTDYL

SEQ ID NO: 775 - protein, 8 aa, synthetic construct
INPGSGST

SEQ ID NO: 776 - protein, 10 aa, synthetic construct
ARLEGNYVRY

SEQ ID NO: 777 - protein, 6 aa, synthetic construct
GNIHNY

SEQ ID NO: 778 - skipped

SEQ ID NO: 779 - protein, 9 aa, synthetic construct
QHFWSTPWT

SEQ ID NO: 780 - protein, 8 aa, synthetic construct
GYAFTDYL

SEQ ID NO: 781 - protein, 8 aa, synthetic construct
INPSSGST

SEQ ID NO: 782 - protein, 10 aa, synthetic construct
ARLEGNYVRY

SEQ ID NO: 783 - protein, 6 aa, synthetic construct
GNIHKY

SEQ ID NO: 784 - skipped

SEQ ID NO: 785 - protein, 9 aa, synthetic construct
QHFWSTPWT

SEQ ID NO: 786 - protein, 8 aa, synthetic construct
GYAFTDYL

SEQ ID NO: 787 - protein, 8 aa, synthetic construct
INPGSGST

SEQ ID NO: 788 - protein, 10 aa, synthetic construct
ARLEGNYVSY

SEQ ID NO: 789 - protein, 6 aa, synthetic construct
GNIHNY

SEQ ID NO: 790 - skipped

SEQ ID NO: 791 - protein, 9 aa, synthetic construct
QHFWSTPWT

SEQ ID NO: 792 - protein, 8 aa, synthetic construct
GYAFTDYL

SEQ ID NO: 793 - protein, 8 aa, synthetic construct
INPGSGST

SEQ ID NO: 794 - protein, 10 aa, synthetic construct
ARLEGQYVGY

SEQ ID NO: 795 - protein, 6 aa, synthetic construct
GNIHNY

SEQ ID NO: 796 - skipped

SEQ ID NO: 797 - protein, 9 aa, synthetic construct
QHFWSTPWT

SEQ ID NO: 798 - protein, 8 aa, synthetic construct
GYAFTDYL

SEQ ID NO: 799 - protein, 8 aa, synthetic construct
INPGSGST

SEQ ID NO: 800 - protein, 10 aa, synthetic construct
ARLEGNFVSY

SEQ ID NO: 801 - protein, 6 aa, synthetic construct
GNIHNY

SEQ ID NO: 802 - skipped

SEQ ID NO: 803 - protein, 9 aa, synthetic construct
QHFWSTPWT

SEQ ID NO: 804 - protein, 8 aa, synthetic construct
GYAFTDYL

SEQ ID NO: 805 - protein, 8 aa, synthetic construct
INPGSGST

SEQ ID NO: 806 - protein, 10 aa, synthetic construct
ARLEGNYVAY

SEQ ID NO: 807 - protein, 6 aa, synthetic construct
GNIHNY

SEQ ID NO: 808 - skipped

SEQ ID NO: 809 - protein, 9 aa, synthetic construct
QHFWSTPWT

SEQ ID NO: 810 - protein, 8 aa, synthetic construct
GYAFTDYL

SEQ ID NO: 811 - protein, 8 aa, synthetic construct
INPGSGST

SEQ ID NO: 812 - protein, 10 aa, synthetic construct
ARLEGNFVGY

SEQ ID NO: 813 - protein, 6 aa, synthetic construct
GNIHNY

SEQ ID NO: 814 - skipped

SEQ ID NO: 815 - protein, 9 aa, synthetic construct
QHFWSTPWT

SEQ ID NO: 816 - protein, 8 aa, synthetic construct
IDPYNGGT

SEQ ID NO: 817 - protein, 11 aa, synthetic construct
ATTDGYSLFTY

SEQ ID NO: 818 - protein, 6 aa, synthetic construct
QDISNY

SEQ ID NO: 819 - skipped

SEQ ID NO: 820 - protein, 9 aa, synthetic construct
QQGNTLPFT

SEQ ID NO: 821 - protein, 8 aa, synthetic construct
GYAFTKYN

SEQ ID NO: 822 - protein, 8 aa, synthetic construct
IDPYFGGT

SEQ ID NO: 823 - protein, 11 aa, synthetic construct
ATTDGYSLFTY

SEQ ID NO: 824 - protein, 6 aa, synthetic construct
QDISNY

SEQ ID NO: 825 - skipped

SEQ ID NO: 826 - protein, 9 aa, synthetic construct
LQGNTLPFT

SEQ ID NO: 827 - protein, 8 aa, synthetic construct
GYAFTKYN

SEQ ID NO: 828 - protein, 8 aa, synthetic construct
IDPYFGGT

SEQ ID NO: 829 - protein, 11 aa, synthetic construct
ATTDSYSLFTY

SEQ ID NO: 830 - protein, 6 aa, synthetic construct
QDISNY

SEQ ID NO: 831 - skipped

SEQ ID NO: 832 - protein, 9 aa, synthetic construct
LQGNTLPFT

SEQ ID NO: 833 - protein, 8 aa, synthetic construct
GYAFTKYN

SEQ ID NO: 834 - protein, 8 aa, synthetic construct
IDPYFGGT

SEQ ID NO: 835 - protein, 11 aa, synthetic construct
ATTEGYSLFTY

SEQ ID NO: 836 - protein, 6 aa, synthetic construct
QDISNY

SEQ ID NO: 837 - skipped

SEQ ID NO: 838 - protein, 9 aa, synthetic construct
LQGNTLPFT

SEQ ID NO: 839 - protein, 8 aa, synthetic construct
GYAFTKYN

SEQ ID NO: 840 - protein, 8 aa, synthetic construct
IDPYFGGT

SEQ ID NO: 841 - protein, 11 aa, synthetic construct
ATTDAYSLFTY

SEQ ID NO: 842 - protein, 6 aa, synthetic construct
QDISNY

SEQ ID NO: 843 - skipped

SEQ ID NO: 844 - protein, 9 aa, synthetic construct
LQGNTLPFT

SEQ ID NO: 845 - protein, 8 aa, synthetic construct
GYAFTKYN

SEQ ID NO: 846 - protein, 8 aa, synthetic construct
IDPYFGGT

SEQ ID NO: 847 - protein, 11 aa, synthetic construct
ATTDGYSLFTY

SEQ ID NO: 848 - protein, 6 aa, synthetic construct
QDISNY

SEQ ID NO: 849 - skipped

SEQ ID NO: 850 - protein, 9 aa, synthetic construct
QQGNTLPFT

SEQ ID NO: 851 - protein, 8 aa, synthetic construct
GYAFTKYN

SEQ ID NO: 852 - protein, 8 aa, synthetic construct
IDPYFGGT

SEQ ID NO: 853 - protein, 11 aa, synthetic construct
ATTDGYSLFTY

SEQ ID NO: 854 - protein, 6 aa, synthetic construct
QDISNY

SEQ ID NO: 855 - skipped

SEQ ID NO: 856 - protein, 9 aa, synthetic construct
QQGNTLPFT

SEQ ID NO: 857 - protein, 8 aa, synthetic construct
GYAFTKYN

SEQ ID NO: 858 - protein, 8 aa, synthetic construct
IDPYFGGT

SEQ ID NO: 859 - protein, 11 aa, synthetic construct
ATTDSYSLFTY

SEQ ID NO: 860 - protein, 6 aa, synthetic construct
QDISNY

SEQ ID NO: 861 - skipped

SEQ ID NO: 862 - protein, 9 aa, synthetic construct
LQGNTLPFT

SEQ ID NO: 863 - protein, 8 aa, synthetic construct
GYAFTKYN

SEQ ID NO: 864 - protein, 8 aa, synthetic construct
IDPYFGGT

SEQ ID NO: 865 - protein, 11 aa, synthetic construct
ATTDSYSLFTY

SEQ ID NO: 866 - protein, 6 aa, synthetic construct
QDISNY

SEQ ID NO: 867 - skipped

SEQ ID NO: 868 - protein, 9 aa, synthetic construct
LQGNTLPFT

SEQ ID NO: 869 - protein, 8 aa, synthetic construct
GYAFTKYN

SEQ ID NO: 870 - protein, 8 aa, synthetic construct
IDPYFGGT

SEQ ID NO: 871 - protein, 11 aa, synthetic construct
ATTDSYSLFTY

SEQ ID NO: 872 - protein, 6 aa, synthetic construct
QDISNY

SEQ ID NO: 873 - skipped

SEQ ID NO: 874 - protein, 9 aa, synthetic construct
LQGNTLPFT

SEQ ID NO: 875 - protein, 8 aa, synthetic construct
GYAFTKYN

SEQ ID NO: 876 - protein, 8 aa, synthetic construct
IDPYFGGT

SEQ ID NO: 877 - protein, 11 aa, synthetic construct
ATTDSYSLFTY

SEQ ID NO: 878 - protein, 6 aa, synthetic construct
QDISNY

SEQ ID NO: 879 - skipped

SEQ ID NO: 880 - protein, 9 aa, synthetic construct
LQGNTLPFT

SEQ ID NO: 881 - protein, 8 aa, synthetic construct
GYAFTKYN

SEQ ID NO: 882 - protein, 8 aa, synthetic construct
IDPYFGGT

SEQ ID NO: 883 - protein, 11 aa, synthetic construct
ATTDGYSLFTY

SEQ ID NO: 884 - protein, 6 aa, synthetic construct
QDISNY

SEQ ID NO: 885 - skipped

SEQ ID NO: 886 - protein, 9 aa, synthetic construct
LQGNTLPFT

SEQ ID NO: 887 - protein, 8 aa, synthetic construct
GYAFTKYN

SEQ ID NO: 888 - protein, 8 aa, synthetic construct
IDPYFGGT

SEQ ID NO: 889 - protein, 11 aa, synthetic construct
ATTDGYSLFTY

SEQ ID NO: 890 - protein, 6 aa, synthetic construct
QDISNY

SEQ ID NO: 891 - skipped

SEQ ID NO: 892 - protein, 9 aa, synthetic construct
LQGNTLPFT

SEQ ID NO: 893 - protein, 8 aa, synthetic construct
GYAFTKYN

SEQ ID NO: 894 - protein, 8 aa, synthetic construct
IDPYFGGT

SEQ ID NO: 895 - protein, 11 aa, synthetic construct
ATTDSYSLFTY

SEQ ID NO: 896 - protein, 6 aa, synthetic construct
QDISNY

SEQ ID NO: 897 - skipped

SEQ ID NO: 898 - protein, 9 aa, synthetic construct
LQGNTLPFT

SEQ ID NO: 899 - protein, 8 aa, synthetic construct
GYAFTKYN

SEQ ID NO: 900 - protein, 8 aa, synthetic construct
IDPYFGGT

SEQ ID NO: 901 - protein, 11 aa, synthetic construct
ATTDSYSLFTY

SEQ ID NO: 902 - protein, 6 aa, synthetic construct
QDISNY

SEQ ID NO: 903 - skipped

SEQ ID NO: 904 - protein, 9 aa, synthetic construct
LQGNTLPFT

SEQ ID NO: 905 - protein, 8 aa, synthetic construct
GYAFTKYN

SEQ ID NO: 906 - protein, 8 aa, synthetic construct
IDPYFGGT

SEQ ID NO: 907 - protein, 11 aa, synthetic construct
ATTDGYSLFTY

SEQ ID NO: 908 - protein, 6 aa, synthetic construct
QDISNY

SEQ ID NO: 909 - skipped

SEQ ID NO: 910 - protein, 9 aa, synthetic construct
LQGNTLPFT

SEQ ID NO: 911 - protein, 8 aa, synthetic construct
GYAFTKYN

SEQ ID NO: 912 - protein, 8 aa, synthetic construct
IDPYFGGT

SEQ ID NO: 913 - protein, 11 aa, synthetic construct
ATTDGYSLFTY

SEQ ID NO: 914 - protein, 6 aa, synthetic construct
QDISNY

SEQ ID NO: 915 - skipped

SEQ ID NO: 916 - protein, 9 aa, synthetic construct
LQGNTLPFT

SEQ ID NO: 917 - protein, 8 aa, synthetic construct
GYAFTKYN

SEQ ID NO: 918 - protein, 8 aa, synthetic construct
IDPYFGGT

SEQ ID NO: 919 - protein, 11 aa, synthetic construct
ATTDSYSLFTY

SEQ ID NO: 920 - protein, 6 aa, synthetic construct
QDISNY

SEQ ID NO: 921 - skipped

SEQ ID NO: 922 - protein, 9 aa, synthetic construct
LQGNTLPFT

SEQ ID NO: 923 - protein, 8 aa, synthetic construct
GYAFTKYN

SEQ ID NO: 924 - protein, 8 aa, synthetic construct
IDPYFGGT

SEQ ID NO: 925 - protein, 11 aa, synthetic construct
ATTDGYSLFTY

SEQ ID NO: 926 - protein, 6 aa, synthetic construct
QDISNY

SEQ ID NO: 927 - skipped

SEQ ID NO: 928 - protein, 9 aa, synthetic construct
LQGNTLPFT

SEQ ID NO: 929 - protein, 8 aa, synthetic construct
GYAFTKYN

SEQ ID NO: 930 - protein, 8 aa, synthetic construct
IDPYFGGT

SEQ ID NO: 931 - protein, 11 aa, synthetic construct
ATTDGYSLFTY

SEQ ID NO: 932 - protein, 6 aa, synthetic construct
QDISNY

SEQ ID NO: 933 - skipped

SEQ ID NO: 934 - protein, 9 aa, synthetic construct
LQGNTLPFT

SEQ ID NO: 935 - protein, 5 aa, synthetic construct
KYNIY

SEQ ID NO: 936 - protein, 17 aa, synthetic construct
YIDPYNGGTVYNQKFKG

SEQ ID NO: 937 - protein, 9 aa, synthetic construct
TDGYSLFTY

SEQ ID NO: 938 - protein, 11 aa, synthetic construct
RASQDISNYLN

SEQ ID NO: 939 - protein, 7 aa, synthetic construct
YTSRLHS

SEQ ID NO: 940 - protein, 9 aa, synthetic construct
QQGNTLPFT

SEQ ID NO: 941 - protein, 5 aa, synthetic construct
KYNFY

SEQ ID NO: 942 - protein, 17 aa, synthetic construct
YIDPYFGGTVYNQKFKG

SEQ ID NO: 943 - protein, 9 aa, synthetic construct
TDGYSLFTY

SEQ ID NO: 944 - protein, 11 aa, synthetic construct
RASQDISNYLN

SEQ ID NO: 945 - protein, 7 aa, synthetic construct
YTSRLHS

SEQ ID NO: 946 - protein, 9 aa, synthetic construct
LQGNTLPFT

SEQ ID NO: 947 - protein, 5 aa, synthetic construct
KYNFY

SEQ ID NO: 948 - protein, 17 aa, synthetic construct
YIDPYFGGTVYNQKFKG

SEQ ID NO: 949 - protein, 9 aa, synthetic construct
TDSYSLFTY

SEQ ID NO: 950 - protein, 11 aa, synthetic construct
RASQDISNYLN

SEQ ID NO: 951 - protein, 7 aa, synthetic construct
YTSRLHS

SEQ ID NO: 952 - protein, 9 aa, synthetic construct
LQGNTLPFT

SEQ ID NO: 953 - protein, 5 aa, synthetic construct
KYNFY

SEQ ID NO: 954 - protein, 17 aa, synthetic construct
YIDPYFGGTVYNQKFKG

SEQ ID NO: 955 - protein, 9 aa, synthetic construct
TEGYSLFTY

SEQ ID NO: 956 - protein, 11 aa, synthetic construct
RASQDISNYLN

SEQ ID NO: 957 - protein, 7 aa, synthetic construct
YTSRLHS

SEQ ID NO: 958 - protein, 9 aa, synthetic construct
LQGNTLPFT

SEQ ID NO: 959 - protein, 5 aa, synthetic construct
KYNFY

SEQ ID NO: 960 - protein, 17 aa, synthetic construct
YIDPYFGGTVYNQKFKG

SEQ ID NO: 961 - protein, 9 aa, synthetic construct
TDAYSLFTY

SEQ ID NO: 962 - protein, 11 aa, synthetic construct
RASQDISNYLN

SEQ ID NO: 963 - protein, 7 aa, synthetic construct
YTSRLHS

SEQ ID NO: 964 - protein, 9 aa, synthetic construct
LQGNTLPFT

SEQ ID NO: 965 - protein, 5 aa, synthetic construct
KYNIY

SEQ ID NO: 966 - protein, 17 aa, synthetic construct
YIDPYFGGTVYNHKFKG

SEQ ID NO: 967 - protein, 9 aa, synthetic construct
TDGYSLFTY

SEQ ID NO: 968 - protein, 11 aa, synthetic construct
RASQDISNYLN

SEQ ID NO: 969 - protein, 7 aa, synthetic construct
YTSRLHS

SEQ ID NO: 970 - protein, 9 aa, synthetic construct
QQGNTLPFT

SEQ ID NO: 971 - protein, 5 aa, synthetic construct
KYNIY

SEQ ID NO: 972 - protein, 17 aa, synthetic construct
YIDPYFGGTVYNQKFKG

SEQ ID NO: 973 - protein, 9 aa, synthetic construct
TDGYSLFTY

SEQ ID NO: 974 - protein, 11 aa, synthetic construct
RASQDISNYLN

SEQ ID NO: 975 - protein, 7 aa, synthetic construct
YTSRLHS

SEQ ID NO: 976 - protein, 9 aa, synthetic construct
QQGNTLPFT

SEQ ID NO: 977 - protein, 5 aa, synthetic construct
KYNFY

SEQ ID NO: 978 - protein, 17 aa, synthetic construct
YIDPYFGGTVYNQKFKG

SEQ ID NO: 979 - protein, 9 aa, synthetic construct
TDSYSLFTY

SEQ ID NO: 980 - protein, 11 aa, synthetic construct
RASQDISNYLN

SEQ ID NO: 981 - protein, 7 aa, synthetic construct
YTSRLHS

SEQ ID NO: 982 - protein, 9 aa, synthetic construct
LQGNTLPFT

SEQ ID NO: 983 - protein, 5 aa, synthetic construct
KYNFY

SEQ ID NO: 984 - protein, 17 aa, synthetic construct
YIDPYFGGTVYNQKFKG

SEQ ID NO: 985 - protein, 9 aa, synthetic construct
TDSYSLFTY

SEQ ID NO: 986 - protein, 11 aa, synthetic construct
RASQDISNYLN

SEQ ID NO: 987 - protein, 7 aa, synthetic construct
YTSRLHS

SEQ ID NO: 988 - protein, 9 aa, synthetic construct
LQGNTLPFT

SEQ ID NO: 989 - protein, 5 aa, synthetic construct
KYNFY

SEQ ID NO: 990 - protein, 17 aa, synthetic construct
YIDPYFGGTVYNQKFKG

SEQ ID NO: 991 - protein, 9 aa, synthetic construct
TDSYSLFTY

SEQ ID NO: 992 - protein, 11 aa, synthetic construct
RASQDISNYLN

SEQ ID NO: 993 - protein, 7 aa, synthetic construct
YTSRLHS

SEQ ID NO: 994 - protein, 9 aa, synthetic construct
LQGNTLPFT

SEQ ID NO: 995 - protein, 5 aa, synthetic construct
KYNFY

SEQ ID NO: 996 - protein, 17 aa, synthetic construct
YIDPYFGGTVYNQKFKG

SEQ ID NO: 997 - protein, 9 aa, synthetic construct
TDSYSLFTY

SEQ ID NO: 998 - protein, 11 aa, synthetic construct
RASQDISNYLN

SEQ ID NO: 999 - protein, 7 aa, synthetic construct
YTSRLHS

SEQ ID NO: 1000 - protein, 9 aa, synthetic construct
LQGNTLPFT

SEQ ID NO: 1001 - protein, 5 aa, synthetic construct
KYNFY

SEQ ID NO: 1002 - protein, 17 aa, synthetic construct
YIDPYFGGTVYNQKFKG

SEQ ID NO: 1003 - protein, 9 aa, synthetic construct
TDGYSLFTY

SEQ ID NO: 1004 - protein, 11 aa, synthetic construct
RASQDISNYLN

SEQ ID NO: 1005 - protein, 7 aa, synthetic construct
YTSRLHS

SEQ ID NO: 1006 - protein, 9 aa, synthetic construct
LQGNTLPFT

SEQ ID NO: 1007 - protein, 5 aa, synthetic construct
KYNFY

SEQ ID NO: 1008 - protein, 17 aa, synthetic construct
YIDPYFGGTVYNQKFKG

SEQ ID NO: 1009 - protein, 9 aa, synthetic construct
TDGYSLFTY

SEQ ID NO: 1010 - protein, 11 aa, synthetic construct
RASQDISNYLN

SEQ ID NO: 1011 - protein, 7 aa, synthetic construct
YTSRLHS

SEQ ID NO: 1012 - protein, 9 aa, synthetic construct
LQGNTLPFT

SEQ ID NO: 1013 - protein, 5 aa, synthetic construct
KYNFY

SEQ ID NO: 1014 - protein, 17 aa, synthetic construct
YIDPYFGGTVYNQKFKG

SEQ ID NO: 1015 - protein, 9 aa, synthetic construct
TDSYSLFTY

SEQ ID NO: 1016 - protein, 11 aa, synthetic construct
RASQDISNYLN

SEQ ID NO: 1017 - protein, 7 aa, synthetic construct
YTSRLHS

SEQ ID NO: 1018 - protein, 9 aa, synthetic construct
LQGNTLPFT

SEQ ID NO: 1019 - protein, 5 aa, synthetic construct
KYNFY

SEQ ID NO: 1020 - protein, 17 aa, synthetic construct
YIDPYFGGTVYNQKFKG

SEQ ID NO: 1021 - protein, 9 aa, synthetic construct
TDSYSLFTY

SEQ ID NO: 1022 - protein, 11 aa, synthetic construct
RASQDISNYLN

SEQ ID NO: 1023 - protein, 7 aa, synthetic construct
YTSRLHS

SEQ ID NO: 1024 - protein, 9 aa, synthetic construct
LQGNTLPFT

SEQ ID NO: 1025 - protein, 5 aa, synthetic construct
KYNFY

SEQ ID NO: 1026 - protein, 17 aa, synthetic construct
YIDPYFGGTVYNQKFKG

SEQ ID NO: 1027 - protein, 9 aa, synthetic construct
TDGYSLFTY

SEQ ID NO: 1028 - protein, 11 aa, synthetic construct
RASQDISNYLN

SEQ ID NO: 1029 - protein, 7 aa, synthetic construct
YTSRLHS

SEQ ID NO: 1030 - protein, 9 aa, synthetic construct
LQGNTLPFT

SEQ ID NO: 1031 - protein, 5 aa, synthetic construct
KYNFY

SEQ ID NO: 1032 - protein, 17 aa, synthetic construct
YIDPYFGGTVYNQKFKG

SEQ ID NO: 1033 - protein, 9 aa, synthetic construct
TDGYSLFTY

SEQ ID NO: 1034 - protein, 11 aa, synthetic construct
RASQDISNYLN

SEQ ID NO: 1035 - protein, 7 aa, synthetic construct
YTSRLHS

SEQ ID NO: 1036 - protein, 9 aa, synthetic construct
LQGNTLPFT

SEQ ID NO: 1037 - protein, 5 aa, synthetic construct
KYNFY

SEQ ID NO: 1038 - protein, 17 aa, synthetic construct
YIDPYFGGTVYNQKFKG

SEQ ID NO: 1039 - protein, 9 aa, synthetic construct
TDSYSLFTY

SEQ ID NO: 1040 - protein, 11 aa, synthetic construct
RASQDISNYLN

SEQ ID NO: 1041 - protein, 7 aa, synthetic construct
YTSRLHS

SEQ ID NO: 1042 - protein, 9 aa, synthetic construct
LQGNTLPFT

SEQ ID NO: 1043 - protein, 5 aa, synthetic construct
KYNFY

SEQ ID NO: 1044 - protein, 17 aa, synthetic construct
YIDPYFGGTVYNQKFKG

SEQ ID NO: 1045 - protein, 9 aa, synthetic construct
TDGYSLFTY

SEQ ID NO: 1046 - protein, 11 aa, synthetic construct
RASQDISNYLN

SEQ ID NO: 1047 - protein, 7 aa, synthetic construct
YTSRLHS

SEQ ID NO: 1048 - protein, 9 aa, synthetic construct
LQGNTLPFT

SEQ ID NO: 1049 - protein, 5 aa, synthetic construct
KYNFY

SEQ ID NO: 1050 - protein, 17 aa, synthetic construct
YIDPYFGGTVYNQKFKG

SEQ ID NO: 1051 - protein, 9 aa, synthetic construct
TDGYSLFTY

SEQ ID NO: 1052 - protein, 11 aa, synthetic construct
RASQDISNYLN

SEQ ID NO: 1053 - protein, 7 aa, synthetic construct
YTSRLHS

SEQ ID NO: 1054 - protein, 9 aa, synthetic construct
LQGNTLPFT

SEQ ID NO: 1055 - protein, 7 aa, synthetic construct
GYAFTKY

SEQ ID NO: 1056 - protein, 6 aa, synthetic construct
DPYNGG

SEQ ID NO: 1057 - protein, 9 aa, synthetic construct
TDGYSLFTY

SEQ ID NO: 1058 - protein, 11 aa, synthetic construct
RASQDISNYLN

SEQ ID NO: 1059 - protein, 7 aa, synthetic construct
YTSRLHS

SEQ ID NO: 1060 - protein, 9 aa, synthetic construct
QQGNTLPFT

SEQ ID NO: 1061 - protein, 7 aa, synthetic construct
GYAFTKY

SEQ ID NO: 1062 - protein, 6 aa, synthetic construct
DPYFGG

SEQ ID NO: 1063 - protein, 9 aa, synthetic construct
TDGYSLFTY

SEQ ID NO: 1064 - protein, 11 aa, synthetic construct
RASQDISNYLN

SEQ ID NO: 1065 - protein, 7 aa, synthetic construct
YTSRLHS

SEQ ID NO: 1066 - protein, 9 aa, synthetic construct
LQGNTLPFT

SEQ ID NO: 1067 - protein, 7 aa, synthetic construct
GYAFTKY

SEQ ID NO: 1068 - protein, 6 aa, synthetic construct
DPYFGG

SEQ ID NO: 1069 - protein, 9 aa, synthetic construct
TDSYSLFTY

SEQ ID NO: 1070 - protein, 11 aa, synthetic construct
RASQDISNYLN

SEQ ID NO: 1071 - protein, 7 aa, synthetic construct
YTSRLHS

SEQ ID NO: 1072 - protein, 9 aa, synthetic construct
LQGNTLPFT

SEQ ID NO: 1073 - protein, 7 aa, synthetic construct
GYAFTKY

SEQ ID NO: 1074 - protein, 6 aa, synthetic construct
DPYFGG

SEQ ID NO: 1075 - protein, 9 aa, synthetic construct
TEGYSLFTY

SEQ ID NO: 1076 - protein, 11 aa, synthetic construct
RASQDISNYLN

SEQ ID NO: 1077 - protein, 7 aa, synthetic construct
YTSRLHS

SEQ ID NO: 1078 - protein, 9 aa, synthetic construct
LQGNTLPFT

SEQ ID NO: 1079 - protein, 7 aa, synthetic construct
GYAFTKY

SEQ ID NO: 1080 - protein, 6 aa, synthetic construct
DPYFGG

SEQ ID NO: 1081 - protein, 9 aa, synthetic construct
TDAYSLFTY

SEQ ID NO: 1082 - protein, 11 aa, synthetic construct
RASQDISNYLN

SEQ ID NO: 1083 - protein, 7 aa, synthetic construct
YTSRLHS

SEQ ID NO: 1084 - protein, 9 aa, synthetic construct
LQGNTLPFT

SEQ ID NO: 1085 - protein, 7 aa, synthetic construct
GYAFTKY

SEQ ID NO: 1086 - protein, 6 aa, synthetic construct
DPYFGG

SEQ ID NO: 1087 - protein, 9 aa, synthetic construct
TDGYSLFTY

SEQ ID NO: 1088 - protein, 11 aa, synthetic construct
RASQDISNYLN

SEQ ID NO: 1089 - protein, 7 aa, synthetic construct
YTSRLHS

SEQ ID NO: 1090 - protein, 9 aa, synthetic construct
QQGNTLPFT

SEQ ID NO: 1091 - protein, 7 aa, synthetic construct
GYAFTKY

SEQ ID NO: 1092 - protein, 6 aa, synthetic construct
DPYFGG

SEQ ID NO: 1093 - protein, 9 aa, synthetic construct
TDGYSLFTY

SEQ ID NO: 1094 - protein, 11 aa, synthetic construct
RASQDISNYLN

SEQ ID NO: 1095 - protein, 7 aa, synthetic construct
YTSRLHS

SEQ ID NO: 1096 - protein, 9 aa, synthetic construct
QQGNTLPFT

SEQ ID NO: 1097 - protein, 7 aa, synthetic construct
GYAFTKY

SEQ ID NO: 1098 - protein, 6 aa, synthetic construct
DPYFGG

SEQ ID NO: 1099 - protein, 9 aa, synthetic construct
TDSYSLFTY

SEQ ID NO: 1100 - protein, 11 aa, synthetic construct
RASQDISNYLN

SEQ ID NO: 1101 - protein, 7 aa, synthetic construct
YTSRLHS

SEQ ID NO: 1102 - protein, 9 aa, synthetic construct
LQGNTLPFT

SEQ ID NO: 1103 - protein, 7 aa, synthetic construct
GYAFTKY

SEQ ID NO: 1104 - protein, 6 aa, synthetic construct
DPYFGG

SEQ ID NO: 1105 - protein, 9 aa, synthetic construct
TDSYSLFTY

SEQ ID NO: 1106 - protein, 11 aa, synthetic construct
RASQDISNYLN

SEQ ID NO: 1107 - protein, 7 aa, synthetic construct
YTSRLHS

SEQ ID NO: 1108 - protein, 9 aa, synthetic construct
LQGNTLPFT

SEQ ID NO: 1109 - protein, 7 aa, synthetic construct
GYAFTKY

SEQ ID NO: 1110 - protein, 6 aa, synthetic construct
DPYFGG

SEQ ID NO: 1111 - protein, 9 aa, synthetic construct
TDSYSLFTY

SEQ ID NO: 1112 - protein, 11 aa, synthetic construct
RASQDISNYLN

SEQ ID NO: 1113 - protein, 7 aa, synthetic construct
YTSRLHS

SEQ ID NO: 1114 - protein, 9 aa, synthetic construct
LQGNTLPFT

SEQ ID NO: 1115 - protein, 7 aa, synthetic construct
GYAFTKY

SEQ ID NO: 1116 - protein, 6 aa, synthetic construct
DPYFGG

SEQ ID NO: 1117 - protein, 9 aa, synthetic construct
TDSYSLFTY

SEQ ID NO: 1118 - protein, 11 aa, synthetic construct
RASQDISNYLN

SEQ ID NO: 1119 - protein, 7 aa, synthetic construct
YTSRLHS

SEQ ID NO: 1120 - protein, 9 aa, synthetic construct
LQGNTLPFT

SEQ ID NO: 1121 - protein, 7 aa, synthetic construct
GYAFTKY

SEQ ID NO: 1122 - protein, 6 aa, synthetic construct
DPYFGG

SEQ ID NO: 1123 - protein, 9 aa, synthetic construct
TDGYSLFTY

SEQ ID NO: 1124 - protein, 11 aa, synthetic construct
RASQDISNYLN

SEQ ID NO: 1125 - protein, 7 aa, synthetic construct
YTSRLHS

SEQ ID NO: 1126 - protein, 9 aa, synthetic construct
LQGNTLPFT

SEQ ID NO: 1127 - protein, 7 aa, synthetic construct
GYAFTKY

SEQ ID NO: 1128 - protein, 6 aa, synthetic construct
DPYFGG

SEQ ID NO: 1129 - protein, 9 aa, synthetic construct
TDGYSLFTY

SEQ ID NO: 1130 - protein, 11 aa, synthetic construct
RASQDISNYLN

SEQ ID NO: 1131 - protein, 7 aa, synthetic construct
YTSRLHS

SEQ ID NO: 1132 - protein, 9 aa, synthetic construct
LQGNTLPFT

SEQ ID NO: 1133 - protein, 7 aa, synthetic construct
GYAFTKY

SEQ ID NO: 1134 - protein, 6 aa, synthetic construct
DPYFGG

SEQ ID NO: 1135 - protein, 9 aa, synthetic construct
TDSYSLFTY

SEQ ID NO: 1136 - protein, 11 aa, synthetic construct
RASQDISNYLN

SEQ ID NO: 1137 - protein, 7 aa, synthetic construct
YTSRLHS

SEQ ID NO: 1138 - protein, 9 aa, synthetic construct
LQGNTLPFT

SEQ ID NO: 1139 - protein, 7 aa, synthetic construct
GYAFTKY

SEQ ID NO: 1140 - protein, 6 aa, synthetic construct
DPYFGG

SEQ ID NO: 1141 - protein, 9 aa, synthetic construct
TDSYSLFTY

SEQ ID NO: 1142 - protein, 11 aa, synthetic construct
RASQDISNYLN

SEQ ID NO: 1143 - protein, 7 aa, synthetic construct
YTSRLHS

SEQ ID NO: 1144 - protein, 9 aa, synthetic construct
LQGNTLPFT

SEQ ID NO: 1145 - protein, 7 aa, synthetic construct
GYAFTKY

SEQ ID NO: 1146 - protein, 6 aa, synthetic construct
DPYFGG

SEQ ID NO: 1147 - protein, 9 aa, synthetic construct
TDGYSLFTY

SEQ ID NO: 1148 - protein, 11 aa, synthetic construct
RASQDISNYLN

SEQ ID NO: 1149 - protein, 7 aa, synthetic construct
YTSRLHS

SEQ ID NO: 1150 - protein, 9 aa, synthetic construct
LQGNTLPFT

SEQ ID NO: 1151 - protein, 7 aa, synthetic construct
GYAFTKY

SEQ ID NO: 1152 - protein, 6 aa, synthetic construct
DPYFGG

SEQ ID NO: 1153 - protein, 9 aa, synthetic construct
TDGYSLFTY

SEQ ID NO: 1154 - protein, 11 aa, synthetic construct
RASQDISNYLN

SEQ ID NO: 1155 - protein, 7 aa, synthetic construct
YTSRLHS

SEQ ID NO: 1156 - protein, 9 aa, synthetic construct
LQGNTLPFT

SEQ ID NO: 1157 - protein, 7 aa, synthetic construct
GYAFTKY

SEQ ID NO: 1158 - protein, 6 aa, synthetic construct
DPYFGG

SEQ ID NO: 1159 - protein, 9 aa, synthetic construct
TDSYSLFTY

SEQ ID NO: 1160 - protein, 11 aa, synthetic construct
RASQDISNYLN

SEQ ID NO: 1161 - protein, 7 aa, synthetic construct
YTSRLHS

SEQ ID NO: 1162 - protein, 9 aa, synthetic construct
LQGNTLPFT

SEQ ID NO: 1163 - protein, 7 aa, synthetic construct
GYAFTKY

SEQ ID NO: 1164 - protein, 6 aa, synthetic construct
DPYFGG

SEQ ID NO: 1165 - protein, 9 aa, synthetic construct
TDGYSLFTY

SEQ ID NO: 1166 - protein, 11 aa, synthetic construct
RASQDISNYLN

SEQ ID NO: 1167 - protein, 7 aa, synthetic construct
YTSRLHS

SEQ ID NO: 1168 - protein, 9 aa, synthetic construct
LQGNTLPFT

SEQ ID NO: 1169 - protein, 7 aa, synthetic construct
GYAFTKY

SEQ ID NO: 1170 - protein, 6 aa, synthetic construct
DPYFGG

SEQ ID NO: 1171 - protein, 9 aa, synthetic construct
TDGYSLFTY

SEQ ID NO: 1172 - protein, 11 aa, synthetic construct
RASQDISNYLN

SEQ ID NO: 1173 - protein, 7 aa, synthetic construct
YTSRLHS

SEQ ID NO: 1174 - protein, 9 aa, synthetic construct
LQGNTLPFT

SEQ ID NO: 1175 - protein, 8 aa, synthetic construct
GYAFTKYN

SEQ ID NO: 1176 - protein, 8 aa, synthetic construct
IDPYNGGT

SEQ ID NO: 1177 - protein, 11 aa, synthetic construct
ATTDGYSLFTY

SEQ ID NO: 1178 - protein, 6 aa, synthetic construct
QDISNY

SEQ ID NO: 1179 - skipped

SEQ ID NO: 1180 - protein, 9 aa, synthetic construct
QQGNTLPFT

SEQ ID NO: 1181 - protein, 8 aa, synthetic construct
GYAFTKYN

SEQ ID NO: 1182 - protein, 8 aa, synthetic construct
IDPYFGGT

SEQ ID NO: 1183 - protein, 11 aa, synthetic construct
ATTDGYSLFTY

SEQ ID NO: 1184 - protein, 6 aa, synthetic construct
QDISNY

SEQ ID NO: 1185 - skipped

SEQ ID NO: 1186 - protein, 9 aa, synthetic construct
LQGNTLPFT

SEQ ID NO: 1187 - protein, 8 aa, synthetic construct
GYAFTKYN

SEQ ID NO: 1188 - protein, 8 aa, synthetic construct
IDPYFGGT

SEQ ID NO: 1189 - protein, 11 aa, synthetic construct
ATTDSYSLFTY

SEQ ID NO: 1190 - protein, 6 aa, synthetic construct
QDISNY

SEQ ID NO: 1191 - skipped

SEQ ID NO: 1192 - protein, 9 aa, synthetic construct
LQGNTLPFT

SEQ ID NO: 1193 - protein, 8 aa, synthetic construct
GYAFTKYN

SEQ ID NO: 1194 - protein, 8 aa, synthetic construct
IDPYFGGT

SEQ ID NO: 1195 - protein, 11 aa, synthetic construct
ATTEGYSLFTY

SEQ ID NO: 1196 - protein, 6 aa, synthetic construct
QDISNY

SEQ ID NO: 1197 - skipped

SEQ ID NO: 1198 - protein, 9 aa, synthetic construct
LQGNTLPFT

SEQ ID NO: 1199 - protein, 8 aa, synthetic construct
GYAFTKYN

SEQ ID NO: 1200 - protein, 8 aa, synthetic construct
IDPYFGGT

SEQ ID NO: 1201 - protein, 11 aa, synthetic construct
ATTDAYSLFTY

SEQ ID NO: 1202 - protein, 6 aa, synthetic construct
QDISNY

SEQ ID NO: 1203 - skipped

SEQ ID NO: 1204 - protein, 9 aa, synthetic construct
LQGNTLPFT

SEQ ID NO: 1205 - protein, 8 aa, synthetic construct
GYAFTKYN

SEQ ID NO: 1206 - protein, 8 aa, synthetic construct
IDPYFGGT

SEQ ID NO: 1207 - protein, 11 aa, synthetic construct
ATTDGYSLFTY

SEQ ID NO: 1208 - protein, 6 aa, synthetic construct
QDISNY

SEQ ID NO: 1209 - skipped

SEQ ID NO: 1210 - protein, 9 aa, synthetic construct
QQGNTLPFT

SEQ ID NO: 1211 - protein, 8 aa, synthetic construct
GYAFTKYN

SEQ ID NO: 1212 - protein, 8 aa, synthetic construct
IDPYFGGT

SEQ ID NO: 1213 - protein, 11 aa, synthetic construct
ATTDGYSLFTY

SEQ ID NO: 1214 - protein, 6 aa, synthetic construct
QDISNY

SEQ ID NO: 1215 - skipped

SEQ ID NO: 1216 - protein, 9 aa, synthetic construct
QQGNTLPFT

SEQ ID NO: 1217 - protein, 8 aa, synthetic construct
GYAFTKYN

SEQ ID NO: 1218 - protein, 8 aa, synthetic construct
IDPYFGGT

SEQ ID NO: 1219 - protein, 11 aa, synthetic construct
ATTDSYSLFTY

SEQ ID NO: 1220 - protein, 6 aa, synthetic construct
QDISNY

SEQ ID NO: 1221 - skipped

SEQ ID NO: 1222 - protein, 9 aa, synthetic construct
LQGNTLPFT

SEQ ID NO: 1223 - protein, 8 aa, synthetic construct
GYAFTKYN

SEQ ID NO: 1224 - protein, 8 aa, synthetic construct
IDPYFGGT

SEQ ID NO: 1225 - protein, 11 aa, synthetic construct
ATTDSYSLFTY

SEQ ID NO: 1226 - protein, 6 aa, synthetic construct
QDISNY

SEQ ID NO: 1227 - skipped

SEQ ID NO: 1228 - protein, 9 aa, synthetic construct
LQGNTLPFT

SEQ ID NO: 1229 - protein, 8 aa, synthetic construct
GYAFTKYN

SEQ ID NO: 1230 - protein, 8 aa, synthetic construct
IDPYFGGT

SEQ ID NO: 1231 - protein, 11 aa, synthetic construct
ATTDSYSLFTY

SEQ ID NO: 1232 - protein, 6 aa, synthetic construct
QDISNY

SEQ ID NO: 1233 - skipped

SEQ ID NO: 1234 - protein, 9 aa, synthetic construct
LQGNTLPFT

SEQ ID NO: 1235 - protein, 8 aa, synthetic construct
GYAFTKYN

SEQ ID NO: 1236 - protein, 8 aa, synthetic construct
IDPYFGGT

SEQ ID NO: 1237 - protein, 11 aa, synthetic construct
ATTDSYSLFTY

SEQ ID NO: 1238 - protein, 6 aa, synthetic construct
QDISNY

SEQ ID NO: 1239 - skipped

SEQ ID NO: 1240 - protein, 9 aa, synthetic construct
LQGNTLPFT

SEQ ID NO: 1241 - protein, 8 aa, synthetic construct
GYAFTKYN

SEQ ID NO: 1242 - protein, 8 aa, synthetic construct
IDPYFGGT

SEQ ID NO: 1243 - protein, 11 aa, synthetic construct
ATTDGYSLFTY

SEQ ID NO: 1244 - protein, 6 aa, synthetic construct
QDISNY

SEQ ID NO: 1245 - skipped

SEQ ID NO: 1246 - protein, 9 aa, synthetic construct
LQGNTLPFT

SEQ ID NO: 1247 - protein, 8 aa, synthetic construct
GYAFTKYN

SEQ ID NO: 1248 - protein, 8 aa, synthetic construct
IDPYFGGT

SEQ ID NO: 1249 - protein, 11 aa, synthetic construct
ATTDGYSLFTY

SEQ ID NO: 1250 - protein, 6 aa, synthetic construct
QDISNY

SEQ ID NO: 1251 - skipped

SEQ ID NO: 1252 - protein, 9 aa, synthetic construct
LQGNTLPFT

SEQ ID NO: 1253 - protein, 8 aa, synthetic construct
GYAFTKYN

SEQ ID NO: 1254 - protein, 8 aa, synthetic construct
IDPYFGGT

SEQ ID NO: 1255 - protein, 11 aa, synthetic construct
ATTDSYSLFTY

SEQ ID NO: 1256 - protein, 6 aa, synthetic construct
QDISNY

SEQ ID NO: 1257 - skipped

SEQ ID NO: 1258 - protein, 9 aa, synthetic construct
LQGNTLPFT

SEQ ID NO: 1259 - protein, 8 aa, synthetic construct
GYAFTKYN

SEQ ID NO: 1260 - protein, 8 aa, synthetic construct
IDPYFGGT

SEQ ID NO: 1261 - protein, 11 aa, synthetic construct
ATTDSYSLFTY

SEQ ID NO: 1262 - protein, 6 aa, synthetic construct
QDISNY

SEQ ID NO: 1263 - skipped

SEQ ID NO: 1264 - protein, 9 aa, synthetic construct
LQGNTLPFT

SEQ ID NO: 1265 - protein, 8 aa, synthetic construct
GYAFTKYN

SEQ ID NO: 1266 - protein, 8 aa, synthetic construct
IDPYFGGT

SEQ ID NO: 1267 - protein, 11 aa, synthetic construct
ATTDGYSLFTY

SEQ ID NO: 1268 - protein, 6 aa, synthetic construct
QDISNY

SEQ ID NO: 1269 - skipped

SEQ ID NO: 1270 - protein, 9 aa, synthetic construct
LQGNTLPFT

SEQ ID NO: 1271 - protein, 8 aa, synthetic construct
GYAFTKYN

SEQ ID NO: 1272 - protein, 8 aa, synthetic construct
IDPYFGGT

SEQ ID NO: 1273 - protein, 11 aa, synthetic construct
ATTDGYSLFTY

SEQ ID NO: 1274 - protein, 6 aa, synthetic construct
QDISNY

SEQ ID NO: 1275 - skipped

SEQ ID NO: 1276 - protein, 9 aa, synthetic construct
LQGNTLPFT

SEQ ID NO: 1277 - protein, 8 aa, synthetic construct
GYAFTKYN

SEQ ID NO: 1278 - protein, 8 aa, synthetic construct
IDPYFGGT

SEQ ID NO: 1279 - protein, 11 aa, synthetic construct
ATTDSYSLFTY

SEQ ID NO: 1280 - protein, 6 aa, synthetic construct
QDISNY

SEQ ID NO: 1281 - skipped

SEQ ID NO: 1282 - protein, 9 aa, synthetic construct
LQGNTLPFT

SEQ ID NO: 1283 - protein, 8 aa, synthetic construct
GYAFTKYN

SEQ ID NO: 1284 - protein, 8 aa, synthetic construct
IDPYFGGT

SEQ ID NO: 1285 - protein, 11 aa, synthetic construct
ATTDGYSLFTY

SEQ ID NO: 1286 - protein, 6 aa, synthetic construct
QDISNY

SEQ ID NO: 1287 - skipped

SEQ ID NO: 1288 - protein, 9 aa, synthetic construct
LQGNTLPFT

SEQ ID NO: 1289 - protein, 8 aa, synthetic construct
GYAFTKYN

SEQ ID NO: 1290 - protein, 8 aa, synthetic construct
IDPYFGGT

SEQ ID NO: 1291 - protein, 11 aa, synthetic construct
ATTDGYSLFTY

SEQ ID NO: 1292 - protein, 6 aa, synthetic construct
QDISNY

SEQ ID NO: 1293 - skipped

SEQ ID NO: 1294 - protein, 9 aa, synthetic construct
LQGNTLPFT

SEQ ID NO: 1295 - protein, 13 aa, synthetic construct
KASGYAFTKYNIY

SEQ ID NO: 1296 - protein, 10 aa, synthetic construct
YIDPYNGGTV

SEQ ID NO: 1297 - protein, 11 aa, synthetic construct
ATTDGYSLFTY

SEQ ID NO: 1298 - protein, 11 aa, synthetic construct
RASQDISNYLN

SEQ ID NO: 1299 - protein, 8 aa, synthetic construct
YYTSRLHS

SEQ ID NO: 1300 - protein, 9 aa, synthetic construct
QQGNTLPFT

SEQ ID NO: 1301 - protein, 13 aa, synthetic construct
KASGYAFTKYNFY

SEQ ID NO: 1302 - protein, 10 aa, synthetic construct
YIDPYFGGTV

SEQ ID NO: 1303 - protein, 11 aa, synthetic construct
ATTDGYSLFTY

SEQ ID NO: 1304 - protein, 11 aa, synthetic construct
RASQDISNYLN

SEQ ID NO: 1305 - protein, 8 aa, synthetic construct
YYTSRLHS

SEQ ID NO: 1306 - protein, 9 aa, synthetic construct
LQGNTLPFT

SEQ ID NO: 1307 - protein, 13 aa, synthetic construct
KASGYAFTKYNFY

SEQ ID NO: 1308 - protein, 10 aa, synthetic construct
YIDPYFGGTV

SEQ ID NO: 1309 - protein, 11 aa, synthetic construct
ATTDSYSLFTY

SEQ ID NO: 1310 - protein, 11 aa, synthetic construct
RASQDISNYLN

SEQ ID NO: 1311 - protein, 8 aa, synthetic construct
YYTSRLHS

SEQ ID NO: 1312 - protein, 9 aa, synthetic construct
LQGNTLPFT

SEQ ID NO: 1313 - protein, 13 aa, synthetic construct
KASGYAFTKYNFY

SEQ ID NO: 1314 - protein, 10 aa, synthetic construct
YIDPYFGGTV

SEQ ID NO: 1315 - protein, 11 aa, synthetic construct
ATTEGYSLFTY

SEQ ID NO: 1316 - protein, 11 aa, synthetic construct
RASQDISNYLN

SEQ ID NO: 1317 - protein, 8 aa, synthetic construct
YYTSRLHS

SEQ ID NO: 1318 - protein, 9 aa, synthetic construct
LQGNTLPFT

SEQ ID NO: 1319 - protein, 13 aa, synthetic construct
KASGYAFTKYNFY

SEQ ID NO: 1320 - protein, 10 aa, synthetic construct
YIDPYFGGTV

SEQ ID NO: 1321 - protein, 11 aa, synthetic construct
ATTDAYSLFTY

SEQ ID NO: 1322 - protein, 11 aa, synthetic construct
RASQDISNYLN

SEQ ID NO: 1323 - protein, 8 aa, synthetic construct
YYTSRLHS

SEQ ID NO: 1324 - protein, 9 aa, synthetic construct
LQGNTLPFT

SEQ ID NO: 1325 - protein, 13 aa, synthetic construct
KASGYAFTKYNIY

SEQ ID NO: 1326 - protein, 10 aa, synthetic construct
YIDPYFGGTV

SEQ ID NO: 1327 - protein, 11 aa, synthetic construct
ATTDGYSLFTY

SEQ ID NO: 1328 - protein, 11 aa, synthetic construct
RASQDISNYLN

SEQ ID NO: 1329 - protein, 8 aa, synthetic construct
YYTSRLHS

SEQ ID NO: 1330 - protein, 9 aa, synthetic construct
QQGNTLPFT

SEQ ID NO: 1331 - protein, 13 aa, synthetic construct
KASGYAFTKYNIY

SEQ ID NO: 1332 - protein, 10 aa, synthetic construct
YIDPYFGGTV

SEQ ID NO: 1333 - protein, 11 aa, synthetic construct
ATTDGYSLFTY

SEQ ID NO: 1334 - protein, 11 aa, synthetic construct
RASQDISNYLN

SEQ ID NO: 1335 - protein, 8 aa, synthetic construct
YYTSRLHS

SEQ ID NO: 1336 - protein, 9 aa, synthetic construct
QQGNTLPFT

SEQ ID NO: 1337 - protein, 13 aa, synthetic construct
KASGYAFTKYNFY

SEQ ID NO: 1338 - protein, 10 aa, synthetic construct
YIDPYFGGTV

SEQ ID NO: 1339 - protein, 11 aa, synthetic construct
ATTDSYSLFTY

SEQ ID NO: 1340 - protein, 11 aa, synthetic construct
RASQDISNYLN

SEQ ID NO: 1341 - protein, 8 aa, synthetic construct
YYTSRLHS

SEQ ID NO: 1342 - protein, 9 aa, synthetic construct
LQGNTLPFT

SEQ ID NO: 1343 - protein, 13 aa, synthetic construct
KASGYAFTKYNFY

SEQ ID NO: 1344 - protein, 10 aa, synthetic construct
YIDPYFGGTV

SEQ ID NO: 1345 - protein, 11 aa, synthetic construct
ATTDSYSLFTY

SEQ ID NO: 1346 - protein, 11 aa, synthetic construct
RASQDISNYLN

SEQ ID NO: 1347 - protein, 8 aa, synthetic construct
YYTSRLHS

SEQ ID NO: 1348 - protein, 9 aa, synthetic construct
LQGNTLPFT

SEQ ID NO: 1349 - protein, 13 aa, synthetic construct
KASGYAFTKYNFY

SEQ ID NO: 1350 - protein, 10 aa, synthetic construct
YIDPYFGGTV

SEQ ID NO: 1351 - protein, 11 aa, synthetic construct
ATTDSYSLFTY

SEQ ID NO: 1352 - protein, 11 aa, synthetic construct
RASQDISNYLN

SEQ ID NO: 1353 - protein, 8 aa, synthetic construct
YYTSRLHS

SEQ ID NO: 1354 - protein, 9 aa, synthetic construct
LQGNTLPFT

SEQ ID NO: 1355 - protein, 13 aa, synthetic construct
KASGYAFTKYNFY

SEQ ID NO: 1356 - protein, 10 aa, synthetic construct
YIDPYFGGTV

SEQ ID NO: 1357 - protein, 11 aa, synthetic construct
ATTDSYSLFTY

SEQ ID NO: 1358 - protein, 11 aa, synthetic construct
RASQDISNYLN

SEQ ID NO: 1359 - protein, 8 aa, synthetic construct
YYTSRLHS

SEQ ID NO: 1360 - protein, 9 aa, synthetic construct
LQGNTLPFT

SEQ ID NO: 1361 - protein, 13 aa, synthetic construct
KASGYAFTKYNFY

SEQ ID NO: 1362 - protein, 10 aa, synthetic construct
YIDPYFGGTV

SEQ ID NO: 1363 - protein, 11 aa, synthetic construct
ATTDGYSLFTY

SEQ ID NO: 1364 - protein, 11 aa, synthetic construct
RASQDISNYLN

SEQ ID NO: 1365 - protein, 8 aa, synthetic construct
YYTSRLHS

SEQ ID NO: 1366 - protein, 9 aa, synthetic construct
LQGNTLPFT

SEQ ID NO: 1367 - protein, 13 aa, synthetic construct
KASGYAFTKYNFY

SEQ ID NO: 1368 - protein, 10 aa, synthetic construct
YIDPYFGGTV

SEQ ID NO: 1369 - protein, 11 aa, synthetic construct
ATTDGYSLFTY

SEQ ID NO: 1370 - protein, 11 aa, synthetic construct
RASQDISNYLN

SEQ ID NO: 1371 - protein, 8 aa, synthetic construct
YYTSRLHS

SEQ ID NO: 1372 - protein, 9 aa, synthetic construct
LQGNTLPFT

SEQ ID NO: 1373 - protein, 13 aa, synthetic construct
KASGYAFTKYNFY

SEQ ID NO: 1374 - protein, 10 aa, synthetic construct
YIDPYFGGTV

SEQ ID NO: 1375 - protein, 11 aa, synthetic construct
ATTDSYSLFTY

SEQ ID NO: 1376 - protein, 11 aa, synthetic construct
RASQDISNYLN

SEQ ID NO: 1377 - protein, 8 aa, synthetic construct
YYTSRLHS

SEQ ID NO: 1378 - protein, 9 aa, synthetic construct
LQGNTLPFT

SEQ ID NO: 1379 - protein, 13 aa, synthetic construct
KASGYAFTKYNFY

SEQ ID NO: 1380 - protein, 10 aa, synthetic construct
YIDPYFGGTV

SEQ ID NO: 1381 - protein, 11 aa, synthetic construct
ATTDSYSLFTY

SEQ ID NO: 1382 - protein, 11 aa, synthetic construct
RASQDISNYLN

SEQ ID NO: 1383 - protein, 8 aa, synthetic construct
YYTSRLHS

SEQ ID NO: 1384 - protein, 9 aa, synthetic construct
LQGNTLPFT

SEQ ID NO: 1385 - protein, 13 aa, synthetic construct
KASGYAFTKYNFY

SEQ ID NO: 1386 - protein, 10 aa, synthetic construct
YIDPYFGGTV

SEQ ID NO: 1387 - protein, 11 aa, synthetic construct
ATTDGYSLFTY

SEQ ID NO: 1388 - protein, 11 aa, synthetic construct
RASQDISNYLN

SEQ ID NO: 1389 - protein, 8 aa, synthetic construct
YYTSRLHS

SEQ ID NO: 1390 - protein, 9 aa, synthetic construct
LQGNTLPFT

SEQ ID NO: 1391 - protein, 13 aa, synthetic construct
KASGYAFTKYNFY

SEQ ID NO: 1392 - protein, 10 aa, synthetic construct
YIDPYFGGTV

SEQ ID NO: 1393 - protein, 11 aa, synthetic construct
ATTDGYSLFTY

SEQ ID NO: 1394 - protein, 11 aa, synthetic construct
RASQDISNYLN

SEQ ID NO: 1395 - protein, 8 aa, synthetic construct
YYTSRLHS

SEQ ID NO: 1396 - protein, 9 aa, synthetic construct
LQGNTLPFT

SEQ ID NO: 1397 - protein, 13 aa, synthetic construct
KASGYAFTKYNFY

SEQ ID NO: 1398 - protein, 10 aa, synthetic construct
YIDPYFGGTV

SEQ ID NO: 1399 - protein, 11 aa, synthetic construct
ATTDSYSLFTY

SEQ ID NO: 1400 - protein, 11 aa, synthetic construct
RASQDISNYLN

SEQ ID NO: 1401 - protein, 8 aa, synthetic construct
YYTSRLHS

SEQ ID NO: 1402 - protein, 9 aa, synthetic construct
LQGNTLPFT

SEQ ID NO: 1403 - protein, 13 aa, synthetic construct
KASGYAFTKYNFY

SEQ ID NO: 1404 - protein, 10 aa, synthetic construct
YIDPYFGGTV

SEQ ID NO: 1405 - protein, 11 aa, synthetic construct
ATTDGYSLFTY

SEQ ID NO: 1406 - protein, 11 aa, synthetic construct
RASQDISNYLN

SEQ ID NO: 1407 - protein, 8 aa, synthetic construct
YYTSRLHS

SEQ ID NO: 1408 - protein, 9 aa, synthetic construct
LQGNTLPFT

SEQ ID NO: 1409 - protein, 13 aa, synthetic construct
KASGYAFTKYNFY

SEQ ID NO: 1410 - protein, 10 aa, synthetic construct
YIDPYFGGTV

SEQ ID NO: 1411 - protein, 11 aa, synthetic construct
ATTDGYSLFTY

SEQ ID NO: 1412 - protein, 11 aa, synthetic construct
RASQDISNYLN

SEQ ID NO: 1413 - protein, 8 aa, synthetic construct
YYTSRLHS

SEQ ID NO: 1414 - protein, 9 aa, synthetic construct
LQGNTLPFT

SEQ ID NO: 1415 - protein, 238 aa, synthetic construct
MGWSCIILFLVATATGVHSDIVMTQSPLSLPVTPGEPASISCRSSKSLLHSNLNTYLYWFLQKPGQSPQILIYRMSNLASGVPDRFSGSGSGTAFTLKISRVEAEDVGVYYCMQHLEYPFTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1416 - protein, 470 aa, synthetic construct
MGWSLILLFLVAVATRVLSQVQLVESGGGLVKPGGSLRLSCAASGFTFSNYAMSWIRQAPGKGLEWVSYISPGGDYIYYADSVKGRFTISRDNAKNSLYLQMNSLRAEDTAVYYCTTDRRHYGSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1417 - protein, 238 aa, synthetic construct
MGWSCIILFLVATATGVHSDVLMTQTPLSLPVSLGDQASISCRSSQHIVHSNENTYLEWYLQKPGQSPKLLIYKVSNRFSGVPDRFSGSGSGTDFTLKISRVEAEDLGVYYCFQGSHVPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1418 - protein, 471 aa, synthetic construct
MGWSLILLFLVAVATRVLSQGQMQQSGAELVKPGASVKLSCKTSGFTFSDNYISWLKQKPGQSLEWIAWIYAGTGGSSYNQKFRDKAQLTVDTSSRTAYMQLSSLTTEDSAIYYCARHDYYGTSGAWFAYWGRGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1419 - protein, 234 aa, Homo sapiens
MGWSCIILFLVATATGVHSEIILTQSPTTMAASPGEKITITCSASSSISSHYLHWYQQKSGFSPKLLIYRTSNLASGVPARFSGSGSGTSYSLTIGTMEAEDVATYYCQQGSSLPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1420 - protein, 466 aa, Homo sapiens
MGWSLILLFLVAVATRVLSEIQLQQSGPELVKPGASVKVSCKASGYAFTSQNIYWVKQSHGKSLEWIGYIEPYNVVPMYNPKFKGKATLTVDKSSSSAYIHLNSLTSEDSAIYYCARSGSSNFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1421 - protein, 233 aa, synthetic construct
MGWSCIILFLVATATGVHSDIQMTQSPASLSASVGETVTITCRASGNIHSYLAWYQQKQGKSPQLLVYYAETLADGVPSRFSGRGSGTQYSLKINSLQPEDFGSYFCQQFWTTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1422 - protein, 466 aa, synthetic construct
MGWSLILLFLVAVATRVLSQVQLQQSGAELVRPGTSVKVSCKASGYAFTNYLIEWVKQRPGQGLEWIGAINPGSGATNYNEKFKDKARLTADKSSNTAYLQFSSLTSDDSAVYFCARFLGNYFDNWGQGATLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1423 - protein, 233 aa, synthetic construct
MGWSCIILFLVATATGVHSDIVLTQTTSYLSVSLGGRVTIACKASAHINNWLAWYQQKPGNAPRLLISDATSLETGVPSRFSGSGSGKDYTLSITSLQTEDVATYYCQHYWSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1424 - protein, 465 aa, synthetic construct
MGWSLILLFLVAVATRVLSEVQLEESGPGLVKPSQSLSLTCTVTGYSITSDYAWNWIRQFPGNKLEWMGYISYSGSTNYNPSLKSRISITRDTSKNQFFLQLNSVITEDTATYYCARGGTYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 1425 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 1426 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSRVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 1427 - protein, 388 aa, synthetic construct [sequence omitted]

SEQ ID NO: 1428 - protein, 385 aa, synthetic construct
MGLGPVFLLLAGIFPFAPPGAAAEPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPSGKVLVLQSHWQTFHVSAVAAAAAAIFVIIIFYVRCCKKKTSAAEGPELVSLQVLDQHPVGTSDHRDATQLGFQPLMSALGSTGSTEGA

SEQ ID NO: 1429 - protein, 332 aa, synthetic construct
MGLGPVFLLLAGIFPFAPPGAAAEPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPSGKVLVLQSHWQTFHVSAVAAGCCYFCYYYFLCPLL

SEQ ID NO: 1430 - protein, 383 aa, synthetic construct [sequence omitted]

SEQ ID NO: 1431 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 1432 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSTVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 1433 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSTAPPMVNVTHSEASEGNITVTCRASGFYPRNIALTWRQDGVSLNHNAQQWGGILPDQNGTYQTWVATRIRQGEEQRFACYMEHSGNHSTHPVPS

SEQ ID NO: 1434 - protein, 328 aa, synthetic construct
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSTVPPMVNVTCSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 1435 - protein, 445 aa, synthetic construct
MGWSCIILFLVATATGVHSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSGSGSTVPPMVNVTRSEASEGNITVTCRASGFYPWNITLSWRQDGVSLSHDTQQWGDVLPDGNGTYQTWVATRICQGEEQRFTCYMEHSGNHSTHPVPSTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 1436 - protein, 445 aa, synthetic construct
MGWSCIILFLVATATGVHSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSGSGSRVPPMVNVTRSEASEGNITVTCRASSFYPRNITLTWRQDGVSLSHDTQQWGDVLPDGNGTYQTWVATRICQGEEQRFTCYMEHSGNHSTHPVPSRVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 1437 - protein, 444 aa, synthetic construct
MGWSCIILFLVATATGVHSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSGSGSTVPPMVNVTRSEASEGNITVTCRASSFYPRNIILTWRQDGVSLSHDTQQWGDVLPDGNGTYQTWVATRICRGEEQRFTCYMEHSGNHSTHPVPSTVPPMVNVTRSEASEGNITVTCRASSFYPRNIILTWRQDGVSLSHDTQQWGDVLPDGNGTYQTWVATRICRGEEQRFTCYMEHSGNHSTHPVP

SEQ ID NO: 1438 - protein, 445 aa, synthetic construct
MGWSCIILFLVATATGVHSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGGSGSGSTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSTVPPMVNVTCSEVSEGNITVTCRASSFYPRNITLTWRQDGVSLSHNTQQWGDVLPDGNGTYQTWVATRIRQGEEQRFTCYMEHSGNHGTHPVPS

SEQ ID NO: 1439 - protein, 306 aa, synthetic construct
EPHSLRYNLTVLSGDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPSGKVLVLQSHWQHHHHHHGLNDIFEAQKIEWHE

SEQ ID NO: 1440 - protein, 306 aa, synthetic construct
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPSGKVLVLQSHWQHHHHHHGLNDIFEAQKIEWHE

SEQ ID NO: 1441 - protein, 306 aa, synthetic construct
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPSGKVLVLQSHWQHHHHHHGLNDIFEAQKIEWHE

SEQ ID NO: 1442 - protein, 306 aa, synthetic construct
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRTVPPMVNVTRSEASEGNITVTCRASSFYPRNITLTWRQDGVSLSHDTQQWGDVLPDGNGTYQTWVATRICQGEEQRFTCYMEHSGNHSTHPVPSGKVLVLQSHWQHHHHHHGLNDIFEAQKIEWHE

SEQ ID NO: 1443 - protein, 307 aa, synthetic construct
EPHSLRYNLMVLSQDGSVQSGFLAEGHLDGQPFLRYDRQKRRAKPQGQWAEDVLGAKTWDTETEDLTENGQDLRRTLTHIKDQKGGLHSLQEIRVCEIHEDSSTRGSRHFYYDGELFLSQNLETQESTVPQSSRAQTLAMNVTNFWKEDAMKTKTHYRAMQADCLQKLQRYLKSGVAIRRTVPPMVNVTCSEVSEGNITVTCRASSFYPRNITLTWRQDGVSLSHNTQQWGDVLPDGNGTYQTWVATRIRQGEEQRFTCYMEHSGNHGTHPVPSGKALVLQSQRTDHHHHHHGLNDIFEAQKIEWHE

SEQ ID NO: 1444 - protein, 238 aa, synthetic construct
MGWSCIILFLVATATGVHSDIVMTQTPLSSPVTLGQPASISCRSSQSLVHRDGNTYLSWFLQRPGQAPRLLIYRISNRFSGVPDRFSGSGAGTDFTLKISRVEAEDVGVYYCMQATQIPNTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1445 - protein, 460 aa, synthetic construct
MGWSLILLFLVAVATRVLSEVQLLESGGGLVQPGGSLRLSCAASGFTFSSYGLTWIRQAPGKGLEWVSSISGSGNNTYYADSVKGRFTISRDKVKKTLYLQMDSLTVGDTAVYYCLGVGQGHGIPVIVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK